(19) United States
(12) Patent Application Publication
(10) Pub. No.: US 2026/0103765 A1
(43) Pub. Date: Apr. 16, 2026

(54) METHODS AND COMPOSITIONS FOR RAPID PATHOGEN DETECTION

(71) Applicants: The Brigham and Women's Hospital, Inc., Boston, MA (US); The Broad Institute, Inc., Cambridge, MA (US); The General Hospital Corporation, Boston, MA (US)

(72) Inventors: David Roach, Boston, MA (US); Roby Bhattacharyya, Boston, MA (US); Josephine Bagnall, Cambridge, MA (US); Miriam Bern, Cambridge, MA (US); Ishay Ben Zion, Cambridge, MA (US); Noam Shoresh, Cambridge, MA (US)

(73) Assignees: The Brigham and Women's Hospital, Inc., Boston, MA (US); The Broad Institute, Inc., Cambridge, MA (US); The General Hospital Corporation, Boston, MA (US)

(21) Appl. No.: 19/355,113

(22) Filed: Oct. 10, 2025

Related U.S. Application Data
(60) Provisional application No. 63/706,345, filed on Oct. 11, 2024.

(51) Int. Cl. C12Q 1/689

(52) U.S. Cl. CPC C12Q 1/689; C12Q 2600/106; C12Q 2600/156

(57) ABSTRACT

Compositions and methods for detecting bacterial pathogens containing microbial resistance genes in a sample, as well as methods for selecting a treatment for human infections involving such organisms. The methods involve carrying out a series of reactions in parallel where each reaction involves detecting a bacterial species or resistance gene in a sample using a CRISPR-Cas13a reaction. In some embodiments, the methods involve culturing a blood sample to increase the concentration of bacteria in the sample and/or direct detection of bacterial pathogens and/or microbial resistance genes in a blood sample.

### CROSS-REFERENCE TO RELATED APPLICATION

This application claims priority to and the benefit of U.S. Provisional Application No. 63/706,345, filed Oct. 11, 2024, the entire contents of which are incorporated herein by reference.

### STATEMENT AS TO FEDERALLY SPONSORED RESEARCH

The claimed invention was made with government support under Grants No. T32AI007061-44 and UM1AI104681 awarded by the National Institutes of Health. The government has certain rights in the claimed invention.

### SEQUENCE LISTING

The instant application contains a Sequence Listing which has been filed electronically as an XML file entitled 167741-055801-US_SL.xml created on Oct. 8, 2025, and having a size of 155,042 bytes. The content of the Sequence Listing is incorporated herein in its entirety.

### BACKGROUND

Antimicrobial resistant (AMR) organisms are considered by the World Health Organization to be one of the ten greatest health care threats facing humanity. The burden of bacterial infections and AMR is most severe in low-and-middle income countries, where the medical infrastructure necessary for their effective diagnosis and treatment is lacking. In 2019 alone, there were an estimated 1.27 million global deaths due to resistant bacterial infections and it is projected to reach as many as 10 million deaths annually by 2050, resulting in increasing calls to improve diagnostic strategies for containing the epidemic.

Accordingly, there is a critical need for a low-cost method to rapidly detect pathogens, such as bacterial species and their AMR genes, in clinical samples.

### SUMMARY

As described below, the present disclosure provides compositions and methods for detecting bacterial pathogens containing microbial resistance genes in a sample, as well as methods for selecting a treatment for human infections involving such organisms. In an embodiment, the methods of detection involve carrying out a series of reactions in parallel, where each reaction involves detecting a bacterial species or resistance gene in a sample using a CRISPR-Cas13a reaction. In some embodiments, the methods involve culturing a blood sample to increase the concentration of bacteria in the sample and/or direct detection of bacterial pathogens and/or microbial resistance genes in a blood sample. Disclosed herein, in some embodiments, are method and compositions for detecting a pathogen in a sample. In some embodiments, the methods disclosed herein include contacting a sample with a composition, where the composition includes a reagent mix, at least one pair of recombinase polymerase amplification (RPA) primers, and at least one CRISPR guide RNA (crRNA) designed to correspond to at least one target; determining the presence or absence of the at least one target; and correlating the presence or absence of the at least one target with the presence or absence of a pathogen, thereby detecting a pathogen in the sample.

In one aspect, the present disclosure provides a method for characterizing a bacterium and a bacterial antimicrobial resistance (AMR) gene in a sample. The method involves a) splitting a bacterial sample into two containers, a first container containing a set of primers suitable for amplifying a target polynucleotide capable of identifying a bacterial species or taxonomic group in the sample, and a second container containing a set of primers suitable for amplifying a specific AMR target gene. One primer of each set of primers contains a T7 promoter sequence. DNA from the bacterial sample is contacted with the primers under conditions that amplify the target polynucleotides, thereby producing target amplicons in each container. The method further involves b) contacting the target amplicons with a T7 RNA polymerase under conditions suitable for RNA production, thereby producing target RNAs in each container. The method also involves c) contacting the target RNA in each container with a Type VI CRISPR polypeptide, guide polynucleotides that specifically bind the target RNA, and a detection construct that binds the target RNA under conditions suitable for Type VI CRISPR polypeptide activity, thereby cutting the target RNAs. The method also involves d) detecting a signal in each container, the signal produced by the detection construct upon binding of the detection construct to the target RNA. Detection of a detectable signal in the first container identifies the bacterial species and/or taxonomic group present in the first container, and detection of a signal in the second container identifies the presence of an AMR gene in the second container.

In another aspect, the disclosure provides a method for identification of antimicrobial resistant (AMR) genes and microbial species of interest in a blood sample. The method involves a) contacting each of two or more aliquots of a positive blood culture diluted in a composition containing water with a unique set of primers in separate containers under conditions suitable for recombinase polymerase amplification (RPA). A first set of primers is suitable for amplifying a target nucleotide capable of identifying a microbial species or taxonomic group. A second set of primers is suitable for amplifying a target nucleotide specific to an AMR gene. One primer of each set of primers contains a T7 promoter sequence. The method also involves b) contacting any amplicons produced in a) in each container with a T7 RNA polymerase under conditions suitable for RNA production. The method further involves c) contacting the RNA produced in b) in each container with a Type VI CRISPR polypeptide in complex with a guide polynucleotide capable of binding to RNA produced in b) in the container, and a detection construct containing RNA, under conditions suitable for nuclease activity of the Type VI CRISPR polypeptide to be activated by binding of the guide polynucleotide to the RNA produced in b). The method also involves d) detecting any detectable signal produced by the detection construct. The presence of a detectable signal in the first container and the second container identifies the blood sample as containing the microbial species or group of microbial species and the AMR gene.

In another aspect, the disclosure provides a set of primers and one or more guide polynucleotides for use in a method for identifying microbial resistant bacteria (AMR) in a sample. The set of primers and one or more guide polynucleotides contains a) a first set of primers containing a set of sequences selected from one or more of:
GAAATTAATACGACTCACTATAGGGATATGGCGGTGAGTATTATCGTCAGGAACAACAT C (SEQ ID NO: 67) and GAGATGACCATTTGTCGGCATCAACATCTTTGTAG (SEQ ID NO: 81);
GAAATTAATACGACTCACTATAGGGGGTGACGCCCTGCCGCTGCAGGTAACCCATAA (SEQ ID NO: 68) and ATCTTCACGCTCCAGTACGCTGTAGCTGGCTTT (SEQ ID NO: 82);
GAAATTAATACGACTCACTATAGGGATGTTCTATCCGCTGGTGTCGCTGGA (SEQ ID NO: 69) and ACCGCTGCCCTTCTTGATGTCGTAG (SEQ ID NO: 83);
GAAATTAATACGACTCACTATAGGGAATGGGCGTATGATTATACCCAGGTAA (SEQ ID NO: 70) and AGCAGTTTGGCAAAGTGCGCTTGTGCAG (SEQ ID NO: 84);
GAAATTAATACGACTCACTATAGGGGTTGAAAACCGTCGCTTTTACGCTGAAAAA (SEQ ID NO: 71) and TCCATTCGGCTTCTTTATTCGCCACCTGGTC (SEQ ID NO: 85);
GAAATTAATACGACTCACTATAGGGGACCATCCTGTACGCGCTGGGCTGGACGCAGC (SEQ ID NO: 72) and CACCACGATTTCATCAGGCTGACGTGGAACTTC (SEQ ID NO: 86);
GAAATTAATACGACTCACTATAGGGCGGTACGCCTACTCGTCGAGCGTGAACGTG (SEQ ID NO: 73) and CGTCACTTCCATACGCAAGGGTTGTTCATC (SEQ ID NO: 87);
GAAATTAATACGACTCACTATAGGGGGCACTTACGCGAAGTGATTGGTGGTGAC (SEQ ID NO: 74) and CACAGTAATGGCGAAACCATGAAGCCTAC (SEQ ID NO: 88); and
GAAATTAATACGACTCACTATAGGGGAAGAGCAGCGCGTTGCCGATGCCTGGAAAG (SEQ ID NO: 75) and CTTTCGGCAGCGCCAGTTCGTCGTTGTGGT (SEQ ID NO: 89). The set of primers and one or more guide polynucleotides also contains b) a second set of primers containing a set of sequences selected from one or more of:
GAAATTAATACGACTCACTATAGGGCGTCTAGTTCTGCTGTCTTGTCTCTCATGG (SEQ ID NO: 76) and CAAAGTCCTGTTCGAGTTTAGCGAATGGTT (SEQ ID NO: 90);
GAAATTAATACGACTCACTATAGGGAATGTCTGGCAGCACACTTCCTATCTCGAC (SEQ ID NO: 77) and TGATCTCCTGCTTGATCCAGTTGAGGATCT (SEQ ID NO: 91);
GAAATTAATACGACTCACTATAGGGATAAAACCGGCAGCGGTGGCTATGG (SEQ ID NO: 78) and GCTAATACATCGCGACGGCTTTCTGCCTTA (SEQ ID NO: 92); and
GAAATTAATACGACTCACTATAGGGTTAAAATTCCCAATAGCTTGATCGCCCTCG (SEQ ID NO: 79) and ATAAACAGGCACAACTGAATATTTCATCGC (SEQ ID NO: 93). The set of primers and one or more guide polynucleotides also contains c) a guide polynucleotide containing a nucleotide sequence selected from one or more of:

(SEQ ID NO: 95)
CGGCGCGACGACGGGCUUCCCGCAAGCAGUUUUAGUCCCCUUCGUUUUU
GGGGUAGUCUAAAUCCCCUAUAGUGAGUCGUAUUAAUUUC;
(SEQ ID NO: 96)
GUGAUGAGACCAUGGCGGCGGUAUCGCUGUUUUAGUCCCCUUCGUUUUU
GGGGUAGUCUAAAUCCCCUAUAGUGAGUCGUAUUAAUUUC;
(SEQ ID NO: 97)
UUCUAUAUGCGCGGCAAGAACGACUUCCGUUUUAGUCCCCUUCGUUUUU
GGGGUAGUCUAAAUCCCCUAUAGUGAGUCGUAUUAAUUUC;
(SEQ ID NO: 98)
AGUCAACUAUUAGGCCAAAGCUAUGAUCGUUUUAGUCCCCUUCGUUUUU
GGGGUAGUCUAAAUCCCCUAUAGUGAGUCGUAUUAAUUUC;
(SEQ ID NO: 99)
AUGGGUGAAAUCGUCACCGAUCGUCUGGGUUUUAGUCCCCUUCGUUUUU
GGGGUAGUCUAAAUCCCCUAUAGUGAGUCGUAUUAAUUUC;
(SEQ ID NO: 100)
UGUCCGACCUGCUGCCCGGCUACCUGACGUUUUAGUCCCCUUCGUUUUU
GGGGUAGUCUAAAUCCCCUAUAGUGAGUCGUAUUAAUUUC;
(SEQ ID NO: 101)
AUUGGCAAUUACACGCUUCACUGCUGACGUUUUAGUCCCCUUCGUUUUU
GGGGUAGUCUAAAUCCCCUAUAGUGAGUCGUAUUAAUUUC;
(SEQ ID NO: 102)
CAGCCAACACGUCUCGACUUAAACCGUGGUUUUAGUCCCCUUCGUUUUU
GGGGUAGUCUAAAUCCCCUAUAGUGAGUCGUAUUAAUUUC;
(SEQ ID NO: 103)
UGUGGUACCUACCACAUGCACUCGCUGGGUUUUAGUCCCCUUCGUUUUU
GGGGUAGUCUAAAUCCCCUAUAGUGAGUCGUAUUAAUUUC;
(SEQ ID NO: 104)
GCUGGCUGGCUUUUCUGCCACCGCGCUGGUUUUAGUCCCCUUCGUUUUU
GGGGUAGUCUAAAUCCCCUAUAGUGAGUCGUAUUAAUUUC;
(SEQ ID NO: 105)
CAACGGUUUGAUCGUCAGGGAUGGCGGCGUUUUAGUCCCCUUCGUUUUU
GGGGUAGUCUAAAUCCCCUAUAGUGAGUCGUAUUAAUUUC;
(SEQ ID NO: 106)
AUCGUGCGCCGCUGAUUCUGGUCACUUAGUUUUAGUCCCCUUCGUUUUU
GGGGUAGUCUAAAUCCCCUAUAGUGAGUCGUAUUAAUUUC;
(SEQ ID NO: 107)
CCAAGUCUUUAAGUGGGAUGGACAGACGGUUUUAGUCCCCUUCGUUUUU
GGGGUAGUCUAAAUCCCCUAUAGUGAGUCGUAUUAAUUUC;
and
(SEQ ID NO: 108)
GGCCGUCCGUUAAUUUCCCUUGCAUACAGUUUUAGUCCCCUUCGUUUUU
GGGGUAGUCUAAAUCCCCUAUAGUGAGUCGUAUUAAUUUC.

In another aspect, the present disclosure provides a kit for use in a method for identifying microbial resistant bacteria (AMR) in a blood sample. In one embodiment, the kit contains a) a first set of primers containing a set of sequences selected from one or more of:
GAAATTAATACGACTCACTATAGGGATATGGCGGTGAGTATTATCGTCAGGAACAACAT C (SEQ ID NO: 67) and GAGATGACCATTTGTCGGCATCAACATCTTTGTAG (SEQ ID NO: 81);
GAAATTAATACGACTCACTATAGGGGGTGACGCCCTGCCGCTGCAGGTAACCCATAA (SEQ ID NO: 68) and ATCTTCACGCTCCAGTACGCTGTAGCTGGCTTT (SEQ ID NO: 82);
GAAATTAATACGACTCACTATAGGGATGTTCTATCCGCTGGTGTCGCTGGA (SEQ ID NO: 69) and ACCGCTGCCCTTCTTGATGTCGTAG (SEQ ID NO: 83);
GAAATTAATACGACTCACTATAGGGAATGGGCGTATGATTATACCCAGGTAA (SEQ ID NO: 70) and AGCAGTTTGGCAAAGTGCGCTTGTGCAG (SEQ ID NO: 84);
GAAATTAATACGACTCACTATAGGGGTTGAAAACCGTCGCTTTTACGCTGAAAAA (SEQ ID NO: 71) and TCCATTCGGCTTCTTTATTCGCCACCTGGTC (SEQ ID NO: 85);
GAAATTAATACGACTCACTATAGGGGACCATCCTGTACGCGCTGGGCTGGACGCAGC (SEQ ID NO: 72) and CACCACGATTTCATCAGGCTGACGTGGAACTTC (SEQ ID NO: 86);
GAAATTAATACGACTCACTATAGGGCGGTACGCCTACTCGTCGAGCGTGAACGTG (SEQ ID NO: 73) and CGTCACTTCCATACGCAAGGGTTGTTCATC (SEQ ID NO: 87);
GAAATTAATACGACTCACTATAGGGGGCACTTACGCGAAGTGATTGGTGGTGAC (SEQ ID NO: 74) and CACAGTAATGGCGAAACCATGAAGCCTAC (SEQ ID NO: 88);
GAAATTAATACGACTCACTATAGGGGAAGAGCAGCGCGTTGCCGATGCCTGGAAAG (SEQ ID NO: 75) and CTTTCGGCAGCGCCAGTTCGTCGTTGTGGT (SEQ ID NO: 89);
GAAATTAATACGACTCACTATAGGGACAAAGGTCAACCAATGACATTCAGACTAT (SEQ ID NO: 20) and GGACCATATTTCTCTACACCTTTTTTAGGA (SEQ ID NO: 32);
GAAATTAATACGACTCACTATAGGGAACAAGAAGCAGCGCGTAAA (SEQ ID NO: 21) and CCCCTGTTTACCTTTGGCTT (SEQ ID NO: 33);
GAAATTAATACGACTCACTATAGGGTAATCCATTTACCACTTCAACATTACAACAAGA (SEQ ID NO: 22) and TCAATACCTTCATATAATTGTTGCGCGAGCAT (SEQ ID NO: 34);
GAAATTAATACGACTCACTATAGGGGATTTAGTTGACGCGCAACAAGCACGTCGTATT (SEQ ID NO: 23) and CCTTCAATGGACCAATACTCTTCAGGTTTA (SEQ ID NO: 35);
GAAATTAATACGACTCACTATAGGGGAATTTAGTTGATGCACAACAAGCGCGTCGT (SEQ ID NO: 24) and AAGCAACTGATTGTACTCGACCAGCTGACAATC (SEQ ID NO: 36);
GAAATTAATACGACTCACTATAGGGACGTGTTCAGTCAGTAGCTCTTCGTTTAGTTA (SEQ ID NO: 25) and TTATATCTAAATTCCCCTTCAATAGTCCAATAT (SEQ ID NO: 37);
GAAATTAATACGACTCACTATAGGGAAAGGCTTTATGTCTGTATACGTCGAAGCGAA (SEQ ID NO: 26) and CAATCTTTGTTGCTGTCACCATCTCCCCTTCT (SEQ ID NO: 38);
GAAATTAATACGACTCACTATAGGGCCATCTTTTGATACAGTCCATGACTTAGCCATT (SEQ ID NO: 27) and GTGACAAATGGAGTTGACGGATTGTTAGAAA (SEQ ID NO: 39);
GAAATTAATACGACTCACTATAGGGCCATCTTTTGATACAGTCCATGACTTAGCCATT (SEQ ID NO: 27) and GTGACAAATGGAGTTGACGGATTGTTAGAAA (SEQ ID NO: 39);
GAAATTAATACGACTCACTATAGGGCCATCTTTTGATACAGTCCATGACTTAGCCATT (SEQ ID NO: 27) and GTGACAAATGGAGTTGACGGATTGTTAGAAA (SEQ ID NO: 39);
GAAATTAATACGACTCACTATAGGGAATGTTACCAGATATGGAGGAAGGAGAAAGT (SEQ ID NO: 28) and ACTTGCTTCTGAAAACCTTGCAGGAGGTTGA (SEQ ID NO: 40);
GAAATTAATACGACTCACTATAGGGGTTGATATATTGTTCTGGGATGTAGGATG (SEQ ID NO: 29) and TTCTACTACAAGATTGAACGTATCCGCGAA (SEQ ID NO: 41);
GAAATTAATACGACTCACTATAGGGACAGCAAGCTCGTCGTACCTTAGACAGAATCG (SEQ ID NO: 30) and ATATTCTTCTGGAACAAATTCTCGGATCTCTT (SEQ ID NO: 42); and
GAAATTAATACGACTCACTATAGGGGAGATTACTAAAGAAGCGGTAAAAGCGGCATT (SEQ ID NO: 31) and AACAGATTGCACACGACCAGCACTCAAACCT (SEQ ID NO: 43). In another embodiment the kit contains b) a second set of primers containing a set of sequences selected from one or more of:
GAAATTAATACGACTCACTATAGGGCGTCTAGTTCTGCTGTCTTGTCTCTCATGG (SEQ ID NO: 76) and CAAAGTCCTGTTCGAGTTTAGCGAATGGTT (SEQ ID NO: 90);
GAAATTAATACGACTCACTATAGGGAATGTCTGGCAGCACACTTCCTATCTCGAC (SEQ ID NO: 77) and TGATCTCCTGCTTGATCCAGTTGAGGATCT (SEQ ID NO: 91);
GAAATTAATACGACTCACTATAGGGATAAAACCGGCAGCGGTGGCTATGG (SEQ ID NO: 78) and GCTAATACATCGCGACGGCTTTCTGCCTTA (SEQ ID NO: 92); and
GAAATTAATACGACTCACTATAGGGTTAAAATTCCCAATAGCTTGATCGCCCTCG (SEQ ID NO: 79) and ATAAACAGGCACAACTGAATATTTCATCGC (SEQ ID NO: 93). In another embodiment, the kit contains c) a guide polynucleotide containing a nucleotide sequence selected from one or more of:

(SEQ ID NO: 95)
CGGCGCGACGACGGGCUUCCCGCAAGCAGUUUUAGUCCCCUUCGUUUUU
GGGGUAGUCUAAAUCCCCUAUAGUGAGUCGUAUUAAUUUC;
(SEQ ID NO: 96)
GUGAUGAGACCAUGGCGGCGGUAUCGCUGUUUUAGUCCCCUUCGUUUUU
GGGGUAGUCUAAAUCCCCUAUAGUGAGUCGUAUUAAUUUC;
(SEQ ID NO: 97)
UUCUAUAUGCGCGGCAAGAACGACUUCCGUUUUAGUCCCCUUCGUUUUU
GGGGUAGUCUAAAUCCCCUAUAGUGAGUCGUAUUAAUUUC;
(SEQ ID NO: 98)
AGUCAACUAUUAGGCCAAAGCUAUGAUCGUUUUAGUCCCCUUCGUUUUU
GGGGUAGUCUAAAUCCCCUAUAGUGAGUCGUAUUAAUUUC;
(SEQ ID NO: 99)
AUGGGUGAAAUCGUCACCGAUCGUCUGGGUUUUAGUCCCCUUCGUUUUU
GGGGUAGUCUAAAUCCCCUAUAGUGAGUCGUAUUAAUUUC;
(SEQ ID NO: 100)
UGUCCGACCUGCUGCCCGGCUACCUGACGUUUUAGUCCCCUUCGUUUUU
GGGGUAGUCUAAAUCCCCUAUAGUGAGUCGUAUUAAUUUC;
(SEQ ID NO: 101)
AUUGGCAAUUACACGCUUCACUGCUGACGUUUUAGUCCCCUUCGUUUUU
GGGGUAGUCUAAAUCCCCUAUAGUGAGUCGUAUUAAUUUC;
(SEQ ID NO: 102)
CAGCCAACACGUCUCGACUUAAACCGUGGUUUUAGUCCCCUUCGUUUUU
GGGGUAGUCUAAAUCCCCUAUAGUGAGUCGUAUUAAUUUC;
(SEQ ID NO: 103)
UGUGGUACCUACCACAUGCACUCGCUGGGUUUUAGUCCCCUUCGUUUUU
GGGGUAGUCUAAAUCCCCUAUAGUGAGUCGUAUUAAUUUC;
(SEQ ID NO: 104)
GCUGGCUGGCUUUUCUGCCACCGCGCUGGUUUUAGUCCCCUUCGUUUUU
GGGGUAGUCUAAAUCCCCUAUAGUGAGUCGUAUUAAUUUC;
(SEQ ID NO: 105)
CAACGGUUUGAUCGUCAGGGAUGGCGGCGUUUUAGUCCCCUUCGUUUUU
GGGGUAGUCUAAAUCCCCUAUAGUGAGUCGUAUUAAUUUC;
(SEQ ID NO: 106)
AUCGUGCGCCGCUGAUUCUGGUCACUUAGUUUUAGUCCCCUUCGUUUUU
GGGGUAGUCUAAAUCCCCUAUAGUGAGUCGUAUUAAUUUC;
(SEQ ID NO: 107)
CCAAGUCUUUAAGUGGGAUGGACAGACGGUUUUAGUCCCCUUCGUUUUU
GGGGUAGUCUAAAUCCCCUAUAGUGAGUCGUAUUAAUUUC;
and
(SEQ ID NO: 108)
GGCCGUCCGUUAAUUUCCCUUGCAUACAGUUUUAGUCCCCUUCGUUUUU
GGGGUAGUCUAAAUCCCCUAUAGUGAGUCGUAUUAAUUUC.

In another aspect, the disclosure provides a method of treating a selected subject having an antimicrobial resistant bacterial infection. The method involves administering an agent capable of reducing the survival or proliferation of bacteria of the bacterial infection to the selected subject. The subject is selected by a method involving a) splitting a biological sample from a subject into two containers. One container contains a first set of primers suitable for amplifying a target polynucleotide capable of identifying a bacterial species or taxonomic group. A second container contains a second set of primers suitable for amplifying a target AMR gene. One primer of each set of primers contains a T7 promoter sequence. The biological sample contains bacteria. The method for selecting the subject also involves b) incubating the biological sample under conditions suitable for recombinase polymerase amplification (RPA) to produce amplicons. The method for selecting the subject also involves b) contacting the amplicons with a T7 RNA polymerase to produce RNA. The method for selecting the subject also involves c) contacting the RNA with a Type VI CRISPR polypeptide, guide polynucleotides capable of specifically binding the targets, and a detection construct containing RNA, under conditions suitable for Type VI CRISPR polypeptide nuclease activity, thereby cutting the RNA. The method for selecting the subject alto involves d) detecting a detectable signal produced by the detection construct, where the presence of a detectable signal in the first container and the second container selects the subject for administration of a drug for treating an AMR infection.

In another aspect, the disclosure provides a method for selecting a subject for administration of an agent for treating a an antimicrobial resistant (AMR) infection. The method involves a) splitting a biological sample from a subject into two containers. One container contains a first set of primers suitable for amplifying a target polynucleotide capable of identifying a microbial species or taxonomic group. A second container contains a second set of primers suitable for amplifying a target AMR gene. One primer of each set of primers contains a T7 promoter sequence. The biological sample contains bacteria. The method also involves b) incubating the biological sample under conditions suitable for recombinase polymerase amplification (RPA) to produce amplicons. The method also involves b) contacting the amplicons with a T7 RNA polymerase to produce RNA. The method further involves c) contacting the RNA with a Type VI CRISPR polypeptide, guide polynucleotides capable of specifically binding the targets, and a detection construct containing RNA, under conditions suitable for Type VI CRISPR polypeptide nuclease activity, thereby cutting the RNA. The method also involves d) detecting a detectable signal produced by the detection construct. The presence of a detectable signal in the first container and the second container selects the subject for administration of a drug for treating an AMR infection.

In any aspect of the disclosure, or embodiments thereof, the sample is a biological sample or environmental sample. In any aspect of the disclosure, or embodiments thereof, the biological sample is a urine sample, blood sample, stool sample, or fluid sample obtained from a lung of a subject. In any aspect of the disclosure, or embodiments thereof, the environmental sample is waste water.

In any aspect of the disclosure, or embodiments thereof, the positive blood culture is diluted at least about 10-fold in the composition containing water.

In any aspect of the disclosure, or embodiments thereof, the method further involves a heat lysis step prior to a).

In any aspect of the disclosure, or embodiments thereof, the first set of primers amplify a fragment of a gene selected from one or more of: chuA from E. coli, topA from Kpneumoniae, C. freundii, P. mirabilis, or A. baumanii, lasB from P aeruginosa, and luxS from S. marcescens; and/or where the second set of primers amplify a fragment of an antimicrobial resistance gene selected from one or more of AMP-C, bla_{IMP}, bla_{VIM}, bla_{KPC-1}, bla_{KPC-2}, bla_{KPC-3}, bla_{NDM-1}, bla_{OXA-48}, bla_{OXA-232}, bla_{CTX-M-15}, bla_{SHV-12}, bla_{SHV-2}, mecA, vanA, vanB, and vanC.

In any aspect of the disclosure, or embodiments thereof, the Type VI CRISPR polypeptide is a Cas13a polypeptide.

In any aspect of the disclosure, or embodiments thereof, a) the first set of primers contains a set of sequences selected from one or more of:

(SEQ ID NO: 67)
GAAATTAATACGACTCACTATAGGGATATGGCGGTGAGTATTATCGTCA
GGAACAACATC
and
(SEQ ID NO: 81)
GAGATGACCATTTGTCGGCATCAACATCTTTGTAG;
(SEQ ID NO: 68)
GAAATTAATACGACTCACTATAGGGGGTGACGCCCTGCCGCTGCAGGTA
ACCCATAA
and
(SEQ ID NO: 82
ATCTTCACGCTCCAGTACGCTGTAGCTGGCTTT;
(SEQ ID NO: 69)
GAAATTAATACGACTCACTATAGGGATGTTCTATCCGCTGGTGTCGCTG
GA
and
(SEQ ID NO: 83)
ACCGCTGCCCTTCTTGATGTCGTAG;
(SEQ ID NO: 70)
GAAATTAATACGACTCACTATAGGGAATGGGCGTATGATTATACCCAGG
TAA
and
(SEQ ID NO: 84)
AGCAGTTTGGCAAAGTGCGCTTGTGCAG;
(SEQ ID NO: 71)
GAAATTAATACGACTCACTATAGGGGTTGAAAACCGTCGCTTTTACGCT
GAAAAA
and
(SEQ ID NO: 85)
TCCATTCGGCTTCTTTATTCGCCACCTGGTC;
(SEQ ID NO: 72)
GAAATTAATACGACTCACTATAGGGGACCATCCTGTACGCGCTGGGCTG
GACGCAGC
and
(SEQ ID NO: 86)
CACCACGATTTCATCAGGCTGACGTGGAACTTC;
(SEQ ID NO: 73)
GAAATTAATACGACTCACTATAGGGCGGTACGCCTACTCGTCGAGCGTG
AACGTG
and
(SEQ ID NO: 87)
CGTCACTTCCATACGCAAGGGTTGTTCATG;
(SEQ ID NO: 74)
GAAATTAATACGACTCACTATAGGGGGCACTTACGCGAAGTGATTGGTG
GTGAC
and
(SEQ ID NO: 88)
CACAGTAATGGCGAAACCATGAAGCCTAC;
(SEQ ID NO: 75)
GAAATTAATACGACTCACTATAGGGGAAGAGCAGCGCGTTGCCGATGCC
TGGAAAG
and
(SEQ ID NO: 89)
CTTTCGGCAGCGCCAGTTCGTCGTTGTGGT;
(SEQ ID NO: 20)
GAAATTAATACGACTCACTATAGGGACAAAGGTCAACCAATGACATTCA
GACTAT
and
(SEQ ID NO: 32)
GGACCATATTTCTCTACACCTTTTTTAGGA;
(SEQ ID NO: 21)
GAAATTAATACGACTCACTATAGGGAACAAGAAGCAGCGCGTAAA
and
(SEQ ID NO: 33)
CCCCTGTTTACCTTTGGCTT;
(SEQ ID NO: 22)
GAAATTAATACGACTCACTATAGGGTAATCCATTTACCACTTCAACATT
ACAACAAGA
and
(SEQ ID NO: 34)
TCAATACCTTCATATAATTGTTGCGCGAGCAT;
(SEQ ID NO: 23)
GAAATTAATACGACTCACTATAGGGGATTTAGTTGACGCGCAACAAGCA
CGTCGTATT
and
(SEQ ID NO: 35)
CCTTCAATGGACCAATACTCTTCAGGTTTA;
(SEQ ID NO: 24)
GAAATTAATACGACTCACTATAGGGGAATTTAGTTGATGCACAACAAGC
GCGTCGT
and
(SEQ ID NO: 36)
AAGCAACTGATTGTACTCGACCAGCTGACAATC;
(SEQ ID NO: 25)
GAAATTAATACGACTCACTATAGGGACGTGTTCAGTCAGTAGCTCTTCG
TTTAGTTA
and
(SEQ ID NO: 37)
TTATATCTAAATTCCCCTTCAATAGTCCAATAT;
(SEQ ID NO: 26)
GAAATTAATACGACTCACTATAGGGAAAGGCTTTATGTCTGTATACGTC
GAAGCGAA
and
(SEQ ID NO: 38)
CAATCTTTGTTGCTGTCACCATCTCCCCTTCT;
(SEQ ID NO: 27)
GAAATTAATACGACTCACTATAGGGCCATCTTTTGATACAGTCCATGAC
TTAGCCATT
and
(SEQ ID NO: 39)
GTGACAAATGGAGTTGACGGATTGTTAGAAA;
(SEQ ID NO: 27)
GAAATTAATACGACTCACTATAGGGCCATCTTTTGATACAGTCCATGAC
TTAGCCATT
and
(SEQ ID NO: 39)
GTGACAAATGGAGTTGACGGATTGTTAGAAA;
(SEQ ID NO: 27)
GAAATTAATACGACTCACTATAGGGCCATCTTTTGATACAGTCCATGAC
TTAGCCATT
and
(SEQ ID NO: 39)
GTGACAAATGGAGTTGACGGATTGTTAGAAA;
(SEQ ID NO: 28)
GAAATTAATACGACTCACTATAGGGAATGTTACCAGATATGGAGGAAGG
AGAAAGT
and
(SEQ ID NO: 40)
ACTTGCTTCTGAAAACCTTGCAGGAGGTTGA;
(SEQ ID NO: 29)
GAAATTAATACGACTCACTATAGGGGTTGATATATTGTTCTGGGATGTA
GGATG
and
(SEQ ID NO: 41)
TTCTACTACAAGATTGAACGTATCCGCGAA;
(SEQ ID NO: 30)
GAAATTAATACGACTCACTATAGGGACAGCAAGCTCGTCGTACCTTAGA
CAGAATCG
and
(SEQ ID NO: 42)
ATATTCTTCTGGAACAAATTCTCGGATCTCTT;
and
(SEQ ID NO: 31)
GAAATTAATACGACTCACTATAGGGGAGATTACTAAAGAAGCGGTAAAA
GCGGCATT
and
(SEQ ID NO: 43)
AACAGATTGCACACGACCAGCACTCAAACCT.

In any aspect of the disclosure, or embodiments thereof, the second set of primers contains a set of sequences selected from one or more of:
GAAATTAATACGACTCACTATAGGGCGTCTAGTTCTGCTGTCTTGTCTCTCATGG (SEQ ID NO: 76) and CAAAGTCCTGTTCGAGTTTAGCGAATGGTT (SEQ ID NO: 90);
GAAATTAATACGACTCACTATAGGGAATGTCTGGCAGCACACTTCCTATCTCGAC (SEQ ID NO: 77) and TGATCTCCTGCTTGATCCAGTTGAGGATCT (SEQ ID NO: 91);
GAAATTAATACGACTCACTATAGGGATAAAACCGGCAGCGGTGGCTATGG (SEQ ID NO: 78) and GCTAATACATCGCGACGGCTTTCTGCCTTA (SEQ ID NO: 92); and
GAAATTAATACGACTCACTATAGGGTTAAAATTCCCAATAGCTTGATCGCCCTCG (SEQ ID NO: 79) and ATAAACAGGCACAACTGAATATTTCATCGC (SEQ ID NO: 93); and/or

In any aspect of the disclosure, or embodiments thereof, the guide polynucleotides each contain a nucleotide sequence selected from one or more of:

(SEQ ID NO: 95)
CGGCGCGACGACGGGCUUCCCGCAAGCAGUUUUAGUCCCCUUCGUUUUU
GGGGUAGUCUAAAUCCCCUAUAGUGAGUCGUAUUAAUUUC;
(SEQ ID NO: 96)
GUGAUGAGACCAUGGCGGCGGUAUCGCUGUUUUAGUCCCCUUCGUUUUU
GGGGUAGUCUAAAUCCCCUAUAGUGAGUCGUAUUAAUUUC;
(SEQ ID NO: 97)
UUCUAUAUGCGCGGCAAGAACGACUUCCGUUUUAGUCCCCUUCGUUUUU
GGGGUAGUCUAAAUCCCCUAUAGUGAGUCGUAUUAAUUUC;
(SEQ ID NO: 98)
AGUCAACUAUUAGGCCAAAGCUAUGAUCGUUUUAGUCCCCUUCGUUUUU
GGGGUAGUCUAAAUCCCCUAUAGUGAGUCGUAUUAAUUUC;
(SEQ ID NO: 99)
AUGGGUGAAAUCGUCACCGAUCGUCUGGGUUUUAGUCCCCUUCGUUUUU
GGGGUAGUCUAAAUCCCCUAUAGUGAGUCGUAUUAAUUUC;
(SEQ ID NO: 100)
UGUCCGACCUGCUGCCCGGCUACCUGACGUUUUAGUCCCCUUCGUUUUU
GGGGUAGUCUAAAUCCCCUAUAGUGAGUCGUAUUAAUUUC;
(SEQ ID NO: 101)
AUUGGCAAUUACACGCUUCACUGCUGACGUUUUAGUCCCCUUCGUUUUU
GGGGUAGUCUAAAUCCCCUAUAGUGAGUCGUAUUAAUUUC;
(SEQ ID NO: 102)
CAGCCAACACGUCUCGACUUAAACCGUGGUUUUAGUCCCCUUCGUUUUU
GGGGUAGUCUAAAUCCCCUAUAGUGAGUCGUAUUAAUUUC;
(SEQ ID NO: 103)
UGUGGUACCUACCACAUGCACUCGCUGGGUUUUAGUCCCCUUCGUUUUU
GGGGUAGUCUAAAUCCCCUAUAGUGAGUCGUAUUAAUUUC;
(SEQ ID NO: 104)
GCUGGCUGGCUUUUCUGCCACCGCGCUGGUUUUAGUCCCCUUCGUUUUU
GGGGUAGUCUAAAUCCCCUAUAGUGAGUCGUAUUAAUUUC;
(SEQ ID NO: 105)
CAACGGUUUGAUCGUCAGGGAUGGCGGCGUUUUAGUCCCCUUCGUUUUU
GGGGUAGUCUAAAUCCCCUAUAGUGAGUCGUAUUAAUUUC;
(SEQ ID NO: 106)
AUCGUGCGCCGCUGAUUCUGGUCACUUAGUUUUAGUCCCCUUCGUUUUU
GGGGUAGUCUAAAUCCCCUAUAGUGAGUCGUAUUAAUUUC;
(SEQ ID NO: 107)
CCAAGUCUUUAAGUGGGAUGGACAGACGGUUUUAGUCCCCUUCGUUUUU
GGGGUAGUCUAAAUCCCCUAUAGUGAGUCGUAUUAAUUUC;
and
(SEQ ID NO: 108)
GGCCGUCCGUUAAUUUCCCUUGCAUACAGUUUUAGUCCCCUUCGUUUUU
GGGGUAGUCUAAAUCCCCUAUAGUGAGUCGUAUUAAUUUC.

In any aspect of the disclosure, or embodiments thereof, each container contains magnesium acetate.

In any aspect of the disclosure, or embodiments thereof, the detectable signal is produced by a fluorophore or a dye. In any aspect of the disclosure, or embodiments thereof, the detectable signal is detected using a lateral flow strip or a fluorimeter. In any aspect of the disclosure, or embodiments thereof, the biological sample is a blood sample.

In any aspect of the disclosure, or embodiments thereof, the bacterium is a Gram negative bacteria selected from one or more of E. coli, K. pneumoniae, E. cloacae, C. freundii, S. marcescens, S. maltophila, P. mirabilis, A. baumanii, K. oxytoca, P. aeruginosa, Vibrio cholerae, Salmonella enterica, Salmonella typhi, Citrobacter koseri, Klebsiella aerogenes, and Bacteroides fragilis, a member of the genus Citrobacter, and Enterobacter aerogenes. In any aspect of the disclosure, or embodiments thereof, the bacterium is a Gram positive bacteria selected from one or more of S. Aureus, S. hemolyticus, S. capitis, S. hominis, S. pettenkoferi, S. lugdunensis, S. epidermidis E. faecalis, E. faecium, S. anginosus complex, S. agalactiae, S. pneumoniae, Streptococcus bovis, S. pyogenes, S. constellatus, S. intermedius, S. mitis, S. mutans, S. salivarius, S. oralis, S. sanguinis, S. gordonii, S. gallolyticus, S. pneumoniae, S. anginosus, and S. agalactiae.

In any aspect of the disclosure, or embodiments thereof, the bacteria contain an AMR gene selected from one or more of AMP-C, bla_{VIM}, bla_{IMP}, bla_{KPC-1}, bla_{KPC-2}, bla_{KPC-3}, bla_{NDM-1}, bla_{OXA-48}, bla_{CTX-M-15}, mecA, vanA, vanB, and vanC, and where:
a) when the AMR gene is bla_{CTX-M-15}, the drug is selected from one or more of meropenem, imipenem, ertapenem, colistin, ceftazidime-avibactam, cefiderocol, cetazidime-avibactam-aztreonam, and meropenem-vaborbactam;
b) when the AMR gene is bla_{KPC-1}, bla_{KPC-2}, or bla_{KPC-3}, the drug is selected from one or more of colistin, ceftazidime-avibactam, cefiderocol, cetazidime-avibactam-aztreonam, and meropenem-vaborbactam;
c) when the AMR gene is bla_{NDM-1}, the drug is selected from one or more of colistin, and cetazidime-avibactam-aztreonam;
d) when the AMR gene is bla_{OXA-48}, the drug is selected from one or more of colistin, ceftazidime-avibactam, cefiderocol, cetazidime-avibactam-aztreonam, and meropenem-vaborbactam;
e) when the AMR gene is bla_{VIM}, the drug is cetazidime-avibactam-aztreonam;
f) when the AMR gene is bla_{IMP}, the drug is selected from one or more of colistin and cetazidime-avibactam-aztreonam;
g) when the AMR gene is mecA, the drug is selected from one or more of vancomycin, linezolid, daptomycin, and dalbavancin;
h) when the AMP-C gene is mecA, the drug is selected from one or more of cefepime, meropenem, imipenem, ertapenem, colistin, ceftazidime-avibactam, cefiderocol, cetazidime-avibactam-aztreonam, and meropenem-vaborbactam; and
i) when the AMP-C gene is vanA, vanB, or vanC, the drug is selected from one or more of linezolid, daptomycin, and dalbavancin.

In any aspect of the disclosure, or embodiments thereof, the biological sample is a blood sample that is diluted at least about 10-fold in a composition containing water. In any aspect of the disclosure, or embodiments thereof, the composition containing water is distilled or deionized water.

In any aspect of the disclosure, or embodiments thereof, the first set of primers amplify a fragment of a gene selected from one or more of: chuA from E. coli, topA from Kpneumoniae, C. freundii, P. mirabilis, or A. baumanii, lasB from P aeruginosa, and luxS from S. marcescens.

In any aspect of the disclosure, or embodiments thereof, the Type VI CRISPR polypeptide is a Cas13a polypeptide.

In any aspect of the disclosure, or embodiments thereof, the first set of primers contains a set of sequences selected from one or more of:

(SEQ ID NO: 67)
GAAATTAATACGACTCACTATAGGGATATGGCGGTGAGTATTATCGTCA
GGAACAACATC
and
(SEQ ID NO: 81)
GAGATGACCATTTGTCGGCATCAACATCTTTGTAG;
GAAATTAATACGACTCACTATAGGGGGTGACGCCCTGCCGCTGCAGGTA
ACCCATAA
and
(SEQ ID NO: 82)
ATCTTCACGCTCCAGTACGCTGTAGCTGGCTTT;
(SEQ ID NO: 69)
GAAATTAATACGACTCACTATAGGGATGTTCTATCCGCTGGTGTCGCTG
GA
and
(SEQ ID NO: 83)
ACCGCTGCCCTTCTTGATGTCGTAG;
(SEQ ID NO: 70)
GAAATTAATACGACTCACTATAGGGAATGGGCGTATGATTATACCCAGG
TAA
and
(SEQ ID NO: 84)
AGCAGTTTGGCAAAGTGCGCTTGTGCAG;
(SEQ ID NO: 71)
GAAATTAATACGACTCACTATAGGGGTTGAAAACCGTCGCTTTTACGCT
GAAAAA
and
(SEQ ID NO: 85)
TCCATTCGGCTTCTTTATTCGCCACCTGGTC;
(SEQ ID NO: 72)
GAAATTAATACGACTCACTATAGGGGACCATCCTGTACGCGCTGGGCTG
GACGCAGC
and
(SEQ ID NO: 86)
CACCACGATTTCATCAGGCTGACGTGGAACTTC;
(SEQ ID NO: 73)
GAAATTAATACGACTCACTATAGGGCGGTACGCCTACTCGTCGAGCGTG
AACGTG
and
(SEQ ID NO: 87)
CGTCACTTCCATACGCAAGGGTTGTTCATC;
(SEQ ID NO: 74)
GAAATTAATACGACTCACTATAGGGGGCACTTACGCGAAGTGATTGGTG
GTGAC
and
(SEQ ID NO: 88)
CACAGTAATGGCGAAACCATGAAGCCTAC;
(SEQ ID NO: 75)
GAAATTAATACGACTCACTATAGGGGAAGAGCAGCGCGTTGCCGATGCC
TGGAAAG
and
(SEQ ID NO: 89)
CTTTCGGCAGCGCCAGTTCGTCGTTGTGGT;
(SEQ ID NO: 20)
GAAATTAATACGACTCACTATAGGGACAAAGGTCAACCAATGACATTCA
GACTAT
and
(SEQ ID NO: 32)
GGACCATATTTCTCTACACCTTTTTTAGGA;
(SEQ ID NO: 21
GAAATTAATACGACTCACTATAGGGAACAAGAAGCAGCGCGTAAA
and
(SEQ ID NO: 33)
CCCCTGTTTACCTTTGGCTT;
(SEQ ID NO: 22)
GAAATTAATACGACTCACTATAGGGTAATCCATTTACCACTTCAACATT
ACAACAAGA
and
(SEQ ID NO: 34)
TCAATACCTTCATATAATTGTTGCGCGAGCAT;
(SEQ ID NO: 23)
GAAATTAATACGACTCACTATAGGGGATTTAGTTGACGCGCAACAAGCA
CGTCGTATT
and
(SEQ ID NO: 35)
CCTTCAATGGACCAATACTCTTCAGGTTTA;
(SEQ ID NO: 24)
GAAATTAATACGACTCACTATAGGGGAATTTAGTTGATGCACAACAAGC
GCGTCGT
and
(SEQ ID NO: 36)
AAGCAACTGATTGTACTCGACCAGCTGACAATC;
(SEQ ID NO: 25)
GAAATTAATACGACTCACTATAGGGACGTGTTCAGTCAGTAGCTCTTCG
TTTAGTTA
and
(SEQ ID NO: 37)
TTATATCTAAATTCCCCTTCAATAGTCCAATAT;
(SEQ ID NO: 26)
GAAATTAATACGACTCACTATAGGGAAAGGCTTTATGTCTGTATACGTC
GAAGCGAA
and
(SEQ ID NO: 38)
CAATCTTTGTTGCTGTCACCATCTCCCCTTCT;
(SEQ ID NO: 27)
GAAATTAATACGACTCACTATAGGGCCATCTTTTGATACAGTCCATGAC
TTAGCCATT
and
(SEQ ID NO: 39)
GTGACAAATGGAGTTGACGGATTGTTAGAAA;
(SEQ ID NO: 27)
GAAATTAATACGACTCACTATAGGGCCATCTTTTGATACAGTCCATGAC
TTAGCCATT
and
(SEQ ID NO: 39)
GTGACAAATGGAGTTGACGGATTGTTAGAAA;
(SEQ ID NO: 27)
GAAATTAATACGACTCACTATAGGGCCATCTTTTGATACAGTCCATGAC
TTAGCCATT
and
(SEQ ID NO: 39)
GTGACAAATGGAGTTGACGGATTGTTAGAAA;
(SEQ ID NO: 28)
GAAATTAATACGACTCACTATAGGGAATGTTACCAGATATGGAGGAAGG
AGAAAGT
and
(SEQ ID NO: 40)
ACTTGCTTCTGAAAACCTTGCAGGAGGTTGA;
(SEQ ID NO: 29)
GAAATTAATACGACTCACTATAGGGGTTGATATATTGTTCTGGGATGTA
GGATG
and
(SEQ ID NO: 41)
TTCTACTACAAGATTGAACGTATCCGCGAA;
(SEQ ID NO: 30)
GAAATTAATACGACTCACTATAGGGACAGCAAGCTCGTCGTACCTTAGA
CAGAATCG
and
(SEQ ID NO: 42)
ATATTCTTCTGGAACAAATTCTCGGATCTCTT;
and
(SEQ ID NO: 31)
GAAATTAATACGACTCACTATAGGGGAGATTACTAAAGAAGCGGTAAAA
GCGGCATT
and
(SEQ ID NO: 43)
AACAGATTGCACACGACCAGCACTCAAACCT;

In any aspect of the disclosure, or embodiments thereof, the second set of primers contains a set of sequences selected from one or more of:

(SEQ ID NO: 76)
GAAATTAATACGACTCACTATAGGGCGTCTAGTTCTGCTGTCTTGTCTC
TCATGG
and
(SEQ ID NO: 90)
CAAAGTCCTGTTCGAGTTTAGCGAATGGTT;
(SEQ ID NO: 77)
GAAATTAATACGACTCACTATAGGGAATGTCTGGCAGCACACTTCCTAT
CTCGAC
and
(SEQ ID NO: 91)
TGATCTCCTGCTTGATCCAGTTGAGGATCT;;
(SEQ ID NO: 78)
GAAATTAATACGACTCACTATAGGGATAAAACCGGCAGCGGTGGCTATG
G
and
(SEQ ID NO: 92)
GCTAATACATCGCGACGGCTTTCTGCCTTA;
and
(SEQ ID NO: 79)
GAAATTAATACGACTCACTATAGGGTTAAAATTCCCAATAGCTTGATCG
CCCTCG
and
(SEQ ID NO: 93)
ATAAACAGGCACAACTGAATATTTCATCGC.

In any aspect of the disclosure, or embodiments thereof, the guide polynucleotides each contain a nucleotide sequence selected from one or more of:

(SEQ ID NO: 95)
CGGCGCGACGACGGGCUUCCCGCAAGCAGUUUUAGUCCCCUUCGUUUUU
GGGGUAGUCUAAAUCCCCUAUAGUGAGUCGUAUUAAUUUC;
(SEQ ID NO: 96)
GUGAUGAGACCAUGGCGGCGGUAUCGCUGUUUUAGUCCCCUUCGUUUUU
GGGGUAGUCUAAAUCCCCUAUAGUGAGUCGUAUUAAUUUC;
(SEQ ID NO: 97)
UUCUAUAUGCGCGGCAAGAACGACUUCCGUUUUAGUCCCCUUCGUUUUU
GGGGUAGUCUAAAUCCCCUAUAGUGAGUCGUAUUAAUUUC;
(SEQ ID NO: 98)
AGUCAACUAUUAGGCCAAAGCUAUGAUCGUUUUAGUCCCCUUCGUUUUU
GGGGUAGUCUAAAUCCCCUAUAGUGAGUCGUAUUAAUUUC;
(SEQ ID NO: 99)
AUGGGUGAAAUCGUCACCGAUCGUCUGGGUUUUAGUCCCCUUCGUUUUU
GGGGUAGUCUAAAUCCCCUAUAGUGAGUCGUAUUAAUUUC;
(SEQ ID NO: 100)
UGUCCGACCUGCUGCCCGGCUACCUGACGUUUUAGUCCCCUUCGUUUUU
GGGGUAGUCUAAAUCCCCUAUAGUGAGUCGUAUUAAUUUC;
(SEQ ID NO: 101)
AUUGGCAAUUACACGCUUCACUGCUGACGUUUUAGUCCCCUUCGUUUUU
GGGGUAGUCUAAAUCCCCUAUAGUGAGUCGUAUUAAUUUC;
(SEQ ID NO: 102)
CAGCCAACACGUCUCGACUUAAACCGUGGUUUUAGUCCCCUUCGUUUUU
GGGGUAGUCUAAAUCCCCUAUAGUGAGUCGUAUUAAUUUC;
(SEQ ID NO: 103)
UGUGGUACCUACCACAUGCACUCGCUGGGUUUUAGUCCCCUUCGUUUUU
GGGGUAGUCUAAAUCCCCUAUAGUGAGUCGUAUUAAUUUC;
(SEQ ID NO: 104)
GCUGGCUGGCUUUUCUGCCACCGCGCUGGUUUUAGUCCCCUUCGUUUUU
GGGGUAGUCUAAAUCCCCUAUAGUGAGUCGUAUUAAUUUC;
(SEQ ID NO: 105)
CAACGGUUUGAUCGUCAGGGAUGGCGGCGUUUUAGUCCCCUUCGUUUUU
GGGGUAGUCUAAAUCCCCUAUAGUGAGUCGUAUUAAUUUC;
(SEQ ID NO: 106)
AUCGUGCGCCGCUGAUUCUGGUCACUUAGUUUUAGUCCCCUUCGUUUUU
GGGGUAGUCUAAAUCCCCUAUAGUGAGUCGUAUUAAUUUC;
(SEQ ID NO: 107)
CCAAGUCUUUAAGUGGGAUGGACAGACGGUUUUAGUCCCCUUCGUUUUU
GGGGUAGUCUAAAUCCCCUAUAGUGAGUCGUAUUAAUUUC;
and
(SEQ ID NO: 108)
GGCCGUCCGUUAAUUUCCCUUGCAUACAGUUUUAGUCCCCUUCGUUUUU
GGGGUAGUCUAAAUCCCCUAUAGUGAGUCGUAUUAAUUUC.

In any aspect of the disclosure, or embodiments thereof, a) involves splitting the biological sample into at least three or more containers, where at least between 2 and 9 of the containers contain unique sets of primers suitable for amplifying a target nucleotide capable of identifying a unique microbial species or taxonomic group, and where between at least 1 and 4 of the containers contain unique sets of primers suitable for amplifying a target nucleotide specific to an AMR gene.

In any aspect of the disclosure, or embodiments thereof, the amplification and nuclease reactions are carried out in parallel.

In some embodiments, the disclosure provides for a method of selecting a treatment of a subject having a pathogenic infection, including: obtaining a sample from the subject; contacting the sample with a composition, wherein the composition includes a reagent mix, at least one pair of recombinase polymerase amplification (RPA) primers, and at least one CRISPR guide RNA (crRNA) designed to correspond to at least one target; determining the presence or absence of the at least one target; correlating the presence or absence of the at least one target with the presence or absence of a pathogen, thereby detecting a pathogen; and selecting a treatment for the subject based on the correlation.

In some embodiments, the pathogen is selected from the group including a bacterium, virus, fungus, protozoa, and parasite.

In some embodiments, the method takes between about 60 minutes and 90 minutes.

In some embodiments, the sample is a blood culture sample. In a related embodiment, the blood culture sample is from a subject having, at risk of having, or suspected of having an infection.

In some embodiments, the pathogen is a gram-negative bacterial pathogen, a gram-positive bacterial pathogen, or both. In related embodiments, the gram-negative bacterial pathogen is selected from the group including, but not limited to: E. coli, K. pneumoniae, E. cloacae, C. freundii, S. marcescens, S. maltophila, P. mirabilis, A. baumanii, K. oxytoca, P. aeruginosa, Vibrio cholerae, Salmonella enterica, Salmonella typhi, Citrobacter koseri, Klebsiella aerogenes, and Bacteroides fragilis, a member of the genus Citrobacter, and Enterobacter aerogenes. In related embodiments, the gram-positive bacterial pathogen is selected from the group including, but not limited to: S. Aureus, S. hemolyticus, S. capitis, S. hominis, S. pettenkoferi, S. lugdunensis, and S. epidermidis. In related embodiments, the gram-positive bacterial pathogen is selected from the group including, but not limited to: E. faecalis, E. faecium, S. anginosus complex, S. agalactiae, S. pneumoniae, Streptococcus bovis, S. pyogenes, S. constellatus, S. intermedius, S. mitis, S. mutans, S. salivarius, S. oralis, S. sanguinis, S. gordonii, S. gallolyticus, S. pneumoniae, S. anginosus, and S. agalactiae.

In some embodiments, methods of the disclosure include that prior to contacting the blood culture with the composition, the blood culture sample is lysed.

In some embodiments, the lysis step includes, but is not limited to, one or more of the following: diluting the sample, diluting the sample to a 1:10 dilution, adding rehydrated achromopeptidase (ACP), heating the sample by boiling, heating the sample by boiling for about 10 minutes, use of a detergent, enzymatic or any combination thereof.

In some embodiments, the at least one target is an antimicrobial resistance (AMR) gene. In related embodiments, the antimicrobial resistance (AMR) gene selected from the group consisting of: AMP-C, bla_{IMP}, bla_{VIM}, bla_{KPC}, bla_{NDM}, bla_{OXA-48}, bla_{CTX-M}, bla_{SHV-12}, bla_{SHV-2}, mecA, vanA, vanB, and vanC.

In some embodiments, methods of the disclosure include about 1 target, about 2 targets, about 3 targets, about 4 targets, about 5 targets, about 6 targets, about 7 targets, about 8 targets, about 9 targets, about 10 targets, about 11 targets, about 12 targets, about 13 targets, about 14 targets, about 15 targets, about 16 targets, about 17 targets, about 18 targets, about 19 targets, about 20 targets, about 21 targets, about 22 targets, about 23 targets, or about 24 targets.

In some embodiments, the determining step includes a lateral flow assay.

In some embodiments, the determining step includes spectrophotometric detection.

In some embodiments, at least one CRISPR guide RNA (crRNA) includes a sequence having about 28 base pairs followed by the sequence gttttagtccccttcgtttttggggtagtctaaatcccctatagtgagtegtattaatttc (SEQ ID NO: 1).

In some embodiments, the reagent mix includes, but is not limited to, an assay-buffer, dNTPs, a Cas13, rNTPs, a T7 polymerase, an RNAse inhibitor, and an RNAse reporter.

In some embodiments, the reagent mix includes an RNA-targeting effector protein, wherein the RNA-targeting effector protein is suspended in a salt-free storage buffer.

In some embodiments, the Cas13 is suspended in a salt-free storage buffer. In related embodiments, the salt-free storage buffer includes tris hydrochloride (Tris-HCL), glycerol, and nuclease-free water. In related embodiments, the salt-free storage buffer includes, but is not limited to, one or more of: Tris-HCL at a pH of about 7.5, a concentration of Tris-HCL of about 50 mM, about 5% glycerol by volume, or any combination thereof.

In some embodiments, the at least one pair of recombinase polymerase amplification (RPA) primers are at a concentration of about 10 mM, about 15 mM, or about 20 mM.

In some embodiments, the reagent mix includes an assay-buffer including, but not limited to, one or more of: TwistAmp® liquid basic 2× Buffer (TwistDx™), TwistAmp® liquid basic 20× Core Reaction Mix; TwistAmp® liquid basic 10× E-mix; a cleavage-buffer; or any combination thereof.

In some embodiments, the reagent mix includes a cleavage-buffer including tris hydrochloride (Tris-HCL), dithiothreitol (DTT), and nuclease-free water. In related embodiments, the cleavage-buffer includes, but is not limited to, one or more of: Tris-HCL at a pH of about 7.5, a concentration of Tris-HCL of about 1M, about 40% Tris-HCL by volume, a concentration of DTT of about 100 mM, about 10% DTT by volume, about 50% nuclease-free water, or any combination thereof.

In some embodiments, the disclosure provides for a pathogen detection system including a reagent mix, at least one pair of recombinase polymerase amplification (RPA) primers, and at least one CRISPR guide RNA (crRNA) designed to correspond to at least one target.

In some embodiments, the pathogen is selected from the group consisting of a bacterium, virus, fungus, protozoa, and parasite. In related embodiments, the pathogen is a gram-negative bacterial pathogen, a gram-positive bacterial pathogen, or both. In related embodiments, the gram-negative bacterial pathogen is selected from the group including, but not limited to: A. baumanii, E. coli, K. pneumoniae, E. cloacae, C. freundii, S. marcescens, S. maltophila, P. mirabilis, A. baumanii, K. oxytoca, P. aeruginosa, Vibrio cholerae, Salmonella enterica, Salmonella typhi, Citrobacter koseri, Klebsiella aerogenes, and Bacteroides fragilis, a member of the genus Citrobacter, and Enterobacter aerogenes. In related embodiments, the gram-positive bacterial pathogen is selected from the group including, but not limited to: S. Aureus, S. hemolyticus, S. capitis, S. hominis, S. pettenkoferi, S. lugdunensis, and S. epidermidis. In some embodiments, the gram-positive bacterial pathogen is selected from the group including, but not limited to: E. faecalis, E. faecium, S. anginosus complex, S. agalactiae, S. pneumoniae, Streptococcus pyogenes, Streptococcus bovis, Streptococcus constellatus, S. intermedius, Streptococcus intermedius, Streptococcus mitis, Streptococcus mutans, Streptococcus salivarius, Streptococcus oralis, Streptococcus sanguinis, Streptococcus gordonii, Streptococcus gallolyticus, S. pneumoniae, S. pyogenes, Streptococcus bovis, Streptococcus anginosus, and S. agalactiae.

In some embodiments, the pathogen detection system of the disclosure includes having at least one target where the target is an antimicrobial resistance (AMR) gene. In related embodiments, the antimicrobial resistance (AMR) gene selected from the group consisting of: AMP-C, bla_{IMP}, bla_{VIM}, bla_{KPC}, bla_{NDM}, bla_{OXA-48}, bla_{CTX-M}, bla_{SHV-12}, bla_{SHV-2}, mecA, vanA, vanB, and vanC.

In some embodiments, the pathogen detection system includes about 1 target, about 2 targets, about 3 targets, about 4 targets, about 5 targets, about 6 targets, about 7 targets, about 8 targets, about 9 targets, about 10 targets, about 11 targets, about 12 targets, about 13 targets, about 14 targets, about 15 targets, about 16 targets, about 17 targets, about 18 targets, about 19 targets, about 20 targets, about 21 targets, about 22 targets, about 23 targets, or about 24 targets.

In some embodiments, the at least one CRISPR guide RNA (crRNA) includes a sequence having about 28 base pairs followed by the sequence gttttagtccccttcgtttttggggtagtctaaatcccctatagtgagtcgtattaatttc (SEQ ID NO: 1). In related embodiments, the reagent mix includes an assay-buffer, dNTPs, a Cas13, rNTPs, a T7 polymerase, an RNAse inhibitor, and an RNAse reporter. In related embodiments, the reagent mix includes an RNA-targeting effector protein, wherein the RNA-targeting effector protein is suspended in a salt-free storage buffer. In related embodiments, the Cas13 is suspended in a salt-free storage buffer.

In some embodiments, the salt-free storage buffer includes tris hydrochloride (Tris-HCL), glycerol, and nuclease-free water. In related embodiments, the salt-free storage buffer includes, but is not limited to, one or more of: Tris-HCL at a pH of about 7.5, a concentration of Tris-HCL of about 50 mM, about 5% glycerol by volume, or any combination thereof.

In some embodiments, pathogen detection systems of the present disclosure include at least one pair of recombinase polymerase amplification (RPA) primers with a concentration of about 10 mM, about 15 mM, or about 20 mM.

In some embodiments, the reagent mix includes an assay-buffer including, but not limited to, one or more of: TwistAmp® liquid basic 2× Buffer (TwistDx™), TwistAmp® liquid basic 20× Core Reaction Mix; TwistAmp® liquid basic 10× E-mix; a cleavage-buffer; or any combination thereof.

In some embodiments, the reagent mix includes a cleavage-buffer including tris hydrochloride (Tris-HCL), dithiothreitol (DTT), and nuclease-free water. In related embodiments, the cleavage-buffer includes, but is not limited to, one or more of: Tris-HCL at a pH of about 7.5, a concentration of Tris-HCL of about 1M, about 40% Tris-HCL by volume, a concentration of DTT of about 100 mM, about 10% DTT by volume, about 50% nuclease-free water, or any combination thereof.

In any aspect of the disclosure, or embodiments thereof, the amplification of the target polynucleotide is carried out using an isothermal amplification reaction. In any aspect of the disclosure, or embodiments thereof, the isothermal amplification reaction is recombinase polymerase amplification (RPA).

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. In some embodiments, an agent is a drug for treating a microbial infection, such as a microbial infection caused by a microbe (e.g., a prokaryotic organism) containing a microbial resistance gene. In some embodiments, an agent is a Type VI CRISPR protein, a guide RNA, an RNA-based detection construct, and/or a primer. In various embodiments, an agent is one of those agents listed in Table A.

By “ameliorate” is meant decrease, suppress, attenuate, diminish, arrest, or stabilize the development or progression of a disease.

By “AmpC beta-lactamase (AMP-C) polypeptide” is meant a protein having AmpC beta-lactamase activity and with at least 85% sequence identity to GenBank® Ref. Seq. Accession No. AFA35105.1, which is provided below, or a functional fragment thereof:

>AFA35105.1 AmpC [Acinetobacter baumannii]

(SEQ ID NO: 109)
MRFKKISCLLLSPLFIFSTSIYAGNTPKDQEIKKLVDQNFKPLLEKYDV
PGMAVGVIQNNKKYEMYYGLQSVQDKKAVNRSTIFELGSVSKLFTATAG
GYAKNKGKISFDDTPGKYWKELKNTPIDQVNLLQLATYTSGNLALQFPD
EVQTDQQVLTFFKDWQPKNPIGEYRQYSNPSIGLFGKVVALSMNKPFDQ
VLEKTIFPALGLKHSYVNVPKTQMQNYAFGYNQENQPIRVNPGPLDAPA
YGVKSTLPDMLSFIHANLNPQKYPADIQRAINETHQGFYQVNTMYQALG
WEEFSYPATLQTLLDSNSEQIVMKPNKVTAISKEPSVKMYHKTGSTNGF
GTYVVFIPKENIGLVMLTNKRIPNEERIKAAYAVLDAIKK

By “AmpC beta-lactamase (AMP-C) polynucleotide” is meant a nucleotide molecule encoding an AmpC beta-lactamase polypeptide. A representative AmpC beta-lactamase polynucleotide sequence is provided below (GenBank® Ref. Seq. GQ406245.5:60706-61857):

>GQ406245.5:60706-61857 Acinetobacter baumannii strain D2 KL1b capsule biosynthesis gene cluster and multiple antibiotic resistance island AbaR6

(SEQ ID NO: 110)
ATGCGATTTAAAAAAATTTCTTGTCTACTTTTATCCCCGCTTTTTATTTTTAGTACCTCAATTT
ATGCGGGCAATACACCAAAAGACCAAGAAATTAAAAAACTGGTAGATCAAAACTTTAAACCGTT
ATTAGAAAAATATGATGTGCCAGGTATGGCTGTGGGTGTTATTCAAAATAATAAAAAGTATGAA
ATGTATTATGGTCTTCAATCTGTTCAAGATAAAAAAGCCGTAAATCGCAGTACCATTTTTGAGC
TAGGTTCTGTCAGTAAATTATTTACCGCGACAGCAGGTGGATATGCAAAAAATAAAGGAAAAAT
CTCTTTTGACGATACGCCTGGTAAATATTGGAAAGAACTAAAAAACACACCGATTGACCAAGTT
AACTTACTTCAACTCGCGACGTATACAAGTGGTAACCTTGCCTTGCAGTTTCCAGATGAAGTAC
AAACAGACCAACAAGTTTTAACTTTTTTCAAAGACTGGCAACCTAAAAACCCAATCGGTGAATA
CAGACAATATTCAAATCCAAGTATTGGCCTATTTGGAAAGGTTGTGGCTTTGTCTATGAATAAA
CCTTTCGACCAAGTCTTAGAAAAAACAATTTTTCCGGCCCTTGGCTTAAAACATAGCTATGTAA
ATGTACCTAAGACCCAGATGCAAAACTATGCATTTGGTTATAACCAAGAAAATCAGCCGATTCG
AGTTAACCCCGGCCCACTCGATGCCCCTGCATATGGCGTCAAATCGACACTACCCGACATGTTG
AGTTTTATTCATGCCAACCTTAACCCACAGAAATATCCGGCTGATATTCAACGGGCAATTAATG
AAACACATCAAGGGTTCTATCAAGTAAATACCATGTATCAGGCACTCGGTTGGGAAGAGTTTTC
TTATCCGGCAACGTTACAAACTTTATTAGACAGTAATTCAGAACAGATTGTGATGAAACCTAAT
AAAGTGACTGCTATTTCAAAGGAACCTTCAGTTAAGATGTACCATAAAACTGGCTCAACCAACG
GTTTCGGAACATATGTAGTGTTTATTCCTAAAGAAAATATTGGCTTAGTCATGTTAACCAATAA
ACGTATTCCAAATGAAGAGCGCATTAAGGCAGCTTATGCTGTGCTGGATGCAATAAAGAAATAA

By “alteration” is meant a change positively or negative compared to a reference. In some embodiments, an alteration is a change in the structure, expression levels, or activity of a polynucleotide or polypeptide as detected by standard art known methods and/or those described herein. The alteration can be an increase or a decrease. As used herein, an alteration includes a 10% change in expression levels, a 25% change, a 40% change, and a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “biological sample” is meant a sample obtained from a biological subject. In embodiments, the sample is a tissue or fluid. In embodiments, the biological fluid is blood, plasma, serum, urine, biological secretion (e.g., mucous, phlegm, vaginal secretion), seminal fluid, fluid obtained from the lung or another organ.

By “cefotaximase 15 (CTX-M-15; bla_{CTX-M-15}) polypeptide” is meant a protein having cefotaxime-hydrolyzing beta-lactamase activity and with at least 85% sequence identity to NCBI® Ref. Seq. Accession No. WP_000239590.1, which is provided below, or a functional fragment thereof:

>WP 000239590.1 MULTISPECIES: extended-spectrum class A beta-lactamase CTX-M-15 [Bacteria]

(SEQ ID NO: 2)
MVKKSLRQFTLMATATVTLLLGSVPLYAQTADVQQKLAELERQSGGRLG
VALINTADNSQILYRADERFAMCSTSKVMAAAAVLKKSESEPNLLNORV
EIKKSDLVNYNPIAEKHVNGTMSLAELSAAALQYSDNVAMNKLIAHVGG
PASVTAFARQLGDETFRLDRTEPTLNTAIPGDPRDTTSPRAMAQTLRNL
TLGKALGDSQRAQLVTWMKGNTTGAASIQAGLPASWVVGDKTGSGGYGT
TNDIAVIWPKDRAPLILVTYFTQPQPKAESRRDVLASAAKIVTDGL

By “cefotaximase 15 (CTX-M-15; bla_{CTX-M-15}) polynucleotide” is meant a nucleotide molecule encoding a cefotaxime-hydrolyzing beta-lactamase polypeptide. A representative cefotaxime-hydrolyzing beta-lactamase polynucleotide sequence is provided below (NCBI® Ref. Seq. NZ_JABCMT010000035.1: c4224-3349):

>NZ JABCMT010000035.1: c4224-3349 Escherichia coli strain ME2L-20-10 NODE_35_length_11092_cov_13.291290, whole genome shotgun sequence

(SEQ ID NO: 3)
ATGGTTAAAAAATCACTGCGCCAGTTCACGCTGATGGCGACGGCAACCGTCACGCTGTTGTTAG
GAAGTGTGCCGCTGTATGCGCAAACGGCGGACGTACAGCAAAAACTTGCCGAATTAGAGCGGCA
GTCGGGAGGCAGACTGGGTGTGGCATTGATTAACACAGCAGATAATTCGCAAATACTTTATCGT
GCTGATGAGCGCTTTGCGATGTGCAGCACCAGTAAAGTGATGGCCGCGGCCGCGGTGCTGAAGA
AAAGTGAAAGCGAACCGAATCTGTTAAATCAGCGAGTTGAGATCAAAAAATCTGACCTTGTTAA
CTATAATCCGATTGCGGAAAAGCACGTCAATGGGACGATGTCACTGGCTGAGCTTAGCGCGGCC
GCGCTACAGTACAGCGATAACGTGGCGATGAATAAGCTGATTGCTCACGTTGGCGGCCCGGCTA
GCGTCACCGCGTTCGCCCGACAGCTGGGAGACGAAACGTTCCGTCTCGACCGTACCGAGCCGAC
GTTAAACACCGCCATTCCGGGCGATCCGCGTGATACCACTTCACCTCGGGCAATGGCGCAAACT
CTGCGGAATCTGACGCTGGGTAAAGCATTGGGCGACAGCCAACGGGCGCAGCTGGTGACATGGA
TGAAAGGCAATACCACCGGTGCAGCGAGCATTCAGGCTGGACTGCCTGCTTCCTGGGTTGTGGG
GGATAAAACCGGCAGCGGTGGCTATGGCACCACCAACGATATCGCGGTGATCTGGCCAAAAGAT
CGTGCGCCGCTGATTCTGGTCACTTACTTCACCCAGCCTCAACCTAAGGCAGAAAGCCGTCGCG
ATGTATTAGCGTCGGCGGCTAAAATCGTCACCGACGGTTTGTAA

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include a disease caused by a microbial pathogen (e.g., a blood-born pathogen). In some embodiments, the disease is a urinary tract infection. In some cases, the disease is caused by a prokaryotic organism, such as a gram-positive or a gram-negative bacterium.

By “effective amount” is meant the amount of an agent required to ameliorate the symptoms of a disease relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “elastase from Pseudomonas aeruginosa (lasB from P. aeruginosa) polypeptide” is meant a protein having elastase activity and with at least 85% sequence identity to NCBI® Ref. Seq. Accession No. WP_416059117.1, which is provided below, or a functional fragment thereof:

>WP 416059117.1 M4 family elastase LasB [Pseudomonas aeruginosa]

(SEQ ID NO: 4)
MKKVSTLDLLFVAIMGVSPAAFAADLIDVSKLPSKAAQGAPGPVTLQAAV
GAGGADELKAIRSTTLPNGKQVTRYEQFHNGVRVVGEAITEVKGPGKSVA
AQRSGHFVANIAADLPGSTTAAVSAEQVLAQAKSLKAQGRKTENDKVELV
IRLGENNIAQLVYNVSYLIPGEGLSRPHFVIDAKTGEVLDQWEGLAHAEA
GGPGGNQKIGKYTYGSDYGPLTVNDRCEMDDGNVITVDMNGSTDDSKTTP
FRFACPTNTYKQVNGAYSPLNDAHFFGGVVFKLYRDWFGTSPLTHKLYMK
VHYGRSVENAYWDGTAMLFGDGATMFYPLVSLDVAAHEVSHGFTEQNSGL
IYRGQSGGMNEAFSDMAGEAAEFYMRGKNDFLIGYDIKKGSGALRYMDQP
SRDGRSIDNASQYYNGIDVHHSSGVYNRAFYLLANSPGWDTRKAFEVFVD
ANRYYWTATSNYNSGACGVIRSAQNRNYSAADVTRAFSTVGVTCPSAL

By “elastase from Pseudomonas aeruginosa (lasB from P. aeruginosa) polynucleotide” is meant a nucleotide molecule encoding an elastase polypeptide. A representative elastase polynucleotide sequence is provided below (NCBI® Ref. Seq. NZ_CP180620.1: c4170966-4169470):

>NZ_CP180620.1: c4170966-4169470 Pseudomonas aeruginosa strain UPMP2478 chromosome

(SEQ ID NO: 5)
ATGAAGAAGGTTTCTACGCTTGACCTGTTGTTCGTTGCGATCATGGGTGTTTCGCCGGCCGCTT
TTGCCGCCGACCTGATCGACGTGTCCAAACTCCCCAGCAAGGCTGCCCAGGGCGCGCCCGGCCC
GGTCACCTTGCAAGCCGCGGTCGGCGCTGGCGGTGCCGATGAACTGAAAGCGATCCGCAGCACG
ACCCTGCCCAACGGCAAGCAGGTCACCCGCTACGAGCAATTCCACAACGGCGTACGGGTGGTCG
GCGAAGCCATCACCGAAGTCAAGGGTCCCGGCAAGAGCGTGGCGGCGCAGCGCAGCGGCCATTT
CGTCGCCAACATCGCCGCCGACCTGCCGGGCAGCACCACCGCGGCGGTATCCGCCGAGCAGGTG
CTGGCCCAGGCCAAGAGCCTGAAGGCCCAGGGCCGCAAGACCGAGAATGACAAAGTGGAACTGG
TGATCCGCCTGGGCGAGAACAACATCGCCCAACTGGTCTACAACGTCTCCTACCTGATTCCCGG
CGAGGGACTGTCGCGGCCGCATTTCGTCATCGACGCCAAGACCGGCGAAGTGCTCGATCAGTGG
GAAGGCCTGGCCCACGCCGAGGCGGGCGGCCCCGGCGGCAACCAGAAGATCGGCAAGTACACCT
ACGGTAGCGACTACGGTCCGCTGACCGTCAACGACCGCTGCGAGATGGACGACGGCAACGTCAT
CACCGTCGACATGAACGGCAGCACCGACGACAGCAAGACCACGCCGTTCCGCTTCGCCTGCCCG
ACCAACACCTACAAGCAGGTCAACGGCGCCTATTCGCCGCTGAACGACGCGCATTTCTTCGGCG
GCGTGGTGTTCAAACTGTACCGGGACTGGTTCGGCACCAGCCCGCTGACCCACAAGCTGTACAT
GAAGGTGCACTACGGGCGCAGCGTGGAGAACGCCTACTGGGACGGCACGGCGATGCTCTTCGGC
GACGGCGCCACCATGTTCTATCCGCTGGTGTCGCTGGACGTGGCGGCCCACGAGGTCAGCCACG
GCTTTACCGAGCAGAACTCCGGGCTGATCTACCGCGGGCAATCAGGCGGAATGAACGAAGCGTT
CTCCGACATGGCCGGCGAGGCAGCCGAGTTCTACATGCGCGGCAAGAACGACTTCCTGATCGGC
TACGACATCAAGAAGGGCAGCGGTGCGCTGCGCTACATGGACCAGCCCAGCCGCGACGGGCGAT
CCATCGACAACGCGTCGCAGTACTACAACGGCATCGACGTGCACCACTCCAGCGGCGTGTACAA
CCGTGCGTTCTACCTGTTGGCCAATTCGCCGGGCTGGGATACCCGCAAGGCCTTCGAGGTGTTC
GTCGACGCCAACCGCTACTACTGGACCGCCACCAGCAACTACAACAGCGGCGCCTGCGGGGTGA
TTCGCTCGGCGCAGAACCGCAACTACTCGGCGGCTGACGTCACCCGGGCGTTCAGCACCGTCGG
CGTGACCTGCCCGAGCGCGTTGTAA

By “fragment” is meant a portion of a polypeptide or nucleic acid molecule. In embodiments, portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In various embodiments, the fragment is a functional fragment.

By “imipenemase metallo-beta-lactamase (IMP; bla_{IMP}) polypeptide” is meant a protein having imipenemase metallo-beta-lactamase activity and with at least 85% sequence identity to GenBank® Ref. Seq. Accession No. UGK57400.1, which is provided below, or a functional fragment thereof:

>UGK57400.1 Subclass B1 beta-lactamase IMP family (plasmid) [Pseudomonas aeruginosa]

(SEQ ID NO: 111)
MKKLFVLCVCFFCSITAAGAALPDLKIEKLEEGVFVHTSFEEVNGWGVVT
KHGLVVLVNTDAYLIDTPFTATDTEKLVNWFVERGYEIKGTISSHFHSDS
TGGIEWLNSQSIPTYASELTNELLKKSGKVQAKYSFSEVSYWLVKNKIEV
FYPGPGHTQDNLVVWLPESKILFGGCFIKPHGLGNLGDANLEAWPKSAKI
LMSKYGKAKLVVSSHSEKGDASLMKRTWEQALKGLKESKKTSSPSN

By “imipenemase metallo-beta-lactamase (IMP; bla_{IMP}) polynucleotide” is meant a nucleotide molecule encoding an imipenemase metallo-beta-lactamase polypeptide. A representative imipenemase metallo-beta-lactamase polynucleotide sequence is provided below (GenBank® Ref. Seq. MZ581331.1:23341-24081):

>MZ581331.1:23341-24081 Pseudomonas aeruginosa strain 175 plasmid pCHUAC-04_IMP, complete sequence

(SEQ ID NO: 112)
ATGAAGAAATTATTTGTTTTATGTGTATGCTTCTTTTGTAGCATTACTGCCGCAGGAGCGGCTT
TACCTGATTTAAAAATCGAGAAGCTTGAAGAAGGTGTTTTTGTTCATACATCGTTCGAAGAGGT
TAACGGTTGGGGGGTTGTTACTAAACACGGTTTAGTGGTGCTTGTAAACACAGACGCCTATCTA
ATTGACACTCCATTTACTGCTACAGACACTGAAAAATTAGTCAATTGGTTTGTGGAGCGCGGCT
ATGAAATCAAAGGCACTATTTCATCACATTTCCATAGCGACAGCACAGGAGGAATAGAGTGGCT
TAATTCTCAATCTATTCCCACGTATGCATCTGAATTAACAAATGAACTTTTGAAAAAATCCGGT
AAGGTACAAGCTAAATATTCATTTAGCGAAGTTAGCTATTGGCTAGTTAAAAATAAAATTGAAG
TTTTCTACCCTGGCCCAGGTCACACTCAAGATAACCTAGTGGTTTGGTTGCCTGAAAGTAAAAT
TTTATTCGGTGGTTGCTTTATTAAACCTCACGGTCTTGGCAATTTAGGTGACGCAAATTTAGAA
GCTTGGCCAAAGTCCGCCAAAATATTAATGTCTAAATATGGCAAAGCAAAGCTTGTTGTTTCAA
GTCATAGTGAAAAAGGGGACGCATCACTAATGAAACGTACATGGGAACAAGCCCTTAAAGGGCT
TAAAGAAAGTAAAAAAACATCATCACCAAGTAACTAA

By “increase” is meant to alter positively relative to a reference. An increase may be by 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more.

The terms “isolated,” “purified,” or “biologically pure” refer to material that is free to varying degrees from components which normally accompany it as found in its native state. “Isolate” denotes a degree of separation from an original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “Klebsiella pneumonia carbapenemase 1 (KPC-1; bla_{KPC-1}) polypeptide” is meant a protein having carbapenem-hydrolyzing beta-lactamase activity and with at least 85% sequence identity to GenBank® Ref. Seq. Accession No. AAG13410.1, which is provided below, or a functional fragment thereof:

>AAG13410.1 carbepenem-hydrolyzing beta-lactamase KPC-1 [Klebsiella pneumoniae]

(SEQ ID NO: 6)
MSLYRRLVLLSCLSWPLAGFSATALTNLVAEPFAKLEQDFGGSIGVYAMDTGSGATVSYRAEER
FPLCSSFKGFLAAAVLARSQQQAGLLDTPIRYGKNALVPWSPISEKYLTTGMTVAELSAAAVQY
SDNAAANLLLKELGGPAGLTAFMRSIGDTTFRLDRWELELNSAIPSDARDTSSPRAVTESLOKL
TLGSALAAPQRQQFVDWLKGNTTGNHRIRAAVPADWAVGDKTGTCGVYGTANDYAVVWPTGRAP
IVLAVYTRAPNKDDKHSEAVIAAAARLALEGLGVNGQ

By “Klebsiella pneumonia carbapenemase 1 (KPC-1; bla_{KPC-1}) polynucleotide” is meant a nucleotide molecule encoding a carbapenem-hydrolyzing beta-lactamase polypeptide. A representative carbapenem-hydrolyzing beta-lactamase polynucleotide sequence is provided below (GenBank® Ref. Seq. AF297554.1).

>AF297554.1 Klebsiella pneumoniae carbepenem-hydrolyzing beta-lactamase KPC-1 gene, complete cds

(SEQ ID NO: 7)
CGGGGCAGTTACAGCCGTTACAGCCTCTGGAGAGGGAGCGGCTTGCCGCTCGGTGATAATCCCA
GCTGTAGCGGCCTGATTACATCCGGCCGCTACACCTAGCTCCACCTTCAAACAAGGAATATCGT
TGATGTCACTGTATCGCCGTCTAGTTCTGCTGTCTTGTCTCTCATGGCCGCTGGCTGGCTTTTC
TGCCACCGCGCTGACCAACCTCGTCGCGGAACCATTCGCTAAACTCGAACAGGACTTTGGCGGC
TCCATCGGTGTGTACGCGATGGATACCGGCTCAGGCGCAACTGTAAGTTACCGCGCTGAGGAGC
GCTTCCCACTGTGCAGCTCATTCAAGGGCTTTCTTGCTGCCGCTGTGCTGGCTCGCAGCCAGCA
GCAGGCCGGCTTGCTGGACACACCCATCCGTTACGGCAAAAATGCGCTGGTTCCGTGGTCACCC
ATCTCGGAAAAATATCTGACAACAGGCATGACGGTGGCGGAGCTGTCCGCGGCCGCCGTGCAAT
ACAGTGATAACGCCGCCGCCAATTTGTTGCTGAAGGAGTTGGGCGGCCCGGCCGGGCTGACGGC
CTTCATGCGCTCTATCGGCGATACCACGTTCCGTCTGGACCGCTGGGAGCTGGAGCTGAACTCC
GCCATCCCAAGCGATGCGCGCGATACCTCATCGCCGCGCGCCGTGACGGAAAGCTTACAAAAAC
TGACACTGGGCTCTGCACTGGCTGCGCCGCAGCGGCAGCAGTTTGTTGATTGGCTAAAGGGAAA
CACGACCGGCAACCACCGCATCCGCGCGGCGGTGCCGGCAGACTGGGCAGTCGGAGACAAAACC
GGAACCTGCGGAGTGTATGGCACGGCAAATGACTATGCCGTCGTCTGGCCCACTGGGCGCGCAC
CTATTGTGTTGGCCGTCTACACCCGGGCGCCTAACAAGGATGACAAGCACAGCGAGGCCGTCAT
CGCCGCTGCGGCTAGACTCGCGCTCGAGGGATTGGGCGTCAACGGGCAGTAAGGCTCTGAAAAT
CATCTATTGGCCCACCACCGCCGCCCTTGCGGGCGGCATGGATTACCAACCACTGTCACATTTA
GGCTAGGAGTCTGCGCGGCAGAGCCGTGTGACCGGTTTTCTGTAGAGCACTGACGATGGCGGCG
GCGCTCTCTGCAATTGGCAAGGCGTCGGCGCCAAGGATACCAATCTTGCGGCGCGCGGCGTGTT
ATGACGACTGGGGTGCATTTGAGCCGCCCCATTTAACCTTCGCCCTCACAGATACGCCATTCGC
CTCAAATTTAGCGCCATGCAGACGAGCTTCCACTCGGCTTGCACCTTGTCCAGGCCCCTCATGC
TGAACTGACGCAATCCCATCACCGCCTTGATCA

By “Klebsiella pneumonia carbapenemase 2 (KPC-2; bla_{KPC-2}) polypeptide” is meant a protein having carbapenem-hydrolyzing beta-lactamase activity and with at least 85% sequence identity to GenBank® Ref. Seq. Accession No. QBC88522.1, which is provided below, or a functional fragment thereof:

>QBC88522.1 Carbepenem-hydrolyzing beta-lactamase KPC2 (plasmid) [Klebsiella pneumoniae]

(SEQ ID NO: 8)
MSLYRRLVLLSCLSWPLAGFSATALTNLVAEPFAKLEQDFGGSIGVYAMDTGSGATVSYRAEER
FPLCSSFKGFLAAAVLARSQQQAGLLDTPIRYGKNALVPWSPISEKYLTTGMTVAELSAAAVQY
SDNAAANLLLKELGGPAGLTAFMRSIGDTTFRLDRWELELNSAIPGDARDTSSPRAVTESLQKL
TLGSALAAPQRQQFVDWLKGNTTGNHRIRAAVPADWAVGDKTGTCGVYGTANDYAVVWPTGRAP
IVLAVYTRAPNKDDKHSEAVIAAAARLALEGLGVNGQ

By “Klebsiella pneumonia carbapenemase 2 (KPC-2; bla_{KPC-2}) polynucleotide” is meant a nucleotide molecule encoding a carbapenem-hydrolyzing beta-lactamase polypeptide. A representative carbapenem-hydrolyzing beta-lactamase polynucleotide sequence is provided below (GenBank® Ref. Seq. MK264769.1:6675-7556):

>MK264769.1:6675-7556 Klebsiella pneumoniae strain B11 plasmid pKPB11, complete sequence

(SEQ ID NO: 9)
ATGTCACTGTATCGCCGTCTAGTTCTGCTGTCTTGTCTCTCATGGCCGCTGGCTGGCTTTTCTG
CCACCGCGCTGACCAACCTCGTCGCGGAACCATTCGCTAAACTCGAACAGGACTTTGGCGGCTC
CATCGGTGTGTACGCGATGGATACCGGCTCAGGCGCAACTGTAAGTTACCGCGCTGAGGAGCGC
TTCCCACTGTGCAGCTCATTCAAGGGCTTTCTTGCTGCCGCTGTGCTGGCTCGCAGCCAGCAGC
AGGCCGGCTTGCTGGACACACCCATCCGTTACGGCAAAAATGCGCTGGTTCCGTGGTCACCCAT
CTCGGAAAAATATCTGACAACAGGCATGACGGTGGCGGAGCTGTCCGCGGCCGCCGTGCAATAC
AGTGATAACGCCGCCGCCAATTTGTTGCTGAAGGAGTTGGGCGGCCCGGCCGGGCTGACGGCCT
TCATGCGCTCTATCGGCGATACCACGTTCCGTCTGGACCGCTGGGAGCTGGAGCTGAACTCCGC
CATCCCAGGCGATGCGCGCGATACCTCATCGCCGCGCGCCGTGACGGAAAGCTTACAAAAACTG
ACACTGGGCTCTGCACTGGCTGCGCCGCAGCGGCAGCAGTTTGTTGATTGGCTAAAGGGAAACA
CGACCGGCAACCACCGCATCCGCGCGGCGGTGCCGGCAGACTGGGCAGTCGGAGACAAAACCGG
AACCTGCGGAGTGTATGGCACGGCAAATGACTATGCCGTCGTCTGGCCCACTGGGCGCGCACCT
ATTGTGTTGGCCGTCTACACCCGGGCGCCTAACAAGGATGACAAGCACAGCGAGGCCGTCATCG
CCGCTGCGGCTAGACTCGCGCTCGAGGGATTGGGCGTCAACGGGCAGTAA

By “Klebsiella pneumonia carbapenemase 3 (KPC-3; bla_{KPC-3}) polypeptide” is meant a protein having carbapenem-hydrolyzing beta-lactamase activity and with at least 85% sequence identity to GenBank® Ref. Seq. Accession No. AEV55249.1, which is provided below, or a functional fragment thereof:

>AEV55249.1 KPC-3 (plasmid) [Klebsiella pneumoniae]

(SEQ ID NO: 10)
MSLYRRLVLLSCLSWPLAGFSATALTNLVAEPFAKLEQDFGGSIGVYAMDTGSGATVSYRAEER
FPLCSSFKGFLAAAVLARSQQQAGLLDTPIRYGKNALVPWSPISEKYLTTGMTVAELSAAAVQY
SDNAAANLLLKELGGPAGLTAFMRSIGDTTFRLDRWELELNSAIPGDARDTSSPRAVTESLOKL
TLGSALAAPQRQQFVDWLKGNTTGNHRIRAAVPADWAVGDKTGTCGVYGTANDYAVVWPTGRAP
IVLAVYTRAPNKDDKYSEAVIAAAARLALEGLGVNGQ

By “Klebsiella pneumonia carbapenemase 3 (KPC-3; bla_{KPC-3}) polynucleotide” is meant a nucleotide molecule encoding a carbapenem-hydrolyzing beta-lactamase polypeptide. A representative carbapenem-hydrolyzing beta-lactamase polynucleotide sequence is provided below (GenBank® Ref. Seq. JN233705.2: c9555-8674):

>JN233705.2: c9555-8674 Klebsiella pneumoniae strain ST258 plasmid pKpQIL-IT, complete sequence

(SEQ ID NO: 11)
ATGTCACTGTATCGCCGTCTAGTTCTGCTGTCTTGTCTCTCATGGCCGCTGGCTGGCTTTTCTG
CCACCGCGCTGACCAACCTCGTCGCGGAACCATTCGCTAAACTCGAACAGGACTTTGGCGGCTC
CATCGGTGTGTACGCGATGGATACCGGCTCAGGCGCAACTGTAAGTTACCGCGCTGAGGAGCGC
TTCCCACTGTGCAGCTCATTCAAGGGCTTTCTTGCTGCCGCTGTGCTGGCTCGCAGCCAGCAGC
AGGCCGGCTTGCTGGACACACCCATCCGTTACGGCAAAAATGCGCTGGTTCCGTGGTCACCCAT
CTCGGAAAAATATCTGACAACAGGCATGACGGTGGCGGAGCTGTCCGCGGCCGCCGTGCAATAC
AGTGATAACGCCGCCGCCAATTTGTTGCTGAAGGAGTTGGGGGGCCCGGCCGGGCTGACGGCCT
TCATGCGCTCTATCGGCGATACCACGTTCCGTCTGGACCGCTGGGAGCTGGAGCTGAACTCCGC
CATCCCAGGCGATGCGCGCGATACCTCATCGCCGCGCGCCGTGACGGAAAGCTTACAAAAACTG
ACACTGGGCTCTGCACTGGCTGCGCCGCAGCGGCAGCAGTTTGTTGATTGGCTAAAGGGAAACA
CGACCGGCAACCACCGCATCCGCGCGGCGGTGCCGGCAGACTGGGCAGTCGGAGACAAAACCGG
AACCTGCGGAGTGTATGGCACGGCAAATGACTATGCCGTCGTCTGGCCCACTGGGCGCGCACCT
ATTGTGTTGGCCGTCTACACCCGGGCGCCTAACAAGGATGACAAGTACAGCGAGGCCGTCATCG
CCGCTGCGGCTAGACTCGCGCTCGAGGGATTGGGCGTCAACGGGCAGTAA

By “marker” is meant any protein or polynucleotide having an alteration in expression level or activity that is associated with a developmental state, condition, disease, or disorder. As used herein, “detection construct” refers to a molecule that mediates the production of a detectable signal, or a detectable change or increase in signal, only when cleaved or otherwise deactivated by a nuclease. In some embodiments, a detection construct is an RNA-based detection construct as described herein. In some embodiments, the RNA-based detection construct contains an RNA molecule containing a fluorescent dye and a quencher (e.g., bound at opposite ends of the RNA molecule) and cleavage of the RNA-based detection construct results in production of a detectable fluorescent signal by the fluorescent dye. The nuclease may be a Type VI CRISPR polypeptide described herein.

By “New Delhi metallo-beta-lactamase 1 (NDM-1; bla_{NDM-1}) polypeptide” is meant a protein having carbapenem-hydrolyzing beta-lactamase activity and with at least 85% sequence identity to NCBI® Ref. Seq. Accession No. WP_004201164.1, which is provided below, or a functional fragment thereof:

>WP_004201164.1 MULTISPECIES: subclass B1 metallo-beta-lactamase NDM-1 [Pseudomonadota]

(SEQ ID NO: 12)
MELPNIMHPVAKLSTALAAALMLSGCMPGEIRPTIGQQMETGDQRFGDLVFRQLAPNVWOHTSY
LDMPGFGAVASNGLIVRDGGRVLVVDTAWTDDQTAQILNWIKQEINLPVALAVVTHAHQDKMGG
MDALHAAGIATYANALSNQLAPQEGMVAAQHSLTFAANGWVEPATAPNFGPLKVFYPGPGHTSD
NITVGIDGTDIAFGGCLIKDSKAKSLGNLGDADTEHYAASARAFGAAFPKASMIVMSHSAPDSR
AAITHTARMADKLR

By “New Delhi metallo-beta-lactamase 1 (NDM-1; bla_{NDM-1}) polynucleotide” is meant a nucleotide molecule encoding a carbapenem-hydrolyzing beta-lactamase polypeptide. A representative carbapenem-hydrolyzing beta-lactamase polynucleotide sequence is provided below (NCBI® Ref. Seq. NC_015872.1: c33506-32694):

>NC_015872.1: c33506-32694 Escherichia coli plasmid p271A, complete sequence

(SEQ ID NO: 13)
ATGGAATTGCCCAATATTATGCACCCGGTCGCGAAGCTGAGCACCGCATTAGCCGCTGCATTGA
TGCTGAGCGGGTGCATGCCCGGTGAAATCCGCCCGACGATTGGCCAGCAAATGGAAACTGGCGA
CCAACGGTTTGGCGATCTGGTTTTCCGCCAGCTCGCACCGAATGTCTGGCAGCACACTTCCTAT
CTCGACATGCCGGGTTTCGGGGCAGTCGCTTCCAACGGTTTGATCGTCAGGGATGGCGGCCGCG
TGCTGGTGGTCGATACCGCCTGGACCGATGACCAGACCGCCCAGATCCTCAACTGGATCAAGCA
GGAGATCAACCTGCCGGTCGCGCTGGCGGTGGTGACTCACGCGCATCAGGACAAGATGGGCGGT
ATGGACGCGCTGCATGCGGCGGGGATTGCGACTTATGCCAATGCGTTGTCGAACCAGCTTGCCC
CGCAAGAGGGGATGGTTGCGGCGCAACACAGCCTGACTTTCGCCGCCAATGGCTGGGTCGAACC
AGCAACCGCGCCCAACTTTGGCCCGCTCAAGGTATTTTACCCCGGCCCCGGCCACACCAGTGAC
AATATCACCGTTGGGATCGACGGCACCGACATCGCTTTTGGTGGCTGCCTGATCAAGGACAGCA
AGGCCAAGTCGCTCGGCAATCTCGGTGATGCCGACACTGAGCACTACGCCGCGTCAGCGCGCGC
GTTTGGTGCGGCGTTCCCCAAGGCCAGCATGATCGTGATGAGCCATTCCGCCCCCGATAGCCGC
GCCGCAATCACTCATACGGCCCGCATGGCCGACAAGCTGCGCTGA

As used herein, “obtaining” as in “obtaining an agent” includes synthesizing, purchasing, or otherwise acquiring the agent.

By “outer membrane heme/hemoglobin receptor from Escherichia coli (chuA from E. coli) polypeptide” is meant a protein having iron-regulated haem-transport activity and with at least 85% sequence identity to GenBank® Ref. Seq. Accession No. AAG58641.1, which is provided below, or a functional fragment thereof:

>AAG58641.1 outer membrane heme/hemoglobin receptor [Escherichia coli 0157:H7 str. EDL933]

(SEQ ID NO: 14)
MSRPQFTSLRLSLLALAVSATLPTFAFATETMTVTATGNARSSFEAPMMVSVIDTSAPENQTAT
SATDLLRHVPGITLDGTGRTNGQDVNMRGYDHRGVLVLVDGVROGTDTGHLNGTFLDPALIKRV
EIVRGPSALLYGSGALGGVISYDTVDAKDLLQEGQSSGFRVFGTGGTGDHSLGLGASAFGRTEN
LDGIVAWSSRDRGDLRQSNGETAPNDESINNMLAKGTWQIDSAQSLSGLVRYYNNDAREPKNPQ
TVGASESSNPMVDRSTIQRDAQLSYKLAPQGNDWLNADAKIYWSEVRINAQNTGSSGEYREQIT
KGARLENRSTLFADSFASHLLTYGGEYYRQEQHPGGATTGFPQAKIDFSSGWLQDEITLRDLPI
TLLGGTRYDSYRGSSDGYKDVDADKWSSRAGMTINPTNWLMLFGSYAQAFRAPTMGEMYNDSKH
FSIGRFYTNYWVPNPNLRPETNETQEYGFGLRFDDLMLSNDALEFKASYFDTKAKDYISTTVDF
AAATTMSYNVPNAKIWGWDVMTKYTTDLFSLDVAYNRTRGKDTDTGEYISSINPDTVTSTLNIP
IAHSGFSVGWVGTFADRSTHISSSYSKQPGYGVNDFYVSYQGQQALKGMTTTLVLGNAFDKEYW
SPQGIPQDGRNGKIFVSYQW

By “outer membrane heme/hemoglobin receptor from Escherichia coli (chuA from E. coli) polynucleotide” is meant a nucleotide molecule encoding an iron-regulated haem-transport polypeptide. A representative iron-regulated haem-transport polynucleotide sequence is provided below (GenBank® Ref. Seq. AE005174.2: c4458673-4456691).

>AE005174.2: c4458673-4456691 Escherichia coli 0157:H7 str. EDL933 genome

(SEQ ID NO: 15)
ATGTCACGTCCGCAATTTACCTCGTTGCGTTTGAGTTTATTGGCCTTAGCTGTTTCTGCCACCT
TGCCAACGTTTGCTTTTGCTACTGAAACCATGACCGTTACGGCAACGGGGAATGCCCGTAGTTC
CTTCGAAGCGCCTATGATGGTCAGCGTCATCGACACTTCCGCTCCTGAAAATCAAACGGCTACT
TCAGCCACCGATCTGCTGCGTCATGTTCCTGGAATTACTCTGGATGGTACCGGACGAACCAACG
GTCAGGATGTAAATATGCGTGGCTATGATCATCGCGGCGTGCTGGTTCTTGTCGATGGTGTTCG
TCAGGGAACGGATACCGGACACCTGAATGGCACTTTTCTCGATCCGGCGCTGATCAAGCGTGTT
GAGATTGTTCGTGGACCTTCAGCATTACTGTATGGCAGTGGCGCGCTGGGTGGAGTGATCTCCT
ACGATACGGTCGATGCAAAAGATTTATTGCAGGAAGGACAAAGCAGTGGTTTTCGTGTCTTTGG
TACTGGCGGCACGGGGGACCATAGCCTGGGATTAGGCGCGAGCGCGTTTGGGCGAACTGAAAAT
CTGGATGGTATTGTGGCCTGGTCCAGTCGCGATCGGGGTGATTTACGCCAGAGCAATGGTGAAA
CCGCGCCGAATGACGAGTCCATTAATAACATGCTGGCGAAAGGGACCTGGCAAATTGATTCAGC
CCAGTCTCTGAGCGGTTTAGTGCGTTACTACAACAACGACGCGCGTGAACCAAAAAATCCGCAG
ACCGTTGGGGCTTCTGAAAGCAGCAACCCGATGGTTGATCGTTCAACAATTCAACGCGATGCGC
AGCTTTCTTATAAACTCGCCCCGCAGGGCAACGACTGGTTAAATGCAGATGCAAAAATTTATTG
GTCGGAAGTCCGTATTAATGCGCAAAACACGGGGAGTTCCGGCGAGTATCGTGAACAGATAACA
AAAGGAGCCAGGCTGGAGAACCGTTCCACTCTCTTTGCCGACAGTTTCGCTTCTCACTTACTGA
CATATGGCGGTGAGTATTATCGTCAGGAACAACATCCGGGCGGCGCGACGACGGGCTTCCCGCA
AGCAAAAATCGATTTTAGCTCCGGCTGGCTACAGGATGAGATCACCTTACGCGATCTGCCGATT
ACCCTGCTTGGCGGAACCCGCTATGACAGTTATCGCGGTAGCAGTGACGGTTACAAAGATGTTG
ATGCCGACAAATGGTCATCTCGTGCGGGGATGACTATCAATCCGACTAACTGGCTGATGTTATT
TGGCTCATATGCCCAGGCATTCCGCGCCCCGACGATGGGCGAAATGTATAACGATTCTAAGCAC
TTCTCGATTGGTCGCTTCTATACCAACTATTGGGTGCCAAACCCGAACTTACGTCCGGAAACTA
ACGAAACTCAGGAGTACGGTTTTGGGCTGCGTTTTGATGACCTGATGTTGTCCAATGATGCTCT
GGAATTTAAAGCCAGCTACTTTGATACCAAAGCGAAGGATTACATCTCCACGACCGTCGATTTC
GCGGCGGCGACGACTATGTCGTATAACGTCCCGAACGCCAAAATCTGGGGCTGGGATGTGATGA
CGAAATATACCACTGATCTGTTTAGCCTTGATGTGGCCTATAACCGTACCCGCGGCAAAGACAC
CGATACCGGCGAATACATCTCCAGCATTAACCCGGATACTGTTACCAGCACTCTGAATATTCCG
ATCGCTCACAGTGGCTTCTCTGTTGGGTGGGTTGGTACGTTTGCCGATCGCTCAACACATATCA
GCAGCAGTTACAGCAAACAACCAGGCTATGGCGTGAATGATTTCTACGTCAGTTATCAAGGACA
ACAGGCGCTCAAAGGTATGACCACTACTTTGGTGTTGGGTAACGCTTTCGACAAAGAGTACTGG
TCGCCGCAAGGCATCCCACAGGATGGTCGTAACGGAAAAATTTTCGTGAGTTATCAATGGTAA

By “oxacillinase 48 (OXA-48; bla_{OXA-48}) polypeptide” is meant a protein having oxacillin-hydrolyzing beta-lactamase activity and with at least 85% sequence identity to NCBI® Ref. Seq. Accession No. WP_015059991.1, which is provided below, or a functional fragment thereof:

>WP_015059991.1 MULTISPECIES: OXA-48 family carbapenem-hydrolyzing class D beta-lactamase OXA-48 [Gammaproteobacteria]

(SEQ ID NO: 16)
MRVLALSAVFLVASIIGMPAVAKEWQENKSWNAHFTEHKSQGVVVLWNENKQQGFTNNLKRANQ
AFLPASTFKIPNSLIALDLGVVKDEHQVFKWDGQTRDIATWNRDHNLITAMKYSVVPVYQEFAR
QIGEARMSKMLHAFDYGNEDISGNVDSFWLDGGIRISATEQISFLRKLYHNKLHVSERSQRIVK
QAMLTEANGDYIIRAKTGYSTRIEPKIGWWVGWVELDDNVWFFAMNMDMPTSDGLGLRQAITKE
VLKQEKIIP

By “oxacillinase 48 (OXA-48; bla_{OXA-48}) polynucleotide” is meant a nucleotide molecule encoding an oxacillin-hydrolyzing beta-lactamase polypeptide. A representative oxacillin-hydrolyzing beta-lactamase polynucleotide sequence is provided below (NCBI® Ref. Seq. NZ_AP025039.1: c12252-11455):

>NZ_AP025039.1: c12252-11455 Escherichia coli strain NIHE15-1569 plasmid pNIHE15-1569_oxa, complete sequence

(SEQ ID NO: 17)
ATGCGTGTATTAGCCTTATCGGCTGTGTTTTTGGTGGCATCGATTATCGGAATGCCTGCGGTAG
CAAAGGAATGGCAAGAAAACAAAAGTTGGAATGCTCACTTTACTGAACATAAATCACAGGGCGT
AGTTGTGCTCTGGAATGAGAATAAGCAGCAAGGATTTACCAATAATCTTAAACGGGCGAACCAA
GCATTTTTACCCGCATCTACCTTTAAAATTCCCAATAGCTTGATCGCCCTCGATTTGGGCGTGG
TTAAGGATGAACACCAAGTCTTTAAGTGGGATGGACAGACGCGCGATATCGCCACTTGGAATCG
CGATCATAATCTAATCACCGCGATGAAATATTCAGTTGTGCCTGTTTATCAAGAATTTGCCCGC
CAAATTGGCGAGGCACGTATGAGCAAGATGCTACATGCTTTCGATTATGGTAATGAGGACATTT
CGGGCAATGTAGACAGTTTCTGGCTCGACGGTGGTATTCGAATTTCGGCCACGGAGCAAATCAG
CTTTTTAAGAAAGCTGTATCACAATAAGTTACACGTATCGGAGCGCAGCCAGCGTATTGTCAAA
CAAGCCATGCTGACCGAAGCCAATGGTGACTATATTATTCGGGCTAAAACTGGATACTCGACTA
GAATCGAACCTAAGATTGGCTGGTGGGTCGGTTGGGTTGAACTTGATGATAATGTGTGGTTTTT
TGCGATGAATATGGATATGCCCACATCGGATGGTTTAGGGCTGCGCCAAGCCATCACAAAAGAA
GTGCTCAAACAGGAAAAAATTATTCCCTAG

By “oxacillinase 232 (OXA-232; bla_{OXA-232}) polypeptide” is meant a protein having oxacillin-hydrolyzing beta-lactamase activity and with at least 85% sequence identity to NCBI® Ref Seq. Accession No. WP_043907054.1, which is provided below, or a functional fragment thereof:

>WP 043907054.1 MULTISPECIES: OXA-48 family carbapenem-hydrolyzing class D beta-lactamase OXA-232 [Gammaproteobacteria]

(SEQ ID NO: 18)
MRVLALSAVFLVASIIGMPAVAKEWQENKSWNAHFTEHKSQGVVVLWNENKQQGFTNNLKRANQ
AFLPASTFKIPNSLIALDLGVVKDEHQVFKWDGQTRDIAAWNRDHDLITAMKYSVVPVYQEFAR
QIGEARMSKMLHAFDYGNEDISGNVDSFWLDGGIRISATQQIAFLRKLYHNKLHVSERSQRIVK
QAMLTEANGDYIIRAKTGYSTSIEPKIGWWVGWVELDDNVWFFAMNMDMPTSDGLGLRQAITKE
VLKQEKIIP

By “oxacillinase 232 (OXA-232; bla_{OXA-232}) polynucleotide” is meant a nucleotide molecule encoding an oxacillin-hydrolyzing beta-lactamase polypeptide. A representative oxacillin-hydrolyzing beta-lactamase polynucleotide sequence is provided below (NCBI® Ref. Seq. NG_049528.1:101-898):

>NG_049528.1:101-898 Escherichia coli blaOXA gene for OXA-48 family carbapenem-hydrolyzing class D beta-lactamase OXA-232, complete CDS

(SEQ ID NO: 19)
ATGCGTGTATTAGCCTTATCGGCTGTGTTTTTGGTGGCATCGATTATCGGAATGCCAGCGGTAG
CAAAGGAATGGCAAGAAAACAAAAGTTGGAATGCTCACTTTACTGAACATAAATCACAGGGCGT
AGTTGTGCTCTGGAATGAGAATAAGCAGCAAGGATTTACCAATAATCTTAAACGGGCGAACCAA
GCATTTTTACCCGCATCTACCTTTAAAATTCCCAATAGCTTGATCGCCCTCGATTTGGGCGTGG
TTAAGGATGAACACCAAGTCTTTAAGTGGGATGGACAGACGCGTGATATCGCCGCTTGGAATCG
TGACCATGACTTAATTACCGCGATGAAGTACTCAGTTGTGCCTGTTTATCAAGAATTTGCCCGC
CAAATTGGTGAGGCACGTATGAGTAAAATGCTGCACGCCTTCGATTATGGCAATGAGGATATCT
CGGGCAATGTAGACAGTTTTTGGCTCGATGGTGGTATTCGCATTTCGGCTACCCAGCAAATCGC
TTTTTTACGCAAGCTGTATCACAACAAGCTGCACGTTTCTGAGCGTAGTCAGCGCATCGTGAAA
CAAGCCATGCTGACCGAAGCCAATGGCGACTATATTATTCGGGCTAAAACGGGATACTCGACTA
GTATCGAACCTAAGATTGGCTGGTGGGTTGGTTGGGTTGAACTTGATGATAATGTGTGGTTTTT
TGCGATGAATATGGATATGCCCACATCGGATGGTTTAGGGCTGCGCCAAGCCATCACAAAAGAA
GTGCTCAAACAGGAGAAAATTATTCCCTAG

By “PBP2A transpeptidase (mecA) polypeptide” is meant a protein having methicillin-resistant transpeptidase activity and with at least 85% sequence identity to GenBank® Ref. Seq. Accession No. AGC51118.1, which is provided below, or a functional fragment thereof:

>AGC51118.1 MecA [Staphylococcus aureus]

(SEQ ID NO: 113)
MKKIKIVPLILIVVVVGFGIYFYASKDKEINNTIDAIEDKNFKQVYKDSSYISKSDNGEVEMTE
RPIKIYNSLGVKDINIQDRKIKKVSKNKKRVDAQYKIKTNYGNIDRNVQFNFVKEDGMWKLDWD
HSVIIPGMQKDQSIHIENLKSERGKILDRNNVELANTGTAYEIGIVPKNVSKKDYKAIAKELSI
SEDYIKQQMDQNWVQDDTFVPLKTVKKMDEYLSDFAKKFHLTTNETKSRNYPLEKATSHLLGYV
GPINSEELKQKEYKGYKDDAVIGKKGLEKLYDKKLQHEDGYRVTIVDDNSNTIAHTLIEKKKKD
GKDIQLTIDAKVQKSIYNNMKNDYGSGTAIHPQTGELLALVSTPSYDVYPFMYGMSNEEYNKLT
EDKKEPLLNKFQITTSPGSTQKILTAMIGLNNKTLDDKTSYKIDGKGWQKDKSWGGYNVTRYEV
VNGNIDLKQAIESSDNIFFARVALELGSKKFEKGMKKLGVGEDIPSDYPFYNAQISNKNLDNEI
LLADSGYGQGEILINPVQILSIYSALENNGNINAPHLLKDTKNKVWKKNIISKENINLLTDGMQ
QVVNKTHKEDIYRSYANLIGKSGTAELKMKQGETGRQIGWFISYDKDNPNMMMAINVKDVQDKG
MASYNAKISGKVYDELYENGNKKYDIDE

By “PBP2A transpeptidase (mecA) polynucleotide” is meant a nucleotide molecule encoding a methicillin-resistant transpeptidase polypeptide. A representative methicillin-resistant transpeptidase polynucleotide sequence is provided below (NCBI® Ref. Seq. KC243783.1:1-2007):

>KC243783.1:1-2007 Staphylococcus aureus strain TN/CN/1/12 MecA (mecA) gene, complete cds

(SEQ ID NO: 114)
ATGAAAAAGATAAAAATTGTTCCACTTATTTTAATAGTTGTAGTTGTCGGGTTTGGTATATATT
TTTATGCTTCAAAAGATAAAGAAATTAATAATACTATTGATGCAATTGAAGATAAAAATTTCAA
ACAAGTTTATAAAGATAGCAGTTATATTTCTAAAAGCGATAATGGTGAAGTAGAAATGACTGAA
CGTCCGATAAAAATATATAATAGTTTAGGCGTTAAAGATATAAACATTCAGGATCGTAAAATAA
AAAAAGTATCTAAAAATAAAAAACGAGTAGATGCTCAATATAAAATTAAAACAAACTACGGTAA
CATTGATCGCAACGTTCAATTTAATTTTGTTAAAGAAGATGGTATGTGGAAGTTAGATTGGGAT
CATAGCGTCATTATTCCAGGAATGCAGAAAGACCAAAGCATACATATTGAAAATTTAAAATCAG
AACGTGGTAAAATTTTAGACCGAAACAATGTGGAATTGGCCAATACAGGAACAGCATATGAGAT
AGGCATCGTTCCAAAGAATGTATCTAAAAAAGATTATAAAGCAATCGCTAAAGAACTAAGTATT
TCTGAAGACTATATCAAACAACAAATGGATCAAAATTGGGTACAAGATGATACCTTCGTTCCAC
TTAAAACCGTTAAAAAAATGGATGAATATTTAAGTGATTTCGCAAAAAAATTTCATCTTACAAC
TAATGAAACAAAAAGTCGTAACTATCCTCTAGAAAAAGCGACTTCACATCTATTAGGTTATGTT
GGTCCCATTAACTCTGAAGAATTAAAACAAAAAGAATATAAAGGCTATAAAGATGATGCAGTTA
TTGGTAAAAAGGGACTCGAAAAACTTTACGATAAAAAGCTCCAACATGAAGATGGCTATCGTGT
CACAATCGTTGACGATAATAGCAATACAATCGCACATACATTAATAGAGAAAAAGAAAAAAGAT
GGCAAAGATATTCAACTAACTATTGATGCTAAAGTTCAAAAGAGTATTTATAACAACATGAAAA
ATGATTATGGCTCAGGTACTGCTATCCACCCTCAAACAGGTGAATTATTAGCACTTGTAAGCAC
ACCTTCATATGACGTCTATCCATTTATGTATGGCATGAGTAACGAAGAATATAATAAATTAACC
GAAGATAAAAAAGAACCTCTGCTCAACAAGTTCCAGATTACAACTTCACCAGGTTCAACTCAAA
AAATATTAACAGCAATGATTGGGTTAAATAACAAAACATTAGACGATAAAACAAGTTATAAAAT
CGATGGTAAAGGTTGGCAAAAAGATAAATCTTGGGGTGGTTACAACGTTACAAGATATGAAGTG
GTAAATGGTAATATCGACTTAAAACAAGCAATAGAATCATCAGATAACATTTTCTTTGCTAGAG
TAGCACTCGAATTAGGCAGTAAGAAATTTGAAAAAGGCATGAAAAAACTAGGTGTTGGTGAAGA
TATACCAAGTGATTATCCATTTTATAATGCTCAAATTTCAAACAAAAATTTAGATAATGAAATA
TTATTAGCTGATTCAGGTTACGGACAAGGTGAAATACTGATTAACCCAGTACAGATCCTTTCAA
TCTATAGCGCATTAGAAAATAATGGCAATATTAACGCACCTCACTTATTAAAAGACACGAAAAA
CAAAGTTTGGAAGAAAAATATTATTTCCAAAGAAAATATCAATCTATTAACTGATGGTATGCAA
CAAGTCGTAAATAAAACACATAAAGAAGATATTTATAGATCTTATGCAAACTTAATTGGCAAAT
CCGGTACTGCAGAACTCAAAATGAAACAAGGAGAAACTGGCAGACAAATTGGGTGGTTTATATC
ATATGATAAAGATAATCCAAACATGATGATGGCTATTAATGTTAAAGATGTACAAGATAAAGGA
ATGGCTAGCTACAATGCCAAAATCTCAGGTAAAGTGTATGATGAGCTATATGAGAACGGTAATA
AAAAATACGATATAGATGAATAA

By “positive blood culture” is meant a cultured blood sample showing positive signs of microbial growth. In various embodiments, culturing a blood sample involves adding blood to a bottle containing a medium appropriate for enhancing microbial growth and allowing the bottle to incubate at room temperature or body temperature (e.g., 37 deg. C.) for a set period of time (e.g., 1, 2, 3, 4, or 5 days) or until positive signs of microbial activity are observed (e.g., cloudiness, production of gas, presence of microbial colonies, change in color, hemolysis, etc.). Methods for culturing a blood sample are familiar to one of skill in the art (see, e.g., Weinstein, “Current Blood Culture Methods and Systems: Clinical Concepts, Technology, and Interpretation of Results,” Clinical Infectious Diseases, 23:40-46 (1996)); and Perker, et al., “Diagnosis of bloodstream infections from positive blood cultures and directly from blood samples: recent developments in molecular approaches,” Clinical Microbiology and Infection, 24:944-955 (2018)). In various embodiments, a positive blood culture contains a concentration of a microbial species of interest of at least about 500 CFU/μL, 1,000 CFU/μL, 5,000 CFU/μL, 10,000 CFU/μL, 15,000 CFU/μL, 20,000 CFU/μL, 25,000 CFU/μL, 30,000 CFU/μL, 35,000 CFU/μL, 40,000 CFU/μL, 50,000 CFU/μL, 75,000 CFU/μL, or 100,000 CFU/μL.

As used herein, the terms “prevent,” “preventing,” “prevention,” “prophylactic treatment” and the like refer to reducing the probability of developing, a disorder or condition in a subject, who does not have, but is at risk of or susceptible to developing a disorder or condition.

By “polynucleotide” or “nucleic acid molecule” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the disclosure embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

The term “primer set” means a set of oligonucleotides that may be used, for example, for amplification of a nucleotide molecule or a portion thereof. In some embodiments, the primer set is suitable for use in PCR or an isothermal amplification method (e.g., recombinase polymerase amplification (RPA)). A primer set would consist of at least about 2, 4, 6, 8, 10, 12, 14, 16, 18, 20, 30, 40, 50, 60, 80, 100, 200, 250, 300, 400, 500, 600, or more primers.

By “recombinase polymerase amplification (RPA)” is meant an isothermal nucleic acid amplification method where a recombinase enzyme is used to mediate the binding of primers to a double-stranded DNA molecule to be extended by a strand-displacing DNA polymerase. In various embodiments, RPA involves the use of a UVSX polypeptide as the recombinase enzyme. Non-limiting examples of primers suitable for use in RPA for identification of a target bacterium include those listed in the below table or in Table 1.

TABLE A
Representative RPA primers. Lowercase letters represent a T7 promoter
sequence.

| | | SEQ ID | | SEQ ID |
|---|---|---|---|---|
| Target Species | Forward Primer | NO | Reverse Primer | NO |
| Staphylococcus | gaaattaatacgactcactat | 20 | GGACCATATTTCTCTAC | 32 |
| aureus | agggACAAAGGTCAACCAATG | | ACCTTTTTTAGGA | |
| | ACATTCAGACTAT | | | |
| Staphylococcus | gaaattaatacgactcactat | 21 | CCCCTGTTTACCTTTGG | 33 |
| lugdunensis | agggAACAAGAAGCAGCGCGT | | CTT | |
| | AAA | | | |
| Staphylococcus | gaaattaatacgactcactat | 22 | TCAATACCTTCATATAA | 34 |
| hemolyticus | agggTAATCCATTTACCACTT | | TTGTTGCGCGAGCAT | |
| | CAACATTACAACAAGA | | | |
| Staphylococcus | gaaattaatacgactcactat | 23 | CCTTCAATGGACCAATA | 35 |
| epidermidis | agggGATTTAGTTGACGCGCA | | CTCTTCAGGTTTA | |
| | ACAAGCACGTCGTATT | | | |
| Staphylococcus | gaaattaatacgactcactat | 24 | AAGCAACTGATTGTACT | 36 |
| hominis | agggGAATTTAGTTGATGCAC | | CGACCAGCTGACAATC | |
| | AACAAGCGCGTCGT | | | |
| Staphylococcus | gaaattaatacgactcactat | 25 | TTATATCTAAATTCCCC | 37 |
| capitis | agggACGTGTTCAGTCAGTAG | | TTCAATAGTCCAATAT | |
| | CTCTTCGTTTAGTTA | | | |
| Staphylococcus | gaaattaatacgactcactat | 26 | CAATCTTTGTTGCTGTC | 38 |
| pettenkoferi | agggAAAGGCTTTATGTCTGT | | ACCATCTCCCCTTCT | |
| | ATACGTCGAAGCGAA | | | |
| Streptococcus | gaaattaatacgactcactat | 27 | GTGACAAATGGAGTTGA | 39 |
| constellatus | agggCCATCTTTTGATACAGT | | CGGATTGTTAGAAA | |
| | CCATGACTTAGCCATT | | | |
| Streptococcus | gaaattaatacgactcactat | 27 | GTGACAAATGGAGTTGA | 39 |
| intermedius | agggCCATCTTTTGATACAGT | | CGGATTGTTAGAAA | |
| | CCATGACTTAGCCATT | | | |
| Streptococcus | gaaattaatacgactcactat | 27 | GTGACAAATGGAGTTGA | 39 |
| anginosus | agggCCATCTTTTGATACAGT | | CGGATTGTTAGAAA | |
| | CCATGACTTAGCCATT | | | |
| Streptococcus | gaaattaatacgactcactat | 28 | ACTTGCTTCTGAAAACC | 40 |
| agalactiae | agggAATGTTACCAGATATGG | | TTGCAGGAGGTTGA | |
| | AGGAAGGAGAAAGT | | | |
| Streptococcus | gaaattaatacgactcactat | 29 | TTCTACTACAAGATTGA | 41 |
| pneumoniae | agggGTTGATATATTGTTCTG | | ACGTATCCGCGAA | |
| | GGATGTAGGATG | | | |
| Enterococcus | gaaattaatacgactcactat | 30 | ATATTCTTCTGGAACAA | 42 |
| faecium | agggACAGCAAGCTCGTCGTA | | ATTCTCGGATCTCTT | |
| | CCTTAGACAGAATCG | | | |
| Enterococcus | gaaattaatacgactcactat | 31 | AACAGATTGCACACGAC | 43 |
| faecalis | agggGAGATTACTAAAGAAGC | | CAGCACTCAAACCT | |
| | GGTAAAAGCGGCATT | | | |

By “reduce” is meant to alter negatively relative to a reference. A reduction may be by 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition. A reference may be a sample (e.g., a blood or urine sample) from a subject not containing a target microbe or a sample known to contain or not contain a target nucleic acid molecule(s). A reference may be a subject prior to, during, or after treatment (e.g., with an antimicrobial agent).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween.

By “S-ribosylhomocysteine lyase from Serratia marcescens (luxS from Serratia marcescens) polypeptide” is meant a protein having S-ribosylhomocysteine lyase activity and with at least 85% sequence identity to GenBank® Ref. Seq. Accession No. TEW92082.1, which is provided below, or a functional fragment thereof:

>TEW92082.1 S-ribosylhomocysteine lyase [Serratia marcescens]

(SEQ ID NO: 44)
MPLLDSFTVDHTRMAAPAVRVAKTMKTPHGDTITVFDLRFCRPNLEVMPERGIHTLEHLFAGFM
RDHLNGQGVEIIDISPMGCRTGFYMSLIGVPEEQRVADAWKAAMADVLKVTDQRKIPELNEYQC
GTYHMHSLEEAQEIAKHILDNDVVVNHNDELALPKEKLQELHI

By “S-ribosylhomocysteine lyase from Serratia marcescens (luxS from Serratia marcescens) polynucleotide” is meant a nucleotide molecule encoding a S-ribosylhomocysteine lyase polypeptide. A representative S-ribosylhomocysteine lyase polynucleotide sequence is provided below (GenBank® Ref. Seq. SNQH01000018.1: c19039-18524):

>SNQH01000018.1: c19039-18524 Serratia marcescens strain 13F-69
CONTIG_19_length_190568_cov_33.822643, whole genome shotgun sequence

(SEQ ID NO: 45)
ATGCCATTGCTGGATAGCTTTACCGTCGATCATACCCGTATGGCAGCTCCGGCTGTCCGCGTTG
CGAAAACCATGAAAACGCCTCATGGCGATACCATCACGGTGTTTGATCTGCGCTTTTGCCGCCC
GAACCTGGAAGTGATGCCCGAGCGCGGCATTCATACGCTGGAGCACCTGTTCGCCGGCTTTATG
CGTGACCACCTGAACGGCCAGGGCGTGGAGATTATCGACATCTCCCCGATGGGCTGTCGTACCG
GTTTCTACATGAGCCTGATCGGCGTGCCGGAAGAGCAGCGCGTAGCCGATGCGTGGAAAGCGGC
GATGGCCGACGTGCTGAAAGTGACCGACCAGCGCAAAATCCCTGAGCTGAACGAATACCAGTGC
GGCACTTACCATATGCACTCGCTGGAAGAAGCGCAGGAGATCGCCAAGCACATCCTGGATAACG
ACGTGGTGGTTAACCACAACGACGAGCTGGCGCTGCCGAAAGAGAAGCTGCAGGAACTGCATAT
CTAG

By “SHV beta-lactamase 2 (SHV-2; bla_{SHV-2}) polypeptide” is meant a protein having carbapenem-hydrolyzing beta-lactamase activity and with at least 85% sequence identity to NCBI® Ref. Seq. Accession No. WP_032488413.1, which is provided below, or a functional fragment thereof:

>WP_032488413.1 MULTISPECIES: extended-spectrum class A beta-lactamase SHV-2a [Gammaproteobacteria]

(SEQ ID NO: 46)
MRYIRLCIISLLATLPLAVHASPQPLEQIKQSESQLSGRVGMIEMDLASGRTLTAWRADERFPM
MSTFKVVLCGAVLARVDAGDEQLERKIHYRQQDLVDYSPVSEKHLADGMTVGELCAAAITMSDN
SAANLLLATVGGPAGLTAFLRQIGDNVTRLDRWETELNEALPGDARDTTTPASMAATLRKLLTS
QRLSARSQRQLLQWMVDDRVAGPLIRSVLPAGWFIADKTGASERGARGIVALLGPNNKAERIVV
IYLRDTPASMAERNQQIAGIGAALIEHWQR

By “SHV beta-lactamase 2 (SHV-2; blasHIV-2) polynucleotide” is meant a nucleotide molecule encoding a carbapenem-hydrolyzing beta-lactamase polypeptide. A representative carbapenem-hydrolyzing beta-lactamase polynucleotide sequence is provided below (NCBI® Ref Seq. NZ_JAAJRZ010000063.1:670-1530):

>NZ_JAAJRZ010000063.1:670-1530 Escherichia coli strain ECM_55
NODE_63_length_5197_cov_98.942, whole genome shotgun sequence

(SEQ ID NO: 47)
ATGCGTTATATTCGCCTGTGTATTATCTCCCTGTTAGCCACCCTGCCGCTGGCGGTACACGCCA
GCCCGCAGCCGCTTGAGCAAATTAAACAAAGCGAAAGCCAGCTGTCGGGCCGCGTAGGCATGAT
AGAAATGGATCTGGCCAGCGGCCGCACGCTGACCGCCTGGCGCGCCGATGAACGCTTTCCCATG
ATGAGCACCTTTAAAGTAGTGCTCTGCGGCGCAGTGCTGGCGCGGGTGGATGCCGGTGACGAAC
AGCTGGAGCGAAAGATCCACTATCGCCAGCAGGATCTGGTGGACTACTCGCCGGTCAGCGAAAA
ACACCTTGCCGACGGCATGACGGTCGGCGAACTCTGCGCCGCCGCCATTACCATGAGCGATAAC
AGCGCCGCCAATCTGCTGCTGGCCACCGTCGGCGGCCCCGCAGGATTGACTGCCTTTTTGCGCC
AGATCGGCGACAACGTCACCCGCCTTGACCGCTGGGAAACGGAACTGAATGAGGCGCTTCCCGG
CGACGCCCGCGACACCACTACCCCGGCCAGCATGGCCGCGACCCTGCGCAAGCTGCTGACCAGC
CAGCGTCTGAGCGCCCGTTCGCAACGGCAGCTGCTGCAGTGGATGGTGGACGATCGGGTCGCCG
GACCGTTGATCCGCTCCGTGCTGCCGGCGGGCTGGTTTATCGCCGATAAGACCGGAGCTAGCGA
GCGGGGTGCGCGCGGGATTGTCGCCCTGCTTGGCCCGAATAACAAAGCAGAGCGCATTGTGGTG
ATTTATCTGCGGGATACGCCGGCGAGCATGGCCGAGCGAAATCAGCAAATCGCCGGGATCGGCG
CGGCGCTGATCGAGCACTGGCAACGCTAA

By “SHV beta-lactamase 12 (SHV-12; bla_{SHV-12}) polypeptide” is meant a protein having carbapenem-hydrolyzing beta-lactamase activity and with at least 85% sequence identity to NCBI® Ref. Seq. Accession No. WP_002904004.1, which is provided below, or a functional fragment thereof:

>WP_002904004.1 MULTISPECIES: extended-spectrum class A beta-lactamase SHV-12 [Bacteria]

(SEQ ID NO: 48)
MRYIRLCIISLLATLPLAVHASPQPLEQIKQSESQLSGRVGMIEMDLASGRTLTAWRADERFPM
MSTFKVVLCGAVLARVDAGDEQLERKIHYRQQDLVDYSPVSEKHLADGMTVGELCAAAITMSDN
SAANLLLATVGGPAGLTAFLRQIGDNVTRLDRWETELNEALPGDARDTTTPASMAATLRKLLTS
QRLSARSQRQLLQWMVDDRVAGPLIRSVLPAGWFIADKTGASKRGARGIVALLGPNNKAERIVV
IYLRDTPASMAERNQQIAGIGAALIEHWQR

By “SHV beta-lactamase 12 (SHV-12; bla_{SHV-12}) polynucleotide” is meant a nucleotide molecule encoding a carbapenem-hydrolyzing beta-lactamase polypeptide. A representative carbapenem-hydrolyzing beta-lactamase polynucleotide sequence is provided below (NCBI® Ref. Seq. NC_024967.1:3559-4419).

>NC_024967.1:3559-4419 Escherichia coli plasmid pYD626E, complete sequence

(SEQ ID NO: 49)
ATGCGTTATATTCGCCTGTGTATTATCTCCCTGTTAGCCACCCTGCCGCTGGCGGTACACGCCA
GCCCGCAGCCGCTTGAGCAAATTAAACAAAGCGAAAGCCAGCTGTCGGGCCGCGTAGGCATGAT
AGAAATGGATCTGGCCAGCGGCCGCACGCTGACCGCCTGGCGCGCCGATGAACGCTTTCCCATG
ATGAGCACCTTTAAAGTAGTGCTCTGCGGCGCAGTGCTGGCGCGGGTGGATGCCGGTGACGAAC
AGCTGGAGCGAAAGATCCACTATCGCCAGCAGGATCTGGTGGACTACTCGCCGGTCAGCGAAAA
ACACCTTGCCGACGGCATGACGGTCGGCGAACTCTGTGCCGCCGCCATTACCATGAGCGATAAC
AGCGCCGCCAATCTGCTGCTGGCCACCGTCGGCGGCCCCGCAGGATTGACTGCCTTTTTGCGCC
AGATCGGCGACAACGTCACCCGCCTTGACCGCTGGGAAACGGAACTGAATGAGGCGCTTCCCGG
CGACGCCCGCGACACCACTACCCCGGCCAGCATGGCCGCGACCCTGCGCAAGCTGCTGACCAGC
CAGCGTCTGAGCGCCCGTTCGCAACGGCAGCTGCTGCAGTGGATGGTGGACGATCGGGTCGCCG
GACCGTTGATCCGCTCCGTGCTGCCGGCGGGCTGGTTTATCGCCGATAAGACCGGAGCTAGCAA
ACGGGGTGCGCGCGGGATTGTCGCCCTGCTTGGCCCGAATAACAAAGCAGAGCGCATCGTGGTG
ATTTATCTGCGGGATACGCCGGCGAGCATGGCCGAGCGAAATCAGCAAATCGCCGGGATCGGCG
CGGCGCTGATCGAGCACTGGCAACGCTAA

By “specifically binds” is meant a compound or antibody that recognizes and binds a polypeptide or polynucleotide of the disclosure, but which does not substantially recognize and bind other molecules in a sample, for example, a biological sample, which naturally includes a target polypeptide or polynucleotide of the disclosure.

Nucleic acid molecules useful in the methods of the disclosure include, but are not limited to, any nucleic acid molecule that encodes a polypeptide of the disclosure or a fragment thereof, primers, guide polynucleotides. Such nucleic acid molecules need not be 100% identical with a nucleic acid sequence of interest, but will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid level to the sequence used for comparison. In various embodiments, a polypeptide or polynucleotide suitable for use in compositions or methods of the disclosure comprises an amino acid or polynucleotide sequence having about or at least about 85%, 90%, 95%, 96%, 97%, 98%, 99%, or greater sequence identity to a sequence provided herein.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “taxonomic group” is meant a classifier describing a set of organisms. In one embodiment, the taxonomic group is a genus or a species. In another embodiment, the taxonomic group classifies bacteria based on sequence homology, morphology, or another phenotype or observable characteristic.

By “T7 promoter” is meant a polynucleotide comprising one of the following nucleotide sequences: GAAATTAATACGACTCACTATAGGG (SEQ ID NO: 50), or a functional fragment thereof, that binds a T7 RNA polymerase polypeptide and drives expression of a downstream gene, or a nucleotide having at least 95% identity to GAAATTAATACGACTCACTATAGGG (SEQ ID NO: 50). In one embodiment, a T7 promoter comprises 1, 2, 3, 4, or 5 alterations relative to a T7 promoter reference sequence.

By “T7 RNA polymerase polypeptide” is meant a single-subunit RNA polymerase having at least 85% sequence identity to NCBI® Ref. Seq. Accession No. NP_041960.1, which is provided below, or a functional fragment thereof:

>NP_041960.1 RNA polymerase [Escherichia phage T7]

(SEQ ID NO: 51)
MNTINIAKNDFSDIELAAIPFNTLADHYGERLAREQLALEHESYEMGEARFRKMFERQLKAGEV
ADNAAAKPLITTLLPKMIARINDWFEEVKAKRGKRPTAFQFLQEIKPEAVAYITIKTTLACLTS
ADNTTVQAVASAIGRAIEDEARFGRIRDLEAKHFKKNVEEQLNKRVGHVYKKAFMQVVEADMLS
KGLLGGEAWSSWHKEDSIHVGVRCIEMLIESTGMVSLHRQNAGVVGQDSETIELAPEYAEAIAT
RAGALAGISPMFQPCVVPPKPWTGITGGGYWANGRRPLALVRTHSKKALMRYEDVYMPEVYKAI
NIAQNTAWKINKKVLAVANVITKWKHCPVEDIPAIEREELPMKPEDIDMNPEALTAWKRAAAAV
YRKDKARKSRRISLEFMLEQANKFANHKAIWFPYNMDWRGRVYAVSMFNPQGNDMTKGLLTLAK
GKPIGKEGYYWLKIHGANCAGVDKVPFPERIKFIEENHENIMACAKSPLENTWWAEQDSPFCFL
AFCFEYAGVQHHGLSYNCSLPLAFDGSCSGIQHFSAMLRDEVGGRAVNLLPSETVQDIYGIVAK
KVNEILQADAINGTDNEVVTVTDENTGEISEKVKLGTKALAGQWLAYGVTRSVTKRSVMTLAYG
SKEFGFRQQVLEDTIQPAIDSGKGLMFTQPNQAAGYMAKLIWESVSVTVVAAVEAMNWLKSAAK
LLAAEVKDKKTGEILRKRCAVHWVTPDGFPVWQEYKKPIQTRLNLMFLGQFRLQPTINTNKDSE
IDAHKQESGIAPNFVHSQDGSHLRKTVVWAHEKYGIESFALIHDSFGTIPADAANLFKAVRETM
VDTYESCDVLADFYDQFADQLHESQLDKMPALPAKGNLNLRDILESDFAFA

By “T7 RNA polymerase polynucleotide” is meant a nucleotide molecule encoding a T7 RNA polymerase polypeptide. A representative T7 RNA polymerase polynucleotide sequence is provided below (NCBI® Ref. Seq. NC_001604.1:3171-5822).

>NC_001604.1:3171-5822 Enterobacteria phage T7, complete genome

(SEQ ID NO: 52)
ATGAACACGATTAACATCGCTAAGAACGACTTCTCTGACATCGAACTGGCTGCTATCCCGTTCA
ACACTCTGGCTGACCATTACGGTGAGCGTTTAGCTCGCGAACAGTTGGCCCTTGAGCATGAGTC
TTACGAGATGGGTGAAGCACGCTTCCGCAAGATGTTTGAGCGTCAACTTAAAGCTGGTGAGGTT
GCGGATAACGCTGCCGCCAAGCCTCTCATCACTACCCTACTCCCTAAGATGATTGCACGCATCA
ACGACTGGTTTGAGGAAGTGAAAGCTAAGCGCGGCAAGCGCCCGACAGCCTTCCAGTTCCTGCA
AGAAATCAAGCCGGAAGCCGTAGCGTACATCACCATTAAGACCACTCTGGCTTGCCTAACCAGT
GCTGACAATACAACCGTTCAGGCTGTAGCAAGCGCAATCGGTCGGGCCATTGAGGACGAGGCTC
GCTTCGGTCGTATCCGTGACCTTGAAGCTAAGCACTTCAAGAAAAACGTTGAGGAACAACTCAA
CAAGCGCGTAGGGCACGTCTACAAGAAAGCATTTATGCAAGTTGTCGAGGCTGACATGCTCTCT
AAGGGTCTACTCGGTGGCGAGGCGTGGTCTTCGTGGCATAAGGAAGACTCTATTCATGTAGGAG
TACGCTGCATCGAGATGCTCATTGAGTCAACCGGAATGGTTAGCTTACACCGCCAAAATGCTGG
CGTAGTAGGTCAAGACTCTGAGACTATCGAACTCGCACCTGAATACGCTGAGGCTATCGCAACC
CGTGCAGGTGCGCTGGCTGGCATCTCTCCGATGTTCCAACCTTGCGTAGTTCCTCCTAAGCCGT
GGACTGGCATTACTGGTGGTGGCTATTGGGCTAACGGTCGTCGTCCTCTGGCGCTGGTGCGTAC
TCACAGTAAGAAAGCACTGATGCGCTACGAAGACGTTTACATGCCTGAGGTGTACAAAGCGATT
AACATTGCGCAAAACACCGCATGGAAAATCAACAAGAAAGTCCTAGCGGTCGCCAACGTAATCA
CCAAGTGGAAGCATTGTCCGGTCGAGGACATCCCTGCGATTGAGCGTGAAGAACTCCCGATGAA
ACCGGAAGACATCGACATGAATCCTGAGGCTCTCACCGCGTGGAAACGTGCTGCCGCTGCTGTG
TACCGCAAGGACAAGGCTCGCAAGTCTCGCCGTATCAGCCTTGAGTTCATGCTTGAGCAAGCCA
ATAAGTTTGCTAACCATAAGGCCATCTGGTTCCCTTACAACATGGACTGGCGCGGTCGTGTTTA
CGCTGTGTCAATGTTCAACCCGCAAGGTAACGATATGACCAAAGGACTGCTTACGCTGGCGAAA
GGTAAACCAATCGGTAAGGAAGGTTACTACTGGCTGAAAATCCACGGTGCAAACTGTGCGGGTG
TCGATAAGGTTCCGTTCCCTGAGCGCATCAAGTTCATTGAGGAAAACCACGAGAACATCATGGC
TTGCGCTAAGTCTCCACTGGAGAACACTTGGTGGGCTGAGCAAGATTCTCCGTTCTGCTTCCTT
GCGTTCTGCTTTGAGTACGCTGGGGTACAGCACCACGGCCTGAGCTATAACTGCTCCCTTCCGC
TGGCGTTTGACGGGTCTTGCTCTGGCATCCAGCACTTCTCCGCGATGCTCCGAGATGAGGTAGG
TGGTCGCGCGGTTAACTTGCTTCCTAGTGAAACCGTTCAGGACATCTACGGGATTGTTGCTAAG
AAAGTCAACGAGATTCTACAAGCAGACGCAATCAATGGGACCGATAACGAAGTAGTTACCGTGA
CCGATGAGAACACTGGTGAAATCTCTGAGAAAGTCAAGCTGGGCACTAAGGCACTGGCTGGTCA
ATGGCTGGCTTACGGTGTTACTCGCAGTGTGACTAAGCGTTCAGTCATGACGCTGGCTTACGGG
TCCAAAGAGTTCGGCTTCCGTCAACAAGTGCTGGAAGATACCATTCAGCCAGCTATTGATTCCG
GCAAGGGTCTGATGTTCACTCAGCCGAATCAGGCTGCTGGATACATGGCTAAGCTGATTTGGGA
ATCTGTGAGCGTGACGGTGGTAGCTGCGGTTGAAGCAATGAACTGGCTTAAGTCTGCTGCTAAG
CTGCTGGCTGCTGAGGTCAAAGATAAGAAGACTGGAGAGATTCTTCGCAAGCGTTGCGCTGTGC
ATTGGGTAACTCCTGATGGTTTCCCTGTGTGGCAGGAATACAAGAAGCCTATTCAGACGCGCTT
GAACCTGATGTTCCTCGGTCAGTTCCGCTTACAGCCTACCATTAACACCAACAAAGATAGCGAG
ATTGATGCACACAAACAGGAGTCTGGTATCGCTCCTAACTTTGTACACAGCCAAGACGGTAGCC
ACCTTCGTAAGACTGTAGTGTGGGCACACGAGAAGTACGGAATCGAATCTTTTGCACTGATTCA
CGACTCCTTCGGTACCATTCCGGCTGACGCTGCGAACCTGTTCAAAGCAGTGCGCGAAACTATG
GTTGACACATATGAGTCTTGTGATGTACTGGCTGATTTCTACGACCAGTTCGCTGACCAGTTGC
ACGAGTCTCAATTGGACAAAATGCCAGCACTTCCGGCTAAAGGTAACTTGAACCTCCGTGACAT
CTTAGAGTCGGACTTCGCGTTCGCGTAA

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST®, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST® program may be used, with a probability score between e⁻³ and e⁻¹⁰⁰ indicating a closely related sequence.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “type 1 DNA topoisomerase from Acinetobacter baumannii (topA from A. baumannii) polypeptide” is meant a protein having topoisomerase activity and with at least 85% sequence identity to GenBank® Ref. Seq. Accession No. SSI42396.1, which is provided below, or a functional fragment thereof:

>SSI42396.1 DNA topoisomerase I [Acinetobacter baumannii]

(SEQ ID NO: 53)
MANTSRSASQSTASSASAAHKRALVIVESPAKAKTINKYLGSQYIVKSSVGHVRDLPTGGSKAT
EKKPAARTKLTEAEKEQKANQALINRMGVDPEHGWQAHYEVLPGKENVVAELKKLAKDADAIYL
ATDLDREGEAIAWHLREVIGGDDSRYHRVVFNEITKNAIQEAFKQPTRLDLNRVNAQQARRFLD
RVVGFMVSPLLWEKIARGLSAGRVQSVAVKLVVEREREIRAFIPEEYWQVFADTKAKKDDIRLE
AVKQAGKTLKLKNKAETDALLDVLKGAEYKVAQREDKPTKVNPSAPYITSTLQQAASTRLGFSV
KKTMMLAQRLYEAGFITYMRTDSTFLSDDAVSMVRAHIESQYGEKYLPAKPNRYGNKAGAQEAH
EAIRPSNVALTGDQLAGVERDAQRLYDLIWRQFVACQMTPAEYLSSTLTVEAGNVELKAKGRTL
VFDGFTKVRGANKSDDDIILPAIKVGEILKLEKLDPSQHFTKPPARFTEASLVKELEKRGIGRP
STYAAIISTIQERGYVKLENRRLFAEKMGEIVTDRLDESFNNLMNYAFTADLEGQLDRVATGER
NWKELLDTFYGDFKKRLTNAQGEQGMRRNQPVEVPAVHCPECSRPMQIRTGTTGVFLGCSGYNL
PPKERCKGTLNLTPVESLAALSDDDSAETADLMSKHRCPKCGTAMDSYVIDGGRKLHVCGNNPD
CDGYELEEGEFKIKGYDGPTIPCDKCNGEMQLKTGRFGPYFACTSCDNTRKVLKNGQPAPPRVE
PIKMEHLRSTKHDDYFVLRDGAAGLFLAASKFPKIRETRAPKVAELRSVADQLDPKYQFILQAP
DVDPEGNPTIVKFSRKNQSQYVGSETPEGKQTKWSLIYQDGKWIEG

By “type 1 DNA topoisomerase from Acinetobacter baumannii (topA from A. baumannii) polynucleotide” is meant a nucleotide molecule encoding a topoisomerase polypeptide. A representative topoisomerase polynucleotide sequence is provided below (GenBank® Ref. Seq. UFDQ01000001.1: c191705-189069):

>UFDQ01000001.1: c191705-189069 Acinetobacter baumannii strain 4300STDY6542376 genome assembly, contig: ERS1306195SCcontig000001, whole genome shotgun sequence

(SEQ ID NO: 54)
ATGGCGAATACCTCGCGCTCTGCATCTCAAAGCACAGCATCTTCTGCCTCTGCTGCACATAAAC
GTGCCTTAGTGATTGTGGAGTCGCCTGCCAAAGCGAAAACCATCAACAAATATTTAGGTTCGCA
GTACATTGTTAAGTCTTCTGTAGGTCACGTACGTGATTTGCCAACAGGCGGCAGTAAAGCAACA
GAGAAAAAGCCGGCTGCCCGGACCAAACTCACTGAGGCTGAAAAAGAACAAAAAGCGAATCAGG
CTTTAATCAATCGTATGGGCGTCGATCCGGAACATGGATGGCAAGCGCATTACGAAGTTCTGCC
TGGCAAAGAAAATGTTGTTGCTGAACTCAAGAAACTTGCTAAAGATGCAGATGCAATCTATCTC
GCAACGGACTTGGATAGAGAAGGGGAAGCAATCGCTTGGCACTTACGCGAAGTGATTGGTGGTG
ACGATAGCCGTTATCATCGTGTGGTATTTAACGAAATTACTAAAAATGCCATTCAAGAAGCATT
TAAACAGCCAACACGTCTCGACTTAAACCGTGTTAATGCACAACAAGCACGTCGTTTCTTGGAC
CGTGTAGTAGGCTTCATGGTTTCGCCATTATTATGGGAAAAGATTGCCCGTGGTTTATCGGCAG
GTCGTGTACAGTCTGTAGCGGTAAAGCTTGTTGTTGAACGTGAACGTGAAATTCGTGCTTTTAT
TCCAGAAGAATATTGGCAAGTCTTTGCAGACACTAAAGCTAAAAAAGATGACATTCGTCTTGAG
GCTGTTAAACAGGCGGGTAAAACGCTTAAATTAAAAAATAAAGCTGAAACAGATGCCCTGTTAG
ATGTATTAAAAGGTGCTGAATATAAAGTTGCCCAGCGTGAAGATAAACCAACTAAGGTGAATCC
AAGCGCCCCATACATCACTTCGACTTTGCAACAAGCTGCAAGTACACGTTTAGGTTTCTCTGTA
AAGAAAACCATGATGTTGGCGCAGCGCTTGTATGAAGCTGGTTTCATTACTTATATGCGTACTG
ACTCAACGTTTTTGAGTGATGATGCTGTAAGTATGGTGCGTGCACATATTGAAAGCCAATATGG
CGAAAAATATTTACCAGCTAAGCCAAATCGCTATGGTAATAAAGCAGGTGCTCAAGAAGCCCAC
GAAGCTATTCGTCCATCAAATGTTGCGCTGACTGGTGATCAACTTGCTGGTGTAGAGCGTGATG
CTCAGCGCTTATATGATTTAATTTGGCGTCAGTTCGTAGCTTGCCAAATGACTCCGGCAGAATA
TTTATCTTCAACTTTAACAGTTGAAGCTGGCAATGTGGAGTTAAAGGCAAAAGGCCGTACGCTT
GTATTTGATGGCTTTACTAAAGTTCGTGGTGCCAACAAATCTGATGACGATATCATTTTACCTG
CAATTAAAGTGGGCGAAATTTTAAAGCTAGAAAAGCTAGATCCAAGTCAGCATTTTACCAAACC
ACCAGCTCGTTTTACTGAAGCATCTTTAGTAAAAGAACTTGAAAAACGTGGTATTGGTCGTCCG
TCGACTTATGCTGCCATTATTTCTACGATTCAAGAACGTGGTTACGTTAAGTTAGAAAATCGCC
GTCTCTTTGCCGAGAAAATGGGTGAGATCGTAACGGATCGTCTTGATGAAAGTTTTAATAACCT
AATGAATTATGCATTTACTGCAGATTTAGAAGGTCAGTTGGACCGTGTAGCAACGGGTGAGCGT
AACTGGAAGGAATTGCTAGACACATTCTATGGTGATTTTAAAAAGCGTTTGACCAATGCTCAAG
GTGAGCAGGGAATGCGTCGTAACCAGCCAGTTGAAGTACCAGCAGTACATTGCCCTGAATGTTC
ACGTCCAATGCAGATTCGTACGGGCACTACAGGCGTATTCTTAGGGTGTTCTGGCTATAACTTG
CCACCTAAAGAACGCTGTAAAGGGACCTTGAACTTAACGCCAGTTGAGTCTTTAGCAGCGTTAT
CTGATGACGATAGTGCTGAAACAGCAGACTTGATGTCAAAACATCGTTGTCCGAAATGTGGCAC
GGCAATGGACAGCTACGTCATTGATGGTGGCCGTAAGTTACATGTTTGTGGTAACAACCCGGAT
TGCGATGGCTATGAGCTTGAAGAAGGTGAGTTCAAGATCAAAGGGTATGATGGCCCAACCATTC
CATGTGATAAATGTAATGGTGAAATGCAGCTTAAGACTGGTCGCTTCGGCCCGTATTTTGCTTG
TACAAGCTGTGACAATACTCGTAAAGTCTTGAAAAATGGTCAACCTGCACCACCACGTGTAGAA
CCAATCAAGATGGAACACTTGCGTTCAACCAAACATGATGATTATTTTGTGTTACGTGATGGCG
CGGCTGGTCTGTTCTTGGCTGCAAGTAAGTTCCCGAAAATTCGTGAAACACGTGCACCAAAAGT
TGCTGAGCTTCGTAGCGTAGCTGATCAACTTGATCCAAAATACCAGTTTATTTTACAAGCTCCA
GATGTAGACCCTGAAGGTAACCCAACTATTGTGAAGTTTAGTCGTAAGAACCAATCACAATATG
TGGGTTCTGAAACTCCAGAAGGTAAACAAACCAAATGGAGCCTAATTTATCAAGACGGTAAATG
GATTGAAGGCTAA

By “type 1 DNA topoisomerase from Citrobacter freundii (topA from C. freundii) polypeptide” is meant a protein having topoisomerase activity and with at least 85% sequence identity to GenBank® Ref. Seq. Accession No. QFI30115.1, which is provided below, or a functional fragment thereof:

>QFI30115.1 topoisomerase (plasmid) [Citrobacter freundii]

(SEQ ID NO: 55)
MVFRKLAAKGMSRCAKFQWCFFLLASLALFFLHDGPLVIAGLFITQLSLQMSMTRTGKEVFGVI
FNIQSWRNSGASQQTYFFRWYIGFPISCLLYILSLTNYVYVPGDLYYQFIASSTDDLALVMNRF
AVGVSNILPEILRGASLTLSMMMFVATLLKNVQLAEVRRIYSSIQDGNDSQKILSNMTWEQFEK
IIRLHFELNGYKATLTNTGADGGVDILLQKDGRREMVQCKLWKTSKVGVSVVREIYGVVQANAY
ERGYIITSGFFTQDAWEFALSANVKGTLSLIDGSRLLKIIKDKEIPPPATEIINVGPGENQIYD
LNVIPSCPRCHSKMVRRMSNGNYFYGCSAYPICKGTRNL

By “type 1 DNA topoisomerase from Citrobacter freundii (topA from C. freundii) polynucleotide” is meant a nucleotide molecule encoding a topoisomerase polypeptide. A representative topoisomerase polynucleotide sequence is provided below (GenBank® Ref. Seq. CP042535.1: c72143-71064):

>CP042535.1: c72143-71064 Citrobacter freundii strain E51 plasmid pE51_001, complete sequence

(SEQ ID NO: 56)
ATGGTTTTTAGAAAATTGGCAGCAAAAGGAATGTCCCGATGCGCCAAATTCCAGTGGTGCTTTT
TCCTGCTAGCGTCATTAGCTTTATTCTTTCTACATGATGGCCCGTTGGTCATTGCGGGCCTCTT
TATTACACAATTGTCTCTCCAGATGTCGATGACCCGTACAGGAAAGGAAGTCTTCGGCGTTATT
TTTAATATCCAGTCATGGAGAAACAGTGGTGCTAGCCAGCAAACCTATTTTTTCCGGTGGTATA
TCGGCTTCCCGATTTCCTGCTTGCTATATATTCTTTCTCTTACTAATTATGTGTACGTTCCAGG
CGATTTGTATTATCAGTTTATTGCAAGCAGTACTGATGACCTAGCACTCGTAATGAATCGTTTT
GCTGTAGGCGTGAGTAACATACTCCCTGAAATCCTTCGAGGCGCATCTCTAACCCTTTCGATGA
TGATGTTTGTCGCAACATTGCTAAAAAATGTACAACTTGCTGAAGTACGTCGAATTTACTCCTC
CATTCAGGATGGAAATGATTCCCAGAAAATTCTCTCGAATATGACATGGGAGCAATTCGAAAAG
ATTATAAGACTGCATTTCGAACTCAATGGCTACAAAGCAACTCTGACTAATACAGGAGCTGACG
GCGGAGTCGACATTCTGCTGCAAAAAGATGGTCGCCGCGAAATGGTACAGTGCAAATTATGGAA
AACCAGCAAAGTAGGAGTCTCGGTAGTCCGCGAAATTTACGGTGTAGTGCAAGCCAATGCATAT
GAGCGGGGTTATATCATCACCTCTGGGTTCTTTACTCAGGATGCATGGGAATTCGCTCTTAGCG
CAAATGTTAAAGGAACATTATCGCTTATCGATGGTTCCAGACTTCTGAAAATAATCAAGGATAA
AGAAATCCCCCCGCCTGCCACTGAGATAATAAATGTTGGTCCAGGTGAGAATCAAATATACGAT
CTTAATGTCATACCTAGCTGTCCCCGTTGCCATAGCAAAATGGTTAGAAGAATGTCGAATGGAA
ATTATTTCTATGGTTGCTCGGCTTACCCAATTTGTAAAGGAACCCGTAACCTCTAG

By “type 1 DNA topoisomerase from Klebsiella pneumoniae (topA from K. pneumoniae) polypeptide” is meant a protein having topoisomerase activity and with at least 85% sequence identity to GenBank® Ref. Seq. Accession No. MCU8584025.1, which is provided below, or a functional fragment thereof:

>MCU8584025.1 type I DNA topoisomerase [Klebsiella pneumoniae]

(SEQ ID NO: 57)
MGKALVIVESPAKAKTINKYLGSDYVVKSSVGHIRDLPTSGSAAKKSADSTSTKTAKKPKKDER
GALVNRMGVDPWHNWEAHYEVLPGKEKVVSELKQLAEKADHIYLATDLDREGEAIAWHLREVIG
GDDARYSRVVFNEITKNAIRQAFNKPGELNIDRVNAQQARRFMDRVVGYMVSPLLWKKIARGLS
AGRVQSVAVRLVVEREREIKAFVPEEFWEVDASTTTPSGEALALQVTHQNDKPFRPVNKEQTQA
AVSLLEKARYSVLEREDKPTTSKPGAPFITSTLQQAASTRLGFGVKKTMMMAQRLYEAGYITYM
RTDSTNLSQDAVNMVRGYISDNFGKKYLPESPNQYASKENSQEAHEAIRPSDVNVMAESLKDME
ADAQKLYQLIWRQFVACQMTPAKYDSTTLTVGAGDFRLKARGRILRFDGWTKVMPALRKGDEDR
ILPAVNKGDALTLVELTPAQHFTKPPARFSEASLVKELEKRGIGRPSTYASIISTIQDRGYVRV
ENRRFYAEKMGEIVTDRLEENFRELMNYDFTAQMENSLDQVANHEAEWKAVLDHFFSDFTQQLD
KAEKDPEEGGMRPNQMVLTSIDCPTCGRKMGIRTASTGVFLGCSGYALPPKERCKTTINLVPEN
EVLNVLEGEDAETNALRAKRRCPKCGTAMDSYLIDPKRKLHVCGNNPTCDGYEIEEGEFRIKGY
DGPIVECEKCGSEMHLKMGRFGKYMACTNEECKNTRKILRNGEVAPPKEDPVPLPELPCEKSDA
YFVLRDGAAGVFLAANTFPKSRETRAPLVEELYRFRDRLPEKLRYLADAPQQDPEGNKTMVRFS
RKTKQQYVSSEKDGKATGWSAFYVDGKWVEGKK

By “type 1 DNA topoisomerase from Klebsiella pneumoniae (topA from K. pneumoniae) polynucleotide” is meant a nucleotide molecule encoding a topoisomerase polypeptide. A representative topoisomerase polynucleotide sequence is provided below (GenBank® Ref. Seq. JAOURE010000039.1:42512-45109):

>JAOURE010000039.1:42512-45109 Klebsiella pneumoniae strain P157 contig00071, whole genome shotgun sequence

(SEQ ID NO: 58)
ATGGGTAAAGCTCTTGTCATCGTTGAGTCCCCGGCAAAAGCCAAAACGATCAACAAGTATCTGG
GTAGTGACTACGTGGTGAAATCCAGCGTCGGTCACATCCGCGATTTGCCGACCAGTGGCTCAGC
TGCCAAAAAGAGTGCCGACTCTACCTCCACCAAGACGGCTAAAAAGCCTAAAAAGGATGAACGT
GGCGCTCTCGTCAACCGTATGGGGGTTGACCCGTGGCACAATTGGGAGGCGCACTATGAAGTGT
TGCCTGGTAAAGAGAAGGTCGTCTCTGAACTGAAACAACTGGCTGAAAAAGCCGACCACATCTA
TCTCGCAACCGACCTTGACCGCGAAGGGGAAGCCATTGCATGGCACCTGCGGGAAGTGATTGGG
GGTGATGATGCGCGCTATAGCCGAGTGGTGTTTAACGAAATTACTAAAAACGCGATCCGCCAGG
CATTTAACAAACCGGGTGAGCTGAATATTGATCGTGTTAATGCCCAGCAGGCGCGTCGCTTTAT
GGACCGCGTGGTGGGGTATATGGTTTCGCCGCTGCTATGGAAAAAGATCGCTCGTGGTCTGTCT
GCCGGTCGTGTGCAGTCGGTGGCGGTTCGCCTGGTGGTCGAGCGTGAGCGTGAAATTAAAGCGT
TCGTGCCGGAAGAGTTCTGGGAAGTCGATGCCAGCACGACCACGCCATCTGGTGAAGCGTTGGC
GTTACAGGTGACTCATCAGAACGACAAACCGTTCCGTCCGGTCAACAAAGAACAAACTCAGGCT
GCGGTAAGTCTGCTGGAAAAAGCGCGCTACAGCGTGCTGGAACGTGAAGACAAACCGACAACCA
GTAAACCTGGCGCTCCTTTTATTACCTCTACGCTGCAACAAGCTGCCAGCACCCGTCTTGGATT
TGGCGTGAAAAAAACCATGATGATGGCGCAGCGTTTGTATGAAGCAGGCTATATCACTTACATG
CGTACCGACTCCACTAACCTGAGTCAGGACGCGGTAAATATGGTTCGCGGTTATATCAGCGATA
ATTTTGGTAAGAAATATCTGCCGGAAAGTCCGAATCAGTACGCCAGCAAAGAAAACTCACAGGA
AGCGCACGAAGCGATTCGTCCTTCTGACGTCAATGTGATGGCGGAATCGCTGAAGGATATGGAA
GCAGATGCGCAGAAACTGTACCAGTTAATCTGGCGTCAGTTCGTTGCCTGCCAGATGACCCCAG
CGAAATATGACTCCACGACGCTGACCGTTGGTGCGGGCGATTTCCGCCTGAAAGCACGCGGTCG
TATTTTGCGTTTTGATGGCTGGACAAAAGTGATGCCTGCGTTGCGTAAAGGCGATGAAGATCGC
ATCTTACCAGCAGTTAATAAAGGCGATGCTCTGACGCTCGTTGAACTTACACCAGCCCAGCACT
TTACCAAGCCGCCAGCCCGTTTCAGTGAAGCATCGCTGGTTAAAGAGCTGGAAAAACGCGGTAT
CGGTCGTCCGTCTACCTATGCGTCGATCATTTCGACCATTCAGGATCGTGGCTACGTGCGAGTA
GAAAATCGTCGTTTCTATGCGGAAAAAATGGGCGAAATCGTCACCGATCGCCTTGAAGAAAATT
TCCGCGAGTTAATGAACTACGACTTTACCGCGCAGATGGAAAACAGCCTCGACCAGGTGGCAAA
TCACGAAGCAGAGTGGAAAGCTGTACTGGATCACTTCTTCTCGGATTTCACCCAGCAGTTAGAT
AAAGCTGAAAAAGATCCGGAAGAGGGTGGTATGCGCCCGAACCAGATGGTTCTGACCAGCATTG
ACTGCCCGACTTGTGGTCGCAAAATGGGGATTCGCACAGCGAGCACCGGGGTATTCCTTGGCTG
TTCTGGCTATGCGCTGCCGCCGAAAGAGCGTTGCAAAACCACCATTAACCTGGTGCCGGAAAAC
GAAGTGCTGAACGTGCTGGAAGGCGAAGATGCTGAAACCAACGCGCTGCGCGCAAAACGTCGTT
GCCCGAAATGCGGCACGGCGATGGACAGCTATCTCATCGATCCGAAACGTAAGTTGCATGTCTG
TGGTAATAACCCAACCTGCGACGGTTACGAGATCGAAGAGGGCGAATTCCGCATTAAAGGTTAT
GACGGCCCGATCGTTGAGTGTGAAAAATGTGGCTCTGAAATGCACCTGAAAATGGGGCGTTTCG
GTAAATACATGGCCTGCACCAACGAAGAGTGTAAAAACACACGTAAGATTTTACGTAACGGCGA
AGTGGCACCACCGAAAGAAGATCCGGTGCCATTACCTGAGCTGCCGTGCGAAAAATCAGATGCT
TATTTCGTGCTGCGTGACGGTGCTGCCGGTGTGTTCCTGGCTGCCAACACTTTCCCGAAATCGC
GTGAAACGCGTGCGCCACTGGTGGAAGAGCTTTATCGCTTCCGCGACCGTCTGCCGGAAAAACT
GCGTTATCTGGCCGATGCGCCACAGCAGGATCCGGAAGGTAATAAGACCATGGTTCGCTTTAGC
CGTAAAACCAAACAGCAATATGTCTCTTCGGAAAAAGACGGAAAGGCGACTGGCTGGTCAGCAT
TTTATGTTGATGGCAAATGGGTTGAAGGAAAAAAATAA

By “type 1 DNA topoisomerase from Proteus mirabilis (topA from P. mirabilis) polypeptide” is meant a protein having topoisomerase activity and with at least 85% sequence identity to GenBank® Ref. Seq. Accession No. KXB98732.1, which is provided below, or a functional fragment thereof:

>KXB98732.1 DNA topoisomerase I [Proteus mirabilis]

(SEQ ID NO: 59)
MGKALVIVESPAKAKTINKYLGNDYVVKSSVGHIRDLPKSGSGSQKSENSSATKGVKKVKKDEK
SALVSRMGVDPYNGWHANYQILPGKEKVVAELKALAEKADHIYLATDLDREGEAIAWHLREVIG
GEDDRFSRVVFNEITKNAITQAFQTPGELNIDRVNAQQARRFMDRVVGYMVSPLLWKKVARGLS
AGRVQSVAVRLLVEREREIKAFVPEEYWQLHASLLTPDEQPLRMEVTHYQDNAFKPVSSEETQV
AVKALQNATFIVKNREDRPTSSKPSAPLITSTLQQAASTRLSFGVKKTMMLAQRLYEAGYITYM
RTDSTNLSQDAIQMARDYIHDKFGAKYLPKEPNVYSSKENSQEAHEAIRPSDINVTAESLKDMD
SDAKRLYQLIWDQFVACQMTPAKYDSTTLTVVSGDYKLRAKGRTLRFAGWTKVMPAMRSKDEDK
TLPAVDVGAQLALAELSPTQHFTKPPARFSEATLVKELEKRGIGRPSTYASIISTIQDRGYVKV
ENRRFYAEKMGEIVTDRLEENFSDLMNYDFTAQMEDHLDHVANNQENWKAVLDAFFTDFSQQLE
VAEKDPEEGGMRPNPMVITSIECPTCSRHMGIRTATTGVFLGCSGYALPPKERCKQTINLIPED
ELLNILEGDDAETNALRARRRCPKCGTAMDSYLIDTHRKLHVCGNNPACDGYEVEEGEFRLKGY
EGPVIECDKCGAEMHLKMGRFGKYMGCTNEECKNTRKILKSGEIAPPKEDPVPLPELPCEKSDA
YFVLRDGAAGIFLAANTFPKSRETRAPKVAELVRFKDRLSEKMRYLAQAPVTDPEGNPTIVRFS
RKTKQQYVSSEKEGKATGWSAFYIDGKWVEKSK

By “type 1 DNA topoisomerase from Proteus mirabilis (topA from P. mirabilis) polynucleotide” is meant a nucleotide molecule encoding a topoisomerase polypeptide. A representative topoisomerase polynucleotide sequence is provided below (GenBank® Ref. Seq. KQ961018.1: c9899-7302):

>KQ961018.1: c9899-7302 Proteus mirabilis strain GED7834 genomic scaffold Scaffold443, whole genome shotgun sequence

(SEQ ID NO: 60)
ATGGGTAAAGCTCTTGTTATCGTGGAGTCCCCGGCTAAAGCCAAAACAATCAATAAATATCTTG
GCAATGACTACGTAGTAAAATCTAGCGTGGGTCACATTCGTGATTTGCCAAAAAGTGGTTCTGG
CAGCCAGAAGAGCGAAAACTCATCCGCCACGAAAGGCGTGAAAAAGGTTAAAAAGGATGAGAAA
TCAGCCTTAGTCAGCCGTATGGGTGTCGATCCTTATAATGGTTGGCATGCAAACTACCAAATTC
TGCCGGGTAAAGAGAAGGTAGTCGCTGAACTAAAAGCATTGGCTGAAAAAGCAGATCATATCTA
TCTCGCAACGGACCTTGACCGCGAAGGAGAAGCTATCGCGTGGCATTTACGCGAAGTGATCGGC
GGTGAAGATGATAGATTTAGTCGTGTCGTATTTAATGAAATTACGAAAAATGCCATTACACAAG
CATTCCAAACACCGGGTGAGCTGAATATTGACCGTGTTAACGCGCAACAAGCACGTCGTTTTAT
GGATCGTGTGGTTGGCTATATGGTTTCTCCGCTGTTATGGAAAAAAGTGGCGCGTGGTTTATCT
GCGGGGCGTGTACAGTCAGTTGCGGTACGCCTACTCGTCGAGCGTGAACGTGAAATTAAAGCAT
TTGTGCCAGAAGAGTATTGGCAATTACACGCTTCACTGCTGACGCCCGATGAACAACCCTTGCG
TATGGAAGTGACGCACTATCAAGATAACGCTTTTAAACCAGTCTCCTCTGAGGAAACTCAGGTG
GCGGTTAAGGCGTTACAGAATGCCACTTTCATCGTTAAAAACCGTGAAGATAGACCAACATCCA
GTAAACCAAGTGCCCCTTTGATCACCTCTACATTGCAACAAGCGGCAAGTACTCGCTTAAGCTT
TGGGGTGAAAAAGACCATGATGTTGGCTCAACGTCTTTATGAAGCAGGTTATATCACCTATATG
CGTACTGACTCTACCAACCTGAGTCAGGATGCAATCCAAATGGCACGTGATTATATTCACGATA
AATTTGGTGCAAAATATCTGCCTAAAGAGCCGAATGTTTATTCAAGTAAAGAGAACTCACAAGA
AGCACACGAAGCTATTCGTCCTTCTGATATCAATGTGACGGCTGAATCTTTAAAAGATATGGAT
AGCGACGCGAAGCGTTTATATCAACTGATTTGGGATCAATTTGTCGCCTGTCAAATGACACCGG
CAAAATATGACTCAACCACACTTACGGTTGTATCAGGGGATTATAAACTACGCGCTAAAGGTCG
CACATTACGTTTTGCGGGTTGGACAAAAGTCATGCCAGCAATGCGCAGTAAAGATGAAGATAAA
ACATTACCAGCGGTTGATGTTGGGGCGCAATTAGCGTTAGCTGAGTTATCGCCAACGCAACACT
TTACTAAGCCACCAGCGCGTTTTAGTGAAGCTACATTAGTTAAAGAGCTAGAAAAACGAGGTAT
TGGTCGACCTTCAACTTATGCTTCAATTATCTCTACCATTCAAGATAGAGGTTATGTCAAAGTT
GAAAACCGTCGTTTCTATGCAGAAAAAATGGGTGAGATTGTTACCGATCGCTTAGAAGAAAACT
TTAGTGATTTGATGAACTACGATTTCACGGCTCAAATGGAAGATCATCTCGACCATGTTGCCAA
CAACCAAGAAAACTGGAAAGCGGTATTAGATGCATTCTTCACAGATTTTAGTCAGCAACTAGAA
GTCGCTGAAAAAGATCCAGAAGAAGGAGGAATGCGACCTAACCCTATGGTTATTACTTCTATTG
AATGTCCTACTTGTTCTCGTCATATGGGAATTAGAACAGCGACTACAGGGGTATTCTTAGGGTG
CTCGGGCTATGCTTTACCACCAAAAGAGCGTTGTAAACAAACGATAAATCTTATTCCTGAAGAT
GAATTACTTAATATCTTGGAAGGAGATGATGCGGAAACTAATGCATTACGTGCCCGTCGTCGTT
GTCCGAAATGCGGTACGGCAATGGACAGTTATCTTATTGATACTCATCGTAAATTACATGTTTG
TGGTAATAATCCTGCATGTGATGGTTATGAAGTTGAAGAAGGTGAATTCCGCTTAAAAGGTTAT
GAAGGCCCAGTGATTGAGTGTGATAAATGTGGCGCTGAAATGCATCTAAAAATGGGGCGTTTTG
GCAAGTACATGGGTTGCACCAATGAAGAGTGTAAAAATACACGTAAGATCTTAAAGAGTGGTGA
AATTGCACCACCAAAAGAAGATCCGGTTCCACTACCTGAGTTGCCTTGTGAAAAATCGGATGCT
TACTTTGTGTTACGTGATGGCGCGGCGGGTATTTTCCTTGCGGCCAATACGTTCCCTAAATCAA
GGGAAACCCGTGCACCGAAAGTCGCTGAGTTAGTACGTTTTAAAGATAGATTATCGGAAAAAAT
GCGCTACTTAGCACAAGCACCGGTGACCGATCCTGAAGGGAATCCAACCATAGTACGTTTTAGT
CGTAAAACTAAACAGCAATATGTCTCTTCTGAAAAAGAGGGGAAAGCAACAGGTTGGTCAGCCT
TTTATATTGATGGCAAATGGGTTGAAAAAAGTAAATAA

By “Type VI CRISPR ortholog” or “Type VI CRISPR polypeptide” is meant an RNA nuclease capable of forming a complex with a guide polynucleotide, where binding of the guide polynucleotide of the complex to a target site in a single-stranded RNA molecule results in activation of the RNA nuclease and cleavage thereby of the single-stranded RNA molecule and non-specific cleavage of nearby single-stranded RNA molecules. In various embodiments, a Type VI CRISPR ortholog is a Cas13 protein (e.g., a Cas13a, Cas13b, or Cas13c protein). In various embodiments, a Cas13a polypeptide comprises an amino acid sequence with at least 85% identity to one of the following amino acid sequences, or a fragment thereof having nuclease activity and capable of forming a complex with a guide polynucleotide:

Leptotrichia wadei (Lw2) Cas13a:

(SEQ ID NO: 61)
MKVTKVDGISHKKYIEEGKLVKSTSEENRTSERLSELLSIRLDIYIKNPDNASEEENRIRRENL
KKFFSNKVLHLKDSVLYLKNRKEKNAVQDKNYSEEDISEYDLKNKNSFSVLKKILLNEDVNSEE
LEIFRKDVEAKLNKINSLKYSFEENKANYQKINENNVEKVGGKSKRNIIYDYYRESAKRNDYIN
NVQEAFDKLYKKEDIEKLFFLIENSKKHEKYKIREYYHKIIGRKNDKENFAKIIYEEIQNVNNI
KELIEKIPDMSELKKSQVFYKYYLDKEELNDKNIKYAFCHFVEIEMSQLLKNYVYKRLSNISND
KIKRIFEYQNLKKLIENKLLNKLDTYVRNCGKYNYYLQVGEIATSDFIARNRQNEAFLRNIIGV
SSVAYFSLRNILETENENDITGRMRGKTVKNNKGEEKYVSGEVDKIYNENKQNEVKENLKMFYS
YDFNMDNKNEIEDFFANIDEAISSIRHGIVHFNLELEGKDIFAFKNIAPSEISKKMFQNEINEK
KLKLKIFKQLNSANVFNYYEKDVIIKYLKNTKFNFVNKNIPFVPSFTKLYNKIEDLRNTLKFFW
SVPKDKEEKDAQIYLLKNIYYGEFLNKFVKNSKVFFKITNEVIKINKQRNQKTGHYKYQKFENI
EKTVPVEYLAIIQSREMINNQDKEEKNTYIDFIQQIFLKGFIDYLNKNNLKYIESNNNNDNNDI
FSKIKIKKDNKEKYDKILKNYEKHNRNKEIPHEINEFVREIKLGKILKYTENLNMFYLILKLLN
HKELTNLKGSLEKYQSANKEETFSDELELINLLNLDNNRVTEDFELEANEIGKFLDFNENKIKD
RKELKKFDTNKIYFDGENIIKHRAFYNIKKYGMLNLLEKIADKAKYKISLKELKEYSNKKNEIE
KNYTMQQNLHRKYARPKKDEKFNDEDYKEYEKAIGNIQKYTHLKNKVEFNELNLLQGLLLKILH
RLVGYTSIWERDLRFRLKGEFPENHYIEEIFNFDNSKNVKYKSGQIVEKYINFYKELYKDNVEK
RSIYSDKKVKKLKQEKKDLYIRNYIAHFNYIPHAEISLLEVLENLRKLLSYDRKLKNAIMKSIV
DILKEYGFVATFKIGADKKIEIQTLESEKIVHLKNLKKKKLMTDRNSEELCELVKVMFEYKALE
KRPAATKKAGQAKKKKGSYPYDVPDYAYPYDVPDYAYPYDVPDYA

Leptotrichia wadei F0279 Cas13a:

(SEQ ID NO: 62)
MKVTKVDGISHKKYIEEGKLVKSTSEENRTSERLSELLSIRLDIYIKNPDNASEEENRIRRENL
KKFFSNKVLHLKDSVLYLKNRKEKNAVQDKNYSEEDISEYDLKNKNSFSVLKKILLNEDVNSEE
LEIFRKDVEAKLNKINSLKYSFEENKANYQKINENNVEKVGGKSKRNIIYDYYRESAKRNDYIN
NVQEAFDKLYKKEDIEKLFFLIENSKKHEKYKIREYYHKIIGRKNDKENFAKIIYEEIQNVNNI
KELIEKIPDMSELKKSQVFYKYYLDKEELNDKNIKYAFCHFVEIEMSQLLKNYVYKRLSNISND
KIKRIFEYQNLKKLIENKLLNKLDTYVRNCGKYNYYLQVGEIATSDFIARNRQNEAFLRNIIGV
SSVAYFSLRNILETENENDITGRMRGKTVKNNKGEEKYVSGEVDKIYNENKQNEVKENLKMFYS
YDFNMDNKNEIEDFFANIDEAISSIRHGIVHFNLELEGKDIFAFKNIAPSEISKKMFQNEINEK
KLKLKIFKQLNSANVFNYYEKDVIIKYLKNTKFNFVNKNIPFVPSFTKLYNKIEDLRNTLKFFW
SVPKDKEEKDAQIYLLKNIYYGEFLNKFVKNSKVFFKITNEVIKINKQRNQKTGHYKYQKFENI
EKTVPVEYLAIIQSREMINNQDKEEKNTYIDFIQQIFLKGFIDYLNKNNLKYIESNNNNDNNDI
FSKIKIKKDNKEKYDKILKNYEKHNRNKEIPHEINEFVREIKLGKILKYTENLNMFYLILKLLN
HKELTNLKGSLEKYQSANKEETFSDELELINLLNLDNNRVTEDFELEANEIGKFLDFNENKIKD
RKELKKFDTNKIYFDGENIIKHRAFYNIKKYGMLNLLEKIADKAKYKISLKELKEYSNKKNEIE
KNYTMQQNLHRKYARPKKDEKFNDEDYKEYEKAIGNIQKYTHLKNKVEFNELNLLQGLLLKILH
RLVGYTSIWERDLRFRLKGEFPENHYIEEIFNFDNSKNVKYKSGQIVEKYINFYKELYKDNVEK
RSIYSDKKVKKLKQEKKDLYIRNYIAHFNYIPHAEISLLEVLENLRKLLSYDRKLKNAIMKSIV
DILKEYGFVATFKIGADKKIEIQTLESEKIVHLKNLKKKKLMTDRNSEELCELVKVMFEYKALE

Leptotrichia shahii Cas13a:

(SEQ ID NO: 63)
MGNLFGHKRWYEVRDKKDFKIKRKVKVKRNYDGNKYILNINENNNKEKIDNNKFIRKYINYKKN
DNILKEFTRKFHAGNILFKLKGKEGIIRIENNDDFLETEEVVLYIEAYGKSEKLKALGITKKKI
IDEAIRQGITKDDKKIEIKRQENEEEIEIDIRDEYTNKTLNDCSIILRIIENDELETKKSIYEI
FKNINMSLYKIIEKIIENETEKVFENRYYEEHLREKLLKDDKIDVILTNFMEIREKIKSNLEIL
GFVKFYLNVGGDKKKSKNKKMLVEKILNINVDLTVEDIADFVIKELEFWNITKRIEKVKKVNNE
FLEKRRNRTYIKSYVLLDKHEKFKIERENKKDKIVKFFVENIKNNSIKEKIEKILAEFKIDELI
KKLEKELKKGNCDTEIFGIFKKHYKVNFDSKKFSKKSDEEKELYKIIYRYLKGRIEKILVNEQK
VRLKKMEKIEIEKILNESILSEKILKRVKQYTLEHIMYLGKLRHNDIDMTTVNTDDFSRLHAKE
ELDLELITFFASTNMELNKIFSRENINNDENIDFFGGDREKNYVLDKKILNSKIKIIRDLDFID
NKNNITNNFIRKFTKIGTNERNRILHAISKERDLQGTQDDYNKVINIIQNLKISDEEVSKALNL
DVVFKDKKNIITKINDIKISEENNNDIKYLPSFSKVLPEILNLYRNNPKNEPFDTIETEKIVLN
ALIYVNKELYKKLILEDDLEENESKNIFLQELKKTLGNIDEIDENIIENYYKNAQISASKGNNK
AIKKYQKKVIECYIGYLRKNYEELFDFSDFKMNIQEIKKQIKDINDNKTYERITVKTSDKTIVI
NDDFEYIISIFALLNSNAVINKIRNRFFATSVWLNTSEYQNIIDILDEIMQLNTLRNECITENW
NLNLEEFIQKMKEIEKDFDDFKIQTKKEIFNNYYEDIKNNILTEFKDDINGCDVLEKKLEKIVI
FDDETKFEIDKKSNILQDEQRKLSNINKKDLKKKVDQYIKDKDQEIKSKILCRIIFNSDFLKKY
KKEIDNLIEDMESENENKFQEIYYPKERKNELYIYKKNLFLNIGNPNFDKIYGLISNDIKMADA
KFLFNIDGKNIRKNKISEIDAILKNLNDKLNGYSKEYKEKYIKKLKENDDFFAKNIQNKNYKSF
EKDYNRVSEYKKIRDLVEFNYLNKIESYLIDINWKLAIQMARFERDMHYIVNGLRELGIIKLSG
YNTGISRAYPKRNGSDGFYTTTAYYKFFDEESYKKFEKICYGFGIDLSENSEINKPENESIRNY
ISHFYIVRNPFADYSIAEQIDRVSNLLSYSTRYNNSTYASVFEVFKKDVNLDYDELKKKFKLIG
NNDILERLMKPKKVSVLELESYNSDYIKNLIIELLTKIENTNDTL

By “Type VI CRISPR polynucleotide” is meant a nucleic acid encoding a Type VI CRISPR polypeptide.

By “UVSX recombinase polypeptide” is meant a protein with at least 85% identity to UniProt Accession No. P04529, which is provided below, and capable of functioning in genetic recombination, DNA repair, and/or replication, or a functional fragment thereof. In various embodiments, the UVSX recombinase polypeptide is suitable for use in a recombinase polymerase amplification (RPA) reaction. In some embodiments, a UVSX recombinase polypeptide may be obtained commercially under the trade name TwistAmp® (see, e.g., Stringer, et al., “TwistAmp® Liquid: a versatile amplification method to replace PCR,” Nature Methods, 15, 395 (2018), doi: 10.1038/nmeth.f.407, the disclosure of which is hereby incorporated by reference in its entirety for all purposes.

>sp|P04529|UVSX_BPT4 Recombination and repair protein OS=Enterobacteria phage T4 OX=10665 GN=UVSX PE=1 SV=2

(SEQ ID NO: 64)
MSDLKSRLIKASTSKLTAELTASKFFNEKDVVRTKIPMMNIALSGEITGGMQSGLLILAGPSKS
FKSNFGLTMVSSYMRQYPDAVCLFYDSEFGITPAYLRSMGVDPERVIHTPVQSLEQLRIDMVNQ
LDAIERGEKVVVFIDSLGNLASKKETEDALNEKVVSDMTRAKTMKSLFRIVTPYFSTKNIPCIA
INHTYETQEMFSKTVMGGGTGPMYSADTVFIIGKRQIKDGSDLQGYQFVLNVEKSRTVKEKSKF
FIDVKFDGGIDPYSGLLDMALELGFVVKPKNGWYAREFLDEETGEMIREEKSWRAKDTNCTTFW
GPLFKHQPFRDAIKRAYQLGAIDSNEIVEAEVDELINSKVEKFKSPESKSKSAADLETDLEQLS
DMEEFNE

By “UVSX recombinase polynucleotide” is meant a protein encoding a UVSX recombinase polypeptide.

By “D-alanine-(R)-lactate ligase VanA (vanA) polypeptide” is meant a protein having vancomycin-resistant D-alanine-(R)-lactate ligase activity and with at least 85% sequence identity to GenBank® Ref. Seq. Accession No. ADM24920.1, which is provided below, or a functional fragment thereof:

>ADM24920.1 VanA (plasmid) [Enterococcus faecalis]

(SEQ ID NO: 115)
MNRIKVAILFGGCSEEHDVSVKSAIEIAANINKEKYEPLYIGITKSGVWKMCEKPCAEWENDNC
YSAVLSPDKKMHGLLVKKNHEYEINHVDVAFSALHGKSGEDGSIQGLFELSGIPFVGCDIQSSA
ICMDKSLTYIVAKNAGIATPAFWVINKDDRPVAATFTYPVFVKPARSGSSFGVKKVNSADELDY
AIESARQYDSKILIEQAVSGCEVGCAVLGNSAALVVGEVDQIRLQYGIFRIHQEVEPEKGSENA
VITVPADLSAEERGRIQETAKKIYKALGCRGLARVDMFLQDNGRIVLNEVNTLPGFTSYSRYPR
MMAAAGIALPELIDRLIVLALKG

By “D-alanine-(R)-lactate ligase VanA (vanA) polynucleotide” is meant a nucleotide molecule encoding a vancomycin-resistant D-alanine-(R)-lactate ligase polypeptide. A representative vancomycin-resistant D-alanine-(R)-lactate ligase polynucleotide sequence is provided below (GenBank® Ref. Seq. GQ484956.1:28460-29491):

>GQ484956.1:28460-29491 Enterococcus faecalis plasmid pWZ1668, complete sequence

(SEQ ID NO: 116)
ATGAATAGAATAAAAGTTGCAATACTGTTTGGGGGTTGCTCAGAGGAGCATGACGTATCGGTAA
AATCTGCAATAGAGATAGCCGCTAACATTAATAAAGAAAAATACGAGCCGTTATACATTGGAAT
TACGAAATCTGGTGTATGGAAAATGTGCGAAAAACCTTGCGCGGAATGGGAAAACGACAATTGC
TATTCAGCTGTACTCTCGCCGGATAAAAAAATGCACGGATTACTTGTTAAAAAGAACCATGAAT
ATGAAATCAACCATGTTGATGTAGCATTTTCAGCTTTGCATGGCAAGTCAGGTGAAGATGGATC
CATACAAGGTCTGTTTGAATTGTCCGGTATCCCTTTTGTAGGCTGCGATATTCAAAGCTCAGCA
ATTTGTATGGACAAATCGTTGACATACATCGTTGCGAAAAATGCTGGGATAGCTACTCCCGCCT
TTTGGGTTATTAATAAAGATGATAGGCCGGTGGCAGCTACGTTTACCTATCCTGTTTTTGTTAA
GCCGGCGCGTTCAGGCTCATCCTTCGGTGTGAAAAAAGTCAATAGCGCGGACGAATTGGACTAC
GCAATTGAATCGGCAAGACAATATGACAGCAAAATCTTAATTGAGCAGGCTGTTTCGGGCTGTG
AGGTCGGTTGTGCGGTATTGGGAAACAGTGCCGCGTTAGTTGTTGGCGAGGTGGACCAAATCAG
GCTGCAGTACGGAATCTTTCGTATTCATCAGGAAGTCGAGCCGGAAAAAGGCTCTGAAAACGCA
GTTATAACCGTTCCCGCAGACCTTTCAGCAGAGGAGCGAGGACGGATACAGGAAACGGCAAAAA
AAATATATAAAGCGCTCGGCTGTAGAGGTCTAGCCCGTGTGGATATGTTTTTACAAGATAACGG
CCGCATTGTACTGAACGAAGTCAATACTCTGCCCGGTTTCACGTCATACAGTCGTTATCCCCGT
ATGATGGCCGCTGCAGGTATTGCACTTCCCGAACTGATTGACCGCTTGATCGTATTAGCGTTAA
AGGGGTGA

By “D-alanine-(R)-lactate ligase VanB (vanB) polypeptide” is meant a protein having vancomycin-resistant D-alanine-(R)-lactate ligase activity and with at least 85% sequence identity to GenBank® Ref. Seq. Accession No. AAF72363.1, which is provided below, or a functional fragment thereof:

>AAF72363.1 VanB (plasmid) [Enterococcus faecalis]

(SEQ ID NO: 117)
MNRIKVAIIFGGCSEEHDVSVKSAIEIAANINTEKFDPHYIGITKNGVWKLCKKPCTEWEADSL
PAILSPDRKTHGLLVMKESEYETRRIDVAFPVLHGKCGEDGAIQGLFELSGIPYVGCDIQSSAA
CMDKSLAYILTKNAGIAVPEFQMIDKGDKPEAGALTYPVFVKPARSGSSFGVTKVNGTEELNAA
IEAAGQYDGKILIEQAISGCEVGCAVMGNEDDLIVGEVDQIRLSHGIFRIHQENEPEKGSENAM
ITVPADIPVEERNRVQETAKKVYRVLGCRGLARVDLFLQEDGGIVLNEVNTLPGFTSYSRYPRM
VAAAGITLPALIDSLITLALKR

By “D-alanine-(R)-lactate ligase VanB (vanB) polynucleotide” is meant a nucleotide molecule encoding a vancomycin-resistant D-alanine-(R)-lactate ligase polypeptide. A representative vancomycin-resistant D-alanine-(R)-lactate ligase polynucleotide sequence is provided below (GenBank® Ref. Seq. AF192329.1:28835-29863):

>AF192329.1:28835-29863 Enterococcus faecalis transposon Tn1549, complete sequence

(SEQ ID NO: 118)
ATGAATAGAATAAAAGTCGCAATCATCTTCGGCGGTTGCTCGGAGGAACATGATGTGTCGGTAA
AATCCGCAATAGAAATTGCTGCGAACATTAATACTGAAAAATTCGATCCGCACTACATCGGAAT
TACAAAAAACGGCGTATGGAAGCTATGCAAGAAGCCATGTACGGAATGGGAAGCCGACAGTCTC
CCCGCCATACTCTCCCCGGATAGGAAAACGCATGGGCTGCTTGTCATGAAAGAAAGCGAATACG
AAACACGGCGTATTGATGTGGCTTTCCCGGTTTTGCATGGCAAATGCGGGGAGGATGGTGCGAT
ACAGGGTCTGTTTGAATTGTCTGGTATCCCCTATGTAGGCTGCGATATTCAAAGCTCCGCAGCT
TGCATGGACAAATCACTGGCCTACATTCTTACAAAAAATGCGGGCATCGCCGTTCCCGAATTTC
AAATGATTGATAAAGGTGACAAGCCGGAGGCGGGTGCGCTTACCTACCCTGTCTTTGTGAAGCC
GGCACGGTCAGGTTCGTCCTTTGGCGTAACCAAAGTAAACGGTACGGAAGAACTTAACGCTGCG
ATAGAAGCGGCAGGACAATATGATGGAAAAATCTTAATTGAGCAAGCGATTTCGGGCTGTGAGG
TCGGGTGTGCGGTCATGGGGAACGAGGATGATTTGATTGTCGGCGAAGTGGATCAAATCCGGCT
GAGCCACGGTATCTTCCGCATCCATCAGGAAAACGAGCCGGAAAAAGGCTCAGAAAATGCGATG
ATTACAGTTCCCGCAGACATTCCGGTCGAGGAACGAAATCGGGTGCAGGAAACGGCAAAGAAAG
TATATCGGGTGCTTGGATGCAGAGGGCTTGCCCGTGTTGATCTTTTTTTGCAGGAGGATGGCGG
CATCGTTCTAAATGAGGTCAATACCCTGCCTGGTTTTACATCGTACAGCCGCTACCCACGTATG
GTGGCCGCCGCAGGAATCACGCTTCCTGCACTGATTGACAGCCTGATTACATTGGCGTTAAAGA
GGTGA

By “D-alanine-D-serine ligase VanC (vanC) polypeptide” is meant a protein having vancomycin-resistant D-alanine-D-serine ligase activity and with at least 85% sequence identity to GenBank® Ref. Seq. Accession No. WRO93625.1, which is provided below, or a functional fragment thereof:

>WRO93625.1 D-alanine-D-serine ligase VanC [Enterococcus casseliflavus]

(SEQ ID NO: 119)
MKKIAIIFGGNSPEYTVSLASATSTIEALQSSPYDYDLSLIGIAPDAMDWYLYTGELENIRQDT
WLLDTKHKQKIQPLFEGNGFWLSEEQQTLVPDVLFPIMHGKYGEDGSIQGLFELMKLPYVGCGV
AGSALCMNKWLLHQAAAAIGVQSAPTILLTNQANQQEQIEAFIQTHGFPVFFKPNEAGSSKGIT
KVTCVEEIASALKEAFTYCSAVLLQKNIAGVEIGCGILGNDSLTVGACDAISLVDGFFDFEEKY
QLISAKITVPAPLPETIETKVKEQAQLLYRSLGLKGLARIDFFVTDQGELYLNEINTMPGFTSH
SRYPAMMAAVGLSYQELLQKLLVLAKEEVK

By “D-alanine-D-serine ligase VanC (vanC) polynucleotide” is meant a nucleotide molecule encoding a vancomycin-resistant D-alanine-D-serine ligase polypeptide. A representative vancomycin-resistant D-alanine-D-serine ligase polynucleotide sequence is provided below (GenBank® Ref. Seq. CP141640.1:2462517-2463569):

>CP141640.1:2462517-2463569 Enterococcus casseliflavus strain Dec0527 chromosome, complete genome

(SEQ ID NO: 120)
ATGAAAAAAATCGCCATTATTTTTGGAGGCAATTCACCGGAATACACCGTTTCTTTAGCTTCAG
CAACTAGCACAATCGAAGCACTCCAATCATCTCCCTATGACTACGACCTCTCTTTGATCGGGAT
CGCCCCAGATGCTATGGATTGGTACTTGTATACAGGAGAACTGGAAAACATCCGACAAGACACG
TGGTTGTTGGATACGAAACATAAACAGAAAATACAGCCGCTATTCGAAGGAAACGGCTTTTGGC
TAAGTGAAGAGCAGCAAACGTTGGTACCTGATGTTTTATTTCCCATTATGCATGGCAAATATGG
GGAAGATGGCAGTATCCAAGGATTGTTTGAATTGATGAAGCTGCCTTATGTAGGTTGCGGGGTG
GCCGGTTCTGCCTTATGTATGAACAAATGGCTGCTGCATCAAGCTGCAGCAGCCATTGGCGTAC
AAAGTGCTCCTACGATTCTCTTGACAAATCAAGCCAACCAGCAAGAACAAATCGAAGCTTTTAT
CCAGACCCATGGCTTTCCAGTTTTCTTTAAGCCTAATGAAGCGGGCTCCTCAAAAGGGATCACT
AAAGTCACCTGCGTTGAAGAAATCGCTTCTGCCTTAAAAGAAGCCTTTACTTATTGTTCCGCAG
TGCTCCTACAAAAAAATATTGCCGGTGTTGAGATCGGTTGCGGTATTTTGGGCAACGACTCTTT
GACTGTCGGTGCTTGTGACGCCATTTCATTAGTAGACGGCTTTTTCGATTTTGAAGAAAAGTAC
CAGCTGATCAGCGCCAAAATCACCGTCCCTGCGCCATTGCCTGAAACGATTGAAACCAAGGTCA
AAGAACAAGCTCAGCTGCTCTATCGTAGTCTTGGTCTTAAAGGTCTTGCTCGCATCGACTTTTT
TGTCACGGATCAAGGAGAACTATACTTGAATGAAATCAATACTATGCCGGGCTTTACGAGTCAC
TCCCGCTATCCTGCCATGATGGCAGCGGTCGGCTTATCCTATCAAGAACTACTACAAAAACTGC
TTGTCTTAGCAAAGGAGGAAGTCAAATGA

By “Verona integron-encoded metallo-beta-lactamase (VIM; bla_{VIM}) polypeptide” is meant a protein having Verona integron-encoded metallo-beta-lactamase activity and with at least 85% sequence identity to GenBank® Ref. Seq. Accession No. WCS41564.1, which is provided below, or a functional fragment thereof:

>WCS41564.1 VIM family beta-lactamase [Pseudomonas aeruginosa]

(SEQ ID NO: 121)
MPRASKQQARYAVGRCLMLWSSNDVTQQGSRPKTKLCRTHPHGVLMFKLLSKLLVYLTA
SIMAIASPLAFSVDSSGEYPTVSEIPVGEVRLYQIADGVWSHIATQSFDGAVYPSNGLIVRDGD
ELLLIDTAWGAKNTAALLAEIEKQIGLPVTRAVSTHFHDDRVGGVDVLRAAGVATYASPSTRRL
AEVEGNEIPTHSLEGLSSSGDAVRFGPVELFYPGAAHSTDNLVVYVPSASVLYGGCAIYELSRT
SAGNVADADLAEWPTSIERIQQHYPEAQFVIPGHGLPGGLDLLKHTTNVVKAHTNRSVVE

By “Verona integron-encoded metallo-beta-lactamase (VIM; bla_{VIM}) polynucleotide” is meant a nucleotide molecule encoding a Verona integron-encoded metallo-beta-lactamase polypeptide. A representative Verona integron-encoded metallo-beta-lactamase polynucleotide sequence is provided below (GenBank® Ref. Seq. OP329418.1:15658-16593):

>OP329418.1:15658-16593 Pseudomonas aeruginosa strain Pa873 antibiotic resistance region genomic sequence

(SEQ ID NO: 122)
ATGCCTCGGGCATCCAAGCAGCAAGCGCGTTACGCCGTGGGTCGATGTTTGATGTTATG
GAGCAGCAACGATGTTACGCAGCAGGGCAGTCGCCCTAAAACAAAGTTATGCCGCACTCACCCC
CATGGAGTTTTGATGTTCAAACTTTTGAGTAAGTTATTGGTCTATTTGACCGCGTCTATCATGG
CTATTGCGAGTCCGCTCGCTTTTTCCGTAGATTCTAGCGGTGAGTATCCGACAGTCAGCGAAAT
TCCGGTCGGGGAGGTCCGGCTTTACCAGATTGCCGATGGTGTTTGGTCGCATATCGCAACGCAG
TCGTTTGATGGCGCAGTCTACCCGTCCAATGGTCTCATTGTCCGTGATGGTGATGAGTTGCTTT
TGATTGATACAGCGTGGGGTGCGAAAAACACAGCGGCACTTCTCGCGGAGATTGAGAAGCAAAT
TGGACTTCCTGTAACGCGTGCAGTCTCCACGCACTTTCATGACGACCGCGTCGGCGGCGTTGAT
GTCCTTCGGGCGGCTGGGGTGGCAACGTACGCATCACCGTCGACACGCCGGCTAGCCGAGGTAG
AGGGGAACGAGATTCCCACGCACTCTCTAGAAGGACTCTCATCGAGCGGGGACGCAGTGCGCTT
CGGTCCAGTAGAACTCTTCTATCCTGGTGCTGCGCATTCGACCGACAACTTAGTTGTGTACGTC
CCGTCTGCGAGTGTGCTCTATGGTGGTTGTGCGATTTATGAGTTGTCACGCACGTCTGCGGGGA
ACGTGGCCGATGCCGATCTGGCTGAATGGCCCACCTCCATTGAGCGGATTCAACAACACTACCC
GGAAGCACAGTTCGTCATTCCGGGGCACGGCCTGCCGGGCGGTCTAGACTTGCTCAAGCACACA
ACGAATGTTGTAAAAGCGCACACAAATCGCTCAGTCGTTGAGTAG

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A provides a Schematic diagram of a standard clinical microbiology workflow for positive blood cultures in a well-resourced laboratory (top), demonstrating multiple sequential steps, extensive hands-on time, and reliance on complex instrumentation. In contrast, the Bacteria and AMR Detection by SHERLOCK (BADLOCK) workflow (bottom) demonstrated significantly reduced resource and instrument utilization and provides a much faster turnaround time for clinically actionable information. FIG. 1B provides a schematic diagram of a molecular mechanism of a Specific High-Sensitivity Enzymatic Reporter unLOCKing (SHERLOCK) platform (see also International Patent Application WO 2018/107129, WO 2020/124050, and U.S. Pat. No. 10,266,887, the contents of each of which are hereby incorporated by reference in their entirety for all purposes). Pre-specified gene targets were amplified isothermally using recombinase polymerase amplification (RPA). The forward primer includes a universal T7 tag, enabling transcription of the amplified gene target into RNA. The CRISPR/Cas13a complex then bound its cognate RNA target, activating its enzymatic function and triggering trans-enzymatic cleavage. This results in the indiscriminate cleavage of nearby reporter molecules, producing a detectable signal that is visualized as fluorescence or as a colorimetric readout on a lateral flow strip.

FIG. 2A provides a schematic diagram of an approach to species primer and guide development, showing that certain targets map to individual species, whereas others capture a group of closely related bacteria with similar clinical management pathways. FIG. 2B provides a schematic diagram demonstrating how antimicrobial resistance (AMR) gene targets are designed to capture most sequence variants for a given gene by targeting conserved regions. FIG. 2C provides a schematic diagram of the BADLOCK workflow. Once blood cultures turn positive, a heat lysis step is performed while a Gram stain is concurrently conducted. Gene panel selection is guided by gram-stain morphology, with this experiment focusing exclusively on gram-negative rods. A single master mix is prepared and divided among different recombinase polymerase amplification (RPA) primer and CRISPR guide pairs, each specifying a unique target. The reactions are incubated for 50 minutes, after which fluorescence values are measured to determine target detection. For simplicity, only gram-negative species targets are shown here.

FIG. 3 provides an overview of BADLOCK testing cohorts and associated CRISPR targets. Summary of each experimental group evaluated, including A) species testing on clinical blood culture specimens; B) antimicrobial resistance (AMR) gene testing on both clinical samples and banked laboratory isolates; C) species testing on mock urinary tract infection (UTI) samples created from banked isolates; and D) AMR testing on mock UTI samples also created from banked isolates. Each cohort is annotated with the specific CRISPR targets used, illustrating the number and distribution of species—and resistance gene-level detection across experiment phases. Each sample was tested for all species targets within the target panel simultaneously, which are represented in the boxes above each cohort. “CRO” is the abbreviation of the 3^{rd} generation cephalosporin ceftriaxone.

FIG. 4 provides scatterplots demonstrating the performance of a BADLOCK species panel on positive blood culture clinical samples. Each panel displays a scatterplot of relative fluorescence unit (RFU) values for the corresponding species target across all 194 samples. A confusion matrix summarizing the total counts for each call is included for each species. Ground truth (x-axis) was determined by routine testing in the clinical microbiology laboratory using the MALDI-TOF platform.

FIG. 5 provides scatterplots demonstrating BADLOCK AMR gene testing in a clinical cohort of positive blood culture specimens, limited to those 46 samples resistant to ceftriaxone, with gene content (x-axis) validated by PCR testing or whole-genome sequencing.

FIG. 6 provides scatterplots demonstrating BADLOCK AMR gene panel testing performance from banked clinical laboratory strains with known genetic backgrounds, validated by whole-genome sequencing.

FIG. 7 provides scatterplots demonstrating BADLOCK testing on mock urine samples, including both species and antimicrobial resistance (AMR) gene detection.

FIG. 8 provides a bar graph that proportionally represents the gram-negative rod species from the entire clinical cohort. Species included in BADLOCK are labeled with an asterisk.

FIG. 9 provides bar graphs demonstrating the limit of detection (LOD) analysis of the BADLOCK assay using LB-cultured isolates. Relative fluorescence unit (RFU) values are shown for three representative bacterial species across a serial dilution range measured in CFU/mL. The BADLOCK assay reproducibly detects targets at concentrations of ˜106 CFU/mL (corresponding to 103 CFU/μL), establishing this as the approximate lower limit of detection under these experimental conditions.

FIG. 10 provides a plot demonstrating threshold determination for species and antimicrobial resistance gene targets in the BADLOCK assay. Fluorescence values from technical replicates are shown for species targets using clinical samples, and for antimicrobial resistance (AMR) gene targets using banked clinical strains. The horizontal dashed line indicates the threshold for calling a positive result, defined as the mean plus six times the standard deviation of fluorescence values from 24 off-target samples for species reactions and 12 off-target samples for AMR gene reactions.

FIG. 11 provides a consolidated visualization of BADLOCK species assay runs across all tested blood culture samples collected for this experiment. Each row represents an individual sample, and each column corresponds to a specific BADLOCK target with the assay layout shown in the boxes above and enumerated in the accompanying key. Samples are grouped by the species for which they were known positives as identified by the clinical microbiology laboratory's standard workflow. Each cell in the grid reflects a single target reaction for a given sample, color-coded by accuracy. Overlaid grey bars represent the corresponding relative fluorescence unit (RFU) values. Symbols adjacent to species names indicate the cohort origin of each sample and are harmonized with the legend in FIG. 3. Only the first run for each reaction is shown. Polymicrobial samples appear multiple times—once for each on-panel species present, and again in the “polymicrobial” section.

FIG. 12 provides a consolidated visualization of BADLOCK AMR gene assay runs across all tested samples. Each row represents an individual sample, and each column corresponds to a specific BADLOCK AMR gene target, with the assay layout shown in the boxes above and enumerated in the accompanying key. Samples are grouped by the resistance gene known to be present by either PCR testing (clinical resistant cohort) or whole genome sequencing (banked cohort) (Table 3). Each cell in the grid reflects a single target reaction for a given sample, color-coded by accuracy, with overlaid grey bars representing the corresponding relative fluorescence unit (RFU) values. Symbols adjacent to gene names indicate the cohort origin of each sample and are harmonized with the legend in FIG. 3. Samples containing multiple antimicrobial resistance (AMR) gene targets appear in each group for which a corresponding target was known to be present.

FIG. 13 provides a plot demonstrating the repeat testing of falsely negative samples in the BADLOCK assay. Each point represents a clinical sample that was initially negative by BADLOCK but positive for the indicated species by the reference method. For simplicity, only the discordant targets are shown. Samples are displayed along the x-axis, grouped by species, with shape indicating whether the data correspond to the original or repeat run. The dashed horizontal line marks the species-specific relative fluorescence unit (RFU) positivity threshold. A change in result classification occurs when a point crosses this threshold between runs, marked by a shaded triangle. 5 samples retested as concordant, while 17 remained discordant. Ten of these discordant negatives were in E. coli while 7 were non-E. coli species, and 4 of these were from polymicrobial samples (IDs 172, 126, 34, and 121). One E. coli sample that retested as concordant was 106, which was confirmed to have the chuA gene target by PCR, while the other lacked the target.

FIG. 14 provides a plot demonstrating the repeat testing of discordantly positive samples in the BADLOCK assay. Each point represents a clinical sample that was initially positive by BADLOCK for the indicated species, which was not found by the reference method. For simplicity, only the discordant targets are shown. Samples are displayed on the x-axis and grouped by species, with shape distinguishing original versus repeat reactions. The dashed horizontal line indicates the species-specific relative fluorescence unit (RFU) positivity threshold. A change in result classification occurs when a point crosses this threshold between reactions, marked by a shaded triangle. Upon retesting, 13 samples were corrected, whereas 7 remained discordant. Of these, two samples (IDs 195 and 156) yielded RFU values within 11% of the threshold in both reactions, whereas the remaining four consistently showed strong positive signals above threshold.

FIG. 15 provides a plot demonstrating the repeat testing of discordantly positive samples in the BADLOCK AMR assay. Samples are displayed on the x-axis and grouped by antimicrobial resistance (AMR) target, with shape distinguishing original versus repeat reactions. The dashed horizontal line indicates the target-specific relative fluorescence unit (RFU) positivity threshold. A change in result classification occurs when a point crosses this threshold between reactions, marked by a shaded triangle.

FIG. 16 provides receiver operating characteristic (ROC) curves for each species-specific and antimicrobial resistance (AMR) gene target. Curves were generated by comparing relative fluorescence unit (RFU) values to known true-positive and true-negative classifications after excluding suspected contaminants (defined as samples with repeat RFU values >3-fold lower than the original) and E. coli false negatives lacking the chuA gene as confirmed by PCR. Each curve reflects the performance of a primer-guide target, with the area under the curve (AUC) in red text. Most targets demonstrated high discriminative ability, with AUC values ≥0.97 for the majority of species and AMR genes. The inset table displays the optimal threshold values along with their corresponding performance metrics across the cohort.

FIG. 17 provides lateral flow strips for several representative isolates, with the true species listed in the header. As in the fluorescent readout, each sample was run across all species targets in the order listed at right. Relative fluorescence unit (RFU) values from each BADLOCK reaction using the fluorescent readout are shown below, with true positive values highlighted in gray.

FIGS. 18A to 18D provide images of lateral flow strips used to detect the antimicrobial resistance (AMR) genes CTX-M-15 (FIG. 18A), KPC (FIG. 18B), NDM (FIG. 18C), and OXA-48 (FIG. 18D) across different representative isolates, with the same format as above. Relative fluorescence unit (RFU) values from each BADLOCK reaction are shown below, with true positive values highlighted in gray.

FIG. 19 provides bar graphs demonstrating limit of detection (LOD) for selected bacterial species included in the urine-based BADLOCK testing. Bars represent the lowest concentration (CFU/mL) at which each species was consistently detected using the BADLOCK assay. LOD values were determined using dilution series in pooled human urine, with detection defined as consistent assay positivity across replicates.

FIG. 20 provides a plot demonstrating threshold determination for species and antimicrobial resistance gene targets in the BADLOCK assay on commercial pooled human urine samples spiked with the indicated pathogen at 10\hat{ }5 CFU/mL. Fluorescence values from technical replicates are shown. The horizontal dashed line indicates the threshold for calling a positive result, defined as the mean plus six times the standard deviation of fluorescence values from 12 off-target samples for species reactions and between 9 and 15 off-target samples for antimicrobial resistance (AMR) gene reactions (variation due to some strains containing multiple target genes).

FIG. 21 provides a consolidated visualization of BADLOCK assay runs for mock urinary tract infection (UTI) samples, including both species detection and antimicrobial resistance (AMR) gene testing. Each row represents an individual sample, and each column corresponds to a BADLOCK target with the assay layout shown in the boxes above and listed in the accompanying key. Samples are grouped according to the species or AMR gene known to be present. Each cell in the grid reflects a single reaction for a given sample, color-coded by call, with overlaid grey bars representing the corresponding relative fluorescence unit (RFU) values. Symbols adjacent to each target indicate the cohort or target class (species or resistance gene), harmonized with the legend in FIG. 3. Samples containing multiple targets are shown in each group for which a corresponding target was present. The off-target species are listed next to the sample row.

### DETAILED DESCRIPTION

The present disclosure features compositions and methods for detecting bacterial pathogens in a sample containing microbial resistance genes, and for selecting subjects having an infection with such species for treatment. The methods involve carrying out a series of reactions in parallel where each reaction involves detecting a bacterial species or resistance gene in a sample using a CRISPR-Cas13a reaction. In some embodiments, the methods involve culturing a blood sample to increase the concentration of bacteria in the sample and/or direct detection of bacterial pathogens and/or microbial resistance genes in a blood sample.

The present disclosure is based, at least in part, upon the development, as described in the Examples provided below, of BADLOCK (Bacterial and AMR Detection by Specific High-Sensitivity Enzymatic Reporter Unlocking” (SHERLOCK)), which provides a rapid, low-cost molecular diagnostic platform for direct detection of bacterial pathogens and resistance genes from clinical samples. The methods described herein can be provided as a portable CRISPR assay that enables fast detection of bacterial infections and antimicrobial resistance (AMR) genes. In embodiments, the present methods are particularly well suited for supporting patient care in resource-limited settings. In one embodiment, the methods described herein feature a CRISPR-Cas13a reaction capable of detecting nine exemplary bacterial species and four major resistance genes directly from positive blood culture. It requires only a heat block and supports both fluorescence and paper-based lateral flow readouts. BADLOCK was validated on a prospectively collected clinical cohort of 194 blood culture specimens, supplemented with 69 mock samples generated from banked isolates enriched for targeted resistance genes. Across all cohorts, 2,224 individual reactions were conducted, achieving 97.6% accuracy (2,171/2,224) at the reaction level. At the assay level, 89.5% (274/306) showed perfect or partial concordance with gold-standard species and resistance gene detection, including 255 assays with perfect concordance and 19 with partial concordance (correct detection of at least one pathogen). This included an evaluation of BADLOCK as a potential culture-free diagnostic for urinary tract infections (UTIs), achieving 98.0% reaction-level accuracy. At the assay level, 90.7% (41/43) were perfectly concordant with gold-standard detection of both species and resistance genes, with 2 additional assays showing partial concordance. This represents the first demonstration of the CRISPR-Cas13a diagnostic platform on clinical bloodstream infections to date and supports BADLOCK's use as a practical and scalable solution for rapid pathogen and resistance gene detection in resource-constrained settings.

The present disclosure is also based, at least in part, on the unexpected discovery of methods for rapid detection of pathogens (e.g., bacterial pathogens) directly from blood cultures. The techniques herein provide for parallel processing of a single blood culture to determine the presence or absence of multiple bacterial species within the blood culture, including detection of antimicrobial resistant (AMR) strains of bacterial pathogens in the blood culture. The present disclosure provides for methods for rapid (between about 60 and about 90 minutes) and low-cost detection of pathogens requiring only minimal equipment such as a heat source (e.g., a heat block, boiling water, and the like) to lyse the blood culture, which may then be added to a reagent in multiple tubes to allow parallel processing and detection of multiple bacterial species, and then assayed on a lateral flow test (LFT) to detect bacterial species and/or AMR bacterial strains. As described herein, the present disclosure provides for such rapid detection of bacterial pathogens by using targets such as amino-glycosides, carbapenemases (e.g., IMP, VIM, KPC, NDM-1, OXA-48), extended-spectrum beta-lactamases (ESBLs) (e.g., (CTX-M-15, SHV-12, SHV-2), AMP-C, fluoro-quinolone, Topoisomerase I (TOP 1), Elastase B (LasB), chuA, mecA, vanA, vanB, vanC, a computationally identified stretch of DNA and/or a computationally derived shared gene fragment to detect AMR bacterial strains (e.g., E. coli, K. pneumoniae, E. cloacae, P. aeruginosa, S. marcescens, S. maltophilia, C. freundii, P. mirabilis, A. baumannii, K. oxytoca, S. aureus, S. epidermidis, S. hominis, S. haemolyticus, S. capitis, S. lugdunensis, S. pettenkoferi, Salmonella typhi, Citrobacter koseri, Klebsiella aerogenes, and Bacteroides fragilis, Coagulase-Negative Staphylococci (CoNS), Streptococcus anginosus, Streptococcus bovis, S. pyogenes, Enterococcus faecalis, Enterococcus faecium, Streptococcus pneumoniae, Streptococcus agalactiae, S. constellatus, S. intermedius).

### Antimicrobial-Resistant Organisms

Antimicrobial-resistant (AMR) organism infections represent a global health challenge . . . . One of the most concerning clinical manifestations of AMR is in bloodstream infections (BSIs), which are among the most severe and life-threatening infectious syndromes. BSIs can be caused by a wide range of bacterial pathogens, but the mortality rate is significantly higher when resistant bacteria are involved, and the early identification of these pathogens can improve patient survival. Therefore, a low-cost method to rapidly detect bacterial species and their AMR genes in clinical samples would be an invaluable tool for clinicians.

In various embodiments, a microbial species or taxonomic group targeted for identification using the methods and/or compositions of the present disclosure is a gram-negative species, such as one selected from one or more of E. coli, K. pneumoniae, E. cloacae, P. aeruginosa, S. marcescens, S. maltophilia, C. freundii, P. mirabilis, A. baumannii, K. oxytoca, A. baumanii, Salmonella typhi, Citrobacter koseri, Klebsiella aerogenes, and Bacteroides fragilis. In various embodiments, a microbial species or taxonomic group targeted for identification using the methods and/or compositions of the present disclosure is a gram-positive species, such as one having a cocci and/or clusters morphology (e.g., S. aureus, S. epidermidis, S. hominis, S. haemolyticus, S. capitis, S. lugdunensis, S. pettenkoferi, or Coagulase-Negative Staphylococci (CoNS)) or having a pairs and/or chains morphology (e.g., S. anginosus complex, Streptococcus bovis, S. pyogenes, E. faecalis, E. faecium, CIA Streps, S. pneumoniae, S. agalactiae, S. mitis oralis group, or S. constellatus, S. intermedius). In some embodiments, the methods of the disclosure involve identifying a microbial species or taxonomic group through detection of one or more of the following or fragments thereof: chuA, topA, lasB, contig 23, 400 kb contig, and luxS.

In various embodiments, an antimicrobial resistance gene targeted or identification using the methods and/or compositions of the present disclosure is selected from one or more of AMP-C, VIM, IMP, AMP-C, KPC-1, KPC-2, KPC-3, NDM-1, OXA-48, OXA-232, CTX-M-15, mecA, SHV-12, SHV-2, vanA, vanB, and vanC. In various embodiments, an antimicrobial resistance gene targeted or identification using the methods and/or compositions of the present disclosure is selected from AMP-C, a carbapenemase (bla) (e.g., KPC (e.g., bla_{VIM}, bla_{IMP}, bla_{KPC-1}, bla_{KPC-2}, bla_{KPC-3}), bla_{NDM-1}, bla_{OXA-48}, bla_{OXA-232}), an extended-spectrum beta-lactamase (bla) (ESBL) (e.g., bla_{CTX-M-15}, bla_{SHV-12}, bla_{SHV-2}), a vancomycin resistance gene (e.g., vanA, vanB, or vanC), and a methicillin resistance gene (mecA). Other antibiotic resistance genes are known and may be found for example in the Comprehensive Antibiotic Resistance Database (Jia et al. “CARD 2017: expansion and model-centric curation of the Comprehensive Antibiotic Resistance Database.” Nucleic Acids Research, 45, D566-573).

As described herein, the present disclosure provides for detection of bacterial pathogens and/or antimicrobial resistance genes by using targets such as AMP-C, amino-glycosides, carbapenemases (e.g., VIM, IMP, KPC, NDM-1, OXA-48), extended-spectrum beta-lactamases (ESBLs) (e.g., (CTX-M-15, SHV-12, SHV-2), fluoro-quinolone, Topoisomerase I (TOP1), Elastase B (LasB), chuA, mecA, vanA, vanB, vanC, AMP-C, a computationally identified stretch of DNA and/or a computationally derived shared gene fragment to detect AMR bacterial strains (e.g., E. coli, K. pneumoniae, E. cloacae, P. aeruginosa, S. marcescens, S. maltophilia, C. freundii, P. mirabilis, A. baumannii, K. oxytoca, Ata. baumanii, S. aureus, S. epidermidis, S. hominis, S. haemolyticus, S. capitis, S. lugdunensis, S. pettenkoferi, Salmonella typhi, Citrobacter koseri, Klebsiella aerogenes, and Bacteroides fragilis Coagulase-Negative Staphylococci (CoNS), Streptococcus anginosus, Streptococcus bovis, S. pyogenes, E. faecalis, E. faecium, S. pneumoniae, S. agalactiae, S. constellatus, S. intermedius).

### Treating Antimicrobial-Resistant Infections

In various embodiments, the methods of the present disclosure include selecting a subject identified as containing or otherwise being infected by microbes containing an antimicrobial-resistant (AMR) gene. In an embodiment, such subjects are administered one of the agents listed in the following Table:

TABLE B
Agents for treating AMR infections

| Gene | Agent |
|---|---|
| CTX-M-15 | Meropenem, imipenem, ertapenem, colistin, |
| | ceftazidime-avibactam, cefiderocol, |
| | cetazidime-avibactam-aztreonam, |
| | meropenem-vaborbactam |
| KPC | colistin, ceftazidime-avibactam, |
| | cefiderocol, cetazidime-avibactam- |
| | aztreonam, meropenem-vaborbactam |
| NDM | colistin, cetazidime-avibactam-aztreonam |
| OXA-48- | colistin, ceftazidime-avibactam, |
| like | cefiderocol, cetazidime-avibactam- |
| | aztreonam, meropenem-vaborbactam |
| VIM | colistin, cetazidime-avibactam-aztreonam |
| IMP | colistin, cetazidime-avibactam-aztreonam |
| mecA | Vancomycin, linezolid, daptomycin, |
| | dalbavancin |
| AMP-C | Cefepime, meropenem, imipenem, |
| | ertapenem, colistin, ceftazidime-avibactam, |
| | cefiderocol, cetazidime-avibactam- |
| | aztreonam, meropenem-vaborbactam |
| vanA | linezolid, daptomycin, dalbavancin |
| vanB | linezolid, daptomycin, dalbavancin |
| vanC | linezolid, daptomycin, dalbavancin |

### Bacteria and Antimicrobial Resistance (AMR) Detection by Specific High-Sensitivity Enzymatic Reporter Unlocking (BADLOCK)

The present disclosure provides for a CRISPR-based assay, termed BADLOCK, that can detect bloodstream infections, including species identification and AMR gene content, within, e.g., one hour from the time of positive cultures. In one embodiment, the technology employs paper-based lateral flow test strips (LFTs). In embodiments, detection is achieved within 15 minutes, 30 minutes, 45 minutes, 60 minutes, or 90 minutes from time of identification of a positive culture.

In various embodiments, the methods of the disclosure involve detecting the presence or absence of a target nucleotide in a cultured blood sample. In embodiments, the blood sample is diluted in water or an aqueous solution by 2-, 3-, 4-, 5-, 6-, 7-, 8-, 9-, 10-, 11-, 12-, 13-, 14-, 15-, 16-, 17-, 18-, 19-, 20-, 50-, 100-, or more fold prior to being used as a source of a template for an amplification reaction (e.g., isothermal recombinase polymerase amplification (RPA) reaction).

As disclosed herein, the assay, termed BADLOCK (Bacteria and AMR Detection by SHERLOCK), may have a major impact on clinical care as it provides invaluable clinical information significantly faster than current approaches, a critical metric for improving patient survival. Moreover, the present disclosure provides a model for using gene-based systems for pathogen detection, and its low cost and minimal infrastructure requirements facilitate its wide deployment in low-resource setting around the world that do not currently have diagnostic options available.

In recent years, a molecular test has been developed which has the potential to realize this goal. Specific high-sensitivity enzymatic reporter unlocking, or SHERLOCK, is an assay that harnesses the power of the CRISPR-Cas enzymatic systems to detect nucleic acid sequences of interest, and it can be readily programmed to detect genes from human pathogens (Gootenberg J S, Abudayyeh O O, Lee J W, et al. Nucleic acid detection with CRISPR-Cas13a/C2c2. Science. 2017;356 (6336): 438-442. doi: 10.1126/science.aam9321; Bhattacharyya R P, Thakku S G, Hung D T. Harnessing CRISPR Effectors for Infectious Disease Diagnostics. ACS Infect Dis. 2018; 4 (9): 1278-1282. doi: 10.1021/acsinfecdis.8b00170). Specifically, a gene is targeted by primers and undergoes isothermal recombinase polymerase amplification (RPA) (Piepenburg O, Williams C H, Stemple D L, Armes N A. DNA Detection Using Recombination Proteins. PLOS Biol. 2006;4 (7): e204. doi: 10.1371/journal.pbio.0040204), is then converted to RNA through transcription by a T7 promoter attached to the RPA primers, and this RNA product is recognized by the programmable Cas-bound CRISPR sequence. This then turns on the enzymatic activity of the Cas13 enzyme. Cas13 has nonspecific trans-RNAse activity, meaning that it does not only cut the RNA it is bound to but rather any RNA molecules that are nearby in solution, allowing the creation of functional molecules that are activated when the RNA linking them is cleaved. This can be leveraged to either separate a fluorophore and quencher (resulting in fluorescence) (Arizti-Sanz J, Freije C A, Stanton A C, et al. Streamlined inactivation, amplification, and Cas13-based detection of SARS-COV-2. Nat Commun. 2020; 11:5921. doi: 10.1038/s41467-020-19097-x; de Puig H, Lee R A, Najjar D, et al. Minimally instrumented SHERLOCK (miSHERLOCK) for CRISPR-based point-of-care diagnosis of SARS-COV-2 and emerging variants. Sci Adv. 2021; 7 (32): eabh2944. doi: 10.1126/sciadv.abh2944), or unbind an antibody/antigen complex from an RNA linker molecule (enabling lateral flow readouts) (Arizti-Sanz J, Freije C A, Stanton A C, et al. Streamlined inactivation, amplification, and Cas13-based detection of SARS-COV-2. Nat Commun. 2020; 11:5921. doi: 10.1038/s41467-020-19097-x; Myhrvold C, Freije C A, Gootenberg J S, et al. Field-deployable viral diagnostics using CRISPR-Cas13. Science. 2018; 360 (6387): 444-448. doi: 10.1126/science.aas8836). Importantly, the combination of RPA primers and CRISPR guides provides two layers of targeting that add test specificity. Concordant with WHO recommendations for low resource diagnostics, SHERLOCK is highly portable, has a rapid turnaround time, and does not require costly laboratory infrastructure (Mustafa M I, Makhawi A M. SHERLOCK and DETECTR: CRISPR-Cas Systems as Potential Rapid Diagnostic Tools for Emerging Infectious Diseases. J Clin Microbiol. 2021; 59 (3). doi: 10.1128/JCM.00745-20; Gootenberg J S, Abudayyeh O O, Kellner M J, Joung J, Collins J J, Zhang F. Multiplexed and portable nucleic acid detection platform with Cas13, Cas12a, and Csm6. Science. 2018; 360 (6387): 439-444. doi: 10.1126/science.aaq0179).

As further described in the following Examples, the methods of the present disclosure, herein referred to as “BADLOCK” employ two main classes of gene targets: i) targets capable of discriminating between bacterial species; and ii) specific AMR genes that are the main drivers of resistance around the globe. When these targets are present in a sample, there is first an isothermal (e.g., room-temperature) amplification of the gene loci using recombinase polymerase amplification (RPA). Next, a CRISPR guide binds to the amplified target and turns on the Cas13 enzyme, which enables cleavage of a reporter probe that allows either fluorescent or lateral flow-based readout. Accordingly, as described herein, the present disclosure operationalizes this technology to enable a one-pot test for rapid characterization of the pathogens causing bloodstream infections, with a readout in less than 1.5 hours, wherein the only infrastructure requirement is a single heat block, thereby enabling implementation in low-resource settings.

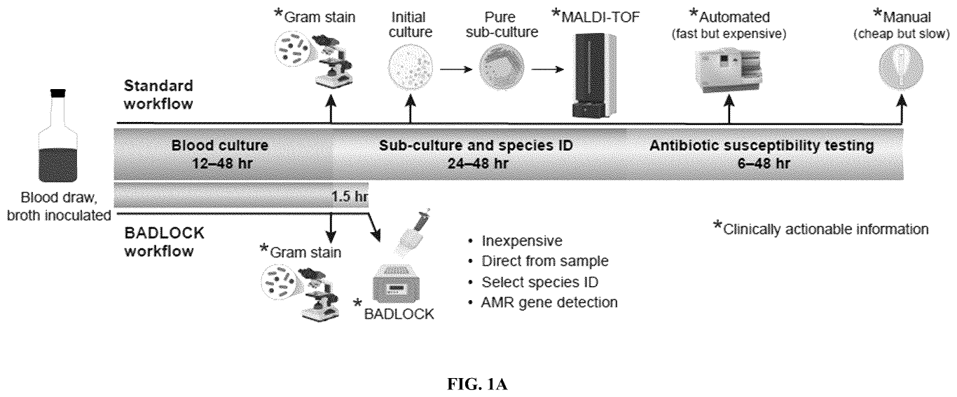

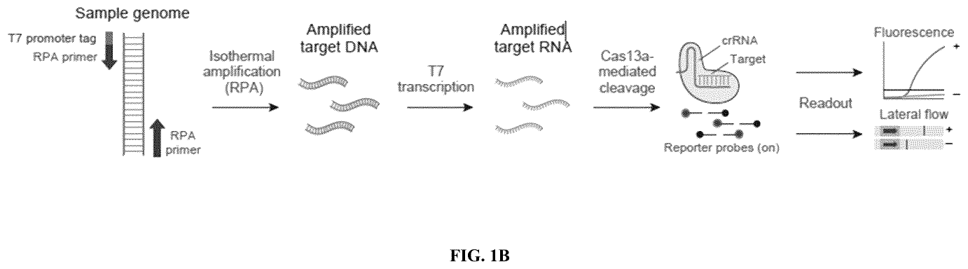

The standard diagnostic workflow for bloodstream infections (BSIs) involves several sequential steps, each requiring substantial time, specialized infrastructure, and trained personnel (FIG. 1A). In brief, blood from a patient with suspected bacteremia is inoculated into liquid culture media and incubated until microbial growth is detected. Positive blood cultures are then sub-cultured onto solid media, where individual bacterial colonies are isolated for species identification. In well-resourced laboratories, this identification is typically performed using advanced mass spectrometry-based techniques, such as MALDI-TOF (Matrix-Assisted Laser Desorption/Ionization—Time of Flight), while in lower-resource settings, traditional biochemical testing remains the primary method. Following species identification, antimicrobial susceptibility testing (AST) is conducted either with automated systems that assess growth inhibition in the presence of antibiotics or with manual methods such as broth microdilution, Kirby-Bauer disc diffusion or E-testing. Depending on bacterial growth rate, the availability of resources, and laboratory workflow efficiency, this entire process can take anywhere from 2 to 7 days, potentially delaying the initiation of targeted antimicrobial therapy.

In recent years, a growing number of alternative approaches have emerged to address the delays inherent in traditional workflows, including rapid, gene-based diagnostic platforms capable of simultaneously detecting bacterial species and antimicrobial resistance (AMR) genes directly from cultures. These methods have demonstrated significant improvements in time-to-appropriate antibiotic therapy and patient survival. However, they can come with substantial challenges, including high costs, a large infrastructural footprint, and the need for sophisticated laboratory facilities and specialized technical training. As a result, these advanced diagnostics are often inaccessible in under-resourced regions, which disproportionately bear the global burden of bacterial infections and associated mortality. This underscores the urgent need to develop rapid, low-cost bacterial diagnostic tools specifically designed for deployment in resource-limited settings.

BADLOCK (Bacterial and AMR Detection by SHERLOCK), enables the rapid identification of diverse bacterial species and key AMR genes directly from blood cultures, has been developed. This approach streamlines the standard clinical microbiology workflow, reducing both the time and complexity required for pathogen identification while simultaneously providing critical information on clinically relevant gene content to guide targeted therapy. Designed in alignment with World Health Organization (WHO) recommendations for diagnostics in low-resource settings, BADLOCK is portable, fast, low-cost, and requires minimal infrastructure, offering a practical and impactful solution for use in low-and middle-income countries. The Examples provided herein demonstrate the use of BADLOCK to accurately identify nine different gram-negative rod (GNR) bacterial species directly from clinical blood cultures, which together account for over 3.3 million global deaths annually. BADLOCK can also detect major epidemic resistance genes, underscoring its potential to address critical diagnostic gaps in global health. BADLOCK's flexible and robust format makes it a promising diagnostic tool for other biospecimens beyond blood cultures.

In various embodiments, the methods of the disclosure involve a heat lysis step wherein cells from a sample are incubated at a temperature of about or at least about 50 deg. C., 55 deg. C., 60 deg. C., 65 deg. C., 70 deg. C., 75 deg. C., 80 deg. C., 85 deg. C., 90 deg. C., 95 deg. C., or 98 deg. C. for about or at least about 1 min, 2 min, 3 min, 4 min, 5 min, 6 min, 7 min, 8 min, 9 min, 10 min, 15 min, 20 min, 25 min, or 30 min and/or for no more than about or at least about 1 min, 2 min, 3 min, 4 min, 5 min, 6 min, 7 min, 8 min, 9 min, 10 min, 15 min, 20 min, 25 min, or 30 min. In some cases, the heat lysis step is carried out prior to the addition of enzymes to a sample (e.g., for recombinase polymerase amplification). In some embodiments, a sample (e.g., a blood sample) is subjected to heat lysis prior to being aliquoted into to a container for recombinase polymerase amplification.

In some embodiments, the methods of the disclosure involve characterizing whether bacteria in a sample are Gram-positive or Gram-negative, characterizing the morphology of the bacteria, and subsequently selecting a panel of primers for use in identifying bacteria in the sample having a similar Gram-stain and morphology (e.g., gram-negative rods; gram-positive cocci in clusters; gram-positive cocci in pairs/chains).

### Type VI CRISPR Orthologs and Systems

The methods of the present disclosure involve the use of a Type VI CRISPR Ortholog (e.g., Cas13a or Cas13b). Representative Type VI CRISPR Orthologs are disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017, and U.S. Provisional Patent Application No. 62/351,662.

Cas13a, alternatively referred to as C2c2, is a representative Type VI CRISPR polypeptide and has been described in Abudayyeh, et al. (2016) “C2c2 is a single-component programmable RNA-guided RNA-targeting CRISPR effector”, Science, DOI: 10.1126/science.aaf5573; and Shmakov, et al. (2015) “Discovery and Functional Characterization of Diverse Class 2 CRISPR-Cas Systems”, Molecular Cell, DOI: dx.doi.org/10.1016/j.molcel.2015.10.008, the contents of each of which are incorporated herein in their entirety by reference for all purposes.

A Type VI CRISPR polypeptide is capable of forming a complex with a guide polynucleotide (e.g., a guide RNA). In various embodiments, the gRNA contains a polynucleotide (e.g., a targeting sequence, such as a spacer) complementary to a target polynucleotide (e.g., an RNA molecule). Non-limiting examples of gRNA sequences are listed in Table 1. A Type VI CRISPR polypeptide in complex with a guide polynucleotide becomes enzymatically activated when the Type VI CRISPR polypeptide-guide polynucleotide complex binds a polynucleotide site targeted by the guide polynucleotide, where activation of the Type VI CRISPR polypeptide results in the Type VI CRISPR polypeptide cleaving a target RNA molecule at a target site and collaterally cleaving cleaving nearby single-stranded RNA molecules in a non-sequence-specific manner.

In particular embodiments, the homologue or orthologue of a Type VI CRISPR polypeptide such as C2c2 as referred to herein has at least about 85%, 90%, 95%, 98%, 99% or greater sequence identity with a Type VI CRISPR polypeptide such as C2c2 (e.g., based on the wild-type sequence, or a functional fragment thereof, of any of Leptotrichia shahii C2c2, Lachnospiraceae bacterium MA2020 C2c2, Lachnospiraceae bacterium NK.4A1 79 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2). In various embodiments, the Type VI CRISPR polypeptide is a nuclease, such as an endonuclease. In some embodiments, the Type VI CRISPR polypeptide comprises two HEPN domains, which are RNase domains. In an embodiment, the Cas protein may be a C2c2 ortholog of an organism of a genus which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

In certain embodiments, the C2c2 protein according to the disclosure is or is derived from one of the orthologues as described in the table below, or is a chimeric protein of two or more of the orthologues as described in the table below, or is a mutant or variant of one of the orthologues as described in the table below (or a chimeric mutant or variant), including dead C2c2, split C2c2, destabilized C2c2, etc. with or without fusion with a heterologous/functional domain.

TABLE C
C2c2 orthologues

| C2c2 orthologue | Multi Letter |
|---|---|
| Leptotrichia shahii | Lsh |
| L. wadei F0279 (Lw2) | Lw2 |
| Listeria seeligeri | Lse |
| Lachnospiraceae bacterium MA2020 | LbM |
| Lachnmpiraceae bacterium NK4A 179 | LbNK.179 |
| Clostridium aminophilum DSM 10710 | Ca |
| Carnobacterium gallinarum DSM 4847 | Cg |
| Carnobacterium gallinarum DSM 4847 | Cg2 |
| Paludibacter propionicigenes WB4 | Pp |
| Listeria weihenstephanensis FSL R9-03 l 7 | Lwei |
| Listeriaceae bacterium FSL M6-0635 | LbFSL |
| Leptotrichia wadei F0279 | Lw |
| Rhodobacter capsulatus SB 1003 | Re |
| Rhodobacter capsulatus R121 | Re |
| Rhodobacter capsulatus DE442 | Re |
| Leptotrichia buccalis C-1013-b | LbuC2c2 |
| Herbinix hemicellulosilytics | HheC2c2 |
| Eubacterium rectale | EreC2c2 |
| Eubacteriaceae bacterium CHKC 1004 | EbaC2c2 |
| Blautia sp. Marseille-P2398 | BsmC2c2 |
| Leptotrichia sp. oral taxon 879 str. F0557 | LspC2c2 |
| Lachnospiraceae bacterium NK4al44 | |
| Chloroflexus aggregans | |
| Demequina aurantiaca | |
| Thalassospira sp. TSLS-1 | |
| Pseudobutyrivibrio sp. OR37 | |
| Butyrivibrio sp. YAB3001 | |
| Blautia sp. Marseille-P2398 | |
| Leptotrichia sp. Marseille-P300 | |
| Bacteroides ihuae | |
| Porphyromonadaceae bacterium KH3CP3RA | |
| Listeria riparia | |
| Insolitispirillum peregrinum | |

Exemplary Cas13b polypeptides suitable for use in embodiments of the disclosure include those disclosed in U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems” and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017), dx.doi.org/10.1016/j.molcel.2016.12.023. In particular embodiments, the Cas13b enzyme is derived from Bergeyella zoohelcum. In certain other example embodiments, the Type VI CRISPR protein is, or comprises an amino acid sequence having at least 85%, 90%, 95%, 98%, 99%, or greater sequence identity to a Cas13b polypeptide from any of the following organisms: Bergeyella zoohelcum, Prevotella intermedia, Prevotella buccae, Alistipes sp. ZOR0009, Prevotella sp. MA2016, Riemerella anatipestifer, Prevotella aurantiaca, Prevotella saccharolytica, Prevotella intermedia, Capnocytophaga canimorsus, Porphyromonas gulae, Prevotella sp. PS-125, Flavobacterium branchiophilum, Porphyromonas gingivalis, and Prevotella intermedia.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In various embodiments, a Type VI CRISPR polypeptide comprises an amino acid sequence with at least about 85%, 90%, 95%, 98%, 99% or greater sequence identity to one of the following SEQ ID NOs from International Patent Application Publication No. WO 2018/107129:3, 4, 8, 9, 11-15, 309, 309-413, 478-566-622.

### Guide Polynucleotides

A guide polynucleotide (e.g., a guide RNA (gRNA) or a single guide RNA (sgRNA)) is a polynucleotide containing a polynucleotide sequence having sufficient complementarity with a target nucleic acid to hybridize with the target nucleic acid and to direct sequence-specific binding of a RNA-targeting complex containing a Type VI CRISPR polypeptide and the guide polynucleotide. In some example embodiments, the degree of complementarity, when optimally aligned using a suitable alignment algorithm, is about or more than about 50%, 60%, 75%, 80%, 85%, 90%, 95%, 97.5%, 99%, or more. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA).

A guide polynucleotide contains a spacer sequence or guide sequence capable of binding a target nucleic acid and a scaffold sequence capable of forming a complex with a Type VI CRISPR polypeptide. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

Methods for designing guide polynucleotides are familiar to one of skill in the art. For example, ADAPT has been developed as a computational tool for CRISPR guide selection (see, e.g., Metsky H C, Welch N L, Pillai P P, et al. Designing sensitive viral diagnostics with machine learning. Nat Biotechnol. Published online Mar. 3, 2022. doi: 10.1038/s41587-022-01213-5, the disclosure of which is hereby incorporated by reference in its entirety for all purposes).

In certain embodiments, guides polynucleotides of the disclosure contain non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the disclosure, a guide nucleic acid contains ribonucleotides and non-ribonucleotides. In one such embodiment, a guide comprises one or more ribonucleotides and one or more deoxyribonucleotides. In an embodiment of the disclosure, the guide comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (4′), N¹-methylpseudouridine (me¹4′), 5-methoxyuridine (5 moU), inosine, 7-methylguanosine. Examples of guide RNA chemical modifications include, without limitation, incorporation of 2′-O-methyl (M), 2′-O-methyl-3′-phosphorothioate (MS), phosphorothioate (PS), S-constrained ethyl(cEt), or 2′-O-methyl-3′-thioPACE (MSP) at one or more terminal nucleotides. Such chemically modified guides can comprise increased stability and increased activity as compared to unmodified guides. Non-limiting examples of chemical modifications suitable for use in guide polynucleotides of the disclosure include those provided in Hendel, 2015, Nat Biotechnol. 33 (9): 985-9, doi: 10.1038/nbt.3290, published online 29 Jun. 2015; Allerson, et al., J. Med. Chem. 2005, 48:901-904; Bramsen, et al., Front. Genet., 2012, 3:154; Deng, et al., PNAS, 2015, 112:11870-11875; Sharma, et al., MedChemComm., 2014, 5:1454-1471; Hendel, et al., Nat. Biotechnol. (2015) 33 (9): 985-989; and Li, et al., Nature Biomedical Engineering, 2017, 1, 0066 DOI: 10.1038/s41551-017-0066. In some embodiments, the 5′ and/or 3′ end of a guide RNA is modified by a variety of functional moieties including fluorescent dyes, polyethylene glycol, cholesterol, proteins, or detection tags (See Kelly et al., 2016, J. Biotech. 233:74-83).

In certain embodiments, a guide comprises ribonucleotides in a region that binds to a target DNA and one or more deoxyribonucleotides and/or nucleotide analogs in a region that binds to a Type VI CRISPR polypeptide. In an embodiment of the disclosure, deoxyribonucleotides and/or nucleotide analogs are incorporated in engineered guide structures, such as, without limitation, 5′ and/or 3′ end, stem-loop regions, and a spacer. In certain embodiments, the modification is not in the 5′-handle of the stem-loop regions.

In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is or are chemically modified. In some embodiments, 3-5 nucleotides at either the 3′ or the 5′ end of a guide is chemically modified. In some embodiments, a 2′-F modification is introduced at the 3′ end of a guide. In certain embodiments, three to five nucleotides at the 5′ and/or the 3′ end of the guide are chemically modified with 2′-O-methyl (M), 2′-O-methyl-3′-phosphorothioate (MS), S-constrained ethyl(cEt), or 2′-O-methyl-3 ‘-thioPACE (MSP). Such modification can enhance genome editing efficiency (see Hendel et al., Nat. Biotechnol. (2015) 33 (9): 985-989). In certain embodiments, all of the phosphodiester bonds of a guide are substituted with phosphorothioates (PS) for enhancing levels of gene disruption. In certain embodiments, more than five nucleotides at the 5’ and/or the 3′ end of the guide are chemically modified with 2′-O-Me. Such chemically modified guide can mediate enhanced levels of gene disruption (see Ragdarm et al., 0215, PNAS, E7110-E71 11).

In an embodiment of the disclosure, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a Type VI CRISPR system (see Lee et al., eLife, 2017, 6: e25312, DOI: 10.7554).

### RNA-Based Detection Constructs

In various aspects, the methods and compositions of the present disclosure comprise RNA-based detection constructs. An RNA-based detection construct comprises a RNA element that is cleavable by a Type VI CRISPR protein. Cleavage of the RNA element releases agents or produces conformational changes that allow a detectable signal to be produced. Prior to cleavage, the detection construct blocks the generation or detection of a positive detectable signal. It will be understood that in certain example embodiments a minimal background signal may be produced in the presence of an active RNA detection construct. A positive detectable signal may be any signal that can be detected using optical, fluorescent, chemiluminescent, electrochemical or other detection methods known in the art.

In certain example embodiments, the detection construct may suppress generation of a gene product. The gene product may be encoded by a reporter construct that is added to the sample. The detection construct may be an interfering RNA involved in a RNA interference pathway, such as a short hairpin RNA (shRNA) or small interfering RNA (siRNA). The detection construct may also comprise microRNA (miRNA). While present, the detection construct suppresses expression of the gene product. The gene product may be a fluorescent protein or other RNA transcript or proteins that would otherwise be detectable by a labeled probe, aptamer, or antibody but for the presence of the detection construct. Cleavage of the detection construct allows for expression and detection of the gene product as the positive detectable signal.

In certain example embodiments, the detection construct may sequester one or more reagents needed to generate a detectable positive signal such that release of the one or more reagents from the detection construct results in generation of the detectable positive signal. The one or more reagents may combine to produce a colorimetric signal, a chemiluminescent signal, a fluorescent signal, or any other detectable signal and may comprise any reagents known to be suitable for such purposes. In certain example embodiments, the one or more reagents are sequestered by RNA aptamers that bind the one or more reagents. The one or more reagents are released when the RNA aptamers are degraded.

In certain other example embodiments, the detection construct binds to an immobilized reagent in solution thereby blocking the ability of the reagent to bind to a separate labeled binding partner that is free in solution. Thus, upon application of a washing step to a sample, the labeled binding partner can be washed out of the sample in the absence of a target molecule. However, if the Type VI CRISPR protein is activated, the detection construct is cleaved to a degree sufficient to interfere with the ability of the detection construct to bind the reagent thereby allowing the labeled binding partner to bind to the immobilized reagent. Thus, the labeled binding partner remains after the wash step indicating the presence of the target molecule in the sample. In certain aspects, the detection construct that binds the immobilized reagent is a RNA aptamer. The immobilized reagent may be a protein and the labeled minding partner may be a labeled antibody. Alternatively, the immobilized reagent may be streptavidin and the labeled binding partner may be labeled biotin. The label on the binding partner used in the above embodiments may be any detectable label known in the art.

In certain example embodiments, the detection construct may comprise a ribozyme. Ribozymes are RNA molecules having catalytic properties. Ribozymes, both naturally and engineered, comprise or consist of RNA that may be targeted by the Type VI CRISPR proteins disclosed herein. The ribozyme may be selected or engineered to catalyze a reaction that either generates a negative detectable signal or prevents generation of a positive control signal. Upon deactivation of the ribozyme by the activated Type VI CRISPR protein the reaction generating a negative control signal, or preventing generation of a positive detectable signal, is removed thereby allowing a positive detectable signal to be generated. In one example embodiment, the ribozyme may catalyze a colorimetric reaction causing a solution to appear as a first color. When the ribozyme is deactivated the solution then turns to a second color, the second color being the detectable positive signal. An example of how ribozymes can be used to catalyze a colorimetric reaction are described in Zhao et al. “Signal amplification of glucosamine-6-phosphate based on ribozyme glmS,” Biosens Bioelectron. 2014; 16:337-42, and provide an example of how such a system could be modified to work in the context of the embodiments disclosed herein. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In certain example embodiments, the one or more reagents is a protein, such as an enzyme, capable of facilitating generation of a detectable signal, such as a colorimetric, chemiluminescent, or fluorescent signal, that is inhibited or sequestered such that the protein cannot generate the detectable signal by the binding of one or more RNA aptamers to the protein. Upon activation of the Type VI CRISPR proteins disclosed herein, the RNA aptamers are cleaved or degraded to an extent that they no longer inhibit the protein's ability to generate the detectable signal. In certain example embodiments, the aptamer is a thrombin inhibitor aptamer. In certain example embodiments the thrombin inhibitor aptamer has a sequence of GGGAACAAAGCUGAAGUACUUACCC (SEQ ID NO: 65). When this aptamer is cleaved, thrombin will become active and will cleave a peptide colorimetric or fluorescent substrate. In certain example embodiments, the colorimetric substrate is para-nitroanilide (pNA) covalently linked to the peptide substrate for thrombin. Upon cleavage by thrombin, pNA is released and becomes yellow in color and easily visible to the eye. In certain example embodiments, the fluorescent substrate is 7-amino-4-methylcoumarin a blue fluorophore that can be detected using a fluorescence detector. Inhibitory aptamers may also be used for horseradish peroxidase (HRP), beta-galactosidase, or calf alkaline phosphatase (CAP) and within the general principals laid out above.

In certain embodiments, RNAse activity is detected colorimetrically via cleavage of enzyme-inhibiting aptamers. One potential mode of converting RNAse activity into a colorimetric signal is to couple the cleavage of an RNA aptamer with the re-activation of an enzyme that is capable of producing a colorimetric output. In the absence of RNA cleavage, the intact aptamer will bind to the enzyme target and inhibit its activity. The advantage of this readout system is that the enzyme provides an additional amplification step: once liberated from an aptamervia collateral activity (e.g. Cas13a collateral activity), the colorimetric enzyme will continue to produce colorimetric product, leading to a multiplication of signal.

In certain embodiments, an existing aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In certain embodiments, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In certain embodiments, RNAse activity is detected colorimetrically via cleavage of RNA-tethered inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to RNAse activity, colorimetric enzyme and inhibitor pairs can be engineered into RNAse sensors. The colorimetric RNAse sensor based upon small-molecule inhibitors involves three components: the colorimetric enzyme, the inhibitor, and a bridging RNA that is covalently linked to both the inhibitor and enzyme, tethering the inhibitor to the enzyme. In the uncleaved configuration, the enzyme is inhibited by the increased local concentration of the small molecule; when the RNA is cleaved (e.g. by Cas13a collateral cleavage), the inhibitor will be released and the colorimetric enzyme will be activated.

In certain embodiments, RNAse activity (e.g., activity of an activated Type VI CRISPR protein) is detected colorimetrically via formation and/or activation of G-quadruplexes. G quadraplexes in DNA can complex with heme (iron (III)-protoporphyrin IX) to form a DNAzyme with peroxidase activity. When supplied with a peroxidase substrate (e.g. ABTS: (2,2′-Azinobis [3-ethylbenzothiazoline-6-sulfonic acid]-diammonium salt)), the G-quadraplex-heme complex in the presence of hydrogen peroxide causes oxidation of the substrate, which then forms a green color in solution. An example G-quadraplex forming DNA sequence is: GGGTAGGGCGGGTTGGGA (SEQ ID NO: 66). By hybridizing an RNA sequence to this DNA aptamer, formation of the G-quadraplex structure will be limited. Upon RNAse collateral activation (e.g. C2c2-complex collateral activation), the RNA staple will be cleaved allowing the G quadraplex to form and heme to bind. This strategy is particularly appealing because color formation is enzymatic, meaning there is additional amplification beyond RNAse activation.

In one example embodiment, the detection construct comprises a detection agent that changes color depending on whether the detection agent is aggregated or dispersed in solution. For example, certain nanoparticles, such as colloidal gold, undergo a visible purple to red color shift as they move from aggregates to dispersed particles. Accordingly, in certain example embodiments, such detection agents may be held in aggregate by one or more bridge molecules. At least a portion of the bridge molecule comprises RNA. Upon activation of the Type VI CRISPR proteins disclosed herein, the RNA portion of the bridge molecule is cleaved allowing the detection agent to disperse and resulting in the corresponding change in color. In certain example embodiments the, bridge molecule is a RNA molecule. In certain example embodiments, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the Al³⁺, Ru³⁺, Zn²⁺, Fe³⁺, Ni²⁺ and Ca²⁺ ions.

When the RNA bridge is cut by the activated Type VI CRISPR protein, the above-described color shift is observed. In certain example embodiments the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate. In certain example embodiments the nanoparticles are modified to include DNA linkers extending from the surface of the nanoparticle. Individual particles are linked together by single-stranded RNA (ssRNA) bridges that hybridize on each end of the RNA to at least a portion of the DNA linkers. Thus, the nanoparticles will form a web of linked particles and aggregate, appearing as a dark precipitate. Upon activation of the Type VI CRISPR proteins disclosed herein, the ssRNA bridge will be cleaved, releasing the AU NPS from the linked mesh and producing a visible red color. Example DNA linkers and RNA bridge sequences are listed below. Thiol linkers on the end of the DNA linkers may be used for surface conjugation to the AuNPS. Other forms of conjugation may be used. In certain example embodiments, two populations of AuNPs may be generated, one for each DNA linker. This will help facilitate proper binding of the ssRNA bridge with proper orientation. In certain example embodiments, a first DNA linker is conjugated by the 3′ end while a second DNA linker is conjugated by the 5′ end.

In certain other example embodiments, the detection construct may comprise an RNA oligonucleotide to which are attached a detectable label and a masking agent of that detectable label. An example of such a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the RNA oligonucleotide may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of this disclosure, only that selection of the fluorophore/quencher pairs ensures masking of the fluorophore. Upon activation of the effector proteins disclosed herein, the RNA oligonucleotide is cleaved thereby severing the proximity between the fluorophore and quencher needed to maintain the contact quenching effect. Accordingly, detection of the fluorophore may be used to determine the presence of a target molecule in a sample.

In certain other example embodiments, the detection construct may comprise one or more RNA oligonucleotides to which are attached one or more metal nanoparticles, such as gold nanoparticles. In some embodiments, the detection construct comprises a plurality of metal nanoparticles crosslinked by a plurality of RNA oligonucleotides forming a closed loop. In one embodiment, the detection construct comprises three gold nanoparticles crosslinked by three RNA oligonucleotides forming a closed loop. In some embodiments, the cleavage of the RNA oligonucleotides by the Type VI CRISPR protein leads to a detectable signal produced by the metal nanoparticles.

In certain other example embodiments, the detection construct may comprise one or more RNA oligonucleotides to which are attached one or more quantum dots. In some embodiments, the cleavage of the RNA oligonucleotides by the Type VI CRISPR protein leads to a detectable signal produced by the quantum dots.

Fluorescence energy transfer (FRET) may be used to generate a detectable positive signal. FRET is a non-radiative process by which a photon from an energetically excited fluorophore (i.e. “donor fluorophore”) raises the energy state of an electron in another molecule (i.e. “the acceptor”) to higher vibrational levels of the excited singlet state. The donor fluorophore returns to the ground state without emitting a fluoresce characteristic of that fluorophore. The acceptor can be another fluorophore or non-fluorescent molecule. If the acceptor is a fluorophore, the transferred energy is emitted as fluorescence characteristic of that fluorophore. If the acceptor is a non-fluorescent molecule the absorbed energy is loss as heat. Thus, in the context of the embodiments disclosed herein, the fluorophore/quencher pair is replaced with a donor fluorophore/acceptor pair attached to the oligonucleotide molecule. When intact, the detection construct generates a first signal (negative detectable signal) as detected by the fluorescence or heat emitted from the acceptor. When the RNA oligonucleotide is cleaved, FRET is disrupted such that fluorescence of the donor fluorophore is now detected (positive detectable signal).

In certain example embodiments, the detection construct comprises the use of intercalating dyes which change their absorbance in response to cleavage of long RNAs to short nucleotides. Several such dyes exist. For example, pyronine-Y will complex with RNA and form a complex that has an absorbance at 572 nm. Cleavage of the RNA results in loss of absorbance and a color change. Methylene blue may be used in a similar fashion, with changes in absorbance at 688 nm upon RNA cleavage. Accordingly, in certain example embodiments the detection construct comprises a RNA and intercalating dye complex that changes absorbance upon the cleavage of RNA by the effector proteins disclosed herein.

In certain example embodiments, the detection construct may comprise an initiator for an HCR reaction (see, e.g. Dirks and Pierce. PNAS 101, 15275-15728 (2004). HCR reactions utilize the potential energy in two hairpin species. When a single-stranded initiator having a portion of complementary to a corresponding region on one of the hairpins is released into the previously stable mixture, it opens a hairpin of one species. This process, in turn, exposes a single-stranded region that opens a hairpin of the other species. This process, in turn, exposes a single stranded region identical to the original initiator. The resulting chain reaction may lead to the formation of a nicked double helix that grows until the hairpin supply is exhausted. Detection of the resulting products may be done on a gel or colorimetrically. Example colorimetric detection methods include, for example, those disclosed in Lu et al. “Ultra-sensitive colorimetric assay system based on the hybridization chain reaction-triggered enzyme cascade amplification ACS Appl Mater Interfaces, 2017, 9 (1): 167-175, Wang et al. “An enzyme-free colorimetric assay using hybridization chain reaction amplification and split aptamers” Analyst 2015, 150, 7657-7662, and Song et al. “Non covalent fluorescent labeling of hairpin DNA probe coupled with hybridization chain reaction for sensitive DNA detection.” Applied Spectroscopy, 70 (4): 686-694 (2016).

In certain example embodiments, the detection construct may comprise a HCR initiator sequence and a cleavable structural element, such as a loop or hairpin, that prevents the initiator from initiating the HCR reaction. Upon cleavage of the structure element by an activated Type VI CRISPR protein, the initiator is then released to trigger the HCR reaction, detection thereof indicating the presence of one or more targets in the sample. In certain example embodiments, the detection construct comprises a hairpin with a RNA loop. When the RNA loop is cut, the initiator can be released to trigger the HCR reaction.

### Signal Detection

A signal produced by a cleaved RNA-based detection construct of the disclosure may be quantified and/or detected using any methods available to one of skill in the art including, e.g., flow strips or optical methods (e.g., fluorimetry). In some embodiments, presence of a target polynucleotide in a sample is detected as an increase in fluorescence or in a colorimetric signal in a sample.

In some embodiments, a lateral flow strip allows for RNAse (e.g., C2c2 activity) detection by color. The RNA-based detection construct is modified to have a first molecule (such as for instance FITC) attached to the 5′ end and a second molecule (such as for instance biotin) attached to the 3′ end (or vice versa). The lateral flow strip is designed to have two capture lines with anti-first molecule (e.g. anti-FITC) antibodies hybridized at the first line and anti-second molecule (e.g. anti-biotin) antibodies at the second downstream line. As the reaction flows down the strip, uncleaved reporter will bind to anti-first molecule antibodies at the first capture line, while cleaved reporters will liberate the second molecule and allow second molecule binding at the second capture line. Second molecule sandwich antibodies, for instance conjugated to nanoparticles, such as gold nanoparticles, will bind any second molecule at the first or second line and result in a strong readout/signal (e.g. color). As more reporter is cleaved, more signal will accumulate at the second capture line and less signal will appear at the first line. In certain aspects, the invention relates to the use of a follow strip as described herein for detecting nucleic acids or polypeptides. In certain aspects, the invention relates to a method for detecting nucleic acids or polypeptides with a flow strip as defined

### Sample Types

Samples sources that may be analyzed using the compositions and methods described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, a swab from skin or a mucosal membrane, or combination thereof. In an example embodiment, the environmental sample is taken from a solid surface, such as a surface used in the preparation of food or other sensitive compositions and materials.

In various embodiments, the methods of the disclosure involve culturing a blood sample to allow for proliferation of microbes within the blood sample to increase the concentration of microbes within the sample to be detected. Methods for culturing blood samples are familiar to the skilled practitioner (see, e.g., Weinstein, “Current Blood Culture Methods and Systems: Clinical Concepts, Technology, and Interpretation of Results,” Clinical Infectious Diseases, 23:40-46 (1996)); and Perker, et al., “Diagnosis of bloodstream infections from positive blood cultures and directly from blood samples: recent developments in molecular approaches,” Clinical Microbiology and Infection, 24:944-955 (2018)).

### Target Amplification

In certain example embodiments, the methods of the disclosure involve amplification of a target polynucleotide (e.g., RNA and/or DNA) prior to detection using a Type VI CRISPR protein. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM).

In certain example embodiments, the RNA or DNA amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the Type VI CRISPR protein and the methods proceed as described above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter.

After, or during, the RPA reaction, a RNA polymerase is added that will produce RNA from the double-stranded DNA templates. The amplified target RNA can then in turn be detected by the Type VI CRISPR system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

A salt, such as magnesium acetate Mg(OAc)₂, magnesium chloride (MgCl₂), potassium chloride (KCl), or sodium chloride (NaCl), may be included in an amplification reaction, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction.

Other components of a biological or chemical reaction may include a cell lysis component in order to break open or lyse a cell for analysis of the materials therein. In some embodiments, the methods of the disclosure involve lysing cell by incubating the cells at a high temperature (e.g., 95 deg. C.) rather than exposing the cells to a lysing agent, such as a detergent. A cell lysis component may include, but is not limited to, a detergent, a salt, such as NaCl, KCl, or ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammomum bromide, nonyl phenoxypolyethoxylethanol (NP-40). Concentrations of detergents may depend on the particular application, and may be specific to the reaction in some cases.

Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification. In certain embodiments, detection of DNA with the methods or systems of the invention requires transcription of the (amplified) DNA into RNA prior to detection.

### Hardware and Software

The present invention also relates to a computer system involved in carrying out the methods of the invention (e.g., relating to data analysis). In some embodiments, data obtained from the methods described herein is analyzed using computer-implemented methods.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Kits

The disclosure also provides kits containing agents of this disclosure for use in the methods of the present disclosure. Kits of the disclosure may include one or more containers comprising an agent for identification of target species and/or genes in a sample, and/or may contain agents (e.g., primers, RNA-based detection constructs, guide RNA(s), Type VI CRISPR protein(s), etc.) for identification of target species and/or genes in a sample. In some embodiments, the kits further include instructions for use in accordance with the methods of this disclosure. In some embodiments, these instructions comprise a description of use of the agent according to any of the methods of this disclosure. In some embodiments, the instructions comprise a description of how to use the reagents of the kit to identify a target species and/or gene(s) in a sample. The kit may further comprise a description of how to analyze and/or interpret data.

Instructions supplied in the kits are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Assay Target Development

Bloodstream infections (BSIs) can be caused by a wide variety of highly diverse bacterial species, posing significant challenges for the development of comprehensive gene-based diagnostics. To address this issue, an assay referred to as “BADLOCK” (Bacterial and Antimicrobial Resistance (AMR) by SHERLOCK) was developed enabling the rapid identification of diverse bacterial species and AMR genes directly from blood cultures. The assay leverages a CRISPR/Cas13a enzymatic platform (FIG. 1B).

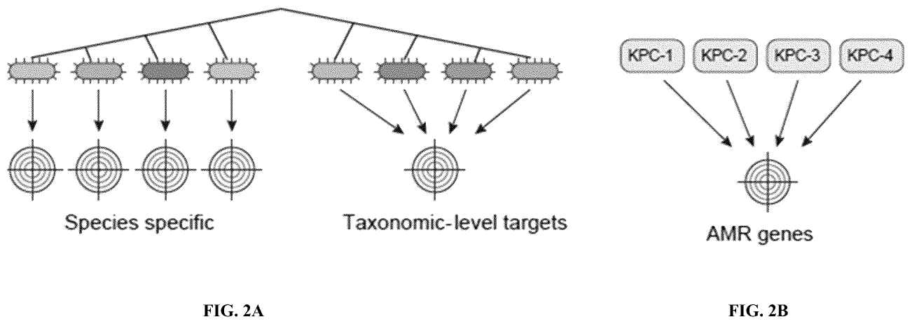

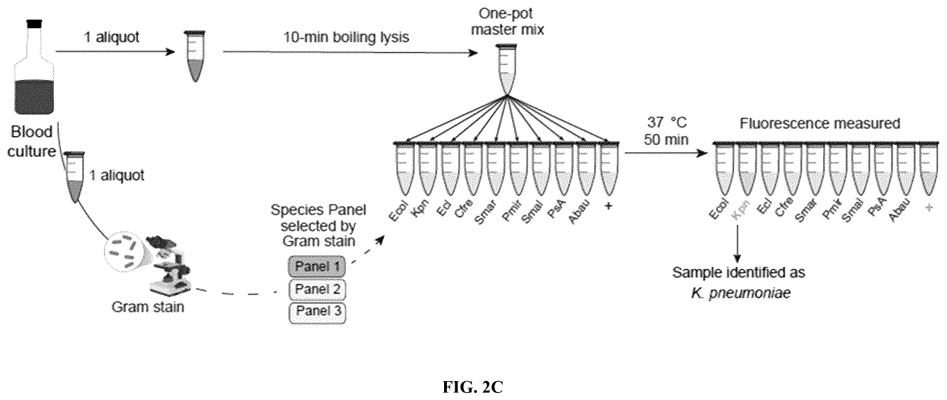

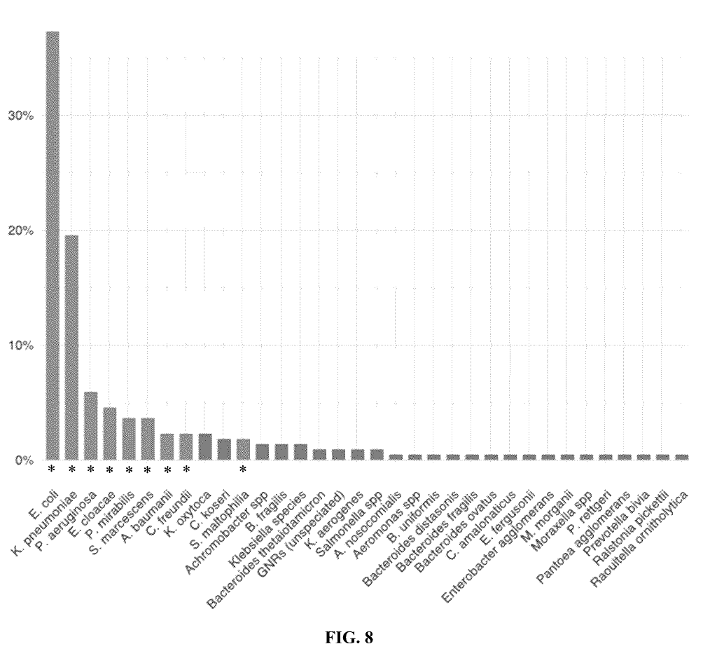

The experiments of the present Example were undertaken focusing on using the BADLOCK assay for detecting species that collectively account for ˜80% of gram-negative bloodstream infections (BSIs) within a clinical cohort containing 194 blood culture specimens (FIG. 8) and for detecting a panel of microbial resistance genes. The species targeted for detection included Escherichia coli, Klebsiella pneumoniae, Enterobacter cloacae complex, Citrobacter freundii, Serratia marcescens, Proteus mirabilis, Pseudomonas aeruginosa, and Acinetobacter baumannii. Stenotrophomonas maltophilia was also included as a target for detection due to its intrinsic resistance to many commonly used antibiotics and unique treatment requirements that would affect clinical management. For the resistance gene panel, widespread beta-lactamase genes that are strongly associated with resistance to broad-spectrum beta-lactams were targeted. These genes are linked to strains that are difficult to treat, resistant to many empiric regimens, and exhibit higher mortality. Specifically, the extended-spectrum beta-lactamase gene bla_{CTX-M-15} and the carbapenemase genes bla_{IMP}, bla_{VIM}, bla_{KPC}, bla_{NDM} and bla_{OXA-48} were target. bla_{VIM} and bla_{IMP} were foregone due to their lower prevalence both globally and within the experiment population. Three distinct strategies for target development were employed: (i) targeting individual pathogenic species; (ii) detecting clusters of closely related species with similar clinical significance, such as the Enterobacter cloacae complex, thereby minimizing the overall panel size; and (iii) identifying conserved regions within clinically relevant antimicrobial resistance (AMR) gene families (FIG. 2).

The target design process had two layers of specificity, which included an isothermal amplification of a target region using recombinase polymerase amplification (RPA) combined with a 28 bp CRISPR-Cas13a target within the amplicon (FIG. 1B). The initial approach focused on the topoisomerase I gene for species-specific targets, as it was identified through a genome-wide scan to be highly conserved within species yet sufficiently divergent between them to enable precise detection with CRISPR guides. Using this strategy, targets were developed for 4 species. In these 4 species, genes were identified that were conserved within and unique to the species of interest (Table 1) were identified. In E. coli, a unique challenge was encountered, since the RPA reagents were produced in the laboratory strain E. coli K12, leading to false positives. As a solution, the chuA gene was targeted, which is present in 80-90% of pathogenic E. coli strains but is absent from E. coli K12, a strategy used by other molecular diagnostic approaches using RPA to detect E. coli. To detect the six species that constitute the taxonomic grouping called the Enterobacter cloacae complex, a computational algorithm was adapted to identify conserved sequences shared within these species but absent from non-target species. For antimicrobial resistance (AMR) gene detection, conserved regions were utilized within the genes of interest. After identifying putative targets, ADAPT (Metsky H C, et al., “Designing sensitive viral diagnostics with machine learning,” Nat Biotechnol, Published online Mar. 3, 2022. doi: 10.1038/s41587-022-01213-5), a computational tool designed for CRISPR guide selection, was utilized to design flanking recombinase polymerase amplification (RPA) primers to amplify DNA segments containing CRISPR target sites were subsequently designed.

TABLE 1
Primer and guide sequences for each target gene included in the panel

| | Target | RPA | SEQ | RPA | SEQ | | SEQ |
|---|---|---|---|---|---|---|---|
| Species | Gene | Forward | ID NO | Reverse | IN NO | Guide RNA | ID NO |
| E. coli | chuA | gaaattaata | 67 | GAGATG | 81 | CGGCGCGACGA | 95 |
| | | cgactcacta | | ACCATT | | CGGGCUUCCCG | |
| | | tagggATATG | | TGTCGG | | CAAGCAguuuu | |
| | | GCGGTGAGTA | | CATCAA | | aguccccuucg | |
| | | TTATCGTCAG | | CATCTT | | uuuuuggggua | |
| | | GAACAACATC | | TGTAG | | gucuaaauccc | |
| | | | | | | cuauagugagu | |
| | | | | | | cguauuaauuu | |
| | | | | | | C | |
| K. | topA | gaaattaata | 68 | ATCTTC | 82 | GUGAUGAGACC | 96 |
| pneumonia | | cgactcacta | | ACGCTC | | AUGGCGGCGGU | |
| | | tagggGGTGA | | CAGTAC | | AUCGCUguuuu | |
| | | CGCCCTGCCG | | GCTGTA | | aguccccuucg | |
| | | CTGCAGGTAA | | GCTGGC | | uuuuuggggua | |
| | | CCCATAA | | TTT | | gucuaaauccc | |
| | | | | | | cuauagugagu | |
| | | | | | | cguauuaauuu | |
| | | | | | | C | |
| P. | lasB | gaaattaata | 69 | ACCGCT | 83 | UUCUAUAUGCG | 97 |
| aeruginosa | | cgactcacta | | GCCCTT | | CGGCAAGAACG | |
| | | tagggATGTT | | CTTGAT | | ACUUCCguuuu | |
| | | CTATCCGCTG | | GTCGTA | | aguccccuucg | |
| | | GTGTCGCTGG | | G | | uuuuuggggua | |
| | | A | | | | gucuaaauccc | |
| | | | | | | cuauagugagu | |
| | | | | | | cguauuaauuu | |
| | | | | | | C | |
| E. cloacae | contig | gaaattaata | 70 | AGCAGT | 84 | AGUCAACUAUU | 98 |
| | 23 | cgactcacta | | TTGGCA | | AGGCCAAAGCU | |
| | | tagggAATGG | | AAGTGC | | AUGAUCguuuu | |
| | | GCGTATGATT | | GCTTGT | | aguccccuucg | |
| | | ATACCCAGGT | | GCAG | | uuuuuggggua | |
| | | AA | | | | gucuaaauccc | |
| | | | | | | cuauagugagu | |
| | | | | | | cguauuaauuu | |
| | | | | | | C | |
| C. freundii | topA | gaaattaata | 71 | TCCATT | 85 | AUGGGUGAAAU | 99 |
| | | cgactcacta | | CGGCTT | | CGUCACCGAUC | |
| | | tagggGTTGA | | CTTTAT | | GUCUGGguuuu | |
| | | AAACCGTCGC | | TCGCCA | | aguccccuucg | |
| | | | | | | uuuuuggggua | |
| | | TTTTACGCTG | | CCTGGT | | gucuaaauccc | |
| | | AAAAA | | C | | cuauagugagu | |
| | | | | | | cguauuaauuu | |
| | | | | | | c | |
| S. | 400 kb | gaaattaata | 72 | CACCAC | 86 | UGUCCGACCUG | 100 |
| maltophilia | contig | cgactcacta | | GATTTC | | CUGCCCGGCUA | |
| | | tagggGACCA | | ATCAGG | | CCUGACguuuu | |
| | | TCCTGTACGC | | CTGACG | | aguccccuucg | |
| | | GCTGGGCTGG | | TGGAAC | | uuuuuggggua | |
| | | ACGCAGC | | TTC | | gucuaaauccc | |
| | | | | | | cuauagugagu | |
| | | | | | | cguauuaauuu | |
| | | | | | | C | |
| P. mirabilis | topA | gaaattaata | 73 | CGTCAC | 87 | AUUGGCAAUUA | 101 |
| | | cgactcacta | | TTCCAT | | CACGCUUCACU | |
| | | tagggCGGTA | | ACGCAA | | GCUGACguuuu | |
| | | CGCCTACTCG | | GGGTTG | | aguccccuucg | |
| | | TCGAGCGTGA | | TTCATC | | uuuuuggggua | |
| | | ACGTG | | | | gucuaaauccc | |
| | | | | | | cuauagugagu | |
| | | | | | | cguauuaauuu | |
| | | | | | | c | |
| A. | topA | gaaattaata | 74 | CACAGT | 88 | CAGCCAACACG | 102 |
| baumannii | | cgactcacta | | AATGGC | | UCUCGACUUAA | |
| | | tagggGGCAC | | GAAACC | | ACCGUGguuuu | |
| | | TTACGCGAAG | | ATGAAG | | aguccccuucg | |
| | | TGATTGGTGG | | CCTAC | | uuuuuggggua | |
| | | TGAC | | | | gucuaaauccc | |
| | | | | | | cuauagugagu | |
| | | | | | | cguauuaauuu | |
| | | | | | | C | |
| S. | luxS | gaaattaata | 75 | CTTTCG | 89 | UGUGGUACCUA | 103 |
| marcescens | | cgactcacta | | GCAGCG | | CCACAUGCACU | |
| | | tagggGAAGA | | CCAGTT | | CGCUGGguuuu | |
| | | GCAGCGCGTT | | CGTCGT | | aguccccuucg | |
| | | GCCGATGCCT | | TGTGGT | | uuuuuggggua | |
| | | GGAAAG | | | | gucuaaauccc | |
| | | | | | | cuauagugagu | |
| | | | | | | cguauuaauuu | |
| | | | | | | c | |
| NA | KPC | gaaattaata | 76 | CAAAGT | 90 | GCUGGCUGGCU | 104 |
| | | cgactcacta | | CCTGTT | | UUUCUGCCACC | |
| | | tagggCGTCT | | CGAGTT | | GCGCUGguuuu | |
| | | AGTTCTGCTG | | TAGCGA | | aguccccuucg | |
| | | TCTTGTCTCT | | ATGGTT | | uuuuuggggua | |
| | | CATGG | | | | gucuaaauccc | |
| | | | | | | cuauagugagu | |
| | | | | | | cguauuaauuu | |
| | | | | | | C | |
| NA | NDM | gaaattaata | 77 | TGATCT | 91 | CAACGGUUUGA | 105 |
| | | cgactcacta | | CCTGCT | | UCGUCAGGGAU | |
| | | tagggAATGT | | TGATCC | | GGCGGCguuuu | |
| | | CTGGCAGCAC | | AGTTGA | | aguccccuucg | |
| | | ACTTCCTATC | | GGATCT | | uuuuuggggua | |
| | | TCGAC | | | | gucuaaauccc | |
| | | | | | | cuauagugagu | |
| | | | | | | cguauuaauuu | |
| | | | | | | C | |
| NA | CTX- | gaaattaata | 78 | GCTAAT | 92 | AUCGUGCGCCG | 106 |
| | M-15 | cgactcacta | | ACATCG | | CUGAUUCUGGU | |
| | | tagggATAAA | | CGACGG | | CACUUAguuuu | |
| | | ACCGGCAGCG | | CTTTCT | | aguccccuucg | |
| | | GTGGCTATGG | | GCCTTA | | uuuuuggggua | |
| | | | | | | gucuaaauccc | |
| | | | | | | cuauagugagu | |
| | | | | | | cguauuaauuu | |
| | | | | | | c | |
| NA | OXA- | gaaattaata | 79 | ATAAAC | 93 | CCAAGUCUUUA | 107 |
| | 48 | cgactcacta | | AGGCAC | | AGUGGGAUGGA | |
| | | tagggTTAAA | | AACTGA | | CAGACGguuuu | |
| | | ATTCCCAATA | | ATATTT | | aguccccuucg | |
| | | GCTTGATCGC | | CATCGC | | uuuuuggggua | |
| | | CCTCG | | | | gucuaaauccc | |
| | | | | | | cuauagugagu | |
| | | | | | | cguauuaauuu | |
| | | | | | | C | |
| Synthetic | NA | gaaattaata | 50 | TAGTTA | 94 | GGCCGUCCGUU | 108 |
| target | | cgactcacta | | TTGCTC | | AAUUUCCCUUG | |
| | | taggg | | AGCGGT | | CAUACAguuuu | |
| | | | | GG | | aguccccuucg | |
| | | | | | | uuuuuggggua | |
| | | | | | | gucuaaauccc | |
| | | | | | | cuauagugagu | |
| | | | | | | cguauuaauuu | |
| | | | | | | C | |

Lowercase letters indicate conserved sequences (T7 promoter sequence for the forward RPA primer, and non-targeting region of the guide sequence, respectively), whereas uppercase letters correspond to a target-specific component.

Each primer and guide combination was first tested on genomic DNA from at least two banked isolates per species of interest to verify functionality and specificity. This process was iterative, with primer and guide combinations refined as needed to ensure specificity and reproducibility. Using this approach, recombinase polymerase amplification (RPA) primers and CRISPR guide targets for all species of interest (Table 1) were successfully developed.

### Example 2: One-Pot Assay Design and Optimization for Use on Blood Cultures

To optimize the assay for clinical microbiology laboratory workflows, where minimizing hands-on technician time is critical, a flexible one-pot approach that integrates recombinase polymerase amplification (RPA) (Lobato I M, O'Sullivan C K. Recombinase polymerase amplification: Basics, applications and recent advances. Trends Anal Chem. 2018; 98:19-35. doi: 10.1016/j.trac.2017.10.015) with Cas13-based detection in a single reaction was developed. A liquid-based RPA format was adopted as a starting point, but initial experiments revealed that the standard liquid-based RPA reaction yielded less robust amplification when combined with the Cas13 detection buffers. To address this, buffer formulations were systematically varied, with different relative concentrations and salt contents tested to identify an optimal mix. The best-performing formulation that maintained the final reaction volume used magnesium acetate from the RPA reaction as the sole magnesium source, incorporated desalted reaction buffers, and utilized an RPA buffer at 0.66× the manufacturer's recommended concentration.

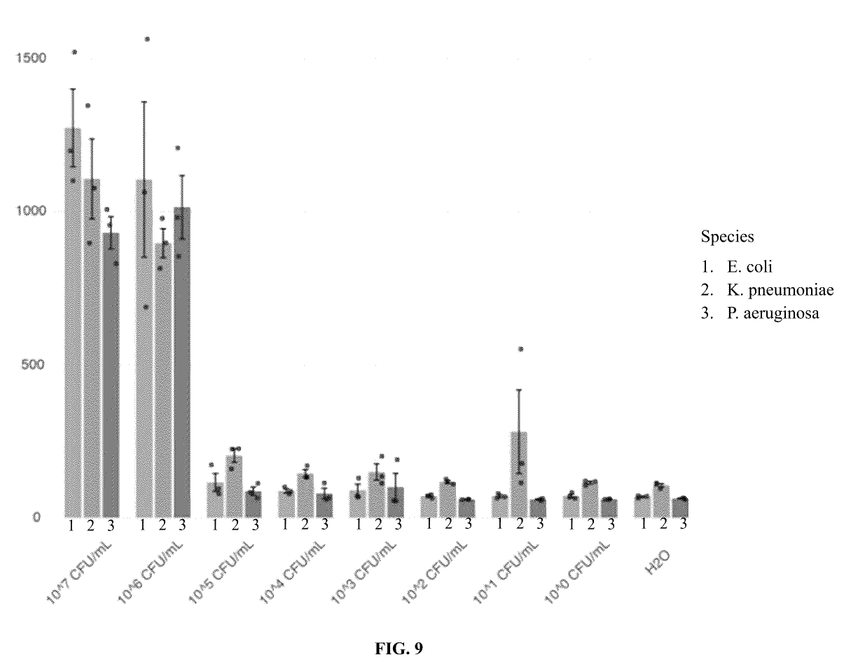

The starting template for the assay was next transitioned from genomic DNA to bacterial cultures in laboratory media. The experiment in the present Example used a simple heat lysis step to simplify workflow. The lower limit of detection (LOD) for broth cultures was determined to be 10³ colony-forming units (CFU) per microliter for three representative species (FIG. 9). Importantly, the intended target biospecimen for the assay was positive blood cultures, which typically contain bacterial concentrations between 10⁶ and 10⁸ CFU/mL. Although bacterial cultures from LB media could be directly loaded into a reaction with reliable results, initial attempts to use undiluted blood culture specimens yielded low signal, likely due to interference from blood components within the sample matrix. To address this issue, serial dilutions were performed and it was identified that a 1:10 dilution of the blood culture reliably produced positive results.

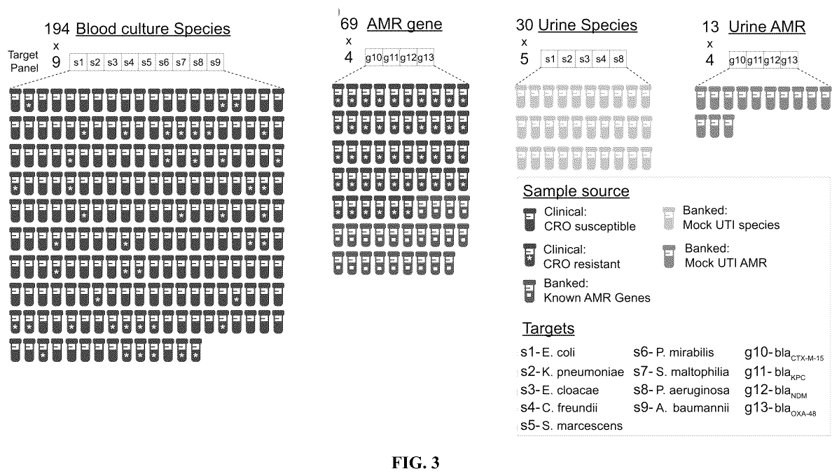

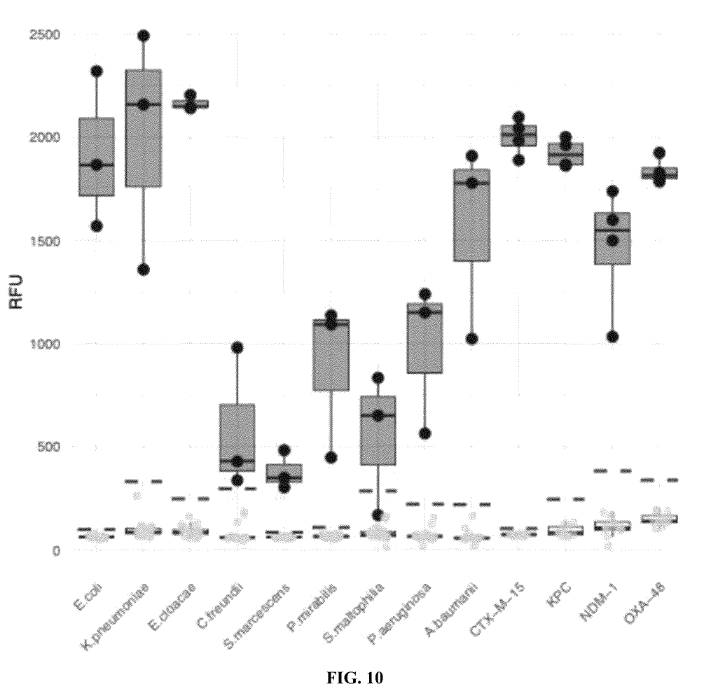

To establish robust thresholds for positivity for each species target in clinical specimens, the threshold was defined as six times the standard deviation above the mean relative fluorescence units (RFU) measured for each CRISPR target in the absence of its corresponding template. Thresholds were calculated using 24 off-target clinical samples for the species targets and 12 off-target banked samples for the AMR gene targets (FIG. 10). To ensure assay viability, a positive control was created using a single-stranded synthetic DNA sequence not predicted by BLAST™ to be in any bacteria (FIG. 2C and Table 1). Each BADLOCK assay was composed of a panel of different gene targets that were run in parallel reactions (FIG. 2C; FIG. 3).

To maintain consistency in terminology, an “assay” was defined as the complete panel of targets run simultaneously, and a “reaction” as an individual target within that panel. “False positive” and “false negative” were used to describe miscalls at the reaction level. At the assay level, “perfect concordance” was defined as cases where all reactions within the assay were called in agreement with the clinical laboratory results, “partial concordance” as cases where at least one true positive was detected alongside one or more miscalls, and “discordant” as cases in which no true positives were detected or, in the case of off-panel species, when at least one false positive was called. In alignment with FDA guidance on reporting diagnostic results for novel assays and consistent with recently published evaluations for other multiplex panels, performance was reported using positive percent agreement (PPA) and negative percent agreement (NPA). These metrics, analogous to sensitivity and specificity, are defined as follows: PPA was the proportion of true positives identified by the assay (concordant positives divided by all positive culture results), and NPA was the proportion of true negatives identified (concordant negatives divided by all negative culture results).

### Example 3: BADLOCK's Species Identification Performance on a Clinical Cohort

BADLOCK was next evaluated on a cohort of frozen aliquots from 194 consecutive positive blood cultures identified as gram-negative rods, collected by the Massachusetts General Hospital (MGH) clinical microbiology laboratory (FIG. 3). These samples had undergone the standard clinical microbiology workflow, including Gram-stain morphology, bacterial species identification, and antimicrobial resistance testing. Among the cohort, 157 specimens matched at least one species included in the assay panel, while 37 specimens contained exclusively off-target species (FIG. 8). The experiments of the present Example endeavored to interrogate polymicrobial samples (that is, when more than one species are causing bacteremia and are grown from culture) as this is a known challenge in clinical microbiology diagnostics. In total, 18 specimens were polymicrobial; 8 contained one on-panel species, 6 contained two, and four contained no on-panel species.

Because each target was processed independently in separate reactions within the assay (FIG. 2C), these 194 samples amounted to 1746 individual reactions; reaction-level calls were correctly made in 97.7% (1705/1746) instances across the cohort (FIG. 4 and FIG. 11). Among the 158 samples containing species targeted by BADLOCK, at least one target was correctly identified in 88.0% (139/158) of cases. Across the cohort on initial testing, there was perfect or partial concordance with subculture and MALDI-TOF in 88.1% (171/194) of cases, with 157 assays (80.9%) demonstrating perfect concordance and 14 (7.2%) with partial concordance. There were a total of 19 false positive reactions (across 17 samples) and 22 false negatives, with two samples containing both (FIG. 11). BADLOCK identified at least one species in 100% (6/6) of polymicrobial cultures containing multiple on-panel species and both species in 50% (3/6) of cases. It identified 62.5% (5/8) in those polymicrobial samples containing a single on-panel species. In the 37 samples containing only off-panel species, BADLOCK detected no organisms in 34 (91.9%), indicating selectivity for intended targets (FIG. 11). Individual performance characteristics for each RPA primer/CRISPR guide combination within the clinical cohort were calculated and varied across targets, with PPA ranging from 60% (S. maltophilia) to 100% (P. aeruginosa and P. mirabilis). The NPA, conversely, was high across the cohort, ranging from 97.7% to 100% across species targets (FIG. 4 and Table 2).

TABLE 2
Performance characteristics for each species target.

| | True | False | True | False | | | | |
|---|---|---|---|---|---|---|---|---|
| Species | (+) | (+) | (−) | (−) | Sensitivity | Specificity | PPA | NPA |
| E. coli | 66 | 1 | 115 | 12 | 0.85 | 0.99 | 0.85 | 0.99 |
| K. | 36 | 0 | 155 | 3 | 0.92 | 1.00 | 0.92 | 1.00 |
| pneumoniae | | | | | | | | |
| E. cloacae | 7 | 2 | 184 | 1 | 0.88 | 0.99 | 0.88 | 0.99 |
| C. freundii | 4 | 2 | 187 | 1 | 0.80 | 0.99 | 0.80 | 0.99 |
| S. | 7 | 4 | 181 | 2 | 0.78 | 0.98 | 0.78 | 0.98 |
| marcescens | | | | | | | | |
| P. mirabilis | 7 | 4 | 183 | 0 | 1.00 | 0.98 | 1.00 | 0.98 |
| S. | 3 | 1 | 188 | 2 | 0.60 | 0.99 | 0.60 | 0.99 |
| maltophilia | | | | | | | | |
| P. | 8 | 1 | 185 | 0 | 1.00 | 0.99 | 1.00 | 0.99 |
| aeruginosa | | | | | | | | |
| A. baumannii | 3 | 4 | 186 | 1 | 0.75 | 0.98 | 0.75 | 0.98 |

### Example 4: Performance on Antimicrobial Resistance Gene Testing

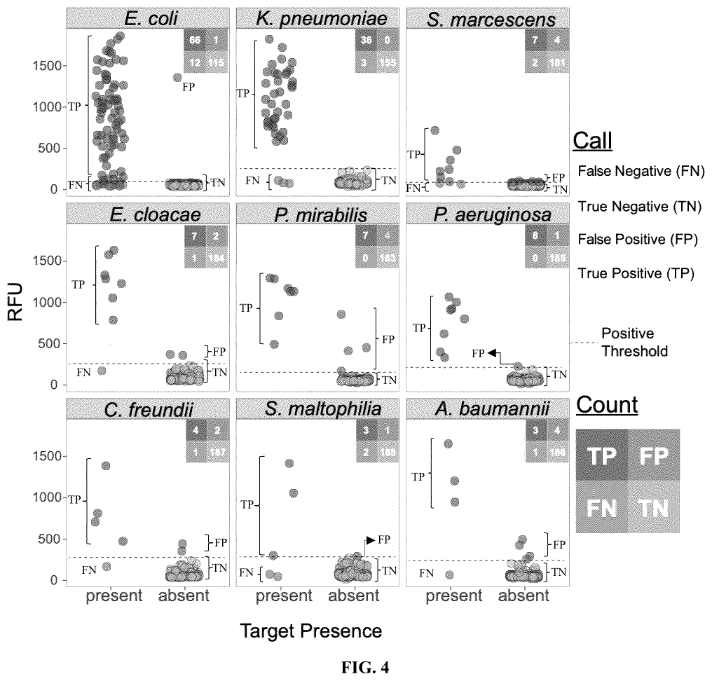

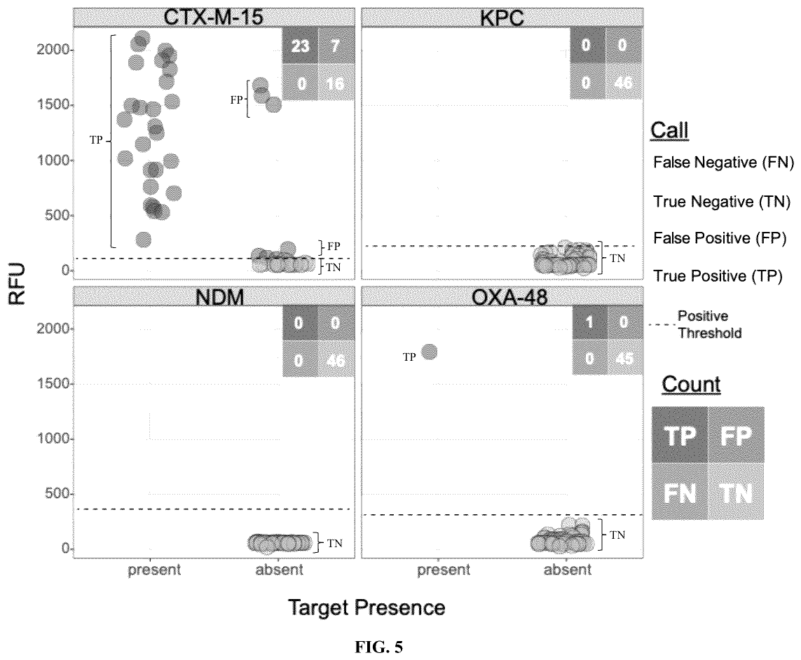

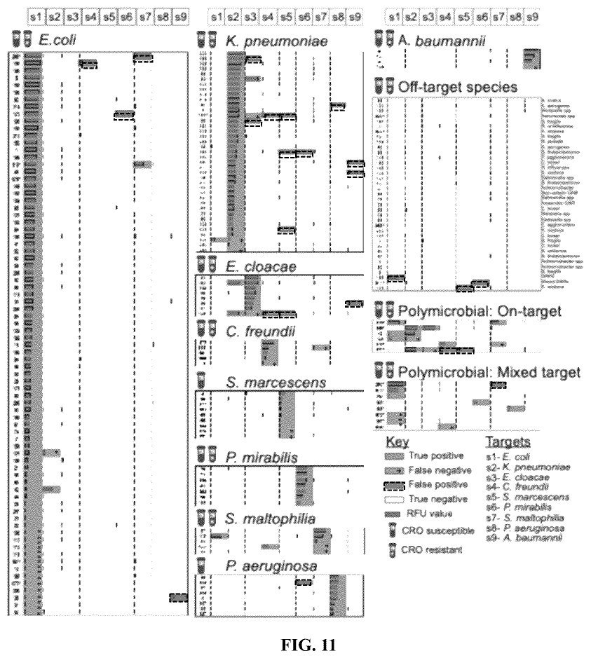

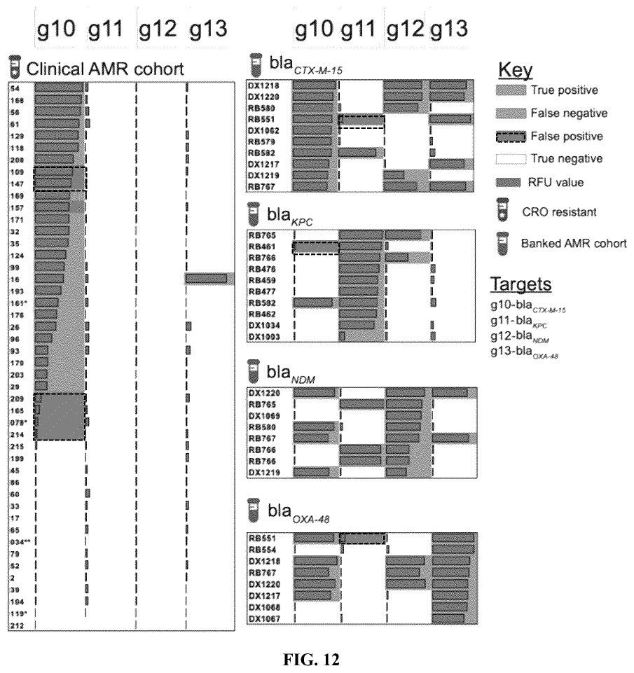

Experiments were undertaken to apply the BADLOCK assay to clinical samples known to be resistant to third-generation cephalosporins (which were more likely to harbor the extended-spectrum beta-lactamase (ESBL) or carbapenemase genes targeted by the assay), resulting in a final testing cohort of 46 clinical samples (FIG. 3B), only one of which was carbapenem-resistant. PCR was performed for all resistance gene targets in the ceftriaxone-resistant cohort to establish a ground truth for gene presence, and long-read whole-genome sequencing was conducted on a subset of nine samples for further validation. 96.2% accuracy (177/184) was achieved at the reaction level, and perfect concordance was achieved in 84.8% (39/46) of assays, with no partially concordant assays and 15.2% (7/46) discordance. 100% PPA was achieved for all antimicrobial resistance (AMR) gene targets and 100% NPA was achieved for all carbapenemase genes but notably had 7 false-positive reactions for the bla_{CTX-M-15} gene target (FIG. 5 and FIG. 12, left panel).

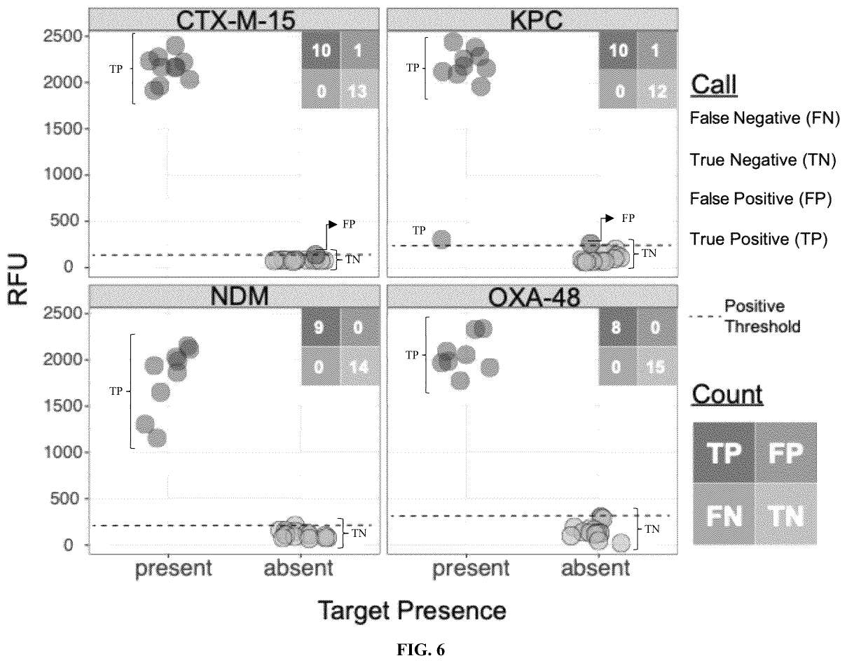

Given the low prevalence of carbapenem resistance in the MGH clinical population, the cohort was supplemented with 23 banked carbapenem-resistant isolates with known beta-lactamase content (FIG. 3B and Table 3), identified through prior sequencing efforts. Within this cohort of banked strains, BADLOCK had a correct reaction-level call in 98.9% (90/92) cases, resulting in perfect concordance in 91.3% (21/23) of assays and partial concordance in the remainder. The only two miscalls were from two false positives just above the threshold line (FIG. 6). Importantly, nine samples contained two on-panel genes, and BADLOCK correctly identified both genes in 100% (9/9) of cases (FIG. 12, right panel).

TABLE 3
Banked clinical isolates with known genomic content

| Sample | | Source of | | | |
|---|---|---|---|---|---|
| ID | Published ID | Strain | Cohorts | Species | AMR genes |
| DX1003 | MGH145 | PMID: | AMR | C. freundii | KPC-2 |
| | | 28096418 | | | |
| DX1034 | DH10Beta + | Bhattacharyya | AMR | E. coli | KPC-3 |
| | pBAD_KPC3 | Lab Strain | | | |
| DX1062 | RB001 + | Bhattacharyya | AMR, Urine | E. coli | CTX-M-15 |
| | CTX-M-15 | Lab Strain | species | | |
| DX1067 | RB001 + | Bhattacharyya | AMR, Urine | E. coli | OXA-48 |
| | OXA-48 | Lab Strain | species, Urine | | |
| | | | AMR | | |
| DX1068 | RB059 + | Bhattacharyya | AMR | E. coli | OXA-48 |
| | OXA-48 | Lab Strain | | | |
| DX1069 | RB001 + | Bhattacharyya | AMR, Urine | E. coli | NDM-1 |
| | NDM-1 | Lab Strain | species, Urine | | |
| | | | AMR | | |
| DX1217 | AR0066 | CDC AR | AMR, Urine | K. pneumoniae | CTX-M-15, |
| | (CDC) | Isolate Bank | species, Urine | | OXA-232 |
| | | | AMR | | |
| DX1218 | AR0068 | CDC AR | AMR | K. pneumoniae | CTX-M-15, |
| | (CDC) | Isolate Bank | | | NDM-1, |
| | | | | | OXA-48 |
| DX1219 | AR0143 | CDC AR | AMR | K. pneumoniae | CTX-M-15, |
| | (CDC) | Isolate Bank | | | NDM-1 |
| DX1220 | AR0153 | CDC AR | AMR | K. pneumoniae | CTX-M-15, |
| | (CDC) | Isolate Bank | | | NDM-1, |
| | | | | | OXA-48 |
| DX1276 | MGH175 | PMID: | Urine species | K. oxytoca | na |
| | | 28096418 | | | |
| RB016 | 2732233 | Bhattacharyya | Urine species | P. aeruginosa | na |
| | | Lab Strain | | | |
| RB017 | B895023 | Bhattacharyya | Urine species | P. aeruginosa | na |
| | | Lab Strain | | | |
| RB018 | 2762085 | Bhattacharyya | Urine species | P. aeruginosa | na |
| | | Lab Strain | | | |
| RB197 | Acinetobacter #1 | Bhattacharyya | Urine species | A. baumanii | na |
| | | Lab Strain | | | |
| RB262 | SBJ-32413; | Bhattacharyya | Urine species | E. cloacae | na |
| | 1349 | Lab Strain | | | |
| RB459 | BIDMC68 | PMID: | AMR | K. pneumoniae | KPC-2 |
| | | 28096418 | | | |
| RB461 | MGH67 | PMID: | AMR | K. pneumoniae | KPC-2 |
| | | 28096418 | | | |
| RB462 | MGH71 | PMID: | AMR | K. pneumoniae | KPC-2 |
| | | 28096418 | | | |
| RB476 | BWH39 | PMID: | AMR, Urine | E. cloacae | KPC-2 |
| | | 28096418 | species, Urine | | |
| | | | AMR | | |
| RB477 | MGH5 | PMID: | AMR, Urine | E. cloacae | KPC-2 |
| | | 28096418 | species, Urine | | |
| | | | AMR | | |
| RB551 | BWH2 | PMID: | AMR, Urine | K. pneumoniae | CTX-M-15, |
| | | 28096418 | AMR | | OXA-48 |
| RB554 | BAA2524 | PMID: | AMR, Urine | K. pneumoniae | OXA-48 |
| | | 28096418 | AMR | | |
| RB574 | MGH281c | PMID: | Urine species | C. freundii | na |
| | | 28096418 | | | |
| RB575 | MGH281 | PMID: | Urine species | C. freundii | na |
| | | 28096418 | | | |
| RB576 | MGH283 | PMID: | Urine species | C. freundii | na |
| | | 28096418 | | | |
| RB579 | IDR1600031102- | Wadsworth | AMR | K. pneumoniae | CTX-M-15 |
| | 01-00 | Lab | | | |
| RB580 | IDR1600014966- | Wadsworth | AMR, Urine | Citrobacter | CTX-M-15, |
| | 01-00 | Lab | AMR | spp | NDM-1 |
| RB582 | IDR 1600037319- | Wadsworth | AMR, Urine | K. pneumoniae | CTX-M-15, |
| | 01-00 | Lab | species, Urine | | KPC-1 |
| | | | AMR | | |
| RB608 | BAC0800002538 | Wadsworth | Urine species | E. aerogenes | na |
| | | Lab | | | |
| RB616 | IDR1300010999 | Wadsworth | Urine species | S. marcescens | na |
| | | Lab | | | |
| RB765 | F3461517 | PMID: | AMR, Urine | E. coli | KPC-2, |
| | | 28096418 | species, Urine | | NDM-1 |
| | | | AMR | | |
| RB766 | F3321027 | PMID: | AMR, Urine | K. pneumoniae | KPC-2, |
| | | 28096418 | species, Urine | | NDM-1 |
| | | | AMR | | |
| RB767 | X1621267 | PMID: | AMR, Urine | E. coli | CTX-M-15, |
| | | 28096418 | AMR | | NDM-1, |
| | | | | | OXA-48 |
| RB796 | MGH013 | PMID: | Urine species | P. mirabilis | na |
| | | 28096418 | | | |

The table includes the testing cohort(s) in which each sample was used, the species name, and the relevant antimicrobial resistance gene(s) of interest.

### Example 5: Investigation of Discrepancies

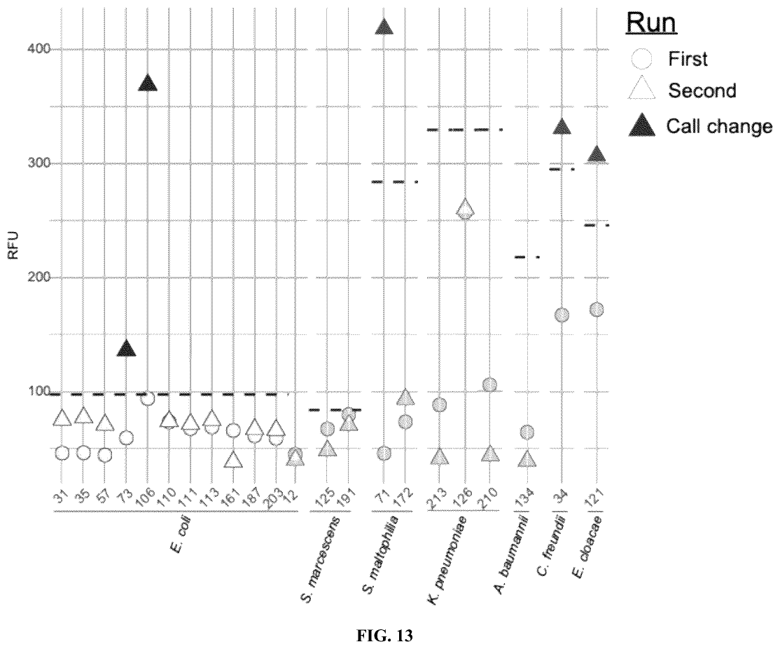

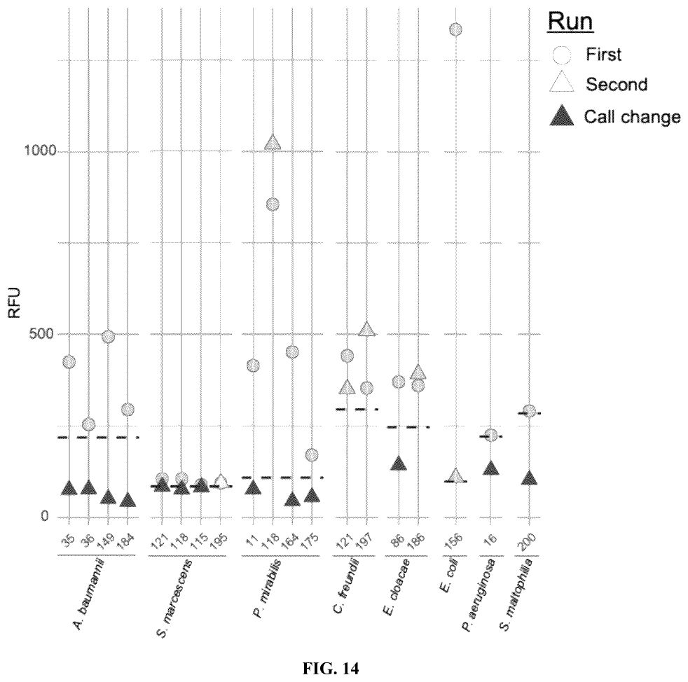

Three distinct error modes contributing to discrepancies were identified: (i) false negatives, where BADLOCK was negative but the gold-standard test was positive; (ii) false negatives due to absence of the molecular target, as seen with the chuA gene in E. coli; and (iii) false positives, where BADLOCK was positive but the gold-standard test was negative. To investigate these, the assay was first repeated for all discordant results in the species testing. In 43.9% (18/41) of cases, this repetition resolved the discrepancy; in the remainder, the discordant 10 call persisted (FIGS. 13 and 14).

False negatives: Of the 22 total discordant negatives, 12 were in E. coli, where a non-conserved gene (chuA) that is not present in the laboratory strain E. coli K12 used to make the RPA reagents, had to be targeted. As such, it was anticipated ˜15% of the E. coli samples to be falsely negative, which was consistent with the final detection rate of 84.6% (66/78). Of the 12 discordant negative E. coli samples, PCR confirmed that the chuA gene was absent in 11. In the remaining case (sample 106), where chuA was present, repeat testing yielded a robust positive result (FIG. 13). The remaining 45.5 percent (10/22) of discordant negatives were in non-E. coli species, and three of these resolved with a single repeat run (FIG. 13). In two cases, the samples were polymicrobial, potentially indicating low-abundance species within the sample (where the blood culture may have signaled positive due to another species).

False positives: Discordant positives were most frequently observed in A. baumannii, S. marcescens, and P. mirabilis, with each species accounting for four cases. A. baumannii had previously been used as a positive control, raising the possibility of amplicon contamination from prior runs as the source of these false-positive calls. This suspicion was supported by the fact that all A. baumannii discordant positives resolved when the assay was repeated at a later time point with fresh reagents (FIG. 14). In all four S. marcescens false positives, and in the P. aeruginosa and S. maltophilia miscalls, the relative fluorescence unit (RFU) values were just above the threshold. All discrepancies but one resolved upon repeat testing, with the single persistent false positive for S. marcescens (sample 195) remaining positive but right at the threshold.

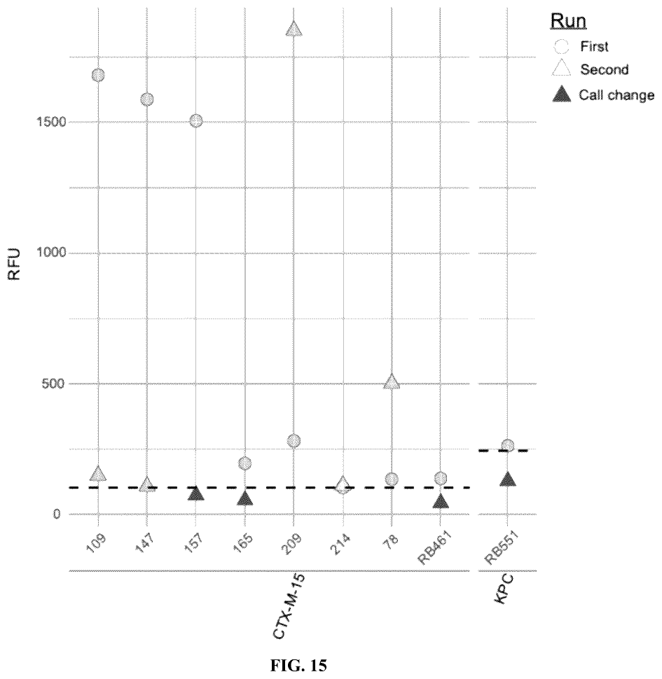

All seven discordant antimicrobial resistance (AMR) results in the clinical sample cohort were false positives for bla_{CTX-M-15}. Of these, three had very high RFU values, while four were only marginally above the positivity threshold (FIG. 5). Two of the three high-RFU samples fully corrected on repeat testing, while one showed reduced signal but remained just above the threshold (FIG. 15). Notably, all three of these samples had been batched and run together during the initial testing, raising suspicion of a cross-contamination event. For the AMR gene testing on banked isolates, the two discordant calls were a single false positive for bla_{CTX-M-15} and one for bla_{KPC}, both of which occurred right at the threshold and corrected upon repeat testing (FIG. 15).

### Example 6: Threshold Optimization

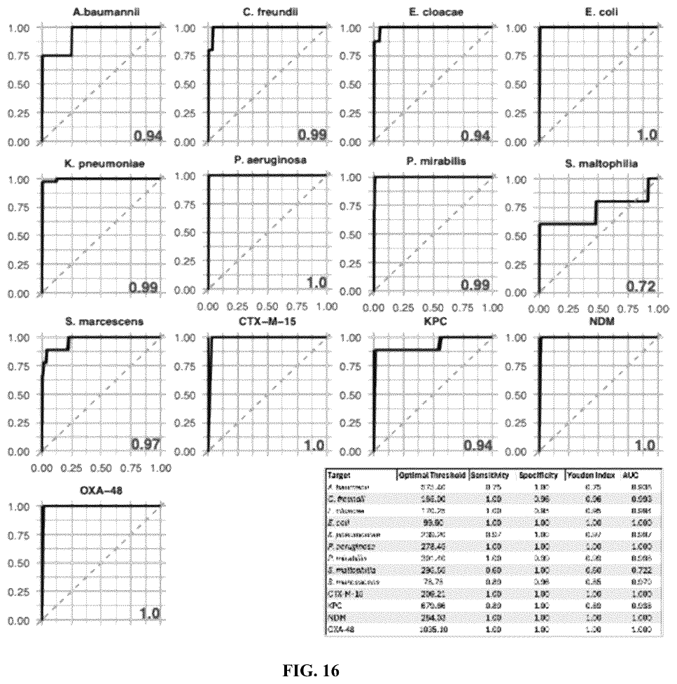

The initial positivity thresholds were initially set at 6 standard deviations above the mean of relative fluorescence unit (RFU) values from off-target samples, but as more data was generated after running the entire sample set, a post hoc receiver-operator curve (ROC) analysis was run to optimize thresholds within the cohort (FIG. 16). ROC curves help identify optimal diagnostic thresholds by visualizing the trade-off between sensitivity and specificity across all possible cutoff values, enabling flexible threshold selection tailored to each diagnostic test. The performance of each species-and gene-specific assay was evaluated, comparing RFU values against known true-positive and true-negative classifications. Prior to analysis, false positive samples suspected to be caused by contamination in the initial reaction were excluded, defined as those with repeat RFU values more than three-fold lower than the initial run (FIG. 14 and FIG. 15), as well as E. coli samples lacking the chuA gene, as these would confound a ROC analysis (i.e., samples lacking the molecular target should not be used to set optimal thresholds for evaluating performance). Most targets demonstrated excellent diagnostic accuracy, with area under the curve (AUC) values ≥0.94 for all but one target. These assays showed near-perfect sensitivity and specificity at their respective optimal thresholds, as defined by Youden's Index (Ruopp M D, et al. Youden Index and Optimal Cut-Point Estimated from Observations Affected by a Lower Limit of Detection. Biom J Biom Z. 2008;50 (3): 419-430.

doi: 10.1002/bimj.200710415). In contrast, the S. maltophilia assay had lower overall discriminative ability (AUC=0.72) due to limited sensitivity despite perfect specificity.

### Example 7: Trial on Lateral Flow Strips

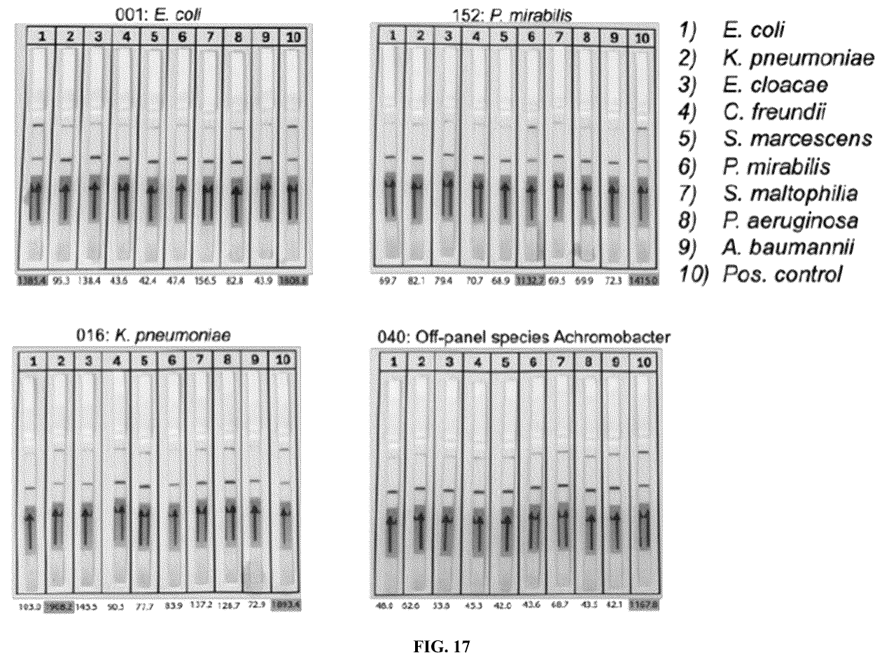

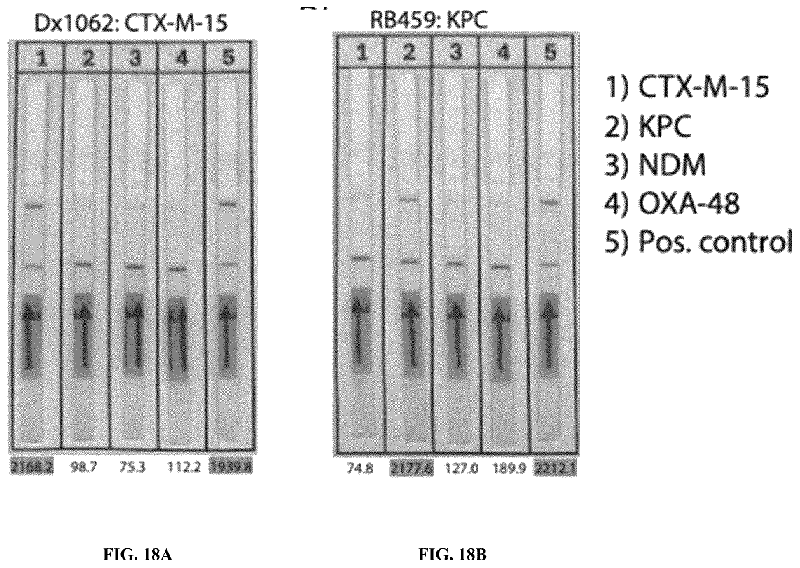

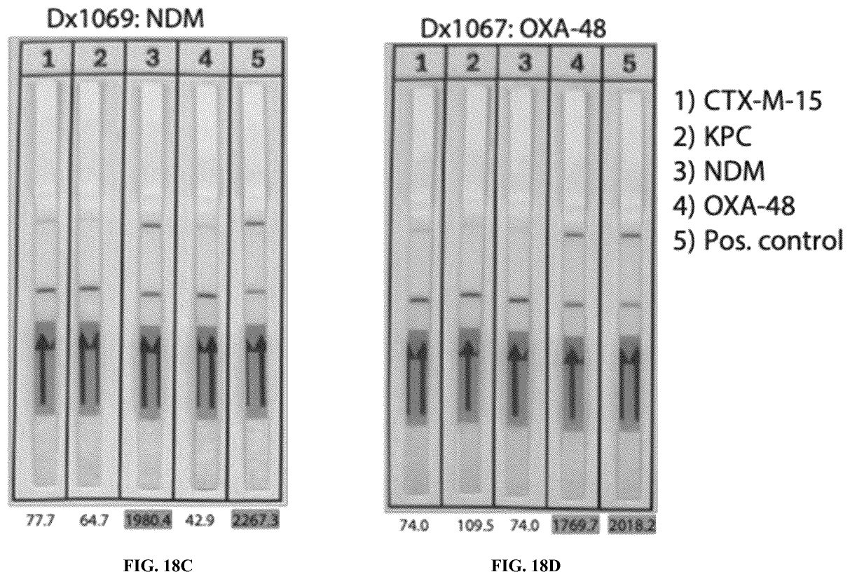

CRISPR-Cas-based diagnostics offer the advantage of lateral flow compatibility, presenting a promising opportunity for low-resource and point-of-care applications. However, the HYBRIDETECT™ lateral flow system, which is the most commonly used platform to demonstrate this functionality, was designed for hybridization-based detection of DNA targets as opposed to enzymatic cleavage detection and was co-opted for use with CRISPR-Cas systems, raising questions about its performance characteristics. To evaluate this, the lateral flow assay was tested on a subset of samples. Some cases exhibited a clear distinction between positive and negative signals despite ubiquitous faint signals in negative strips, such as samples 001 and 152 (FIG. 17) and the AMR genes (FIGS. 18A to 18D).

### Example 8: Proof-of-Concept Test on Mock Urinary Tract Infections

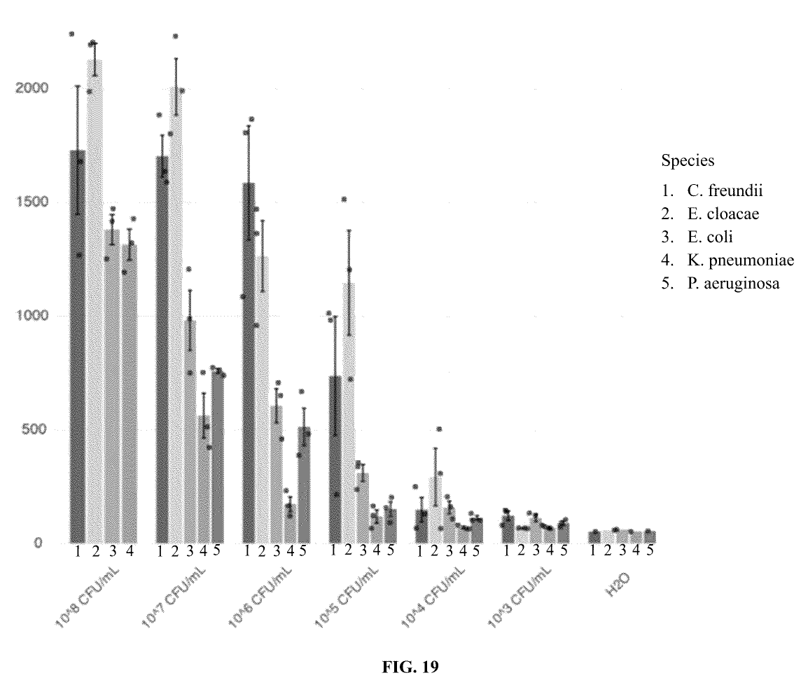

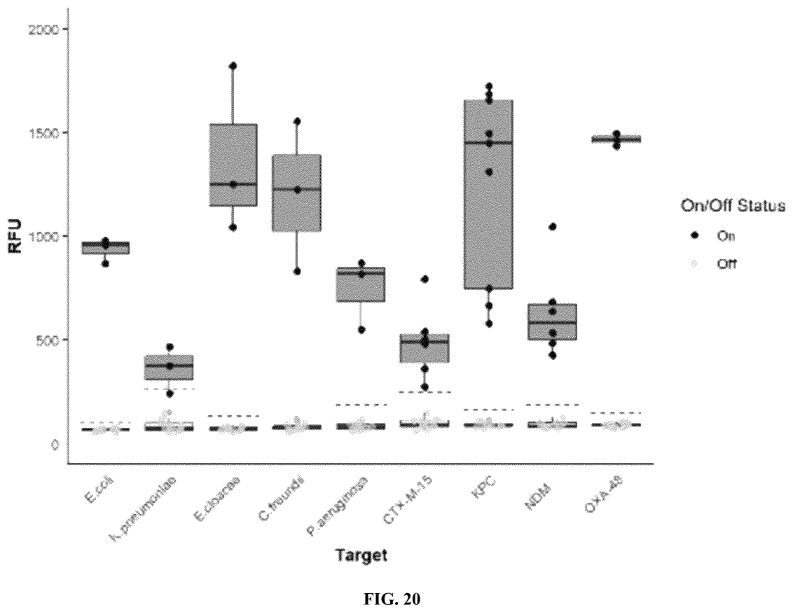

Urinary tract infections (UTIs) have higher concentrations of bacteria than BSIs, potentially removing the need for a culture step and enabling direct-from-sample detection with BADLOCK. To evaluate this, a subset of targets representing common urinary pathogens, including E. coli, K. pneumoniae, E. cloacae, C. freundii, and P. aeruginosa, were tested. Mock UTI samples were generated by culturing individual bacterial isolates and creating a dilution series for each species in filtered and pooled human urine. In the initial tests, BADLOCK successfully detected E. coli, E. cloacae, and C. freundii at concentrations below the established clinical threshold for infection, 10⁵ CFU/mL (FIG. 19), demonstrating promising sensitivity for direct-from-sample application. To improve sensitivity, multiple approaches were tested, including increasing template input volume, modifying reagent mixes, incorporating detergent-based lysis, and employing centrifugal concentration. It was found that using centrifugal concentration significantly enhanced sensitivity, enabling detection of all urine species targets at 10⁵ CFU/mL. Using the updated protocol, detection thresholds for urine samples as described above were re-established (FIG. 20).

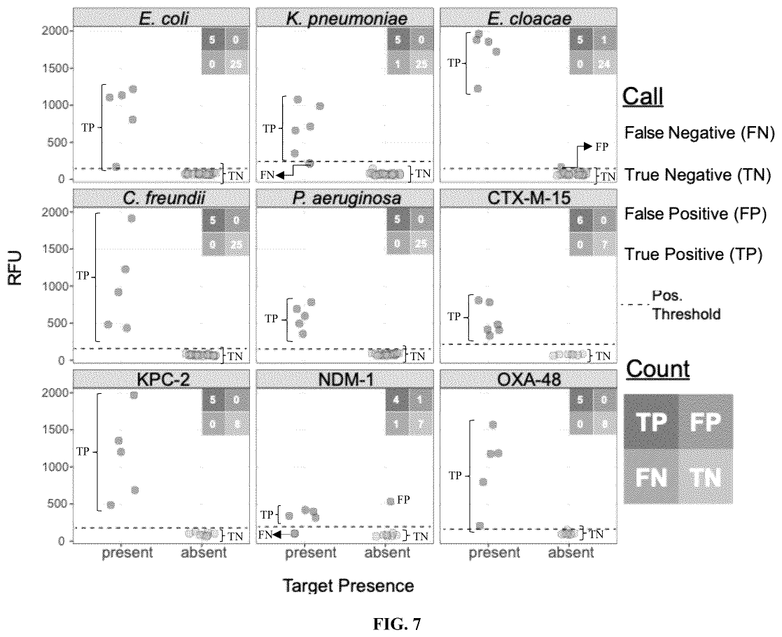

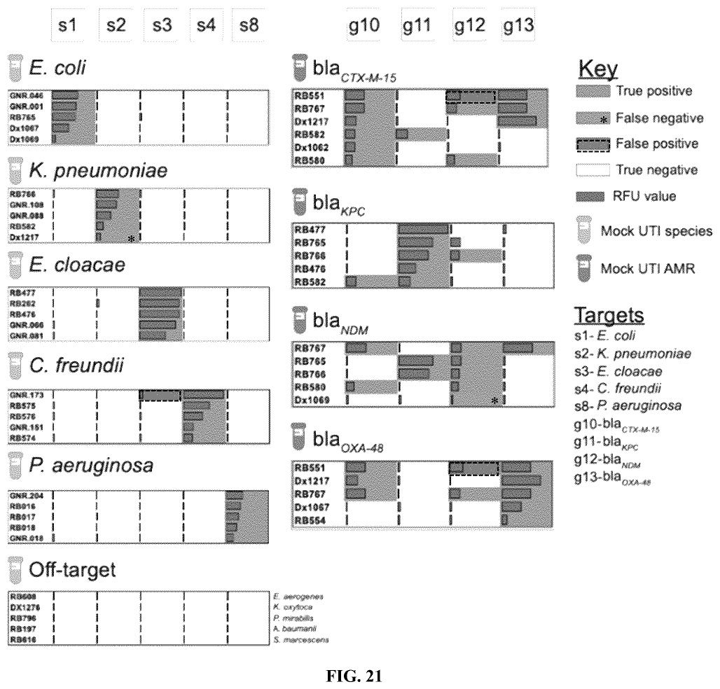

Next, mock UTI samples were generated, including at least five replicates for each gene target (including both species and AMR genes) as well as five samples containing off-panel species, each spiked at 10⁵ CFU/mL. This resulted in a total of 43 assay runs, comprising 30 species assays and 13 AMR gene assays (FIGS. 3C and 3D). At the reaction level, 98.0% concordance (198/202) was achieved, whereas at the assay level, perfect concordance in 90.6% assays (39/43), partial concordance in 4.7% (2/43), and discordant results in 4.7% (2/43) were achieved. The PPA was 100% for all targets except K. pneumoniae and bla_{NDM}, which each had a PPA of 80% (corresponding to a single false negative each). The NPA was similarly high, with 100% for all targets except E. cloacae and bla_{NDM-1}, which had 96.0 and 87.5% NPA, respectively (FIG. 7). Each of the four discrepancies was resolved with a single repeated run. No off-panel species yielded signal above the detection threshold, further supporting the assay's specificity (FIG. 21). These results demonstrated that BADLOCK could be applied directly to a urine matrix at clinically relevant bacterial loads to reliably identify both bacterial species and AMR genes.

The above Examples demonstrate the efficacy of BADLOCK, an inexpensive and adaptable diagnostic system designed to rapidly identify a panel of bacterial species that collectively account for over 3 million global deaths annually, as well as major antimicrobial resistance determinants with significant clinical implications. The utility of the BACLOCK method was validated in a retrospective experiment of 194 clinical blood cultures and in a proof-of-concept demonstration on mock UTI samples. Across all cohorts and targets, 2,224 individual reactions were run with 97.7% accuracy, reflecting the high performance of individual RPA primer and CRISPR guide combinations.

Notably, BADLOCK employs a streamlined workflow that combines target amplification and detection in a single step, making it well-suited for integration into clinical microbiology laboratory workflows. While the present testing utilized a fluorescence-based readout to support high-throughput processing, BADLOCK was also compatible with paper-based lateral flow strips.

The findings in the experiments of the Examples provided above demonstrate the potential of BADLOCK to address key challenges in diagnostic capacity for bloodstream infections, particularly in low-and middle-income countries (LMICs). In many LMICs, the substantial up-front investments required for clinical microbiology laboratories—combined with the need for specialized and highly trained personnel—often limit their integration into clinical workflows. As a result, fewer than 1% of clinical laboratories have antimicrobial resistance (AMR) testing capacity in some regions. This forces reliance on syndrome-driven therapies and broad-spectrum antibiotics, contributing to inappropriate antibiotic use and accelerating antimicrobial resistance in precisely the settings that experience the most resistance. BADLOCK was specifically designed with these limitations in mind, aligning with the REASSURED criteria established by the World Health Organization (WHO) to prioritize affordability, simplicity, and minimal infrastructure requirements. By providing rapid, actionable results in a near-point-of-care format, BADLOCK offers a feasible solution for improving diagnostic capacity in resource-limited settings. Its ability to deliver results with minimal infrastructure may help bridge the critical diagnostic gap in LMICs, supporting more targeted therapy and reducing reliance on the use of empiric and increasingly ineffective antibiotics.

Given its scalability, potential for integration into existing clinical microbiology workflows, and the low cost of components, BADLOCK has the potential for broader adoption in both resource-limited and resource-rich clinical labs.

In summary, a CRISPR-Cas13a-based prototype assay, termed BADLOCK, was developed as a promising step toward expanding rapid, low-cost diagnostic capacity in resource-limited settings. By validating the assay on a large cohort of clinical positive blood cultures, its potential for accurate pathogen detection and resistance characterization was demonstrated. Additionally, its utility as a direct-from-sample diagnostic in urine was explored, highlighting its adaptability and broader application in diagnosing infections from diverse sources. These findings underscore BADLOCK's feasibility as a scalable, accessible diagnostic tool, addressing a critical gap in infectious disease management where rapid and affordable testing is most needed.

The following materials and methods were used in the above Examples.

### CRISPR Guide Design and RPA Primer Validation

CRISPR guides were selected by inputting the desired target genes into ADAPT, which generates a ranked list of putative guide sequences. Once a candidate guide was selected, recombinase polymerase amplification (RPA) primers flanking the target region were designed. Multiple forward and reverse primers were synthesized, and all possible pairwise combinations were tested to identify viable primer sets. These were evaluated using target genomic DNA as the template, with water included as a no-template control. RPA reactions were set up in 0.2 mL PCR strip tubes using the TwistAmp® Liquid Basic Kit (TwistDx™). Each 250 μL reaction mix contained 10 μL of diluted and lysed bacterial template, 165 μL of TwistAmp® 2× Buffer (to yield a final 0.8× concentration), 22 μL of TwistAmp® 20× Core Reaction Mix (1× final concentration), 44 μL of TwistAmp® 10× E-Mix (1× final concentration), and 44 μL of a 4× dNTP mix (containing 2.5 μM of each dNTP; Takara Biosciences) to achieve a final dNTP concentration of 1 μM. Reactions were mixed gently by pipetting and incubated at 37° C. for 50 minutes. Amplification products were purified using QIAquick® column purification (Qiagen) and visualized using Invitrogen™ E-Gel™ General Purpose Agarose Gels, 2% (Thermo Fisher Scientific), pre-cast with SYBR™ Safe DNA stain. A 15 μL aliquot from each RPA reaction was loaded directly into the wells of a 12-well E-Gel cassette alongside 7.5 μL of a 100 bp DNA ladder (New England Biolabs) to assess product size. Gels were run using the E-Gel™ Power Snap Electrophoresis System (Thermo Fisher Scientific, Cat. No. G8100) under default conditions for 10 minutes. Following RPA primer validation, DNA guides were synthesized (Integrated DNA Technologies) with universal T7 transcription tails and subsequently transcribed into RNA, as described in Thakku S G, et al. Multiplexed detection of bacterial nucleic acids using Cas13 in droplet microarrays. Levine B, ed. PNAS Nexus. 2022; 1 (1): pgac021. doi: 10.1093/pnasnexus/pgac021. Each RPA primer and CRISPR guide pair was then tested in a one-pot reaction using target genomic DNA as the template and water as a no-template control (see BADLOCK Assay section below for reaction conditions). Primer-guide pairs that generated high on-target relative fluorescence unit (RFU) values and minimal background signal in the water control were advanced for further validation. This process was repeated iteratively until viable primer-guide combinations were identified for all targets of interest.

E. cloacae Species Complex Guide Design

To identify conserved sequences shared within different sub-species of the E. cloacae complex (ingroup) but absent from non-target species (outgroup), an algorithm was adapted from Thakku S G, et al. Genome-wide tiled detection of circulating Mycobacterium tuberculosis cell-free DNA using Cas13. Nat Commun. 2023; 14 (1): 1803. doi: 10.1038/s41467-023-37183-8. Briefly, for each ingroup or outgroup species, up to 100 genomes which satisfied the criteria were downloaded: complete assembly, latest version, and not anomalous. All the overlapping 100mers (e.g. 1-100, 2-01, etc.) of the reference genome of E. cloacae were then listed, and each 100mer was aligned to all ingroup and outgroup genomes using bowtie2 (Langmead B, Salzberg S L. Fast gapped-read alignment with Bowtie 2. Nat Methods. 2012; 9 (4): 357-359. doi: 10.1038/nmeth. 1923) with score-min=L,0,0 (i.e. exact match) for ingroup and score-min=L,0,−0.6 (i.e. up to ˜10 mismatches) for outgroup. 100mers that aligned, using these parameters, to all ingroup genomes and to none of the outgroup genomes were used downstream for guide design using ADAPT.

### Bacterial Collection and Standard Clinical Typing

Between October 2023-April 2024, all blood cultures taken as part of routine clinical care at Massachusetts General Hospital that signaled positive on the BACTEC™ FX system (Becton Dickinson, Sparks, MD) and were gram-negative rods by routine gram-staining were included. After staining, a 1 mL aliquot of the culture was stored at −80° C. The remainder of the sample then underwent routine clinical processing with solid-media subculturing and species identification via MALDI-TOF mass spectrometry (VITEK® MS, version 3.2 in vitro diagnostic Knowledge Base, bioMérieux, Durham, NC) and antimicrobial susceptibility testing (AST) performed on the VITEK® 2 AST-GN81 Gram Negative Susceptibility Card (bioMérieux), with Clinical and Laboratory Standards Institute (CLSI) breakpoints from the M100-Ed31.

### BADLOCK Assay

To limit reaction interference from blood products, 10 μL of sample was added to 90 μL of nuclease-free water. Heat-lysis of the samples was performed by boiling at 95° C. for 10 minutes. Each BADLOCK run consisted of 10 individual reactions that combine recombinase polymerase amplification (RPA), T7 transcription, and Cas13a mediated detection of the target. To do so, a single master mix was made combining the following reagents: 13 μL of the diluted and lysed bacterial template, 99 μL TwistAmp® liquid basic 2× Buffer (TwistDx™) (0.8× final concentration), 13.2 μL TwistAmp® liquid basic 20× Core Reaction Mix (1× final concentration), 26.4 μL TwistAmp® liquid basic 10× E-mix (1× final concentration), 26.4 μL of a dNTP mix containing each dNTP at 2.5 μM (Takara Biosciences) for an aggregate dNTP concentration of 1 μM, 26.4 μl of salt-free cleavage buffer (40% by volume 1M tris-HCL at pH 7.5, 10% by volume 100 mM DTT, 50% by volume nuclease-free H₂O), Cas13a resuspended in 26.4 μL of salt-free storage buffer (nuclease-free H₂O with a final concentration of 50 mM Tris-HCl at pH 7.5 and 5% glycerol) for a final Cas13a concentration of 45 nM, 10.56 μL of 25 mM rNTPs (New England Biolabs®) for a final concentration of 1 mM, 7.9 μL of 50,000 U/mL T7 polymerase (New England Biolabs®) for a final concentration of 1.5 U/μL, 6.6 μL of 40,000 U/mL RNase inhibitor murine (New England Biolabs®) for a final concentration of 1 U/μL, and 4.125 μL of 8 μM RNase Alert® Reporter V2 (Life Technologies™). This master mix was vigorously mixed and 18.7 microliters were split across each individual reaction well, which contained 0.5 μL of 20 μM RPA forward primer, 0.5 μL of 20 μM RPA reverse primer, and 1 μL of 450 nM CRISPR guide designed around the respective target of interest. Finally, 1 μL of 280 nM magnesium acetate was added to reaction as the sole cofactor and salt added to the mix, for a final concentration of 12.4 nM. The assay was then run at 37° for 50 minutes. When run on spectrophotometry, each sample was checked at a wavelength of 490 nm and the relative fluorescence units (RFU) were recorded. Lateral flow assays were performed using the same reaction conditions; however, instead of Reporter V2, a 14-base poly-uracil oligonucleotide tagged with FAM at the 5′ end and biotin at the 3′ end was used. This reporter was added at the same concentration and volume as in the fluorescence-based assays.

### Creation of Mock UTI Samples and Urine Sample Preparation

Stocks were streaked onto LB agar plates and incubated at 37° C. for 24 hours. A single colony from each plate was inoculated into LB broth and cultured at 37° C. with shaking until the optical density (O.D.) at 600 nm reached 0.250-0.500, as measured using a plate reader. Following growth, each bacterial culture was diluted in pooled, filtered human urine (Innovative Research) to a target concentration of 10⁸ CFU/mL, based on standard O.D. to CFU conversion values. This 10⁸ CFU/mL culture, prepared in a final volume of 100 μL, served as the starting point for a serial dilution series. All serial dilutions were carried out using the same pooled human urine as the diluent to maintain matrix consistency across all experimental conditions. Expected CFU values were confirmed through plating. For samples between 10⁴ to 10⁶ CFU/mL, 1,000 μL was transferred into a 1.5 mL tube and centrifuged at 21,300×g for 1.5 minutes to pellet the cells. Following centrifugation, 980 μL of the supernatant was carefully removed to achieve a 50× concentration of the bacterial sample. The pellet was then resuspended and heat-lysed at 95° C. for 10 minutes, followed by cooling at 4° C. for 2 minutes. This template was then carried forward in the same manner as described above.

### Other Embodiments

From the foregoing description, it will be apparent that variations and modifications may be made to the embodiments and aspects of the disclosure described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for identification of antimicrobial resistant (AMR) genes and microbial species of interest in a blood sample, the method comprising:
a) contacting each of two or more aliquots of a positive blood culture diluted in a composition comprising water with a unique set of primers in separate containers under conditions suitable for recombinase polymerase amplification (RPA), wherein a first set of primers is suitable for amplifying a target nucleotide capable of identifying a microbial species or taxonomic group, and wherein a second set of primers is suitable for amplifying a target nucleotide specific to an AMR gene, wherein one primer of each set of primers comprises a T7 promoter sequence;
b) contacting any amplicons produced in a) in each container with a T7 RNA polymerase under conditions suitable for RNA production;
c) contacting the RNA produced in b) in each container with a Type VI CRISPR polypeptide in complex with a guide polynucleotide capable of binding to RNA produced in b) in the container, and a detection construct comprising RNA, under conditions suitable for nuclease activity of the Type VI CRISPR polypeptide to be activated by binding of the guide polynucleotide to the RNA produced in b);
d) detecting any detectable signal produced by the detection construct, wherein the presence of a detectable signal in the first container and the second container identifies the blood sample as comprising the microbial species or group of microbial species and the AMR gene.

2. The method of claim 1, wherein the positive blood culture is diluted at least about 10-fold in the composition comprising water.

3. The method of claim 1, further comprising a heat lysis step prior to a).

4. The method of claim 1, wherein the first set of primers amplify a fragment of a gene selected from the group consisting of: chuA from E. coli, topA from Kpneumoniae, C. freundii, P. mirabilis, or A. baumanii, lasB from P aeruginosa, and luxS from S. marcescens; and/or wherein the second set of primers amplify a fragment of an antimicrobial resistance gene selected from the group consisting of AMP-C, bla_{IMP}, bla_{VIM}, bla_{KPC-1}, bla_{KPC-2}, bla_{KPC-3}, bla_{NDM-1}, bla_{OXA-48}, bla_{OXA-232}, bla_{CTX-M-15}, bla_{SHV-12}, bla_{SHV-2}, mecA, vanA, vanB, and vanC.

5. The method of claim 1, wherein the Type VI CRISPR polypeptide is a Cas13a polypeptide.

6. The method of claim 1, wherein:
a) the first set of primers comprise a set of sequences selected from the group consisting of:
(SEQ ID NO: 67)
GAAATTAATACGACTCACTATAGGGATATGGCGGTGAGTATTATCGTCAGGAACAACATC
and
(SEQ ID NO: 81)
GAGATGACCATTTGTCGGCATCAACATCTTTGTAG;
(SEQ ID NO: 68)
GAAATTAATACGACTCACTATAGGGGGTGACGCCCTGCCGCTGCAGGTAACCCATAA
and
(SEQ ID NO: 82)
ATCTTCACGCTCCAGTACGCTGTAGCTGGCTTT;
(SEQ ID NO: 69)
GAAATTAATACGACTCACTATAGGGATGTTCTATCCGCTGGTGTCGCTGGA
and
(SEQ ID NO: 83)
ACCGCTGCCCTTCTTGATGTCGTAG;
(SEQ ID NO: 70)
GAAATTAATACGACTCACTATAGGGAATGGGCGTATGATTATACCCAGGTAA
and
(SEQ ID NO: 84)
AGCAGTTTGGCAAAGTGCGCTTGTGCAG;
(SEQ ID NO: 71)
GAAATTAATACGACTCACTATAGGGGTTGAAAACCGTCGCTTTTACGCTGAAAAA
and
(SEQ ID NO: 85)
TCCATTCGGCTTCTTTATTCGCCACCTGGTC;
(SEQ ID NO: 72)
GAAATTAATACGACTCACTATAGGGGACCATCCTGTACGCGCTGGGCTGGACGCAGC
and
(SEQ ID NO: 86)
CACCACGATTTCATCAGGCTGACGTGGAACTTC;
(SEQ ID NO: 73)
GAAATTAATACGACTCACTATAGGGCGGTACGCCTACTCGTCGAGCGTGAACGTG
and
(SEQ ID NO: 87)
CGTCACTTCCATACGCAAGGGTTGTTCATC;
(SEQ ID NO: 74)
GAAATTAATACGACTCACTATAGGGGGCACTTACGCGAAGTGATTGGTGGTGAC
and
(SEQ ID NO: 88)
CACAGTAATGGCGAAACCATGAAGCCTAC;
(SEQ ID NO: 75)
GAAATTAATACGACTCACTATAGGGGAAGAGCAGCGCGTTGCCGATGCCTGGAAAG
and
(SEQ ID NO: 89)
CTTTCGGCAGCGCCAGTTCGTCGTTGTGGT;
(SEQ ID NO: 20)
GAAATTAATACGACTCACTATAGGGACAAAGGTCAACCAATGACATTCAGACTAT
and
(SEQ ID NO: 32)
GGACCATATTTCTCTACACCTTTTTTAGGA;
(SEQ ID NO: 21)
GAAATTAATACGACTCACTATAGGGAACAAGAAGCAGCGCGTAAA
and
(SEQ ID NO: 33)
CCCCTGTTTACCTTTGGCTT;
(SEQ ID NO: 22)
GAAATTAATACGACTCACTATAGGGTAATCCATTTACCACTTCAACATTACAACAAGA
and
(SEQ ID NO: 34)
TCAATACCTTCATATAATTGTTGCGCGAGCAT;
(SEQ ID NO: 23)
GAAATTAATACGACTCACTATAGGGGATTTAGTTGACGCGCAACAAGCACGTCGTATT
and
(SEQ ID NO: 35)
CCTTCAATGGACCAATACTCTTCAGGTTTA;
(SEQ ID NO: 24)
GAAATTAATACGACTCACTATAGGGGAATTTAGTTGATGCACAACAAGCGCGTCGT
and
(SEQ ID NO: 36)
AAGCAACTGATTGTACTCGACCAGCTGACAATC;
(SEQ ID NO: 25)
GAAATTAATACGACTCACTATAGGGACGTGTTCAGTCAGTAGCTCTTCGTTTAGTTA
and
(SEQ ID NO: 37)
TTATATCTAAATTCCCCTTCAATAGTCCAATAT;
(SEQ ID NO: 26)
GAAATTAATACGACTCACTATAGGGAAAGGCTTTATGTCTGTATACGTCGAAGCGAA
and
(SEQ ID NO: 38)
CAATCTTTGTTGCTGTCACCATCTCCCCTTCT;
(SEQ ID NO: 27)
GAAATTAATACGACTCACTATAGGGCCATCTTTTGATACAGTCCATGACTTAGCCATT
and
(SEQ ID NO: 39)
GTGACAAATGGAGTTGACGGATTGTTAGAAA;
(SEQ ID NO: 27)
GAAATTAATACGACTCACTATAGGGCCATCTTTTGATACAGTCCATGACTTAGCCATT
and
(SEQ ID NO: 39)
GTGACAAATGGAGTTGACGGATTGTTAGAAA;
(SEQ ID NO: 27)
GAAATTAATACGACTCACTATAGGGCCATCTTTTGATACAGTCCATGACTTAGCCATT
and
(SEQ ID NO: 39)
GTGACAAATGGAGTTGACGGATTGTTAGAAA;
(SEQ ID NO: 28)
GAAATTAATACGACTCACTATAGGGAATGTTACCAGATATGGAGGAAGGAGAAAGT
and
(SEQ ID NO: 40)
ACTTGCTTCTGAAAACCTTGCAGGAGGTTGA;
(SEQ ID NO: 29)
GAAATTAATACGACTCACTATAGGGGTTGATATATTGTTCTGGGATGTAGGATG
and
(SEQ ID NO: 41)
TTCTACTACAAGATTGAACGTATCCGCGAA;
(SEQ ID NO: 30)
GAAATTAATACGACTCACTATAGGGACAGCAAGCTCGTCGTACCTTAGACAGAATCG
and
(SEQ ID NO: 42)
ATATTCTTCTGGAACAAATTCTCGGATCTCTT;
and
(SEQ ID NO: 31)
GAAATTAATACGACTCACTATAGGGGAGATTACTAAAGAAGCGGTAAAAGCGGCATT
and
(SEQ ID NO: 43)
AACAGATTGCACACGACCAGCACTCAAACCT;
b) the second set of primers comprise a set of sequences selected from the group consisting of:
GAAATTAATACGACTCACTATAGGGCGTCTAGTTCTGCTGTCTTGTCTCTCATGG (SEQ ID NO: 76) and CAAAGTCCTGTTCGAGTTTAGCGAATGGTT (SEQ ID NO: 90);
GAAATTAATACGACTCACTATAGGGAATGTCTGGCAGCACACTTCCTATCTCGAC (SEQ ID NO: 77) and TGATCTCCTGCTTGATCCAGTTGAGGATCT (SEQ ID NO: 91);
GAAATTAATACGACTCACTATAGGGATAAAACCGGCAGCGGTGGCTATGG (SEQ ID NO: 78) and GCTAATACATCGCGACGGCTTTCTGCCTTA (SEQ ID NO: 92); and
GAAATTAATACGACTCACTATAGGGTTAAAATTCCCAATAGCTTGATCGCCCTCG (SEQ ID NO: 79) and ATAAACAGGCACAACTGAATATTTCATCGC (SEQ ID NO: 93); and/or
c) the guide polynucleotides each comprise a nucleotide sequence selected from the group consisting of:
(SEQ ID NO: 95)
CGGCGCGACGACGGGCUUCCCGCAAGCAGUUUUAGUCCCCUUCGUUUUUG
GGGUAGUCUAAAUCCCCUAUAGUGAGUCGUAUUAAUUUC;
(SEQ ID NO: 96)
GUGAUGAGACCAUGGCGGCGGUAUCGCUGUUUUAGUCCCCUUCGUUUUUG
GGGUAGUCUAAAUCCCCUAUAGUGAGUCGUAUUAAUUUC;
(SEQ ID NO: 97)
UUCUAUAUGCGCGGCAAGAACGACUUCCGUUUUAGUCCCCUUCGUUUUUG
GGGUAGUCUAAAUCCCCUAUAGUGAGUCGUAUUAAUUUC;
(SEQ ID NO: 98)
AGUCAACUAUUAGGCCAAAGCUAUGAUCGUUUUAGUCCCCUUCGUUUUUG
GGGUAGUCUAAAUCCCCUAUAGUGAGUCGUAUUAAUUUC;
(SEQ ID NO: 99)
AUGGGUGAAAUCGUCACCGAUCGUCUGGGUUUUAGUCCCCUUCGUUUUUG
GGGUAGUCUAAAUCCCCUAUAGUGAGUCGUAUUAAUUUC;
(SEQ ID NO: 100)
UGUCCGACCUGCUGCCCGGCUACCUGACGUUUUAGUCCCCUUCGUUUUUG
GGGUAGUCUAAAUCCCCUAUAGUGAGUCGUAUUAAUUUC;
(SEQ ID NO: 101)
AUUGGCAAUUACACGCUUCACUGCUGACGUUUUAGUCCCCUUCGUUUUUG
GGGUAGUCUAAAUCCCCUAUAGUGAGUCGUAUUAAUUUC;
(SEQ ID NO: 102
CAGCCAACACGUCUCGACUUAAACCGUGGUUUUAGUCCCCUUCGUUUUUG
GGGUAGUCUAAAUCCCCUAUAGUGAGUCGUAUUAAUUUC);
(SEQ ID NO: 103)
UGUGGUACCUACCACAUGCACUCGCUGGGUUUUAGUCCCCUUCGUUUUUG
GGGUAGUCUAAAUCCCCUAUAGUGAGUCGUAUUAAUUUC;
(SEQ ID NO: 104)
GCUGGCUGGCUUUUCUGCCACCGCGCUGGUUUUAGUCCCCUUCGUUUUUG
GGGUAGUCUAAAUCCCCUAUAGUGAGUCGUAUUAAUUUC;
(SEQ ID NO: 105)
CAACGGUUUGAUCGUCAGGGAUGGCGGCGUUUUAGUCCCCUUCGUUUUUG
GGGUAGUCUAAAUCCCCUAUAGUGAGUCGUAUUAAUUUC;
(SEQ ID NO: 106)
AUCGUGCGCCGCUGAUUCUGGUCACUUAGUUUUAGUCCCCUUCGUUUUUG
GGGUAGUCUAAAUCCCCUAUAGUGAGUCGUAUUAAUUUC;
(SEQ ID NO: 107)
CCAAGUCUUUAAGUGGGAUGGACAGACGGUUUUAGUCCCCUUCGUUUUUG
GGGUAGUCUAAAUCCCCUAUAGUGAGUCGUAUUAAUUUC;
and
(SEQ ID NO: 108)
GGCCGUCCGUUAAUUUCCCUUGCAUACAGUUUUAGUCCCCUUCGUUUUUG
GGGUAGUCUAAAUCCCCUAUAGUGAGUCGUAUUAAUUUC.

7. The method of claim 1, wherein each container comprises magnesium acetate.

8. The method of claim 1, wherein the detectable signal is produced by a fluorophore or a dye.

9. The method of claim 8, wherein the detectable signal is detected using a lateral flow strip or a fluorimeter.

10. A set of primers and one or more guide polynucleotides for use in a method for identifying microbial resistant bacteria (AMR) in a sample, wherein the set of primers and one or more guide polynucleotides comprises:
a) a first set of primers comprising a set of sequences selected from the group consisting of:
(SEQ ID NO: 67)
GAAATTAATACGACTCACTATAGGGATATGGCGGTGAGTATTATCGTCAGGAACAACATC
and
(SEQ ID NO: 81)
GAGATGACCATTTGTCGGCATCAACATCTTTGTAG;
(SEQ ID NO: 68)
GAAATTAATACGACTCACTATAGGGGGTGACGCCCTGCCGCTGCAGGTAACCCATAA
and
(SEQ ID NO: 82)
ATCTTCACGCTCCAGTACGCTGTAGCTGGCTTT;
(SEQ ID NO: 69)
GAAATTAATACGACTCACTATAGGGATGTTCTATCCGCTGGTGTCGCTGGA
and
(SEQ ID NO: 83)
ACCGCTGCCCTTCTTGATGTCGTAG;
(SEQ ID NO: 70)
GAAATTAATACGACTCACTATAGGGAATGGGCGTATGATTATACCCAGGTAA
and
(SEQ ID NO: 84)
AGCAGTTTGGCAAAGTGCGCTTGTGCAG;
(SEQ ID NO: 71)
GAAATTAATACGACTCACTATAGGGGTTGAAAACCGTCGCTTTTACGCTGAAAAA
and
(SEQ ID NO: 85)
TCCATTCGGCTTCTTTATTCGCCACCTGGTC;
(SEQ ID NO: 72)
GAAATTAATACGACTCACTATAGGGGACCATCCTGTACGCGCTGGGCTGGACGCAGC
and
(SEQ ID NO: 86)
CACCACGATTTCATCAGGCTGACGTGGAACTTC;
(SEQ ID NO: 73)
GAAATTAATACGACTCACTATAGGGCGGTACGCCTACTCGTCGAGCGTGAACGTG
and
(SEQ ID NO: 87)
CGTCACTTCCATACGCAAGGGTTGTTCATC;
(SEQ ID NO: 74)
GAAATTAATACGACTCACTATAGGGGGCACTTACGCGAAGTGATTGGTGGTGAC
and
(SEQ ID NO: 88)
CACAGTAATGGCGAAACCATGAAGCCTAC;
and
(SEQ ID NO: 75)
GAAATTAATACGACTCACTATAGGGGAAGAGCAGCGCGTTGCCGATGCCTGGAAAG
and
(SEQ ID NO: 89)
CTTTCGGCAGCGCCAGTTCGTCGTTGTGGT;
b) a second set of primers comprise a set of sequences selected from the group consisting of:
(SEQ ID NO: 76)
GAAATTAATACGACTCACTATAGGGCGTCTAGTTCTGCTGTCTTGTCTCT
CATGG
and
(SEQ ID NO: 90)
CAAAGTCCTGTTCGAGTTTAGCGAATGGTT;
(SEQ ID NO: 77)
GAAATTAATACGACTCACTATAGGGAATGTCTGGCAGCACACTTCCTATC
TCGAC
and
(SEQ ID NO: 91)
TGATCTCCTGCTTGATCCAGTTGAGGATCT;
(SEQ ID NO: 78)
GAAATTAATACGACTCACTATAGGGATAAAACCGGCAGCGGTGGCTATGG
and
(SEQ ID NO: 92)
GCTAATACATCGCGACGGCTTTCTGCCTTA;
and
(SEQ ID NO: 79)
GAAATTAATACGACTCACTATAGGGTTAAAATTCCCAATAGCTTGATCGC
CCTCG
and
(SEQ ID NO: 93)
ATAAACAGGCACAACTGAATATTTCATCGC;
and
c) a guide polynucleotide comprising a nucleotide sequence selected from the group consisting of:
(SEQ ID NO: 95)
CGGCGCGACGACGGGCUUCCCGCAAGCAGUUUUAGUCCCCUUCGUUUUUG
GGGUAGUCUAAAUCCCCUAUAGUGAGUCGUAUUAAUUUC;
(SEQ ID NO: 96)
GUGAUGAGACCAUGGCGGCGGUAUCGCUGUUUUAGUCCCCUUCGUUUUUG
GGGUAGUCUAAAUCCCCUAUAGUGAGUCGUAUUAAUUUC;
(SEQ ID NO: 97)
UUCUAUAUGCGCGGCAAGAACGACUUCCGUUUUAGUCCCCUUCGUUUUUG
GGGUAGUCUAAAUCCCCUAUAGUGAGUCGUAUUAAUUUC;
(SEQ ID NO: 98)
AGUCAACUAUUAGGCCAAAGCUAUGAUCGUUUUAGUCCCCUUCGUUUUUG
GGGUAGUCUAAAUCCCCUAUAGUGAGUCGUAUUAAUUUC;
(SEQ ID NO: 99)
AUGGGUGAAAUCGUCACCGAUCGUCUGGGUUUUAGUCCCCUUCGUUUUUG
GGGUAGUCUAAAUCCCCUAUAGUGAGUCGUAUUAAUUUC;
(SEQ ID NO: 100)
UGUCCGACCUGCUGCCCGGCUACCUGACGUUUUAGUCCCCUUCGUUUUUG
GGGUAGUCUAAAUCCCCUAUAGUGAGUCGUAUUAAUUUC;
(SEQ ID NO: 101)
AUUGGCAAUUACACGCUUCACUGCUGACGUUUUAGUCCCCUUCGUUUUUG
GGGUAGUCUAAAUCCCCUAUAGUGAGUCGUAUUAAUUUC;
(SEQ ID NO: 102)
CAGCCAACACGUCUCGACUUAAACCGUGGUUUUAGUCCCCUUCGUUUUUG
GGGUAGUCUAAAUCCCCUAUAGUGAGUCGUAUUAAUUUC;
(SEQ ID NO: 103)
UGUGGUACCUACCACAUGCACUCGCUGGGUUUUAGUCCCCUUCGUUUUUG
GGGUAGUCUAAAUCCCCUAUAGUGAGUCGUAUUAAUUUC;
(SEQ ID NO: 104)
GCUGGCUGGCUUUUCUGCCACCGCGCUGGUUUUAGUCCCCUUCGUUUUUG
GGGUAGUCUAAAUCCCCUAUAGUGAGUCGUAUUAAUUUC;
(SEQ ID NO: 105)
CAACGGUUUGAUCGUCAGGGAUGGCGGCGUUUUAGUCCCCUUCGUUUUUG
GGGUAGUCUAAAUCCCCUAUAGUGAGUCGUAUUAAUUUC;
(SEQ ID NO: 106)
AUCGUGCGCCGCUGAUUCUGGUCACUUAGUUUUAGUCCCCUUCGUUUUUG
GGGUAGUCUAAAUCCCCUAUAGUGAGUCGUAUUAAUUUC;
(SEQ ID NO: 107)
CCAAGUCUUUAAGUGGGAUGGACAGACGGUUUUAGUCCCCUUCGUUUUUG
GGGUAGUCUAAAUCCCCUAUAGUGAGUCGUAUUAAUUUC;
and
(SEQ ID NO: 108)
GGCCGUCCGUUAAUUUCCCUUGCAUACAGUUUUAGUCCCCUUCGUUUUUG
GGGUAGUCUAAAUCCCCUAUAGUGAGUCGUAUUAAUUUC.

11. A method of treating a selected subject having an antimicrobial resistant bacterial infection, the method comprising administering an agent capable of reducing the survival or proliferation of bacteria of the bacterial infection to the selected subject, wherein the subject is selected by a method comprising:
a) splitting a biological sample from a subject into two containers, wherein one container comprises a first set of primers suitable for amplifying a target polynucleotide capable of identifying a bacterial species or taxonomic group, and a second container comprises a second set of primers suitable for amplifying a target AMR gene, wherein one primer of each set of primers comprises a T7 promoter sequence, and wherein the biological sample comprises bacteria;
b) incubating the biological sample under conditions suitable for recombinase polymerase amplification (RPA) to produce amplicons;
b) contacting the amplicons with a T7 RNA polymerase to produce RNA;
c) contacting the RNA with a Type VI CRISPR polypeptide, guide polynucleotides capable of specifically binding the targets, and a detection construct comprising RNA, under conditions suitable for Type VI CRISPR polypeptide nuclease activity, thereby cutting the RNA;
d) detecting a detectable signal produced by the detection construct, wherein the presence of a detectable signal in the first container and the second container selects the subject for administration of a drug for treating an AMR infection.

12. The method of claim 11, wherein the biological sample is a blood sample.

13. The method of claim 11, wherein:
a) the bacterium is a Gram negative bacteria selected from the group consisting of E. coli, K. pneumoniae, E. cloacae, C. freundii, S. marcescens, S. maltophila, P. mirabilis, A. baumanii, K. oxytoca, P. aeruginosa, Vibrio cholerae, Salmonella enterica, Salmonella typhi, Citrobacter koseri, Klebsiella aerogenes, and Bacteroides fragilis, a member of the genus Citrobacter, and Enterobacter aerogenes; or
b) the bacterium is a Gram positive bacteria selected from the group consisting of S. Aureus, S. hemolyticus, S. capitis, S. hominis, S. pettenkoferi, S. lugdunensis, S. epidermidis E. faecalis, E. faecium, S. anginosus complex, S. agalactiae, S. pneumoniae, Streptococcus bovis, S. pyogenes, S. constellatus, S. intermedius, S. mitis, S. mutans, S. salivarius, S. oralis, S. sanguinis, S. gordonii, S. gallolyticus, S. pneumoniae, S. anginosus, and S. agalactiae.

14. The method of claim 11, wherein the bacteria comprise an AMR gene selected from the group consisting of AMP-C, bla_{VIM}, bla_{IMP}, bla_{KPC-1}, bla_{KPC-2}, bla_{KPC-3}, bla_{NDM-1}, bla_{OXA-48}, bla_{CTX-M-15}, mecA, vanA, vanB, and vanC, and wherein:
a) when the AMR gene is bla_{CTX-M-15}, the drug is selected from the group consisting of meropenem, imipenem, ertapenem, colistin, ceftazidime-avibactam, cefiderocol, cetazidime-avibactam-aztreonam, and meropenem-vaborbactam;
b) when the AMR gene is bla_{KPC-1}, bla_{KPC-2}, or bla_{KPC-3}, the drug is selected from the group consisting of colistin, ceftazidime-avibactam, cefiderocol, cetazidime-avibactam-aztreonam, and meropenem-vaborbactam;
c) when the AMR gene is bla_{NDM-1}, the drug is selected from the group consisting of colistin, and cetazidime-avibactam-aztreonam;
d) when the AMR gene is bla_{OXA-48}, the drug is selected from the group consisting of colistin, ceftazidime-avibactam, cefiderocol, cetazidime-avibactam-aztreonam, and meropenem-vaborbactam;
e) when the AMR gene is bla_{VIM}, the drug is cetazidime-avibactam-aztreonam;
f) when the AMR gene is bla_{IMP}, the drug is selected from the group consisting of colistin and cetazidime-avibactam-aztreonam;
g) when the AMR gene is mecA, the drug is selected from the group consisting of vancomycin, linezolid, daptomycin, and dalbavancin;
h) when the AMP-C gene is mecA, the drug is selected from the group consisting of cefepime, meropenem, imipenem, ertapenem, colistin, ceftazidime-avibactam, cefiderocol, cetazidime-avibactam-aztreonam, and meropenem-vaborbactam; and
i) when the AMP-C gene is vanA, vanB, or vanC, the drug is selected from the group consisting of linezolid, daptomycin, and dalbavancin.

15. The method of claim 11, wherein the biological sample is a blood sample that is diluted at least about 10-fold in a composition comprising water.

16. The method of claim 15, wherein the composition comprising water is distilled or deionized water.

17. The method of claim 11, wherein the first set of primers amplify a fragment of a gene selected from the group consisting of: chuA from E. coli, topA from Kpneumoniae, C. freundii, P. mirabilis, or A. baumanii, lasB from P aeruginosa, and luxS from S. marcescens.

18. The method of claim 11, wherein the Type VI CRISPR polypeptide is a Cas13a polypeptide.

19. The method of claim 18, wherein:
a) the first set of primers comprise a set of sequences selected from the group consisting of:
(SEQ ID NO: 67)
GAAATTAATACGACTCACTATAGGGATATGGCGGTGAGTATTATCGTCAGGAACAACATC
and
(SEQ ID NO: 81)
GAGATGACCATTTGTCGGCATCAACATCTTTGTAG;
(SEQ ID NO: 68)
GAAATTAATACGACTCACTATAGGGGGTGACGCCCTGCCGCTGCAGGTAACCCATAA
and
(SEQ ID NO: 82)
ATCTTCACGCTCCAGTACGCTGTAGCTGGCTTT;
(SEQ ID NO: 69)
GAAATTAATACGACTCACTATAGGGATGTTCTATCCGCTGGTGTCGCTGGA
and
(SEQ ID NO: 83)
ACCGCTGCCCTTCTTGATGTCGTAG;
(SEQ ID NO: 70)
GAAATTAATACGACTCACTATAGGGAATGGGCGTATGATTATACCCAGGTAA
and
(SEQ ID NO: 84)
AGCAGTTTGGCAAAGTGCGCTTGTGCAG;
(SEQ ID NO: 71)
GAAATTAATACGACTCACTATAGGGGTTGAAAACCGTCGCTTTTACGCTGAAAAA
and
(SEQ ID NO: 85)
TCCATTCGGCTTCTTTATTCGCCACCTGGTC;
(SEQ ID NO: 72)
GAAATTAATACGACTCACTATAGGGGACCATCCTGTACGCGCTGGGCTGGACGCAGC
and
(SEQ ID NO: 86)
CACCACGATTTCATCAGGCTGACGTGGAACTTC;
(SEQ ID NO: 73)
GAAATTAATACGACTCACTATAGGGCGGTACGCCTACTCGTCGAGCGTGAACGTG
and
(SEQ ID NO: 87)
CGTCACTTCCATACGCAAGGGTTGTTCATC;
(SEQ ID NO: 74)
GAAATTAATACGACTCACTATAGGGGGCACTTACGCGAAGTGATTGGTGGTGAC
and
(SEQ ID NO: 88)
CACAGTAATGGCGAAACCATGAAGCCTAC;
(SEQ ID NO: 75)
GAAATTAATACGACTCACTATAGGGGAAGAGCAGCGCGTTGCCGATGCCTGGAAAG
and
(SEQ ID NO: 89)
CTTTCGGCAGCGCCAGTTCGTCGTTGTGGT;
(SEQ ID NO: 20)
GAAATTAATACGACTCACTATAGGGACAAAGGTCAACCAATGACATTCAGACTAT
and
(SEQ ID NO: 32)
GGACCATATTTCTCTACACCTTTTTTAGGA;
(SEQ ID NO: 21)
GAAATTAATACGACTCACTATAGGGAACAAGAAGCAGCGCGTAAA
and
(SEQ ID NO: 33)
CCCCTGTTTACCTTTGGCTT;
(SEQ ID NO: 22)
GAAATTAATACGACTCACTATAGGGTAATCCATTTACCACTTCAACATTACAACAAGA
and
(SEQ ID NO: 34)
TCAATACCTTCATATAATTGTTGCGCGAGCAT;
(SEQ ID NO: 23)
GAAATTAATACGACTCACTATAGGGGATTTAGTTGACGCGCAACAAGCACGTCGTATT
and
(SEQ ID NO: 35)
CCTTCAATGGACCAATACTCTTCAGGTTTA;
(SEQ ID NO: 24)
GAAATTAATACGACTCACTATAGGGGAATTTAGTTGATGCACAACAAGCGCGTCGT
and
(SEQ ID NO: 36)
AAGCAACTGATTGTACTCGACCAGCTGACAATC;
(SEQ ID NO: 25)
GAAATTAATACGACTCACTATAGGGACGTGTTCAGTCAGTAGCTCTTCGTTTAGTTA
and
(SEQ ID NO: 37)
TTATATCTAAATTCCCCTTCAATAGTCCAATAT;
(SEQ ID NO: 26)
GAAATTAATACGACTCACTATAGGGAAAGGCTTTATGTCTGTATACGTCGAAGCGAA
and
(SEQ ID NO: 38)
CAATCTTTGTTGCTGTCACCATCTCCCCTTCT;
(SEQ ID NO: 27)
GAAATTAATACGACTCACTATAGGGCCATCTTTTGATACAGTCCATGACTTAGCCATT
and
(SEQ ID NO: 39)
GTGACAAATGGAGTTGACGGATTGTTAGAAA;
(SEQ ID NO: 27)
GAAATTAATACGACTCACTATAGGGCCATCTTTTGATACAGTCCATGACTTAGCCATT
and
(SEQ ID NO: 39)
GTGACAAATGGAGTTGACGGATTGTTAGAAA;
(SEQ ID NO: 27)
GAAATTAATACGACTCACTATAGGGCCATCTTTTGATACAGTCCATGACTTAGCCATT
and
(SEQ ID NO: 39)
GTGACAAATGGAGTTGACGGATTGTTAGAAA;
(SEQ ID NO: 28)
GAAATTAATACGACTCACTATAGGGAATGTTACCAGATATGGAGGAAGGAGAAAGT
and
(SEQ ID NO: 40)
ACTTGCTTCTGAAAACCTTGCAGGAGGTTGA;
(SEQ ID NO: 29)
GAAATTAATACGACTCACTATAGGGGTTGATATATTGTTCTGGGATGTAGGATG
and
(SEQ ID NO: 41)
TTCTACTACAAGATTGAACGTATCCGCGAA;
(SEQ ID NO: 30)
GAAATTAATACGACTCACTATAGGGACAGCAAGCTCGTCGTACCTTAGACAGAATCG
and
(SEQ ID NO: 42)
ATATTCTTCTGGAACAAATTCTCGGATCTCTT;
and
(SEQ ID NO: 31)
GAAATTAATACGACTCACTATAGGGGAGATTACTAAAGAAGCGGTAAAAGCGGCATT
and
(SEQ ID NO: 43)
AACAGATTGCACACGACCAGCACTCAAACCT;
b) the second set of primers comprise a set of sequences selected from the group consisting of:
GAAATTAATACGACTCACTATAGGGCGTCTAGTTCTGCTGTCTTGTCTCTCATGG (SEQ ID NO: 76) and CAAAGTCCTGTTCGAGTTTAGCGAATGGTT (SEQ ID NO: 90);
GAAATTAATACGACTCACTATAGGGAATGTCTGGCAGCACACTTCCTATCTCGAC (SEQ ID NO: 77) and TGATCTCCTGCTTGATCCAGTTGAGGATCT (SEQ ID NO: 91);
GAAATTAATACGACTCACTATAGGGATAAAACCGGCAGCGGTGGCTATGG (SEQ ID NO: 78) and GCTAATACATCGCGACGGCTTTCTGCCTTA (SEQ ID NO: 92); and
GAAATTAATACGACTCACTATAGGGTTAAAATTCCCAATAGCTTGATCGCCCTCG (SEQ ID NO: 79) and ATAAACAGGCACAACTGAATATTTCATCGC (SEQ ID NO: 93); and/or
c) the guide polynucleotides each comprise a nucleotide sequence selected from the group consisting of:
(SEQ ID NO: 95)
CGGCGCGACGACGGGCUUCCCGCAAGCAGUUUUAGUCCCCUUCGUUUUUG
GGGUAGUCUAAAUCCCCUAUAGUGAGUCGUAUUAAUUUC;
(SEQ ID NO: 96)
GUGAUGAGACCAUGGCGGCGGUAUCGCUGUUUUAGUCCCCUUCGUUUUUG
GGGUAGUCUAAAUCCCCUAUAGUGAGUCGUAUUAAUUUC;
(SEQ ID NO: 97)
UUCUAUAUGCGCGGCAAGAACGACUUCCGUUUUAGUCCCCUUCGUUUUUG
GGGUAGUCUAAAUCCCCUAUAGUGAGUCGUAUUAAUUUC;
(SEQ ID NO: 98)
AGUCAACUAUUAGGCCAAAGCUAUGAUCGUUUUAGUCCCCUUCGUUUUUG
GGGUAGUCUAAAUCCCCUAUAGUGAGUCGUAUUAAUUUC;
(SEQ ID NO: 99)
AUGGGUGAAAUCGUCACCGAUCGUCUGGGUUUUAGUCCCCUUCGUUUUUG
GGGUAGUCUAAAUCCCCUAUAGUGAGUCGUAUUAAUUUC;
(SEQ ID NO: 100)
UGUCCGACCUGCUGCCCGGCUACCUGACGUUUUAGUCCCCUUCGUUUUUG
GGGUAGUCUAAAUCCCCUAUAGUGAGUCGUAUUAAUUUC;
(SEQ ID NO: 101)
AUUGGCAAUUACACGCUUCACUGCUGACGUUUUAGUCCCCUUCGUUUUUG
GGGUAGUCUAAAUCCCCUAUAGUGAGUCGUAUUAAUUUC;
(SEQ ID NO: 102)
CAGCCAACACGUCUCGACUUAAACCGUGGUUUUAGUCCCCUUCGUUUUUG
GGGUAGUCUAAAUCCCCUAUAGUGAGUCGUAUUAAUUUC;
(SEQ ID NO: 103)
UGUGGUACCUACCACAUGCACUCGCUGGGUUUUAGUCCCCUUCGUUUUUG
GGGUAGUCUAAAUCCCCUAUAGUGAGUCGUAUUAAUUUC;
(SEQ ID NO: 104)
GCUGGCUGGCUUUUCUGCCACCGCGCUGGUUUUAGUCCCCUUCGUUUUUG
GGGUAGUCUAAAUCCCCUAUAGUGAGUCGUAUUAAUUUC;
(SEQ ID NO: 105)
CAACGGUUUGAUCGUCAGGGAUGGCGGCGUUUUAGUCCCCUUCGUUUUUG
GGGUAGUCUAAAUCCCCUAUAGUGAGUCGUAUUAAUUUC;
(SEQ ID NO: 106)
AUCGUGCGCCGCUGAUUCUGGUCACUUAGUUUUAGUCCCCUUCGUUUUUG
GGGUAGUCUAAAUCCCCUAUAGUGAGUCGUAUUAAUUUC;
(SEQ ID NO: 107)
CCAAGUCUUUAAGUGGGAUGGACAGACGGUUUUAGUCCCCUUCGUUUUUG
GGGUAGUCUAAAUCCCCUAUAGUGAGUCGUAUUAAUUUC;
and
(SEQ ID NO: 108)
GGCCGUCCGUUAAUUUCCCUUGCAUACAGUUUUAGUCCCCUUCGUUUUUG
GGGUAGUCUAAAUCCCCUAUAGUGAGUCGUAUUAAUUUC.

20. The method of claim 11, wherein the a) comprises splitting the biological sample into at least three or more containers, wherein at least between 2 and 9 of the containers contain unique sets of primers suitable for amplifying a target nucleotide capable of identifying a unique microbial species or taxonomic group, and wherein between at least 1 and 4 of the containers contain unique sets of primers suitable for amplifying a target nucleotide specific to an AMR gene.


SEQUENCE LISTING (122 sequences)

SEQ ID NO: 1 - DNA, 61 bp, synthetic construct
  source 1..61: mol_type = other DNA
gttttagtccccttcgtttttggggtagtctaaatcccctatagtgagtcgtattaatttc

SEQ ID NO: 2 - protein, 291 aa, Escherichia coli
MVKKSLRQFTLMATATVTLLLGSVPLYAQTADVQQKLAELERQSGGRLGVALINTADNSQILYRADERFAMCSTSKVMAAAAVLKKSESEPNLLNQRVEIKKSDLVNYNPIAEKHVNGTMSLAELSAAALQYSDNVAMNKLIAHVGGPASVTAFARQLGDETFRLDRTEPTLNTAIPGDPRDTTSPRAMAQTLRNLTLGKALGDSQRAQLVTWMKGNTTGAASIQAGLPASWVVGDKTGSGGYGTTNDIAVIWPKDRAPLILVTYFTQPQPKAESRRDVLASAAKIVTDGL

SEQ ID NO: 3 - DNA, 876 bp, Escherichia coli
  source 1..876: mol_type = other DNA
atggttaaaaaatcactgcgccagttcacgctgatggcgacggcaaccgtcacgctgttgttaggaagtgtgccgctgtatgcgcaaacggcggacgtacagcaaaaacttgccgaattagagcggcagtcgggaggcagactgggtgtggcattgattaacacagcagataattcgcaaatactttatcgtgctgatgagcgctttgcgatgtgcagcaccagtaaagtgatggccgcggccgcggtgctgaagaaaagtgaaagcgaaccgaatctgttaaatcagcgagttgagatcaaaaaatctgaccttgttaactataatccgattgcggaaaagcacgtcaatgggacgatgtcactggctgagcttagcgcggccgcgctacagtacagcgataacgtggcgatgaataagctgattgctcacgttggcggcccggctagcgtcaccgcgttcgcccgacagctgggagacgaaacgttccgtctcgaccgtaccgagccgacgttaaacaccgccattccgggcgatccgcgtgataccacttcacctcgggcaatggcgcaaactctgcggaatctgacgctgggtaaagcattgggcgacagccaacgggcgcagctggtgacatggatgaaaggcaataccaccggtgcagcgagcattcaggctggactgcctgcttcctgggttgtgggggataaaaccggcagcggtggctatggcaccaccaacgatatcgcggtgatctggccaaaagatcgtgcgccgctgattctggtcacttacttcacccagcctcaacctaaggcagaaagccgtcgcgatgtattagcgtcggcggctaaaatcgtcaccgacggtttgtaa

SEQ ID NO: 4 - protein, 498 aa, Pseudomonas aeruginosa
MKKVSTLDLLFVAIMGVSPAAFAADLIDVSKLPSKAAQGAPGPVTLQAAVGAGGADELKAIRSTTLPNGKQVTRYEQFHNGVRVVGEAITEVKGPGKSVAAQRSGHFVANIAADLPGSTTAAVSAEQVLAQAKSLKAQGRKTENDKVELVIRLGENNIAQLVYNVSYLIPGEGLSRPHFVIDAKTGEVLDQWEGLAHAEAGGPGGNQKIGKYTYGSDYGPLTVNDRCEMDDGNVITVDMNGSTDDSKTTPFRFACPTNTYKQVNGAYSPLNDAHFFGGVVFKLYRDWFGTSPLTHKLYMKVHYGRSVENAYWDGTAMLFGDGATMFYPLVSLDVAAHEVSHGFTEQNSGLIYRGQSGGMNEAFSDMAGEAAEFYMRGKNDFLIGYDIKKGSGALRYMDQPSRDGRSIDNASQYYNGIDVHHSSGVYNRAFYLLANSPGWDTRKAFEVFVDANRYYWTATSNYNSGACGVIRSAQNRNYSAADVTRAFSTVGVTCPSAL

SEQ ID NO: 5 - DNA, 1497 bp, Pseudomonas aeruginosa
  source 1..1497: mol_type = other DNA
atgaagaaggtttctacgcttgacctgttgttcgttgcgatcatgggtgtttcgccggccgcttttgccgccgacctgatcgacgtgtccaaactccccagcaaggctgcccagggcgcgcccggcccggtcaccttgcaagccgcggtcggcgctggcggtgccgatgaactgaaagcgatccgcagcacgaccctgcccaacggcaagcaggtcacccgctacgagcaattccacaacggcgtacgggtggtcggcgaagccatcaccgaagtcaagggtcccggcaagagcgtggcggcgcagcgcagcggccatttcgtcgccaacatcgccgccgacctgccgggcagcaccaccgcggcggtatccgccgagcaggtgctggcccaggccaagagcctgaaggcccagggccgcaagaccgagaatgacaaagtggaactggtgatccgcctgggcgagaacaacatcgcccaactggtctacaacgtctcctacctgattcccggcgagggactgtcgcggccgcatttcgtcatcgacgccaagaccggcgaagtgctcgatcagtgggaaggcctggcccacgccgaggcgggcggccccggcggcaaccagaagatcggcaagtacacctacggtagcgactacggtccgctgaccgtcaacgaccgctgcgagatggacgacggcaacgtcatcaccgtcgacatgaacggcagcaccgacgacagcaagaccacgccgttccgcttcgcctgcccgaccaacacctacaagcaggtcaacggcgcctattcgccgctgaacgacgcgcatttcttcggcggcgtggtgttcaaactgtaccgggactggttcggcaccagcccgctgacccacaagctgtacatgaaggtgcactacgggcgcagcgtggagaacgcctactgggacggcacggcgatgctcttcggcgacggcgccaccatgttctatccgctggtgtcgctggacgtggcggcccacgaggtcagccacggctttaccgagcagaactccgggctgatctaccgcgggcaatcaggcggaatgaacgaagcgttctccgacatggccggcgaggcagccgagttctacatgcgcggcaagaacgacttcctgatcggctacgacatcaagaagggcagcggtgcgctgcgctacatggaccagcccagccgcgacgggcgatccatcgacaacgcgtcgcagtactacaacggcatcgacgtgcaccactccagcggcgtgtacaaccgtgcgttctacctgttggccaattcgccgggctgggatacccgcaaggccttcgaggtgttcgtcgacgccaaccgctactactggaccgccaccagcaactacaacagcggcgcctgcggggtgattcgctcggcgcagaaccgcaactactcggcggctgacgtcacccgggcgttcagcaccgtcggcgtgacctgcccgagcgcgttgtaa

SEQ ID NO: 6 - protein, 293 aa, Klebsiella pneumoniae
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPSDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 7 - DNA, 1377 bp, Klebsiella pneumoniae
  source 1..1377: mol_type = other DNA
cggggcagttacagccgttacagcctctggagagggagcggcttgccgctcggtgataatcccagctgtagcggcctgattacatccggccgctacacctagctccaccttcaaacaaggaatatcgttgatgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaagcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaaggctctgaaaatcatctattggcccaccaccgccgcccttgcgggcggcatggattaccaaccactgtcacatttaggctaggagtctgcgcggcagagccgtgtgaccggttttctgtagagcactgacgatggcggcggcgctctctgcaattggcaaggcgtcggcgccaaggataccaatcttgcggcgcgcggcgtgttatgacgactggggtgcatttgagccgccccatttaaccttcgccctcacagatacgccattcgcctcaaatttagcgccatgcagacgagcttccactcggcttgcaccttgtccaggcccctcatgctgaactgacgcaatcccatcaccgccttgatca

SEQ ID NO: 8 - protein, 293 aa, Klebsiella pneumoniae
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKHSEAVIAAAARLALEGLGVNGQ

SEQ ID NO: 9 - DNA, 882 bp, Klebsiella pneumoniae
  source 1..882: mol_type = other DNA
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 10 - protein, 293 aa, Klebsiella pneumoniae
MSLYRRLVLLSCLSWPLAGFSATALTNLVAEPFAKLEQDFGGSIGVYAMDTGSGATVSYRAEERFPLCSSFKGFLAAAVLARSQQQAGLLDTPIRYGKNALVPWSPISEKYLTTGMTVAELSAAAVQYSDNAAANLLLKELGGPAGLTAFMRSIGDTTFRLDRWELELNSAIPGDARDTSSPRAVTESLQKLTLGSALAAPQRQQFVDWLKGNTTGNHRIRAAVPADWAVGDKTGTCGVYGTANDYAVVWPTGRAPIVLAVYTRAPNKDDKYSEAVIAAAARLALEGLGVNGQ

SEQ ID NO: 11 - DNA, 882 bp, Klebsiella pneumoniae
  source 1..882: mol_type = other DNA
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagtacagcgaggccgtcatcgccgctgcggctagactcgcgctcgagggattgggcgtcaacgggcagtaa

SEQ ID NO: 12 - protein, 270 aa, Escherichia coli
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAVVTHAHQDKMGGMDALHAAGIATYANALSNQLAPQEGMVAAQHSLTFAANGWVEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 13 - DNA, 813 bp, Escherichia coli
  source 1..813: mol_type = other DNA
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 14 - protein, 660 aa, Escherichia coli
MSRPQFTSLRLSLLALAVSATLPTFAFATETMTVTATGNARSSFEAPMMVSVIDTSAPENQTATSATDLLRHVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVGASESSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQW

SEQ ID NO: 15 - DNA, 1983 bp, Escherichia coli
  source 1..1983: mol_type = other DNA
atgtcacgtccgcaatttacctcgttgcgtttgagtttattggccttagctgtttctgccaccttgccaacgtttgcttttgctactgaaaccatgaccgttacggcaacggggaatgcccgtagttccttcgaagcgcctatgatggtcagcgtcatcgacacttccgctcctgaaaatcaaacggctacttcagccaccgatctgctgcgtcatgttcctggaattactctggatggtaccggacgaaccaacggtcaggatgtaaatatgcgtggctatgatcatcgcggcgtgctggttcttgtcgatggtgttcgtcagggaacggataccggacacctgaatggcacttttctcgatccggcgctgatcaagcgtgttgagattgttcgtggaccttcagcattactgtatggcagtggcgcgctgggtggagtgatctcctacgatacggtcgatgcaaaagatttattgcaggaaggacaaagcagtggttttcgtgtctttggtactggcggcacgggggaccatagcctgggattaggcgcgagcgcgtttgggcgaactgaaaatctggatggtattgtggcctggtccagtcgcgatcggggtgatttacgccagagcaatggtgaaaccgcgccgaatgacgagtccattaataacatgctggcgaaagggacctggcaaattgattcagcccagtctctgagcggtttagtgcgttactacaacaacgacgcgcgtgaaccaaaaaatccgcagaccgttggggcttctgaaagcagcaacccgatggttgatcgttcaacaattcaacgcgatgcgcagctttcttataaactcgccccgcagggcaacgactggttaaatgcagatgcaaaaatttattggtcggaagtccgtattaatgcgcaaaacacggggagttccggcgagtatcgtgaacagataacaaaaggagccaggctggagaaccgttccactctctttgccgacagtttcgcttctcacttactgacatatggcggtgagtattatcgtcaggaacaacatccgggcggcgcgacgacgggcttcccgcaagcaaaaatcgattttagctccggctggctacaggatgagatcaccttacgcgatctgccgattaccctgcttggcggaacccgctatgacagttatcgcggtagcagtgacggttacaaagatgttgatgccgacaaatggtcatctcgtgcggggatgactatcaatccgactaactggctgatgttatttggctcatatgcccaggcattccgcgccccgacgatgggcgaaatgtataacgattctaagcacttctcgattggtcgcttctataccaactattgggtgccaaacccgaacttacgtccggaaactaacgaaactcaggagtacggttttgggctgcgttttgatgacctgatgttgtccaatgatgctctggaatttaaagccagctactttgataccaaagcgaaggattacatctccacgaccgtcgatttcgcggcggcgacgactatgtcgtataacgtcccgaacgccaaaatctggggctgggatgtgatgacgaaatataccactgatctgtttagccttgatgtggcctataaccgtacccgcggcaaagacaccgataccggcgaatacatctccagcattaacccggatactgttaccagcactctgaatattccgatcgctcacagtggcttctctgttgggtgggttggtacgtttgccgatcgctcaacacatatcagcagcagttacagcaaacaaccaggctatggcgtgaatgatttctacgtcagttatcaaggacaacaggcgctcaaaggtatgaccactactttggtgttgggtaacgctttcgacaaagagtactggtcgccgcaaggcatcccacaggatggtcgtaacggaaaaattttcgtgagttatcaatggtaa

SEQ ID NO: 16 - protein, 265 aa, Escherichia coli
MRVLALSAVFLVASIIGMPAVAKEWQENKSWNAHFTEHKSQGVVVLWNENKQQGFTNNLKRANQAFLPASTFKIPNSLIALDLGVVKDEHQVFKWDGQTRDIATWNRDHNLITAMKYSVVPVYQEFARQIGEARMSKMLHAFDYGNEDISGNVDSFWLDGGIRISATEQISFLRKLYHNKLHVSERSQRIVKQAMLTEANGDYIIRAKTGYSTRIEPKIGWWVGWVELDDNVWFFAMNMDMPTSDGLGLRQAITKEVLKQEKIIP

SEQ ID NO: 17 - DNA, 798 bp, Escherichia coli
  source 1..798: mol_type = other DNA
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactatattattcgggctaaaactggatactcgactagaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 18 - protein, 265 aa, Escherichia coli
MRVLALSAVFLVASIIGMPAVAKEWQENKSWNAHFTEHKSQGVVVLWNENKQQGFTNNLKRANQAFLPASTFKIPNSLIALDLGVVKDEHQVFKWDGQTRDIAAWNRDHDLITAMKYSVVPVYQEFARQIGEARMSKMLHAFDYGNEDISGNVDSFWLDGGIRISATQQIAFLRKLYHNKLHVSERSQRIVKQAMLTEANGDYIIRAKTGYSTSIEPKIGWWVGWVELDDNVWFFAMNMDMPTSDGLGLRQAITKEVLKQEKIIP

SEQ ID NO: 19 - DNA, 798 bp, Escherichia coli
  source 1..798: mol_type = other DNA
atgcgtgtattagccttatcggctgtgtttttggtggcatcgattatcggaatgccagcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgtgatatcgccgcttggaatcgtgaccatgacttaattaccgcgatgaagtactcagttgtgcctgtttatcaagaatttgcccgccaaattggtgaggcacgtatgagtaaaatgctgcacgccttcgattatggcaatgaggatatctcgggcaatgtagacagtttttggctcgatggtggtattcgcatttcggctacccagcaaatcgcttttttacgcaagctgtatcacaacaagctgcacgtttctgagcgtagtcagcgcatcgtgaaacaagccatgctgaccgaagccaatggcgactatattattcgggctaaaacgggatactcgactagtatcgaacctaagattggctggtgggttggttgggttgaacttgatgataatgtgtggttttttgcgatgaatatggatatgcccacatcggatggtttagggctgcgccaagccatcacaaaagaagtgctcaaacaggagaaaattattccctag

SEQ ID NO: 20 - DNA, 55 bp, synthetic construct
  source 1..55: mol_type = other DNA
gaaattaatacgactcactatagggacaaaggtcaaccaatgacattcagactat

SEQ ID NO: 21 - DNA, 45 bp, synthetic construct
  source 1..45: mol_type = other DNA
gaaattaatacgactcactatagggaacaagaagcagcgcgtaaa

SEQ ID NO: 22 - DNA, 58 bp, synthetic construct
  source 1..58: mol_type = other DNA
gaaattaatacgactcactatagggtaatccatttaccacttcaacattacaacaaga

SEQ ID NO: 23 - DNA, 58 bp, synthetic construct
  source 1..58: mol_type = other DNA
gaaattaatacgactcactataggggatttagttgacgcgcaacaagcacgtcgtatt

SEQ ID NO: 24 - DNA, 56 bp, synthetic construct
  source 1..56: mol_type = other DNA
gaaattaatacgactcactataggggaatttagttgatgcacaacaagcgcgtcgt

SEQ ID NO: 25 - DNA, 57 bp, synthetic construct
  source 1..57: mol_type = other DNA
gaaattaatacgactcactatagggacgtgttcagtcagtagctcttcgtttagtta

SEQ ID NO: 26 - DNA, 57 bp, synthetic construct
  source 1..57: mol_type = other DNA
gaaattaatacgactcactatagggaaaggctttatgtctgtatacgtcgaagcgaa

SEQ ID NO: 27 - DNA, 58 bp, synthetic construct
  source 1..58: mol_type = other DNA
gaaattaatacgactcactatagggccatcttttgatacagtccatgacttagccatt

SEQ ID NO: 28 - DNA, 56 bp, synthetic construct
  source 1..56: mol_type = other DNA
gaaattaatacgactcactatagggaatgttaccagatatggaggaaggagaaagt

SEQ ID NO: 29 - DNA, 54 bp, synthetic construct
  source 1..54: mol_type = other DNA
gaaattaatacgactcactataggggttgatatattgttctgggatgtaggatg

SEQ ID NO: 30 - DNA, 57 bp, synthetic construct
  source 1..57: mol_type = other DNA
gaaattaatacgactcactatagggacagcaagctcgtcgtaccttagacagaatcg

SEQ ID NO: 31 - DNA, 57 bp, synthetic construct
  source 1..57: mol_type = other DNA
gaaattaatacgactcactataggggagattactaaagaagcggtaaaagcggcatt

SEQ ID NO: 32 - DNA, 30 bp, synthetic construct
  source 1..30: mol_type = other DNA
ggaccatatttctctacaccttttttagga

SEQ ID NO: 33 - DNA, 20 bp, synthetic construct
  source 1..20: mol_type = other DNA
cccctgtttacctttggctt

SEQ ID NO: 34 - DNA, 32 bp, synthetic construct
  source 1..32: mol_type = other DNA
tcaataccttcatataattgttgcgcgagcat

SEQ ID NO: 35 - DNA, 30 bp, synthetic construct
  source 1..30: mol_type = other DNA
ccttcaatggaccaatactcttcaggttta

SEQ ID NO: 36 - DNA, 33 bp, synthetic construct
  source 1..33: mol_type = other DNA
aagcaactgattgtactcgaccagctgacaatc

SEQ ID NO: 37 - DNA, 33 bp, synthetic construct
  source 1..33: mol_type = other DNA
ttatatctaaattccccttcaatagtccaatat

SEQ ID NO: 38 - DNA, 32 bp, synthetic construct
  source 1..32: mol_type = other DNA
caatctttgttgctgtcaccatctccccttct

SEQ ID NO: 39 - DNA, 31 bp, synthetic construct
  source 1..31: mol_type = other DNA
gtgacaaatggagttgacggattgttagaaa

SEQ ID NO: 40 - DNA, 31 bp, synthetic construct
  source 1..31: mol_type = other DNA
acttgcttctgaaaaccttgcaggaggttga

SEQ ID NO: 41 - DNA, 30 bp, synthetic construct
  source 1..30: mol_type = other DNA
ttctactacaagattgaacgtatccgcgaa

SEQ ID NO: 42 - DNA, 32 bp, synthetic construct
  source 1..32: mol_type = other DNA
atattcttctggaacaaattctcggatctctt

SEQ ID NO: 43 - DNA, 31 bp, synthetic construct
  source 1..31: mol_type = other DNA
aacagattgcacacgaccagcactcaaacct

SEQ ID NO: 44 - protein, 171 aa, Serratia marcescens
MPLLDSFTVDHTRMAAPAVRVAKTMKTPHGDTITVFDLRFCRPNLEVMPERGIHTLEHLFAGFMRDHLNGQGVEIIDISPMGCRTGFYMSLIGVPEEQRVADAWKAAMADVLKVTDQRKIPELNEYQCGTYHMHSLEEAQEIAKHILDNDVVVNHNDELALPKEKLQELHI

SEQ ID NO: 45 - DNA, 516 bp, Serratia marcescens
  source 1..516: mol_type = other DNA
atgccattgctggatagctttaccgtcgatcatacccgtatggcagctccggctgtccgcgttgcgaaaaccatgaaaacgcctcatggcgataccatcacggtgtttgatctgcgcttttgccgcccgaacctggaagtgatgcccgagcgcggcattcatacgctggagcacctgttcgccggctttatgcgtgaccacctgaacggccagggcgtggagattatcgacatctccccgatgggctgtcgtaccggtttctacatgagcctgatcggcgtgccggaagagcagcgcgtagccgatgcgtggaaagcggcgatggccgacgtgctgaaagtgaccgaccagcgcaaaatccctgagctgaacgaataccagtgcggcacttaccatatgcactcgctggaagaagcgcaggagatcgccaagcacatcctggataacgacgtggtggttaaccacaacgacgagctggcgctgccgaaagagaagctgcaggaactgcatatctag

SEQ ID NO: 46 - protein, 286 aa, Escherichia coli
MRYIRLCIISLLATLPLAVHASPQPLEQIKQSESQLSGRVGMIEMDLASGRTLTAWRADERFPMMSTFKVVLCGAVLARVDAGDEQLERKIHYRQQDLVDYSPVSEKHLADGMTVGELCAAAITMSDNSAANLLLATVGGPAGLTAFLRQIGDNVTRLDRWETELNEALPGDARDTTTPASMAATLRKLLTSQRLSARSQRQLLQWMVDDRVAGPLIRSVLPAGWFIADKTGASERGARGIVALLGPNNKAERIVVIYLRDTPASMAERNQQIAGIGAALIEHWQR

SEQ ID NO: 47 - DNA, 861 bp, Escherichia coli
  source 1..861: mol_type = other DNA
atgcgttatattcgcctgtgtattatctccctgttagccaccctgccgctggcggtacacgccagcccgcagccgcttgagcaaattaaacaaagcgaaagccagctgtcgggccgcgtaggcatgatagaaatggatctggccagcggccgcacgctgaccgcctggcgcgccgatgaacgctttcccatgatgagcacctttaaagtagtgctctgcggcgcagtgctggcgcgggtggatgccggtgacgaacagctggagcgaaagatccactatcgccagcaggatctggtggactactcgccggtcagcgaaaaacaccttgccgacggcatgacggtcggcgaactctgcgccgccgccattaccatgagcgataacagcgccgccaatctgctgctggccaccgtcggcggccccgcaggattgactgcctttttgcgccagatcggcgacaacgtcacccgccttgaccgctgggaaacggaactgaatgaggcgcttcccggcgacgcccgcgacaccactaccccggccagcatggccgcgaccctgcgcaagctgctgaccagccagcgtctgagcgcccgttcgcaacggcagctgctgcagtggatggtggacgatcgggtcgccggaccgttgatccgctccgtgctgccggcgggctggtttatcgccgataagaccggagctagcgagcggggtgcgcgcgggattgtcgccctgcttggcccgaataacaaagcagagcgcattgtggtgatttatctgcgggatacgccggcgagcatggccgagcgaaatcagcaaatcgccgggatcggcgcggcgctgatcgagcactggcaacgctaa

SEQ ID NO: 48 - protein, 286 aa, Escherichia coli
MRYIRLCIISLLATLPLAVHASPQPLEQIKQSESQLSGRVGMIEMDLASGRTLTAWRADERFPMMSTFKVVLCGAVLARVDAGDEQLERKIHYRQQDLVDYSPVSEKHLADGMTVGELCAAAITMSDNSAANLLLATVGGPAGLTAFLRQIGDNVTRLDRWETELNEALPGDARDTTTPASMAATLRKLLTSQRLSARSQRQLLQWMVDDRVAGPLIRSVLPAGWFIADKTGASKRGARGIVALLGPNNKAERIVVIYLRDTPASMAERNQQIAGIGAALIEHWQR

SEQ ID NO: 49 - DNA, 861 bp, Escherichia coli
  source 1..861: mol_type = other DNA
atgcgttatattcgcctgtgtattatctccctgttagccaccctgccgctggcggtacacgccagcccgcagccgcttgagcaaattaaacaaagcgaaagccagctgtcgggccgcgtaggcatgatagaaatggatctggccagcggccgcacgctgaccgcctggcgcgccgatgaacgctttcccatgatgagcacctttaaagtagtgctctgcggcgcagtgctggcgcgggtggatgccggtgacgaacagctggagcgaaagatccactatcgccagcaggatctggtggactactcgccggtcagcgaaaaacaccttgccgacggcatgacggtcggcgaactctgtgccgccgccattaccatgagcgataacagcgccgccaatctgctgctggccaccgtcggcggccccgcaggattgactgcctttttgcgccagatcggcgacaacgtcacccgccttgaccgctgggaaacggaactgaatgaggcgcttcccggcgacgcccgcgacaccactaccccggccagcatggccgcgaccctgcgcaagctgctgaccagccagcgtctgagcgcccgttcgcaacggcagctgctgcagtggatggtggacgatcgggtcgccggaccgttgatccgctccgtgctgccggcgggctggtttatcgccgataagaccggagctagcaaacggggtgcgcgcgggattgtcgccctgcttggcccgaataacaaagcagagcgcatcgtggtgatttatctgcgggatacgccggcgagcatggccgagcgaaatcagcaaatcgccgggatcggcgcggcgctgatcgagcactggcaacgctaa

SEQ ID NO: 50 - DNA, 25 bp, synthetic construct
  source 1..25: mol_type = other DNA
gaaattaatacgactcactataggg

SEQ ID NO: 51 - protein, 883 aa, Escherichia phage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 52 - DNA, 2652 bp, Enterobacteria phage T7
  source 1..2652: mol_type = other DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 53 - protein, 878 aa, Acinetobacter baumannii
MANTSRSASQSTASSASAAHKRALVIVESPAKAKTINKYLGSQYIVKSSVGHVRDLPTGGSKATEKKPAARTKLTEAEKEQKANQALINRMGVDPEHGWQAHYEVLPGKENVVAELKKLAKDADAIYLATDLDREGEAIAWHLREVIGGDDSRYHRVVFNEITKNAIQEAFKQPTRLDLNRVNAQQARRFLDRVVGFMVSPLLWEKIARGLSAGRVQSVAVKLVVEREREIRAFIPEEYWQVFADTKAKKDDIRLEAVKQAGKTLKLKNKAETDALLDVLKGAEYKVAQREDKPTKVNPSAPYITSTLQQAASTRLGFSVKKTMMLAQRLYEAGFITYMRTDSTFLSDDAVSMVRAHIESQYGEKYLPAKPNRYGNKAGAQEAHEAIRPSNVALTGDQLAGVERDAQRLYDLIWRQFVACQMTPAEYLSSTLTVEAGNVELKAKGRTLVFDGFTKVRGANKSDDDIILPAIKVGEILKLEKLDPSQHFTKPPARFTEASLVKELEKRGIGRPSTYAAIISTIQERGYVKLENRRLFAEKMGEIVTDRLDESFNNLMNYAFTADLEGQLDRVATGERNWKELLDTFYGDFKKRLTNAQGEQGMRRNQPVEVPAVHCPECSRPMQIRTGTTGVFLGCSGYNLPPKERCKGTLNLTPVESLAALSDDDSAETADLMSKHRCPKCGTAMDSYVIDGGRKLHVCGNNPDCDGYELEEGEFKIKGYDGPTIPCDKCNGEMQLKTGRFGPYFACTSCDNTRKVLKNGQPAPPRVEPIKMEHLRSTKHDDYFVLRDGAAGLFLAASKFPKIRETRAPKVAELRSVADQLDPKYQFILQAPDVDPEGNPTIVKFSRKNQSQYVGSETPEGKQTKWSLIYQDGKWIEG

SEQ ID NO: 54 - DNA, 2637 bp, Acinetobacter baumannii
  source 1..2637: mol_type = other DNA
atggcgaatacctcgcgctctgcatctcaaagcacagcatcttctgcctctgctgcacataaacgtgccttagtgattgtggagtcgcctgccaaagcgaaaaccatcaacaaatatttaggttcgcagtacattgttaagtcttctgtaggtcacgtacgtgatttgccaacaggcggcagtaaagcaacagagaaaaagccggctgcccggaccaaactcactgaggctgaaaaagaacaaaaagcgaatcaggctttaatcaatcgtatgggcgtcgatccggaacatggatggcaagcgcattacgaagttctgcctggcaaagaaaatgttgttgctgaactcaagaaacttgctaaagatgcagatgcaatctatctcgcaacggacttggatagagaaggggaagcaatcgcttggcacttacgcgaagtgattggtggtgacgatagccgttatcatcgtgtggtatttaacgaaattactaaaaatgccattcaagaagcatttaaacagccaacacgtctcgacttaaaccgtgttaatgcacaacaagcacgtcgtttcttggaccgtgtagtaggcttcatggtttcgccattattatgggaaaagattgcccgtggtttatcggcaggtcgtgtacagtctgtagcggtaaagcttgttgttgaacgtgaacgtgaaattcgtgcttttattccagaagaatattggcaagtctttgcagacactaaagctaaaaaagatgacattcgtcttgaggctgttaaacaggcgggtaaaacgcttaaattaaaaaataaagctgaaacagatgccctgttagatgtattaaaaggtgctgaatataaagttgcccagcgtgaagataaaccaactaaggtgaatccaagcgccccatacatcacttcgactttgcaacaagctgcaagtacacgtttaggtttctctgtaaagaaaaccatgatgttggcgcagcgcttgtatgaagctggtttcattacttatatgcgtactgactcaacgtttttgagtgatgatgctgtaagtatggtgcgtgcacatattgaaagccaatatggcgaaaaatatttaccagctaagccaaatcgctatggtaataaagcaggtgctcaagaagcccacgaagctattcgtccatcaaatgttgcgctgactggtgatcaacttgctggtgtagagcgtgatgctcagcgcttatatgatttaatttggcgtcagttcgtagcttgccaaatgactccggcagaatatttatcttcaactttaacagttgaagctggcaatgtggagttaaaggcaaaaggccgtacgcttgtatttgatggctttactaaagttcgtggtgccaacaaatctgatgacgatatcattttacctgcaattaaagtgggcgaaattttaaagctagaaaagctagatccaagtcagcattttaccaaaccaccagctcgttttactgaagcatctttagtaaaagaacttgaaaaacgtggtattggtcgtccgtcgacttatgctgccattatttctacgattcaagaacgtggttacgttaagttagaaaatcgccgtctctttgccgagaaaatgggtgagatcgtaacggatcgtcttgatgaaagttttaataacctaatgaattatgcatttactgcagatttagaaggtcagttggaccgtgtagcaacgggtgagcgtaactggaaggaattgctagacacattctatggtgattttaaaaagcgtttgaccaatgctcaaggtgagcagggaatgcgtcgtaaccagccagttgaagtaccagcagtacattgccctgaatgttcacgtccaatgcagattcgtacgggcactacaggcgtattcttagggtgttctggctataacttgccacctaaagaacgctgtaaagggaccttgaacttaacgccagttgagtctttagcagcgttatctgatgacgatagtgctgaaacagcagacttgatgtcaaaacatcgttgtccgaaatgtggcacggcaatggacagctacgtcattgatggtggccgtaagttacatgtttgtggtaacaacccggattgcgatggctatgagcttgaagaaggtgagttcaagatcaaagggtatgatggcccaaccattccatgtgataaatgtaatggtgaaatgcagcttaagactggtcgcttcggcccgtattttgcttgtacaagctgtgacaatactcgtaaagtcttgaaaaatggtcaacctgcaccaccacgtgtagaaccaatcaagatggaacacttgcgttcaaccaaacatgatgattattttgtgttacgtgatggcgcggctggtctgttcttggctgcaagtaagttcccgaaaattcgtgaaacacgtgcaccaaaagttgctgagcttcgtagcgtagctgatcaacttgatccaaaataccagtttattttacaagctccagatgtagaccctgaaggtaacccaactattgtgaagtttagtcgtaagaaccaatcacaatatgtgggttctgaaactccagaaggtaaacaaaccaaatggagcctaatttatcaagacggtaaatggattgaaggctaa

SEQ ID NO: 55 - protein, 359 aa, Citrobacter freundii
MVFRKLAAKGMSRCAKFQWCFFLLASLALFFLHDGPLVIAGLFITQLSLQMSMTRTGKEVFGVIFNIQSWRNSGASQQTYFFRWYIGFPISCLLYILSLTNYVYVPGDLYYQFIASSTDDLALVMNRFAVGVSNILPEILRGASLTLSMMMFVATLLKNVQLAEVRRIYSSIQDGNDSQKILSNMTWEQFEKIIRLHFELNGYKATLTNTGADGGVDILLQKDGRREMVQCKLWKTSKVGVSVVREIYGVVQANAYERGYIITSGFFTQDAWEFALSANVKGTLSLIDGSRLLKIIKDKEIPPPATEIINVGPGENQIYDLNVIPSCPRCHSKMVRRMSNGNYFYGCSAYPICKGTRNL

SEQ ID NO: 56 - DNA, 1080 bp, Citrobacter freundii
  source 1..1080: mol_type = other DNA
atggtttttagaaaattggcagcaaaaggaatgtcccgatgcgccaaattccagtggtgctttttcctgctagcgtcattagctttattctttctacatgatggcccgttggtcattgcgggcctctttattacacaattgtctctccagatgtcgatgacccgtacaggaaaggaagtcttcggcgttatttttaatatccagtcatggagaaacagtggtgctagccagcaaacctattttttccggtggtatatcggcttcccgatttcctgcttgctatatattctttctcttactaattatgtgtacgttccaggcgatttgtattatcagtttattgcaagcagtactgatgacctagcactcgtaatgaatcgttttgctgtaggcgtgagtaacatactccctgaaatccttcgaggcgcatctctaaccctttcgatgatgatgtttgtcgcaacattgctaaaaaatgtacaacttgctgaagtacgtcgaatttactcctccattcaggatggaaatgattcccagaaaattctctcgaatatgacatgggagcaattcgaaaagattataagactgcatttcgaactcaatggctacaaagcaactctgactaatacaggagctgacggcggagtcgacattctgctgcaaaaagatggtcgccgcgaaatggtacagtgcaaattatggaaaaccagcaaagtaggagtctcggtagtccgcgaaatttacggtgtagtgcaagccaatgcatatgagcggggttatatcatcacctctgggttctttactcaggatgcatgggaattcgctcttagcgcaaatgttaaaggaacattatcgcttatcgatggttccagacttctgaaaataatcaaggataaagaaatccccccgcctgccactgagataataaatgttggtccaggtgagaatcaaatatacgatcttaatgtcatacctagctgtccccgttgccatagcaaaatggttagaagaatgtcgaatggaaattatttctatggttgctcggcttacccaatttgtaaaggaacccgtaacctctag

SEQ ID NO: 57 - protein, 865 aa, Klebsiella pneumoniae
MGKALVIVESPAKAKTINKYLGSDYVVKSSVGHIRDLPTSGSAAKKSADSTSTKTAKKPKKDERGALVNRMGVDPWHNWEAHYEVLPGKEKVVSELKQLAEKADHIYLATDLDREGEAIAWHLREVIGGDDARYSRVVFNEITKNAIRQAFNKPGELNIDRVNAQQARRFMDRVVGYMVSPLLWKKIARGLSAGRVQSVAVRLVVEREREIKAFVPEEFWEVDASTTTPSGEALALQVTHQNDKPFRPVNKEQTQAAVSLLEKARYSVLEREDKPTTSKPGAPFITSTLQQAASTRLGFGVKKTMMMAQRLYEAGYITYMRTDSTNLSQDAVNMVRGYISDNFGKKYLPESPNQYASKENSQEAHEAIRPSDVNVMAESLKDMEADAQKLYQLIWRQFVACQMTPAKYDSTTLTVGAGDFRLKARGRILRFDGWTKVMPALRKGDEDRILPAVNKGDALTLVELTPAQHFTKPPARFSEASLVKELEKRGIGRPSTYASIISTIQDRGYVRVENRRFYAEKMGEIVTDRLEENFRELMNYDFTAQMENSLDQVANHEAEWKAVLDHFFSDFTQQLDKAEKDPEEGGMRPNQMVLTSIDCPTCGRKMGIRTASTGVFLGCSGYALPPKERCKTTINLVPENEVLNVLEGEDAETNALRAKRRCPKCGTAMDSYLIDPKRKLHVCGNNPTCDGYEIEEGEFRIKGYDGPIVECEKCGSEMHLKMGRFGKYMACTNEECKNTRKILRNGEVAPPKEDPVPLPELPCEKSDAYFVLRDGAAGVFLAANTFPKSRETRAPLVEELYRFRDRLPEKLRYLADAPQQDPEGNKTMVRFSRKTKQQYVSSEKDGKATGWSAFYVDGKWVEGKK

SEQ ID NO: 58 - DNA, 2598 bp, Klebsiella pneumoniae
  source 1..2598: mol_type = other DNA
atgggtaaagctcttgtcatcgttgagtccccggcaaaagccaaaacgatcaacaagtatctgggtagtgactacgtggtgaaatccagcgtcggtcacatccgcgatttgccgaccagtggctcagctgccaaaaagagtgccgactctacctccaccaagacggctaaaaagcctaaaaaggatgaacgtggcgctctcgtcaaccgtatgggggttgacccgtggcacaattgggaggcgcactatgaagtgttgcctggtaaagagaaggtcgtctctgaactgaaacaactggctgaaaaagccgaccacatctatctcgcaaccgaccttgaccgcgaaggggaagccattgcatggcacctgcgggaagtgattgggggtgatgatgcgcgctatagccgagtggtgtttaacgaaattactaaaaacgcgatccgccaggcatttaacaaaccgggtgagctgaatattgatcgtgttaatgcccagcaggcgcgtcgctttatggaccgcgtggtggggtatatggtttcgccgctgctatggaaaaagatcgctcgtggtctgtctgccggtcgtgtgcagtcggtggcggttcgcctggtggtcgagcgtgagcgtgaaattaaagcgttcgtgccggaagagttctgggaagtcgatgccagcacgaccacgccatctggtgaagcgttggcgttacaggtgactcatcagaacgacaaaccgttccgtccggtcaacaaagaacaaactcaggctgcggtaagtctgctggaaaaagcgcgctacagcgtgctggaacgtgaagacaaaccgacaaccagtaaacctggcgctccttttattacctctacgctgcaacaagctgccagcacccgtcttggatttggcgtgaaaaaaaccatgatgatggcgcagcgtttgtatgaagcaggctatatcacttacatgcgtaccgactccactaacctgagtcaggacgcggtaaatatggttcgcggttatatcagcgataattttggtaagaaatatctgccggaaagtccgaatcagtacgccagcaaagaaaactcacaggaagcgcacgaagcgattcgtccttctgacgtcaatgtgatggcggaatcgctgaaggatatggaagcagatgcgcagaaactgtaccagttaatctggcgtcagttcgttgcctgccagatgaccccagcgaaatatgactccacgacgctgaccgttggtgcgggcgatttccgcctgaaagcacgcggtcgtattttgcgttttgatggctggacaaaagtgatgcctgcgttgcgtaaaggcgatgaagatcgcatcttaccagcagttaataaaggcgatgctctgacgctcgttgaacttacaccagcccagcactttaccaagccgccagcccgtttcagtgaagcatcgctggttaaagagctggaaaaacgcggtatcggtcgtccgtctacctatgcgtcgatcatttcgaccattcaggatcgtggctacgtgcgagtagaaaatcgtcgtttctatgcggaaaaaatgggcgaaatcgtcaccgatcgccttgaagaaaatttccgcgagttaatgaactacgactttaccgcgcagatggaaaacagcctcgaccaggtggcaaatcacgaagcagagtggaaagctgtactggatcacttcttctcggatttcacccagcagttagataaagctgaaaaagatccggaagagggtggtatgcgcccgaaccagatggttctgaccagcattgactgcccgacttgtggtcgcaaaatggggattcgcacagcgagcaccggggtattccttggctgttctggctatgcgctgccgccgaaagagcgttgcaaaaccaccattaacctggtgccggaaaacgaagtgctgaacgtgctggaaggcgaagatgctgaaaccaacgcgctgcgcgcaaaacgtcgttgcccgaaatgcggcacggcgatggacagctatctcatcgatccgaaacgtaagttgcatgtctgtggtaataacccaacctgcgacggttacgagatcgaagagggcgaattccgcattaaaggttatgacggcccgatcgttgagtgtgaaaaatgtggctctgaaatgcacctgaaaatggggcgtttcggtaaatacatggcctgcaccaacgaagagtgtaaaaacacacgtaagattttacgtaacggcgaagtggcaccaccgaaagaagatccggtgccattacctgagctgccgtgcgaaaaatcagatgcttatttcgtgctgcgtgacggtgctgccggtgtgttcctggctgccaacactttcccgaaatcgcgtgaaacgcgtgcgccactggtggaagagctttatcgcttccgcgaccgtctgccggaaaaactgcgttatctggccgatgcgccacagcaggatccggaaggtaataagaccatggttcgctttagccgtaaaaccaaacagcaatatgtctcttcggaaaaagacggaaaggcgactggctggtcagcattttatgttgatggcaaatgggttgaaggaaaaaaataa

SEQ ID NO: 59 - protein, 865 aa, Proteus mirabilis
MGKALVIVESPAKAKTINKYLGNDYVVKSSVGHIRDLPKSGSGSQKSENSSATKGVKKVKKDEKSALVSRMGVDPYNGWHANYQILPGKEKVVAELKALAEKADHIYLATDLDREGEAIAWHLREVIGGEDDRFSRVVFNEITKNAITQAFQTPGELNIDRVNAQQARRFMDRVVGYMVSPLLWKKVARGLSAGRVQSVAVRLLVEREREIKAFVPEEYWQLHASLLTPDEQPLRMEVTHYQDNAFKPVSSEETQVAVKALQNATFIVKNREDRPTSSKPSAPLITSTLQQAASTRLSFGVKKTMMLAQRLYEAGYITYMRTDSTNLSQDAIQMARDYIHDKFGAKYLPKEPNVYSSKENSQEAHEAIRPSDINVTAESLKDMDSDAKRLYQLIWDQFVACQMTPAKYDSTTLTVVSGDYKLRAKGRTLRFAGWTKVMPAMRSKDEDKTLPAVDVGAQLALAELSPTQHFTKPPARFSEATLVKELEKRGIGRPSTYASIISTIQDRGYVKVENRRFYAEKMGEIVTDRLEENFSDLMNYDFTAQMEDHLDHVANNQENWKAVLDAFFTDFSQQLEVAEKDPEEGGMRPNPMVITSIECPTCSRHMGIRTATTGVFLGCSGYALPPKERCKQTINLIPEDELLNILEGDDAETNALRARRRCPKCGTAMDSYLIDTHRKLHVCGNNPACDGYEVEEGEFRLKGYEGPVIECDKCGAEMHLKMGRFGKYMGCTNEECKNTRKILKSGEIAPPKEDPVPLPELPCEKSDAYFVLRDGAAGIFLAANTFPKSRETRAPKVAELVRFKDRLSEKMRYLAQAPVTDPEGNPTIVRFSRKTKQQYVSSEKEGKATGWSAFYIDGKWVEKSK

SEQ ID NO: 60 - DNA, 2598 bp, Proteus mirabilis
  source 1..2598: mol_type = other DNA
atgggtaaagctcttgttatcgtggagtccccggctaaagccaaaacaatcaataaatatcttggcaatgactacgtagtaaaatctagcgtgggtcacattcgtgatttgccaaaaagtggttctggcagccagaagagcgaaaactcatccgccacgaaaggcgtgaaaaaggttaaaaaggatgagaaatcagccttagtcagccgtatgggtgtcgatccttataatggttggcatgcaaactaccaaattctgccgggtaaagagaaggtagtcgctgaactaaaagcattggctgaaaaagcagatcatatctatctcgcaacggaccttgaccgcgaaggagaagctatcgcgtggcatttacgcgaagtgatcggcggtgaagatgatagatttagtcgtgtcgtatttaatgaaattacgaaaaatgccattacacaagcattccaaacaccgggtgagctgaatattgaccgtgttaacgcgcaacaagcacgtcgttttatggatcgtgtggttggctatatggtttctccgctgttatggaaaaaagtggcgcgtggtttatctgcggggcgtgtacagtcagttgcggtacgcctactcgtcgagcgtgaacgtgaaattaaagcatttgtgccagaagagtattggcaattacacgcttcactgctgacgcccgatgaacaacccttgcgtatggaagtgacgcactatcaagataacgcttttaaaccagtctcctctgaggaaactcaggtggcggttaaggcgttacagaatgccactttcatcgttaaaaaccgtgaagatagaccaacatccagtaaaccaagtgcccctttgatcacctctacattgcaacaagcggcaagtactcgcttaagctttggggtgaaaaagaccatgatgttggctcaacgtctttatgaagcaggttatatcacctatatgcgtactgactctaccaacctgagtcaggatgcaatccaaatggcacgtgattatattcacgataaatttggtgcaaaatatctgcctaaagagccgaatgtttattcaagtaaagagaactcacaagaagcacacgaagctattcgtccttctgatatcaatgtgacggctgaatctttaaaagatatggatagcgacgcgaagcgtttatatcaactgatttgggatcaatttgtcgcctgtcaaatgacaccggcaaaatatgactcaaccacacttacggttgtatcaggggattataaactacgcgctaaaggtcgcacattacgttttgcgggttggacaaaagtcatgccagcaatgcgcagtaaagatgaagataaaacattaccagcggttgatgttggggcgcaattagcgttagctgagttatcgccaacgcaacactttactaagccaccagcgcgttttagtgaagctacattagttaaagagctagaaaaacgaggtattggtcgaccttcaacttatgcttcaattatctctaccattcaagatagaggttatgtcaaagttgaaaaccgtcgtttctatgcagaaaaaatgggtgagattgttaccgatcgcttagaagaaaactttagtgatttgatgaactacgatttcacggctcaaatggaagatcatctcgaccatgttgccaacaaccaagaaaactggaaagcggtattagatgcattcttcacagattttagtcagcaactagaagtcgctgaaaaagatccagaagaaggaggaatgcgacctaaccctatggttattacttctattgaatgtcctacttgttctcgtcatatgggaattagaacagcgactacaggggtattcttagggtgctcgggctatgctttaccaccaaaagagcgttgtaaacaaacgataaatcttattcctgaagatgaattacttaatatcttggaaggagatgatgcggaaactaatgcattacgtgcccgtcgtcgttgtccgaaatgcggtacggcaatggacagttatcttattgatactcatcgtaaattacatgtttgtggtaataatcctgcatgtgatggttatgaagttgaagaaggtgaattccgcttaaaaggttatgaaggcccagtgattgagtgtgataaatgtggcgctgaaatgcatctaaaaatggggcgttttggcaagtacatgggttgcaccaatgaagagtgtaaaaatacacgtaagatcttaaagagtggtgaaattgcaccaccaaaagaagatccggttccactacctgagttgccttgtgaaaaatcggatgcttactttgtgttacgtgatggcgcggcgggtattttccttgcggccaatacgttccctaaatcaagggaaacccgtgcaccgaaagtcgctgagttagtacgttttaaagatagattatcggaaaaaatgcgctacttagcacaagcaccggtgaccgatcctgaagggaatccaaccatagtacgttttagtcgtaaaactaaacagcaatatgtctcttctgaaaaagaggggaaagcaacaggttggtcagccttttatattgatggcaaatgggttgaaaaaagtaaataa

SEQ ID NO: 61 - protein, 1197 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALEKRPAATKKAGQAKKKKGSYPYDVPDYAYPYDVPDYAYPYDVPDYA

SEQ ID NO: 62 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 63 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 64 - protein, 391 aa, Enterobacteria phage T4
MSDLKSRLIKASTSKLTAELTASKFFNEKDVVRTKIPMMNIALSGEITGGMQSGLLILAGPSKSFKSNFGLTMVSSYMRQYPDAVCLFYDSEFGITPAYLRSMGVDPERVIHTPVQSLEQLRIDMVNQLDAIERGEKVVVFIDSLGNLASKKETEDALNEKVVSDMTRAKTMKSLFRIVTPYFSTKNIPCIAINHTYETQEMFSKTVMGGGTGPMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDTNCTTFWGPLFKHQPFRDAIKRAYQLGAIDSNEIVEAEVDELINSKVEKFKSPESKSKSAADLETDLEQLSDMEEFNE

SEQ ID NO: 65 - RNA, 25 nt, Enterobacteria phage T4
  source 1..25: mol_type = other RNA
gggaacaaagctgaagtacttaccc

SEQ ID NO: 66 - DNA, 18 bp, synthetic construct
  source 1..18: mol_type = other DNA
gggtagggcgggttggga

SEQ ID NO: 67 - DNA, 60 bp, synthetic construct
  source 1..60: mol_type = other DNA
gaaattaatacgactcactatagggatatggcggtgagtattatcgtcaggaacaacatc

SEQ ID NO: 68 - DNA, 57 bp, synthetic construct
  source 1..57: mol_type = other DNA
gaaattaatacgactcactatagggggtgacgccctgccgctgcaggtaacccataa

SEQ ID NO: 69 - DNA, 51 bp, synthetic construct
  source 1..51: mol_type = other DNA
gaaattaatacgactcactatagggatgttctatccgctggtgtcgctgga

SEQ ID NO: 70 - DNA, 52 bp, synthetic construct
  source 1..52: mol_type = other DNA
gaaattaatacgactcactatagggaatgggcgtatgattatacccaggtaa

SEQ ID NO: 71 - DNA, 55 bp, synthetic construct
  source 1..55: mol_type = other DNA
gaaattaatacgactcactataggggttgaaaaccgtcgcttttacgctgaaaaa

SEQ ID NO: 72 - DNA, 57 bp, synthetic construct
  source 1..57: mol_type = other DNA
gaaattaatacgactcactataggggaccatcctgtacgcgctgggctggacgcagc

SEQ ID NO: 73 - DNA, 55 bp, synthetic construct
  source 1..55: mol_type = other DNA
gaaattaatacgactcactatagggcggtacgcctactcgtcgagcgtgaacgtg

SEQ ID NO: 74 - DNA, 54 bp, synthetic construct
  source 1..54: mol_type = other DNA
gaaattaatacgactcactatagggggcacttacgcgaagtgattggtggtgac

SEQ ID NO: 75 - DNA, 56 bp, synthetic construct
  source 1..56: mol_type = other DNA
gaaattaatacgactcactataggggaagagcagcgcgttgccgatgcctggaaag

SEQ ID NO: 76 - DNA, 55 bp, synthetic construct
  source 1..55: mol_type = other DNA
gaaattaatacgactcactatagggcgtctagttctgctgtcttgtctctcatgg

SEQ ID NO: 77 - DNA, 55 bp, synthetic construct
  source 1..55: mol_type = other DNA
gaaattaatacgactcactatagggaatgtctggcagcacacttcctatctcgac

SEQ ID NO: 78 - DNA, 50 bp, synthetic construct
  source 1..50: mol_type = other DNA
gaaattaatacgactcactatagggataaaaccggcagcggtggctatgg

SEQ ID NO: 79 - DNA, 55 bp, synthetic construct
  source 1..55: mol_type = other DNA
gaaattaatacgactcactatagggttaaaattcccaatagcttgatcgccctcg

SEQ ID NO: 80 - skipped

SEQ ID NO: 81 - DNA, 35 bp, synthetic construct
  source 1..35: mol_type = other DNA
gagatgaccatttgtcggcatcaacatctttgtag

SEQ ID NO: 82 - DNA, 33 bp, synthetic construct
  source 1..33: mol_type = other DNA
atcttcacgctccagtacgctgtagctggcttt

SEQ ID NO: 83 - DNA, 25 bp, synthetic construct
  source 1..25: mol_type = other DNA
accgctgcccttcttgatgtcgtag

SEQ ID NO: 84 - DNA, 28 bp, synthetic construct
  source 1..28: mol_type = other DNA
agcagtttggcaaagtgcgcttgtgcag

SEQ ID NO: 85 - DNA, 31 bp, synthetic construct
  source 1..31: mol_type = other DNA
tccattcggcttctttattcgccacctggtc

SEQ ID NO: 86 - DNA, 33 bp, synthetic construct
  source 1..33: mol_type = other DNA
caccacgatttcatcaggctgacgtggaacttc

SEQ ID NO: 87 - DNA, 30 bp, synthetic construct
  source 1..30: mol_type = other DNA
cgtcacttccatacgcaagggttgttcatc

SEQ ID NO: 88 - DNA, 29 bp, synthetic construct
  source 1..29: mol_type = other DNA
cacagtaatggcgaaaccatgaagcctac

SEQ ID NO: 89 - DNA, 30 bp, synthetic construct
  source 1..30: mol_type = other DNA
ctttcggcagcgccagttcgtcgttgtggt

SEQ ID NO: 90 - DNA, 30 bp, synthetic construct
  source 1..30: mol_type = other DNA
caaagtcctgttcgagtttagcgaatggtt

SEQ ID NO: 91 - DNA, 30 bp, synthetic construct
  source 1..30: mol_type = other DNA
tgatctcctgcttgatccagttgaggatct

SEQ ID NO: 92 - DNA, 30 bp, synthetic construct
  source 1..30: mol_type = other DNA
gctaatacatcgcgacggctttctgcctta

SEQ ID NO: 93 - DNA, 30 bp, synthetic construct
  source 1..30: mol_type = other DNA
ataaacaggcacaactgaatatttcatcgc

SEQ ID NO: 94 - DNA, 20 bp, synthetic construct
  source 1..20: mol_type = other DNA
tagttattgctcagcggtgg

SEQ ID NO: 95 - RNA, 89 nt, synthetic construct
  source 1..89: mol_type = other RNA
cggcgcgacgacgggcttcccgcaagcagttttagtccccttcgtttttggggtagtctaaatcccctatagtgagtcgtattaatttc

SEQ ID NO: 96 - RNA, 89 nt, synthetic construct
  source 1..89: mol_type = other RNA
gtgatgagaccatggcggcggtatcgctgttttagtccccttcgtttttggggtagtctaaatcccctatagtgagtcgtattaatttc

SEQ ID NO: 97 - RNA, 89 nt, synthetic construct
  source 1..89: mol_type = other RNA
ttctatatgcgcggcaagaacgacttccgttttagtccccttcgtttttggggtagtctaaatcccctatagtgagtcgtattaatttc

SEQ ID NO: 98 - RNA, 89 nt, synthetic construct
  source 1..89: mol_type = other RNA
agtcaactattaggccaaagctatgatcgttttagtccccttcgtttttggggtagtctaaatcccctatagtgagtcgtattaatttc

SEQ ID NO: 99 - RNA, 89 nt, synthetic construct
  source 1..89: mol_type = other RNA
atgggtgaaatcgtcaccgatcgtctgggttttagtccccttcgtttttggggtagtctaaatcccctatagtgagtcgtattaatttc

SEQ ID NO: 100 - RNA, 89 nt, synthetic construct
  source 1..89: mol_type = other RNA
tgtccgacctgctgcccggctacctgacgttttagtccccttcgtttttggggtagtctaaatcccctatagtgagtcgtattaatttc

SEQ ID NO: 101 - RNA, 89 nt, synthetic construct
  source 1..89: mol_type = other RNA
attggcaattacacgcttcactgctgacgttttagtccccttcgtttttggggtagtctaaatcccctatagtgagtcgtattaatttc

SEQ ID NO: 102 - RNA, 89 nt, synthetic construct
  source 1..89: mol_type = other RNA
cagccaacacgtctcgacttaaaccgtggttttagtccccttcgtttttggggtagtctaaatcccctatagtgagtcgtattaatttc

SEQ ID NO: 103 - RNA, 89 nt, synthetic construct
  source 1..89: mol_type = other RNA
tgtggtacctaccacatgcactcgctgggttttagtccccttcgtttttggggtagtctaaatcccctatagtgagtcgtattaatttc

SEQ ID NO: 104 - RNA, 89 nt, synthetic construct
  source 1..89: mol_type = other RNA
gctggctggcttttctgccaccgcgctggttttagtccccttcgtttttggggtagtctaaatcccctatagtgagtcgtattaatttc

SEQ ID NO: 105 - RNA, 89 nt, synthetic construct
  source 1..89: mol_type = other RNA
caacggtttgatcgtcagggatggcggcgttttagtccccttcgtttttggggtagtctaaatcccctatagtgagtcgtattaatttc

SEQ ID NO: 106 - RNA, 89 nt, synthetic construct
  source 1..89: mol_type = other RNA
atcgtgcgccgctgattctggtcacttagttttagtccccttcgtttttggggtagtctaaatcccctatagtgagtcgtattaatttc

SEQ ID NO: 107 - RNA, 89 nt, synthetic construct
  source 1..89: mol_type = other RNA
ccaagtctttaagtgggatggacagacggttttagtccccttcgtttttggggtagtctaaatcccctatagtgagtcgtattaatttc

SEQ ID NO: 108 - RNA, 89 nt, synthetic construct
  source 1..89: mol_type = other RNA
ggccgtccgttaatttcccttgcatacagttttagtccccttcgtttttggggtagtctaaatcccctatagtgagtcgtattaatttc

SEQ ID NO: 109 - protein, 383 aa, Acinetobacter baumannii
MRFKKISCLLLSPLFIFSTSIYAGNTPKDQEIKKLVDQNFKPLLEKYDVPGMAVGVIQNNKKYEMYYGLQSVQDKKAVNRSTIFELGSVSKLFTATAGGYAKNKGKISFDDTPGKYWKELKNTPIDQVNLLQLATYTSGNLALQFPDEVQTDQQVLTFFKDWQPKNPIGEYRQYSNPSIGLFGKVVALSMNKPFDQVLEKTIFPALGLKHSYVNVPKTQMQNYAFGYNQENQPIRVNPGPLDAPAYGVKSTLPDMLSFIHANLNPQKYPADIQRAINETHQGFYQVNTMYQALGWEEFSYPATLQTLLDSNSEQIVMKPNKVTAISKEPSVKMYHKTGSTNGFGTYVVFIPKENIGLVMLTNKRIPNEERIKAAYAVLDAIKK

SEQ ID NO: 110 - DNA, 1152 bp, Acinetobacter baumannii
  source 1..1152: mol_type = other DNA
atgcgatttaaaaaaatttcttgtctacttttatccccgctttttatttttagtacctcaatttatgcgggcaatacaccaaaagaccaagaaattaaaaaactggtagatcaaaactttaaaccgttattagaaaaatatgatgtgccaggtatggctgtgggtgttattcaaaataataaaaagtatgaaatgtattatggtcttcaatctgttcaagataaaaaagccgtaaatcgcagtaccatttttgagctaggttctgtcagtaaattatttaccgcgacagcaggtggatatgcaaaaaataaaggaaaaatctcttttgacgatacgcctggtaaatattggaaagaactaaaaaacacaccgattgaccaagttaacttacttcaactcgcgacgtatacaagtggtaaccttgccttgcagtttccagatgaagtacaaacagaccaacaagttttaacttttttcaaagactggcaacctaaaaacccaatcggtgaatacagacaatattcaaatccaagtattggcctatttggaaaggttgtggctttgtctatgaataaacctttcgaccaagtcttagaaaaaacaatttttccggcccttggcttaaaacatagctatgtaaatgtacctaagacccagatgcaaaactatgcatttggttataaccaagaaaatcagccgattcgagttaaccccggcccactcgatgcccctgcatatggcgtcaaatcgacactacccgacatgttgagttttattcatgccaaccttaacccacagaaatatccggctgatattcaacgggcaattaatgaaacacatcaagggttctatcaagtaaataccatgtatcaggcactcggttgggaagagttttcttatccggcaacgttacaaactttattagacagtaattcagaacagattgtgatgaaacctaataaagtgactgctatttcaaaggaaccttcagttaagatgtaccataaaactggctcaaccaacggtttcggaacatatgtagtgtttattcctaaagaaaatattggcttagtcatgttaaccaataaacgtattccaaatgaagagcgcattaaggcagcttatgctgtgctggatgcaataaagaaataa

SEQ ID NO: 111 - protein, 246 aa, Pseudomonas aeruginosa
MKKLFVLCVCFFCSITAAGAALPDLKIEKLEEGVFVHTSFEEVNGWGVVTKHGLVVLVNTDAYLIDTPFTATDTEKLVNWFVERGYEIKGTISSHFHSDSTGGIEWLNSQSIPTYASELTNELLKKSGKVQAKYSFSEVSYWLVKNKIEVFYPGPGHTQDNLVVWLPESKILFGGCFIKPHGLGNLGDANLEAWPKSAKILMSKYGKAKLVVSSHSEKGDASLMKRTWEQALKGLKESKKTSSPSN

SEQ ID NO: 112 - DNA, 741 bp, Pseudomonas aeruginosa
  source 1..741: mol_type = other DNA
atgaagaaattatttgttttatgtgtatgcttcttttgtagcattactgccgcaggagcggctttacctgatttaaaaatcgagaagcttgaagaaggtgtttttgttcatacatcgttcgaagaggttaacggttggggggttgttactaaacacggtttagtggtgcttgtaaacacagacgcctatctaattgacactccatttactgctacagacactgaaaaattagtcaattggtttgtggagcgcggctatgaaatcaaaggcactatttcatcacatttccatagcgacagcacaggaggaatagagtggcttaattctcaatctattcccacgtatgcatctgaattaacaaatgaacttttgaaaaaatccggtaaggtacaagctaaatattcatttagcgaagttagctattggctagttaaaaataaaattgaagttttctaccctggcccaggtcacactcaagataacctagtggtttggttgcctgaaagtaaaattttattcggtggttgctttattaaacctcacggtcttggcaatttaggtgacgcaaatttagaagcttggccaaagtccgccaaaatattaatgtctaaatatggcaaagcaaagcttgttgtttcaagtcatagtgaaaaaggggacgcatcactaatgaaacgtacatgggaacaagcccttaaagggcttaaagaaagtaaaaaaacatcatcaccaagtaactaa

SEQ ID NO: 113 - protein, 668 aa, Staphylococcus aureus
MKKIKIVPLILIVVVVGFGIYFYASKDKEINNTIDAIEDKNFKQVYKDSSYISKSDNGEVEMTERPIKIYNSLGVKDINIQDRKIKKVSKNKKRVDAQYKIKTNYGNIDRNVQFNFVKEDGMWKLDWDHSVIIPGMQKDQSIHIENLKSERGKILDRNNVELANTGTAYEIGIVPKNVSKKDYKAIAKELSISEDYIKQQMDQNWVQDDTFVPLKTVKKMDEYLSDFAKKFHLTTNETKSRNYPLEKATSHLLGYVGPINSEELKQKEYKGYKDDAVIGKKGLEKLYDKKLQHEDGYRVTIVDDNSNTIAHTLIEKKKKDGKDIQLTIDAKVQKSIYNNMKNDYGSGTAIHPQTGELLALVSTPSYDVYPFMYGMSNEEYNKLTEDKKEPLLNKFQITTSPGSTQKILTAMIGLNNKTLDDKTSYKIDGKGWQKDKSWGGYNVTRYEVVNGNIDLKQAIESSDNIFFARVALELGSKKFEKGMKKLGVGEDIPSDYPFYNAQISNKNLDNEILLADSGYGQGEILINPVQILSIYSALENNGNINAPHLLKDTKNKVWKKNIISKENINLLTDGMQQVVNKTHKEDIYRSYANLIGKSGTAELKMKQGETGRQIGWFISYDKDNPNMMMAINVKDVQDKGMASYNAKISGKVYDELYENGNKKYDIDE

SEQ ID NO: 114 - DNA, 2007 bp, Staphylococcus aureus
  source 1..2007: mol_type = other DNA
atgaaaaagataaaaattgttccacttattttaatagttgtagttgtcgggtttggtatatatttttatgcttcaaaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaatttaaaatcagaacgtggtaaaattttagaccgaaacaatgtggaattggccaatacaggaacagcatatgagataggcatcgttccaaagaatgtatctaaaaaagattataaagcaatcgctaaagaactaagtatttctgaagactatatcaaacaacaaatggatcaaaattgggtacaagatgataccttcgttccacttaaaaccgttaaaaaaatggatgaatatttaagtgatttcgcaaaaaaatttcatcttacaactaatgaaacaaaaagtcgtaactatcctctagaaaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttgacgataatagcaatacaatcgcacatacattaatagagaaaaagaaaaaagatggcaaagatattcaactaactattgatgctaaagttcaaaagagtatttataacaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaactgatggtatgcaacaagtcgtaaataaaacacataaagaagatatttatagatcttatgcaaacttaattggcaaatccggtactgcagaactcaaaatgaaacaaggagaaactggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataa

SEQ ID NO: 115 - protein, 343 aa, Enterococcus faecalis
MNRIKVAILFGGCSEEHDVSVKSAIEIAANINKEKYEPLYIGITKSGVWKMCEKPCAEWENDNCYSAVLSPDKKMHGLLVKKNHEYEINHVDVAFSALHGKSGEDGSIQGLFELSGIPFVGCDIQSSAICMDKSLTYIVAKNAGIATPAFWVINKDDRPVAATFTYPVFVKPARSGSSFGVKKVNSADELDYAIESARQYDSKILIEQAVSGCEVGCAVLGNSAALVVGEVDQIRLQYGIFRIHQEVEPEKGSENAVITVPADLSAEERGRIQETAKKIYKALGCRGLARVDMFLQDNGRIVLNEVNTLPGFTSYSRYPRMMAAAGIALPELIDRLIVLALKG

SEQ ID NO: 116 - DNA, 1032 bp, Enterococcus faecalis
  source 1..1032: mol_type = other DNA
atgaatagaataaaagttgcaatactgtttgggggttgctcagaggagcatgacgtatcggtaaaatctgcaatagagatagccgctaacattaataaagaaaaatacgagccgttatacattggaattacgaaatctggtgtatggaaaatgtgcgaaaaaccttgcgcggaatgggaaaacgacaattgctattcagctgtactctcgccggataaaaaaatgcacggattacttgttaaaaagaaccatgaatatgaaatcaaccatgttgatgtagcattttcagctttgcatggcaagtcaggtgaagatggatccatacaaggtctgtttgaattgtccggtatcccttttgtaggctgcgatattcaaagctcagcaatttgtatggacaaatcgttgacatacatcgttgcgaaaaatgctgggatagctactcccgccttttgggttattaataaagatgataggccggtggcagctacgtttacctatcctgtttttgttaagccggcgcgttcaggctcatccttcggtgtgaaaaaagtcaatagcgcggacgaattggactacgcaattgaatcggcaagacaatatgacagcaaaatcttaattgagcaggctgtttcgggctgtgaggtcggttgtgcggtattgggaaacagtgccgcgttagttgttggcgaggtggaccaaatcaggctgcagtacggaatctttcgtattcatcaggaagtcgagccggaaaaaggctctgaaaacgcagttataaccgttcccgcagacctttcagcagaggagcgaggacggatacaggaaacggcaaaaaaaatatataaagcgctcggctgtagaggtctagcccgtgtggatatgtttttacaagataacggccgcattgtactgaacgaagtcaatactctgcccggtttcacgtcatacagtcgttatccccgtatgatggccgctgcaggtattgcacttcccgaactgattgaccgcttgatcgtattagcgttaaaggggtga

SEQ ID NO: 117 - protein, 342 aa, Enterococcus faecalis
MNRIKVAIIFGGCSEEHDVSVKSAIEIAANINTEKFDPHYIGITKNGVWKLCKKPCTEWEADSLPAILSPDRKTHGLLVMKESEYETRRIDVAFPVLHGKCGEDGAIQGLFELSGIPYVGCDIQSSAACMDKSLAYILTKNAGIAVPEFQMIDKGDKPEAGALTYPVFVKPARSGSSFGVTKVNGTEELNAAIEAAGQYDGKILIEQAISGCEVGCAVMGNEDDLIVGEVDQIRLSHGIFRIHQENEPEKGSENAMITVPADIPVEERNRVQETAKKVYRVLGCRGLARVDLFLQEDGGIVLNEVNTLPGFTSYSRYPRMVAAAGITLPALIDSLITLALKR

SEQ ID NO: 118 - DNA, 1029 bp, Enterococcus faecalis
  source 1..1029: mol_type = other DNA
atgaatagaataaaagtcgcaatcatcttcggcggttgctcggaggaacatgatgtgtcggtaaaatccgcaatagaaattgctgcgaacattaatactgaaaaattcgatccgcactacatcggaattacaaaaaacggcgtatggaagctatgcaagaagccatgtacggaatgggaagccgacagtctccccgccatactctccccggataggaaaacgcatgggctgcttgtcatgaaagaaagcgaatacgaaacacggcgtattgatgtggctttcccggttttgcatggcaaatgcggggaggatggtgcgatacagggtctgtttgaattgtctggtatcccctatgtaggctgcgatattcaaagctccgcagcttgcatggacaaatcactggcctacattcttacaaaaaatgcgggcatcgccgttcccgaatttcaaatgattgataaaggtgacaagccggaggcgggtgcgcttacctaccctgtctttgtgaagccggcacggtcaggttcgtcctttggcgtaaccaaagtaaacggtacggaagaacttaacgctgcgatagaagcggcaggacaatatgatggaaaaatcttaattgagcaagcgatttcgggctgtgaggtcgggtgtgcggtcatggggaacgaggatgatttgattgtcggcgaagtggatcaaatccggctgagccacggtatcttccgcatccatcaggaaaacgagccggaaaaaggctcagaaaatgcgatgattacagttcccgcagacattccggtcgaggaacgaaatcgggtgcaggaaacggcaaagaaagtatatcgggtgcttggatgcagagggcttgcccgtgttgatctttttttgcaggaggatggcggcatcgttctaaatgaggtcaataccctgcctggttttacatcgtacagccgctacccacgtatggtggccgccgcaggaatcacgcttcctgcactgattgacagcctgattacattggcgttaaagaggtga

SEQ ID NO: 119 - protein, 350 aa, Enterococcus casseliflavus
MKKIAIIFGGNSPEYTVSLASATSTIEALQSSPYDYDLSLIGIAPDAMDWYLYTGELENIRQDTWLLDTKHKQKIQPLFEGNGFWLSEEQQTLVPDVLFPIMHGKYGEDGSIQGLFELMKLPYVGCGVAGSALCMNKWLLHQAAAAIGVQSAPTILLTNQANQQEQIEAFIQTHGFPVFFKPNEAGSSKGITKVTCVEEIASALKEAFTYCSAVLLQKNIAGVEIGCGILGNDSLTVGACDAISLVDGFFDFEEKYQLISAKITVPAPLPETIETKVKEQAQLLYRSLGLKGLARIDFFVTDQGELYLNEINTMPGFTSHSRYPAMMAAVGLSYQELLQKLLVLAKEEVK

SEQ ID NO: 120 - DNA, 1053 bp, Enterococcus casseliflavus
  source 1..1053: mol_type = other DNA
atgaaaaaaatcgccattatttttggaggcaattcaccggaatacaccgtttctttagcttcagcaactagcacaatcgaagcactccaatcatctccctatgactacgacctctctttgatcgggatcgccccagatgctatggattggtacttgtatacaggagaactggaaaacatccgacaagacacgtggttgttggatacgaaacataaacagaaaatacagccgctattcgaaggaaacggcttttggctaagtgaagagcagcaaacgttggtacctgatgttttatttcccattatgcatggcaaatatggggaagatggcagtatccaaggattgtttgaattgatgaagctgccttatgtaggttgcggggtggccggttctgccttatgtatgaacaaatggctgctgcatcaagctgcagcagccattggcgtacaaagtgctcctacgattctcttgacaaatcaagccaaccagcaagaacaaatcgaagcttttatccagacccatggctttccagttttctttaagcctaatgaagcgggctcctcaaaagggatcactaaagtcacctgcgttgaagaaatcgcttctgccttaaaagaagcctttacttattgttccgcagtgctcctacaaaaaaatattgccggtgttgagatcggttgcggtattttgggcaacgactctttgactgtcggtgcttgtgacgccatttcattagtagacggctttttcgattttgaagaaaagtaccagctgatcagcgccaaaatcaccgtccctgcgccattgcctgaaacgattgaaaccaaggtcaaagaacaagctcagctgctctatcgtagtcttggtcttaaaggtcttgctcgcatcgacttttttgtcacggatcaaggagaactatacttgaatgaaatcaatactatgccgggctttacgagtcactcccgctatcctgccatgatggcagcggtcggcttatcctatcaagaactactacaaaaactgcttgtcttagcaaaggaggaagtcaaatga

SEQ ID NO: 121 - protein, 311 aa, Pseudomonas aeruginosa
MPRASKQQARYAVGRCLMLWSSNDVTQQGSRPKTKLCRTHPHGVLMFKLLSKLLVYLTASIMAIASPLAFSVDSSGEYPTVSEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEVEGNEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVE

SEQ ID NO: 122 - DNA, 936 bp, Pseudomonas aeruginosa
  source 1..936: mol_type = other DNA
atgcctcgggcatccaagcagcaagcgcgttacgccgtgggtcgatgtttgatgttatggagcagcaacgatgttacgcagcagggcagtcgccctaaaacaaagttatgccgcactcacccccatggagttttgatgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag